SEQUENCE LISTING (171 sequences)

SEQ ID NO: 1 - DNA, 5441 bp, Artificial Sequence
  Nucleotide sequence of Cas9, including a C-terminal SV40 nuclear localization signal, codon pair optimized for expression in Saccharomyces cerevisiae
ttttctttttttgcggtcacccccatgtggcggggaggcagaggagtaggtagagcaacgaatcctactatttatccaaattagtctaggaactctttttctagattttttagatttgagggcaagcgctgttaacgactcagaaatgtaagcactacggagtagaacgagaaatccgccataggtggaaatcctagcaaaatcttgcttaccctagctagcctcaggtaagctagccttagcctgtcaaatttttttcaaaatttggtaagtttctactagcaaagcaaacacggttcaacaaaccgaaaactccactcattatacgtggaaaccgaaacaaaaaaacaaaaaccaaaatactcgccaatgagaaagttgctgcgtttctactttcgaggaagaggaactgagaggattgactacgaaaggggcaaaaacgagtcgtattctcccattattgtctgctaccacgcggtctagtagaataagcaaccagtcaacgctaagacaggtaatcaaaataccagtctgctggctacgggctagtttttacctcttttagaacccactgtaaaagtccgttgtaaagcccgttctcactgttggcgtttttttttttttggtttagtttcttatttttcatttttttctttcatgaccaaaaacaaacaaatctcgcgatttgtactgcggccactggggcgtggccaaaaaaatgacaaatttagaaaccttagtttctgatttttcctgttatgaggagatatgataaaaaatattactgctttattgttttttttttatctactgaaatagagaaacttacccaaggaggaggcaaaaaaaagagtatatatacagcagctaccattcagattttaatatattcttttctcttcttctacactattattataataattttactatattcatttttagcttaaaacctcatagaatattattcttcagtcactcgcttaaatacttatcaaaaatggacaagaaatactctattggtttggatatcgggaccaactccgtcggttgggctgtcatcaccgacgaatacaaggttccatccaagaaattcaaggtcttgggtaacactgacagacactctatcaagaagaatttgatcggtgctttgttgttcgactccggtgaaaccgctgaagctaccagattgaagcgtaccgctcgtcgtagatacactagacgtaaaaaccgtatttgttacttgcaagaaatcttttctaacgaaatggccaaggttgacgactctttcttccacagattggaagaatctttcttggttgaagaagacaagaagcacgaaagacatccaatcttcggtaacatcgttgacgaagttgcttaccacgaaaaataccctaccatctaccatttgagaaagaagttggtcgattccaccgacaaggctgatttgagattgatctatttggccttggctcacatgatcaagttcagaggtcacttcttgattgaaggtgacttgaacccagacaactctgacgtcgacaaattgttcatccaattggtccaaacctacaaccaattattcgaggaaaacccaattaacgcttctggtgttgatgctaaggccatcttatctgcccgtttgtccaagtctagacgtttggaaaacttgattgctcaattgcctggtgaaaagaaaaacggtttgttcggtaacttgatcgctttgtccttgggtttgaccccaaacttcaagtccaacttcgacttggctgaagatgccaagttgcaattgtccaaggacacctacgacgacgacttagacaacttgttggctcaaatcggtgaccaatacgccgacttgttcttggctgccaaaaacttatctgacgctatcttgttgtctgacatcttgagagttaacactgaaattaccaaggctccattgtctgcttctatgatcaaaagatacgacgaacaccaccaagatctgactttgttgaaggctttggttagacaacaattgccagaaaagtacaaggaaatcttcttcgaccaatccaaaaatggttacgccggttacattgacggtggtgcttctcaggaagaattctacaagttcatcaagccaattttggaaaagatggatggtactgaagaattattggttaagttgaacagagaagacttattgagaaagcaacgtaccttcgataacggttctatcccacaccaaatccacttgggtgaattgcacgccattttgagaagacaggaagatttctatccattcctaaaggacaacagagaaaagatcgaaaagatcttaactttcagaatcccatactacgtcggtccattggccagaggtaattctagattcgcttggatgaccagaaagtctgaagaaaccatcaccccatggaacttcgaagaagtcgtcgacaagggtgcttctgcccaatctttcatcgaaagaatgaccaactttgataagaacttgccaaacgagaaggtcttgccaaagcactctttgttgtacgaatacttcaccgtctacaacgaattaaccaaggttaaatacgttactgaaggtatgagaaagccagctttcctatccggtgaacaaaagaaggctattgttgacttgttgtttaagaccaacagaaaggtcactgttaagcaattgaaggaagactacttcaagaagattgaatgtttcgattccgtcgaaatctccggtgttgaagaccgtttcaatgcttctttgggcacctaccacgatttgttaaagatcatcaaggacaaggactttttagataacgaagaaaacgaagacatcttggaagatatcgttttgaccttgactcttttcgaggacagagaaatgattgaagagagattgaagacctacgctcacttgttcgacgataaagttatgaagcaactaaagagaagaagatacactggttggggtagattgtccagaaagttgattaacggtatcagagacaagcaatccggtaagactattttagactttttgaaatccgatggtttcgctaacagaaactttatgcaattgattcacgacgattctttgactttcaaggaagacattcaaaaagcccaagtctctggtcaaggtgattctttgcacgaacacatcgctaacttggctggttctccagctattaagaagggtatcttacaaaccgtcaaggtcgttgatgaattggtcaaagtcatgggtagacacaagccagaaaatattgtcatcgaaatggctagagaaaaccaaactactcaaaagggtcaaaagaactctagagaacgtatgaagagaattgaagaaggtatcaaggagttgggttctcaaattttgaaagaacacccagtcgaaaacactcaattacaaaacgaaaagctatacttgtactacttgcaaaacggtcgtgacatgtacgtcgaccaagaattggatatcaacagattgtctgactacgatgtcgatcatatcgtcccacaatcgttcttgaaggacgattccattgacaacaaagttttgactagatctgacaagaacagaggtaagtctgataacgttccatctgaagaagttgttaagaagatgaagaactactggagacaattgttgaatgctaagttgatcactcaaagaaagttcgacaacttgaccaaggctgaaagaggtggtttgtccgaattggacaaagccggtttcatcaagagacaattagtcgaaactagacaaatcaccaagcatgttgctcaaatcttggattccagaatgaacactaagtacgatgaaaacgacaaactaattagagaagttaaggtcatcactttgaagtctaagttggtttctgacttcagaaaggacttccaattttacaaggtcagagaaatcaacaactaccatcacgctcacgatgcctacttgaacgctgttgtcggtactgccttaatcaaaaagtacccaaagttggaatctgaattcgtttacggtgactacaaggtttacgatgttagaaagatgatcgccaagtctgaacaagaaattggtaaggccactgctaagtacttcttctactctaacatcatgaactttttcaagactgaaatcactttagctaacggtgaaattagaaagcgtccattgattgaaaccaatggtgaaactggtgaaattgtctgggacaagggtagagatttcgctaccgtcagaaaggttttgtctatgccacaagttaacatcgtcaagaagactgaagttcaaactggtggtttctctaaggaatccattttgccaaagagaaactctgacaagttgattgctagaaagaaggactgggatcctaagaagtacggtggtttcgactctccaactgttgcttactccgttttggtcgttgctaaggttgaaaagggtaagtctaagaagttgaagtctgttaaggaattgttgggtatcaccatcatggaaagatcctccttcgaaaagaacccaatcgactttttggaagctaagggttacaaggaagtcaagaaggatttgatcattaagttaccaaaatactccttgttcgaattggaaaacggtagaaagagaatgttggcctccgctggtgaactacaaaaaggtaacgaattggctttaccatctaagtacgttaacttcttgtacttggcttcccactacgaaaagttgaaaggttccccagaagacaacgaacaaaagcaattgtttgttgaacaacacaagcactacttggatgaaattattgaacaaatctccgaattctccaagagagtcattttggctgatgctaacttagataaggttttatccgcttacaacaagcacagagacaaaccaatcagagaacaagctgaaaacatcattcatttgttcactttaaccaacttgggtgctccagctgctttcaaatacttcgacactaccattgacagaaagagatacacttccaccaaagaagttttagatgctactttgattcaccaatctattaccggtttgtacgaaaccagaattgacttgtctcaattgggtggtgattccagagctgatccaaagaagaagagaaaggtgtaaaggagttaaaggcaaagttttcttttctagagccgttcccacaaataattatacgtatatgcttcttttcgtttactatatatctatatttacaagcctttattcactgatgcaatttgtttccaaatacttttttggagatctcataactagatatcatgatggcgcaacttggcgctatcttaattactctggctgccaggcccgtgtagagggccgcaagaccttctgtacgccatatagtctctaagaacttgaacaagtttctagacctattgccgcctttcggatcgctattgtt

SEQ ID NO: 2 - DNA, 11742 bp, Artificial Sequence
  Nucleotide sequence of vector pCSN061
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaacgacattactatatatataatataggaagcatttaatagacagcatcgtaatatatgtgtactttgcagttatgacgccagatggcagtagtggaagatattctttattgaaaaatagcttgtcaccttacgtacaatcttgatccggagcttttctttttttgccgattaagaattaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccttttctttttttgcggtcacccccatgtggcggggaggcagaggagtaggtagagcaacgaatcctactatttatccaaattagtctaggaactctttttctagattttttagatttgagggcaagcgctgttaacgactcagaaatgtaagcactacggagtagaacgagaaatccgccataggtggaaatcctagcaaaatcttgcttaccctagctagcctcaggtaagctagccttagcctgtcaaatttttttcaaaatttggtaagtttctactagcaaagcaaacacggttcaacaaaccgaaaactccactcattatacgtggaaaccgaaacaaaaaaacaaaaaccaaaatactcgccaatgagaaagttgctgcgtttctactttcgaggaagaggaactgagaggattgactacgaaaggggcaaaaacgagtcgtattctcccattattgtctgctaccacgcggtctagtagaataagcaaccagtcaacgctaagacaggtaatcaaaataccagtctgctggctacgggctagtttttacctcttttagaacccactgtaaaagtccgttgtaaagcccgttctcactgttggcgtttttttttttttggtttagtttcttatttttcatttttttctttcatgaccaaaaacaaacaaatctcgcgatttgtactgcggccactggggcgtggccaaaaaaatgacaaatttagaaaccttagtttctgatttttcctgttatgaggagatatgataaaaaatattactgctttattgttttttttttatctactgaaatagagaaacttacccaaggaggaggcaaaaaaaagagtatatatacagcagctaccattcagattttaatatattcttttctcttcttctacactattattataataattttactatattcatttttagcttaaaacctcatagaatattattcttcagtcactcgcttaaatacttatcaaaaatggacaagaaatactctattggtttggatatcgggaccaactccgtcggttgggctgtcatcaccgacgaatacaaggttccatccaagaaattcaaggtcttgggtaacactgacagacactctatcaagaagaatttgatcggtgctttgttgttcgactccggtgaaaccgctgaagctaccagattgaagcgtaccgctcgtcgtagatacactagacgtaaaaaccgtatttgttacttgcaagaaatcttttctaacgaaatggccaaggttgacgactctttcttccacagattggaagaatctttcttggttgaagaagacaagaagcacgaaagacatccaatcttcggtaacatcgttgacgaagttgcttaccacgaaaaataccctaccatctaccatttgagaaagaagttggtcgattccaccgacaaggctgatttgagattgatctatttggccttggctcacatgatcaagttcagaggtcacttcttgattgaaggtgacttgaacccagacaactctgacgtcgacaaattgttcatccaattggtccaaacctacaaccaattattcgaggaaaacccaattaacgcttctggtgttgatgctaaggccatcttatctgcccgtttgtccaagtctagacgtttggaaaacttgattgctcaattgcctggtgaaaagaaaaacggtttgttcggtaacttgatcgctttgtccttgggtttgaccccaaacttcaagtccaacttcgacttggctgaagatgccaagttgcaattgtccaaggacacctacgacgacgacttagacaacttgttggctcaaatcggtgaccaatacgccgacttgttcttggctgccaaaaacttatctgacgctatcttgttgtctgacatcttgagagttaacactgaaattaccaaggctccattgtctgcttctatgatcaaaagatacgacgaacaccaccaagatctgactttgttgaaggctttggttagacaacaattgccagaaaagtacaaggaaatcttcttcgaccaatccaaaaatggttacgccggttacattgacggtggtgcttctcaggaagaattctacaagttcatcaagccaattttggaaaagatggatggtactgaagaattattggttaagttgaacagagaagacttattgagaaagcaacgtaccttcgataacggttctatcccacaccaaatccacttgggtgaattgcacgccattttgagaagacaggaagatttctatccattcctaaaggacaacagagaaaagatcgaaaagatcttaactttcagaatcccatactacgtcggtccattggccagaggtaattctagattcgcttggatgaccagaaagtctgaagaaaccatcaccccatggaacttcgaagaagtcgtcgacaagggtgcttctgcccaatctttcatcgaaagaatgaccaactttgataagaacttgccaaacgagaaggtcttgccaaagcactctttgttgtacgaatacttcaccgtctacaacgaattaaccaaggttaaatacgttactgaaggtatgagaaagccagctttcctatccggtgaacaaaagaaggctattgttgacttgttgtttaagaccaacagaaaggtcactgttaagcaattgaaggaagactacttcaagaagattgaatgtttcgattccgtcgaaatctccggtgttgaagaccgtttcaatgcttctttgggcacctaccacgatttgttaaagatcatcaaggacaaggactttttagataacgaagaaaacgaagacatcttggaagatatcgttttgaccttgactcttttcgaggacagagaaatgattgaagagagattgaagacctacgctcacttgttcgacgataaagttatgaagcaactaaagagaagaagatacactggttggggtagattgtccagaaagttgattaacggtatcagagacaagcaatccggtaagactattttagactttttgaaatccgatggtttcgctaacagaaactttatgcaattgattcacgacgattctttgactttcaaggaagacattcaaaaagcccaagtctctggtcaaggtgattctttgcacgaacacatcgctaacttggctggttctccagctattaagaagggtatcttacaaaccgtcaaggtcgttgatgaattggtcaaagtcatgggtagacacaagccagaaaatattgtcatcgaaatggctagagaaaaccaaactactcaaaagggtcaaaagaactctagagaacgtatgaagagaattgaagaaggtatcaaggagttgggttctcaaattttgaaagaacacccagtcgaaaacactcaattacaaaacgaaaagctatacttgtactacttgcaaaacggtcgtgacatgtacgtcgaccaagaattggatatcaacagattgtctgactacgatgtcgatcatatcgtcccacaatcgttcttgaaggacgattccattgacaacaaagttttgactagatctgacaagaacagaggtaagtctgataacgttccatctgaagaagttgttaagaagatgaagaactactggagacaattgttgaatgctaagttgatcactcaaagaaagttcgacaacttgaccaaggctgaaagaggtggtttgtccgaattggacaaagccggtttcatcaagagacaattagtcgaaactagacaaatcaccaagcatgttgctcaaatcttggattccagaatgaacactaagtacgatgaaaacgacaaactaattagagaagttaaggtcatcactttgaagtctaagttggtttctgacttcagaaaggacttccaattttacaaggtcagagaaatcaacaactaccatcacgctcacgatgcctacttgaacgctgttgtcggtactgccttaatcaaaaagtacccaaagttggaatctgaattcgtttacggtgactacaaggtttacgatgttagaaagatgatcgccaagtctgaacaagaaattggtaaggccactgctaagtacttcttctactctaacatcatgaactttttcaagactgaaatcactttagctaacggtgaaattagaaagcgtccattgattgaaaccaatggtgaaactggtgaaattgtctgggacaagggtagagatttcgctaccgtcagaaaggttttgtctatgccacaagttaacatcgtcaagaagactgaagttcaaactggtggtttctctaaggaatccattttgccaaagagaaactctgacaagttgattgctagaaagaaggactgggatcctaagaagtacggtggtttcgactctccaactgttgcttactccgttttggtcgttgctaaggttgaaaagggtaagtctaagaagttgaagtctgttaaggaattgttgggtatcaccatcatggaaagatcctccttcgaaaagaacccaatcgactttttggaagctaagggttacaaggaagtcaagaaggatttgatcattaagttaccaaaatactccttgttcgaattggaaaacggtagaaagagaatgttggcctccgctggtgaactacaaaaaggtaacgaattggctttaccatctaagtacgttaacttcttgtacttggcttcccactacgaaaagttgaaaggttccccagaagacaacgaacaaaagcaattgtttgttgaacaacacaagcactacttggatgaaattattgaacaaatctccgaattctccaagagagtcattttggctgatgctaacttagataaggttttatccgcttacaacaagcacagagacaaaccaatcagagaacaagctgaaaacatcattcatttgttcactttaaccaacttgggtgctccagctgctttcaaatacttcgacactaccattgacagaaagagatacacttccaccaaagaagttttagatgctactttgattcaccaatctattaccggtttgtacgaaaccagaattgacttgtctcaattgggtggtgattccagagctgatccaaagaagaagagaaaggtgtaaaggagttaaaggcaaagttttcttttctagagccgttcccacaaataattatacgtatatgcttcttttcgtttactatatatctatatttacaagcctttattcactgatgcaatttgtttccaaatacttttttggagatctcataactagatatcatgatggcgcaacttggcgctatcttaattactctggctgccaggcccgtgtagagggccgcaagaccttctgtacgccatatagtctctaagaacttgaacaagtttctagacctattgccgcctttcggatcgctattgttgcggccgccagctgaagcttcgtacgctgcaggtcgacgaattctaccgttcgtataatgtatgctatacgaagttatagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcataacttcgtataatgtatgctatacgaacggtagaattcgaatcagatccactagtggcctatgcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 3 - DNA, 5712 bp, Artificial Sequence
  Nucleotide sequence of vector pRN1120
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacacctaggatccgtcgacactggatggcggcgttagtatcgaatcgacagcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagtactaggggcagggcatgctcatgtagagcgcctgctcgccgtccgaggcggtgccgtcgtacagggcggtgtccaggccgcagagggtgaaccccatccgccggtacgcgtggatcgccggtgcgttgacgttggtgacctccagccagaggtgcccggcgccccgctcgcgggcgaactccgtcgcgagccccatcaacgcgcgcccgaccccgtgcccccggtgctccggggcgacctcgatgtcctcgacggtcagccggcggttccagccggagtacgagacgaccacgaagcccgccaggtcgccgtcgtccccgtacgcgacgaacgtccgggagtccgggtcgccgtcctccccggcgtccgattcgtcgtccgattcgtcgtcggggaacaccttggtcaggggcgggtccaccggcacctcccgcagggtgaagccgtccccggtggcggtgacgcggaagacggtgtcggtggtgaaggacccatccagtgcctcgatggcctcggcgtcccccgggacactggtgcggtaccggtaagccgtgtcgtcaagagtggtcattttacatggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtctgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggcctccatgtcgctggccgggtgacccggcggggacgaggccttaagttcgaacgtacgagctccggcattgcgaataccgctttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttaacaaagatatgctattgaagtgcaagatggaaacgcagaaaatgaaccggggatgcgacgtgcaagattacctatgcaatagatgcaatagtttctccaggaaccgaaatacatacattgtcttccgtaaagcgctagactatatattattatacaggttcaaatatactatctgtttcagggaaaactcccaggttcggatgttcaaaattcaatgatgggtaacaagtacgatcgtaaatctgtaaaacagtttgtcggatattaggctgtatctcctcaaagcgtattcgaatatcattgagaagctgcagcgtcacatcggataataatgatggcagccattgtagaagtgccttttgcatttctagtctctttctcggtctagctagttttactacatcgcgaagatagaatcttagatcacactgcctttgctgagctggatcaatagagtaacaaaagagtggtaaggcctcgttaaaggacaaggacctgagcggaagtgtatcgtacagtagacggagtatactaggtatagtctatagtccgtggaattaattctcatgtttgacagcttatcatcgataatccggagctagcatgcggccgctctagaactagtggatcccccgggctgcaggaattcgatatcaagcttatcgataccgtcgacctcgagggggggcccggtacccagcttttgttccctttagtgagggttaattccgagcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 4 - DNA, 31 bp, Artificial Sequence
  Nucleotide sequence of the forward primer to obtain Pthd3-YFP-TenoI expression cassette
gtgcttagtcaaaaaattagccttttaattc

SEQ ID NO: 5 - DNA, 23 bp, Artificial Sequence
  Nucleotide sequence of the reverse primer to obtain Pthd3-YFP-TenoI expression cassette
gaggggaggaaatgagaaatgag

SEQ ID NO: 6 - DNA, 72 bp, Artificial Sequence
  Nucleotide sequence of the forward primer to attach connector 5 to the Pthd3-YFP-TenoI expression cassette
aagcgacttccaatcgctttgcatatccagtaccacacccacaggcgtttgtgcttagtcaaaaaattagcc

SEQ ID NO: 7 - DNA, 73 bp, Artificial Sequence
  Nucleotide sequence of the reverse primer to attach connector 3 to the Pthd3-YFP-TenoI expression cassette
acttagtatggtctgttggaaaggattgtggcttcgcatacaggctttctgaggggaggaaatgagaaatgag

SEQ ID NO: 8 - DNA, 1730 bp, Artificial Sequence
  Nucleotide sequence of the Pthd3-YFP-TenoI expression cassette flanked by connector 5 (CON5) and connector 3 (CON3); CON5-Pthd3-YFP-TenoI-CON3
aagcgacttccaatcgctttgcatatccagtaccacacccacaggcgtttgtgcttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatgtctaaaggtgaagaattattcactggtgttgtcccaattttggttgaattagatggtgatgttaatggtcacaaattttctgtctccggtgaaggtgaaggtgatgctacttacggtaaattgaccttaaaattgatttgtactactggtaaattgccagttccatggccaaccttagtcactactttaggttatggtttgcaatgttttgctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcactgctgacaaacaaaagaatggtatcaaagctaacttcaaaattagacacaacattgaagatggtggtgttcaattagctgaccattatcaacaaaatactccaattggtgatggtccagtcttgttaccagacaaccattacttatcctatcaatctgccttatccaaagatccaaacgaaaagagagatcacatggtcttgttagaatttgttactgctgctggtattacccatggtatggatgaattgtacaaataaaagcttttgattaagccttctagtccaaaaaacacgtttttttgtcatttatttcattttcttagaatagtttagtttattcattttatagtcacgaatgttttatgattctatatagggttgcaaacaagcatttttcattttatgttaaaacaatttcaggtttaccttttattctgcttgtggtgacgcgtgtatccgcccgctcttttggtcacccatgtatttaattgcataaataattcttaaaagtggagctagtctatttctatttacatacctctcatttctcatttcctcccctccctcagaaagcctgtatgcgaagccacaatcctttccaacagaccatactaagt

SEQ ID NO: 9 - DNA, 72 bp, Artificial Sequence
  Nucleotide sequence of the forward primer to attach a 50 bp genomic DNA flank to connector 5 of YFP expression cassette; CON5-Pthd3-YFP-TenoI-CON3
cttcatgccagcaatagttgcgtgctgagctcaacagtgcccaacccttgaagcgacttccaatcgctttgc

SEQ ID NO: 10 - DNA, 74 bp, Artificial Sequence
  Nucleotide sequence of the reverse primer to attach a 50 bp genomic DNA flank to connector 3 of YFP expression cassette; CON5-Pthd3-YFP-TenoI-CON3
gaaaagcactcctttagtaccactcaacaagttgtctgatgacaaagaatacttagtatggtctgttggaaagg

SEQ ID NO: 11 - DNA, 1830 bp, Artificial Sequence
  Nucleotide sequence of CON5-Pthd3-YFP-TenoI-CON3 expression cassette that contains 50 bp genomic DNA flanks at 5′ and 3′ side for integration in the genome
cttcatgccagcaatagttgcgtgctgagctcaacagtgcccaacccttgaagcgacttccaatcgctttgcatatccagtaccacacccacaggcgtttgtgcttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatgtctaaaggtgaagaattattcactggtgttgtcccaattttggttgaattagatggtgatgttaatggtcacaaattttctgtctccggtgaaggtgaaggtgatgctacttacggtaaattgaccttaaaattgatttgtactactggtaaattgccagttccatggccaaccttagtcactactttaggttatggtttgcaatgttttgctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcactgctgacaaacaaaagaatggtatcaaagctaacttcaaaattagacacaacattgaagatggtggtgttcaattagctgaccattatcaacaaaatactccaattggtgatggtccagtcttgttaccagacaaccattacttatcctatcaatctgccttatccaaagatccaaacgaaaagagagatcacatggtcttgttagaatttgttactgctgctggtattacccatggtatggatgaattgtacaaataaaagcttttgattaagccttctagtccaaaaaacacgtttttttgtcatttatttcattttcttagaatagtttagtttattcattttatagtcacgaatgttttatgattctatatagggttgcaaacaagcatttttcattttatgttaaaacaatttcaggtttaccttttattctgcttgtggtgacgcgtgtatccgcccgctcttttggtcacccatgtatttaattgcataaataattcttaaaagtggagctagtctatttctatttacatacctctcatttctcatttcctcccctccctcagaaagcctgtatgcgaagccacaatcctttccaacagaccatactaagtattctttgtcatcagacaacttgttgagtggtactaaaggagtgcttttc

SEQ ID NO: 12 - DNA, 20 bp, Artificial Sequence
  Nucleotide sequence of the guide sequence (genomic target sequence) of INT1 for Cas9
tattagaaccagggaggtcc

SEQ ID NO: 13 - DNA, 488 bp, Artificial Sequence
  Nucleotide sequence of the complete guide RNA cassette for targeting CAS9 to INT1 locus in the genome that contains homology to vector backbone pRN1120 for homologous recombination
cggagctagcatgcggccgctctagaactagtggatcccccgggctgcagtctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctattagaaccagggaggtccgttttagagctagaaatagcaagttaaaataaggctagtccgttatcaacttgaaaaagtggcaccgagtcggtggtgctttttttgttttttatgtctggggggcccggtacccagcttttgttccctttagtgagggttaattccga

SEQ ID NO: 14 - DNA, 488 bp, Artificial Sequence
  Nucleotide sequence of CTEC-1 comprising a guide RNA cassette (sgRNA) for Cas9 targeting to INT1 and donor DNA on the 3′ side
tctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctattagaaccagggaggtccgttttagagctagaaatagcaagttaaaataaggctagtccgttatcaacttgaaaaagtggcaccgagtcggtggtgctttttttgttttttatgtctacaaatctgcaaccccagcttcataagctttctctcccaccagcaaagcatggacctccctggttctaataatgagcgactgaagttttccaaaagaaac

SEQ ID NO: 15 - DNA, 538 bp, Artificial Sequence
  Nucleotide sequence of CTEC-2 comprising a guide RNA cassette (sgRNA) for Cas9 targeting to INT1, connector A and donor DNA on the 3′ side
tctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctattagaaccagggaggtccgttttagagctagaaatagcaagttaaaataaggctagtccgttatcaacttgaaaaagtggcaccgagtcggtggtgctttttttgttttttatgtctttgcccatcgaacgtacaagtactcctctgttctctccttcctttgctttacaaatctgcaaccccagcttcataagctttctctcccaccagcaaagcatggacctccctggttctaataatgagcgactgaagttttccaaaagaaac

SEQ ID NO: 16 - DNA, 488 bp, Artificial Sequence
  Nucleotide sequence of CTEC-3 comprising a guide RNA cassette (sgRNA) for Cas9 targeting to INT1 and donor DNA on the 5′ side
acaaatctgcaaccccagcttcataagctttctctcccaccagcaaagcatggacctccctggttctaataatgagcgactgaagttttccaaaagaaactctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctattagaaccagggaggtccgttttagagctagaaatagcaagttaaaataaggctagtccgttatcaacttgaaaaagtggcaccgagtcggtggtgctttttttgttttttatgtct

SEQ ID NO: 17 - DNA, 538 bp, Artificial Sequence
  Nucleotide sequence of CTEC-4 comprising a guide RNA cassette (sgRNA) for Cas9 targeting to INT1, connector A and donor DNA on the 5′ side
acaaatctgcaaccccagcttcataagctttctctcccaccagcaaagcatggacctccctggttctaataatgagcgactgaagttttccaaaagaaacttgcccatcgaacgtacaagtactcctctgttctctccttcctttgcttttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctattagaaccagggaggtccgttttagagctagaaatagcaagttaaaataaggctagtccgttatcaacttgaaaaagtggcaccgagtcggtggtgctttttttgttttttatgtct

SEQ ID NO: 18 - DNA, 511 bp, Artificial Sequence
  Nucleotide sequence of CTEC-5 comprising a guide RNA cassette (sgRNA) for Cas9 targeting to INT1, PAM and guide target sequence and donor DNA on the 5′ side
acaaatctgcaaccccagcttcataagctttctctcccaccagcaaagcatggacctccctggttctaataatgagcgactgaagttttccaaaagaaaccctggacctccctggttctaatatctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctattagaaccagggaggtccgttttagagctagaaatagcaagttaaaataaggctagtccgttatcaacttgaaaaagtggcaccgagtcggtggtgctttttttgttttttatgtct

SEQ ID NO: 19 - DNA, 511 bp, Artificial Sequence
  Nucleotide sequence of CTEC-6B comprising a guide RNA cassette (sgRNA) for Cas9 targeting to INT1, PAM and guide target sequence and donor DNA on the 3′ side
tctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctattagaaccagggaggtccgttttagagctagaaatagcaagttaaaataaggctagtccgttatcaacttgaaaaagtggcaccgagtcggtggtgctttttttgttttttatgtctcctggacctccctggttctaataacaaatctgcaaccccagcttcataagctttctctcccaccagcaaagcatggacctccctggttctaataatgagcgactgaagttttccaaaagaaac

SEQ ID NO: 20 - DNA, 499 bp, Artificial Sequence
  Nucleotide sequence of CTEC-1 comprising a guide RNA cassette (sgRNA)  for Cas9 targeting to the YFP gene and donor DNA on the 3′ side
tctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcttagtcactactttaggttagttttagagctagaaatagcaagttaaaataaggctagtccgttatcaacttgaaaaagtggcaccgagtcggtggtgctttttttgttttttatgtctatttgtactactggtaaattgccagttccatggccaaccttagtcactactttagttatggtttgcaatgttttgctagatacccagatcatatgaaacaacatgactttt

SEQ ID NO: 21 - DNA, 549 bp, Artificial Sequence
  Nucleotide sequence of CTEC-2 comprising a guide RNA cassette (sgRNA)  for Cas9 targeting to the YFP gene, connector A and donor DNA on the 3′ side
tctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcttagtcactactttaggttagttttagagctagaaatagcaagttaaaataaggctagtccgttatcaacttgaaaaagtggcaccgagtcggtggtgctttttttgttttttatgtctttgcccatcgaacgtacaagtactcctctgttctctccttcctttgctttatttgtactactggtaaattgccagttccatggccaaccttagtcactactttagttatggtttgcaatgttttgctagatacccagatcatatgaaacaacatgactttt

SEQ ID NO: 22 - DNA, 499 bp, Artificial Sequence
  Nucleotide sequence of CTEC-3 comprising a guide RNA cassette(sgRNA)  for Cas9 targeting to the YFP gene and donor DNA on the 5′ side
atttgtactactggtaaattgccagttccatggccaaccttagtcactactttagttatggtttgcaatgttttgctagatacccagatcatatgaaacaacatgactttttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcttagtcactactttaggttagttttagagctagaaatagcaagttaaaataaggctagtccgttatcaacttgaaaaagtggcaccgagtcggtggtgctttttttgttttttatgtct

SEQ ID NO: 23 - DNA, 549 bp, Artificial Sequence
  Nucleotide sequence of CTEC-4 comprising a guide RNA cassette (sgRNA) for Cas9 targeting to the YFP gene, connector A and donor DNA on the 5′ side
atttgtactactggtaaattgccagttccatggccaaccttagtcactactttagttatggtttgcaatgttttgctagatacccagatcatatgaaacaacatgacttttttgcccatcgaacgtacaagtactcctctgttctctccttcctttgcttttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcttagtcactactttaggttagttttagagctagaaatagcaagttaaaataaggctagtccgttatcaacttgaaaaagtggcaccgagtcggtggtgctttttttgttttttatgtct

SEQ ID NO: 24 - DNA, 522 bp, Artificial Sequence
  Nnucleotide sequence of CTEC-5 comprising a guide RNA cassette (sgRNA) for Cas9 targeting to the YFP gene, PAM and guide target sequence and donor DNA on the 5′ side
atttgtactactggtaaattgccagttccatggccaaccttagtcactactttagttatggtttgcaatgttttgctagatacccagatcatatgaaacaacatgacttttccataacctaaagtagtgactaatctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcttagtcactactttaggttagttttagagctagaaatagcaagttaaaataaggctagtccgttatcaacttgaaaaagtggcaccgagtcggtggtgctttttttgttttttatgtct

SEQ ID NO: 25 - DNA, 522 bp, Artificial Sequence
  Nucleotide sequence of CTEC-6A comprising a guide RNA cassette (sgRNA) for Cas9 targeting to the YFP gene, guide target and PAM sequence and donor DNA on the 3′ side
tctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcttagtcactactttaggttagttttagagctagaaatagcaagttaaaataaggctagtccgttatcaacttgaaaaagtggcaccgagtcggtggtgctttttttgttttttatgtctttagtcactactttaggttatggatttgtactactggtaaattgccagttccatggccaaccttagtcactactttagttatggtttgcaatgttttgctagatacccagatcatatgaaacaacatgactttt

SEQ ID NO: 26 - DNA, 20 bp, Artificial Sequence
  Nucleotide sequence of guide sequence (genomic target sequence) of INT1 for Cas9
tattagaaccagggaggtcc

SEQ ID NO: 27 - DNA, 20 bp, Artificial Sequence
  Nucleotide sequence of guide sequence (genomic target sequence) of YFP for Cas9
ttagtcactactttaggtta

SEQ ID NO: 28 - DNA, 50 bp, Artificial Sequence
  Nucleotide sequence of connector A
ttgcccatcgaacgtacaagtactcctctgttctctccttcctttgcttt

SEQ ID NO: 29 - DNA, 388 bp, Artificial Sequence
  Nucleotide sequence of the complete guide RNA expression cassette for targeting Cas9 to the YFP expression cassette in the genome of CSN009
tctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcttagtcactactttaggttagttttagagctagaaatagcaagttaaaataaggctagtccgttatcaacttgaaaaagtggcaccgagtcggtggtgctttttttgttttttatgtct

SEQ ID NO: 30 - DNA, 388 bp, Artificial Sequence
  Nucleotide sequence of the complete guide RNA expression cassette for targeting Cas9 to the INT1 locus in the genome of CSN001
tctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctattagaaccagggaggtccgttttagagctagaaatagcaagttaaaataaggctagtccgttatcaacttgaaaaagtggcaccgagtcggtggtgctttttttgttttttatgtct

SEQ ID NO: 31 - DNA, 111 bp, Artificial Sequence
  Nucleotide sequence of the YFP donor DNA that is part of CTEC fragments for Cas9 editing
atttgtactactggtaaattgccagttccatggccaaccttagtcactactttagttatggtttgcaatgttttgctagatacccagatcatatgaaacaacatgactttt

SEQ ID NO: 32 - DNA, 100 bp, Artificial Sequence
  Nucleotide sequence of the INT1 donor DNA that is part of CTEC fragments for Cas9 editing
acaaatctgcaaccccagcttcataagctttctctcccaccagcaaagcatggacctccctggttctaataatgagcgactgaagttttccaaaagaaac

SEQ ID NO: 33 - DNA, 28 bp, Artificial Sequence
  Nucleotide sequence of the forward primer to amplify CTEC fragments that contain donor DNA on the 3′ side
tctttgaaaagataatgtatgattatgc

SEQ ID NO: 34 - DNA, 25 bp, Artificial Sequence
  Nucleotide sequence of the forward primer to amplify CTEC fragments that contain the YFP donor DNA on the 5′ side
atttgtactactggtaaattgccag

SEQ ID NO: 35 - DNA, 27 bp, Artificial Sequence
  Nucleotide sequence of the reverse primer to amplify CTEC fragments that contain the YFP donor DNA on the 3′ side
aaaagtcatgttgtttcatatgatctg

SEQ ID NO: 36 - DNA, 28 bp, Artificial Sequence
  Nucleotide sequence of the reverse primer to amplify CTEC fragments that contain donor DNA on the 5′ side
agacataaaaaacaaaaaaagcaccacc

SEQ ID NO: 37 - DNA, 22 bp, Artificial Sequence
  Nucleotide sequence of the forward primer to amplify CTEC fragments that contain the INT1 donor DNA on the 5′ side
acaaatctgcaaccccagcttc

SEQ ID NO: 38 - DNA, 27 bp, Artificial Sequence
  Nucleotide sequence of the reverse primer to amplify CTEC fragments that contain the INT1 donor DNA on the 3′ side
gtttcttttggaaaacttcagtcgctc

SEQ ID NO: 39 - DNA, 26 bp, Artificial Sequence
  Nucleotide sequence of the forward primer to amplify the YFP ORF
atgtctaaaggtgaagaattattcac

SEQ ID NO: 40 - DNA, 26 bp, Artificial Sequence
  Nucleotide sequence of the reverse primer to amplify the YFP ORF
ttttatttgtacaattcatccatacc

SEQ ID NO: 41 - DNA, 26 bp, Artificial Sequence
  Nucleotide sequence of forward primer used for sequencing the YFP ORF
ttttatttgtacaattcatccatacc

SEQ ID NO: 42 - DNA, 24 bp, Artificial Sequence
  Nucleotide sequence of the forward primer to amplify part of the INT1 locus
attaagtaatagatacgcacaacc

SEQ ID NO: 43 - DNA, 21 bp, Artificial Sequence
  Nucleotide sequence of the reverse primer to amplify part of the INT1 locus
ggaatactaccagatcgatcc

SEQ ID NO: 44 - DNA, 24 bp, Artificial Sequence
  Nucleotide sequence of the forward primer used for sequencing part of the INT1 locus
attaagtaatagatacgcacaacc

SEQ ID NO: 45 - DNA, 24 bp, Artificial Sequence
  Nucleotide sequence of the forward primer to amplify the Kl11p-pCSN061 backbone-GND2t PCR fragment
ttttgataagtatttaagcgagtg

SEQ ID NO: 46 - DNA, 22 bp, Artificial Sequence
  Nucleotide sequence of the reverse primer to amplify the Kl11p-pCSN061 backbone-GND2t PCR fragment
aggagttaaaggcaaagttttc

SEQ ID NO: 47 - protein, 1239 aa, Artificial Sequence
  Protein sequence of LbCpf1 (from Lachnospiraceae bacterium ND2006) including a C-terminal NLS
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKHSRADPKKKRKV

SEQ ID NO: 48 - DNA, 3720 bp, Artificial Sequence
  Nucleotide sequence CPO LbCpf1 including a C-terminal NLS
atgtctaagttggaaaaattcaccaactgttactctttgtctaagactttgagattcaaggccatcccagttggtaagacccaagaaaacatcgacaacaagagactattagttgaagatgaaaagagagctgaagactacaagggtgtcaagaaattgttggacagatactacttgtcttttatcaacgacgttttgcattccatcaagctaaagaacttgaataactacatctctttgttcagaaagaagactagaactgaaaaggaaaataaggaattggaaaacttggaaatcaacttgagaaaggaaattgctaaggctttcaagggtaatgaaggttacaagtctttattcaagaaagacatcattgaaaccattttgccagaatttttggatgataaggatgaaattgctttggttaactctttcaacggtttcaccactgctttcactggtttcttcgacaacagagaaaacatgttctccgaggaagctaaatccacttctattgctttcagatgtatcaacgaaaacttgacccgttacatctctaacatggacatttttgaaaaggtcgacgccatctttgacaagcacgaagtccaagaaatcaaggaaaagatcttaaactccgactacgatgtcgaagatttcttcgaaggtgaattcttcaactttgttttaacccaagaaggtatcgatgtctacaacgccattatcggtggttttgtcactgaatctggtgaaaagatcaagggtttgaacgaatacattaacttgtacaaccaaaagaccaaacaaaaattgccaaagttcaagccattgtacaagcaagttttgtctgacagagaatctttgtctttttacggtgaagggtacacctctgacgaagaagtcttggaagtcttcagaaacactttgaacaagaactctgaaatcttctcctccatcaagaagttagaaaagttgttcaagaacttcgatgaatactcttctgctggtatcttcgttaagaacggtccagccatctctaccatttctaaggatatctttggtgaatggaacgtcattagagacaaatggaacgctgaatacgatgacatccatttgaagaaaaaggctgttgtcaccgaaaagtacgaagacgacagaagaaaatccttcaagaagatcggttccttctccttggaacaattacaagaatacgccgatgccgatttgtccgttgtcgaaaaattgaaggaaattattattcaaaaggttgatgaaatttacaaagtttacggttcctctgaaaagttattcgatgctgatttcgtcttggaaaagtctttgaagaagaacgacgctgttgtcgctatcatgaaggacttgttggactctgtcaaatctttcgaaaactatatcaaggccttcttcggtgaaggtaaggaaactaacagagatgaatccttctacggtgactttgtcttggcttacgatattttgttgaaggttgaccacatctacgatgccatcagaaactacgttactcaaaagccatactctaaggacaaattcaagttgtacttccaaaacccacaattcatgggtggttgggataaggacaaggaaactgactacagagctaccattttgagatacggttccaagtactacttggccatcatggacaagaagtacgccaagtgtttgcaaaagattgacaaggacgatgtcaacggtaactacgaaaagattaactacaagttgttgccaggtccaaacaagatgttgccaaaggttttcttctccaaaaagtggatggcttactacaacccatctgaagacatccaaaagatctacaagaacggtactttcaaaaagggtgacatgttcaacttaaacgactgtcacaagttgatcgacttcttcaaggactccatctctagatacccaaaatggtccaacgcttacgatttcaacttctctgaaactgaaaaatacaaggatattgctggtttctaccgtgaagtcgaggaacaaggttataaggtttctttcgaatccgcttctaagaaagaagttgacaaattagtcgaagaaggtaagttgtacatgttccaaatctacaacaaagatttctccgacaagtctcacggtactccaaacttgcacaccatgtacttcaagttgctattcgatgaaaacaaccacggtcaaatcagattgtctggtggtgctgaattgttcatgagacgtgcttctctaaagaaggaagaattagtcgtccacccagctaactctccaattgccaacaagaacccagacaaccctaagaagaccaccactttgtcctacgacgtttacaaggacaagagattctccgaagaccaatacgaattgcacattccaattgctatcaacaagtgtccaaagaacatcttcaagatcaacactgaagtcagagttttgttaaagcacgatgacaacccttacgttattggtatcgaccgtggtgaaagaaatttgttgtacattgttgttgttgacggtaagggtaacatcgttgaacaatactccttgaacgaaatcatcaacaacttcaacggtattagaatcaagactgattaccactctttgttggataagaaggaaaaggaacgttttgaagctcgtcaaaactggacctctattgaaaacatcaaagaattgaaggctggttacatcagtcaagttgtccacaagatctgtgaattggtcgagaagtacgatgccgttattgccttggaagatttgaactctggttttaagaactctcgtgtcaaggttgaaaagcaagtctaccaaaagttcgaaaagatgttaatcgacaaattgaactacatggttgacaagaaatccaacccatgtgctaccggtggtgctttgaaaggttaccaaatcaccaacaaattcgaatctttcaaatctatgtccactcaaaacgggttcatcttctacattccagcttggttgacctccaagatcgacccatctaccggtttcgttaacttgttgaagaccaagtacacttccattgctgattccaagaagttcatctcttctttcgacagaatcatgtacgttccagaagaagacttgttcgaattcgccttggactataagaacttctccagaaccgatgctgactacattaagaaatggaaattgtactcctacggtaacagaatcagaattttcagaaacccaaagaaaaacaacgttttcgattgggaagaagtttgtttgacttctgcctacaaggaattattcaacaaatacggtatcaactaccaacaaggtgatatcagagctttgttgtgtgaacaatctgacaaggctttctactcttccttcatggctttgatgtccttgatgttgcaaatgagaaactccatcactggtagaactgatgtcgacttcctcatttctccagttaagaattctgacggtattttctacgactctagaaattacgaagctcaagaaaacgctattttgccaaagaacgctgatgctaacggtgcttacaatattgctagaaaggttttgtgggctatcggtcaattcaagaaggctgaagacgaaaagctagacaaggtcaagattgctatttctaacaaggaatggttggaatacgctcaaacctccgtcaagcactccagagctgatccaaagaagaagagaaaggtataa

SEQ ID NO: 49 - DNA, 60 bp, Artificial Sequence
  Nucleotide sequence of the forward primer to amplify LbCpf1 expression cassette
cctcatagaatattattcttcagtcactcgcttaaatacttatcaaaaatgtctaagttg

SEQ ID NO: 50 - DNA, 74 bp, Artificial Sequence
  Nucleotide sequence of the reverse primer to amplify LbCpf1 expression cassette
cgtataattatttgtgggaacggctctagaaaagaaaactttgcctttaactcctttatacctttctcttcttc

SEQ ID NO: 51 - DNA, 11322 bp, Artificial Sequence
  Nucleotide sequence of vector pCSN067 encoding LbCpf1
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaacgacattactatatatataatataggaagcatttaatagacagcatcgtaatatatgtgtactttgcagttatgacgccagatggcagtagtggaagatattctttattgaaaaatagcttgtcaccttacgtacaatcttgatccggagcttttctttttttgccgattaagaattaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccttttctttttttgcggtcacccccatgtggcggggaggcagaggagtaggtagagcaacgaatcctactatttatccaaattagtctaggaactctttttctagattttttagatttgagggcaagcgctgttaacgactcagaaatgtaagcactacggagtagaacgagaaatccgccataggtggaaatcctagcaaaatcttgcttaccctagctagcctcaggtaagctagccttagcctgtcaaatttttttcaaaatttggtaagtttctactagcaaagcaaacacggttcaacaaaccgaaaactccactcattatacgtggaaaccgaaacaaaaaaacaaaaaccaaaatactcgccaatgagaaagttgctgcgtttctactttcgaggaagaggaactgagaggattgactacgaaaggggcaaaaacgagtcgtattctcccattattgtctgctaccacgcggtctagtagaataagcaaccagtcaacgctaagacaggtaatcaaaataccagtctgctggctacgggctagtttttacctcttttagaacccactgtaaaagtccgttgtaaagcccgttctcactgttggcgtttttttttttttggtttagtttcttatttttcatttttttctttcatgaccaaaaacaaacaaatctcgcgatttgtactgcggccactggggcgtggccaaaaaaatgacaaatttagaaaccttagtttctgatttttcctgttatgaggagatatgataaaaaatattactgctttattgttttttttttatctactgaaatagagaaacttacccaaggaggaggcaaaaaaaagagtatatatacagcagctaccattcagattttaatatattcttttctcttcttctacactattattataataattttactatattcatttttagcttaaaacctcatagaatattattcttcagtcactcgcttaaatacttatcaaaaatgtctaagttggaaaaattcaccaactgttactctttgtctaagactttgagattcaaggccatcccagttggtaagacccaagaaaacatcgacaacaagagactattagttgaagatgaaaagagagctgaagactacaagggtgtcaagaaattgttggacagatactacttgtcttttatcaacgacgttttgcattccatcaagctaaagaacttgaataactacatctctttgttcagaaagaagactagaactgaaaaggaaaataaggaattggaaaacttggaaatcaacttgagaaaggaaattgctaaggctttcaagggtaatgaaggttacaagtctttattcaagaaagacatcattgaaaccattttgccagaatttttggatgataaggatgaaattgctttggttaactctttcaacggtttcaccactgctttcactggtttcttcgacaacagagaaaacatgttctccgaggaagctaaatccacttctattgctttcagatgtatcaacgaaaacttgacccgttacatctctaacatggacatttttgaaaaggtcgacgccatctttgacaagcacgaagtccaagaaatcaaggaaaagatcttaaactccgactacgatgtcgaagatttcttcgaaggtgaattcttcaactttgttttaacccaagaaggtatcgatgtctacaacgccattatcggtggttttgtcactgaatctggtgaaaagatcaagggtttgaacgaatacattaacttgtacaaccaaaagaccaaacaaaaattgccaaagttcaagccattgtacaagcaagttttgtctgacagagaatctttgtctttttacggtgaagggtacacctctgacgaagaagtcttggaagtcttcagaaacactttgaacaagaactctgaaatcttctcctccatcaagaagttagaaaagttgttcaagaacttcgatgaatactcttctgctggtatcttcgttaagaacggtccagccatctctaccatttctaaggatatctttggtgaatggaacgtcattagagacaaatggaacgctgaatacgatgacatccatttgaagaaaaaggctgttgtcaccgaaaagtacgaagacgacagaagaaaatccttcaagaagatcggttccttctccttggaacaattacaagaatacgccgatgccgatttgtccgttgtcgaaaaattgaaggaaattattattcaaaaggttgatgaaatttacaaagtttacggttcctctgaaaagttattcgatgctgatttcgtcttggaaaagtctttgaagaagaacgacgctgttgtcgctatcatgaaggacttgttggactctgtcaaatctttcgaaaactatatcaaggccttcttcggtgaaggtaaggaaactaacagagatgaatccttctacggtgactttgtcttggcttacgatattttgttgaaggttgaccacatctacgatgccatcagaaactacgttactcaaaagccatactctaaggacaaattcaagttgtacttccaaaacccacaattcatgggtggttgggataaggacaaggaaactgactacagagctaccattttgagatacggttccaagtactacttggccatcatggacaagaagtacgccaagtgtttgcaaaagattgacaaggacgatgtcaacggtaactacgaaaagattaactacaagttgttgccaggtccaaacaagatgttgccaaaggttttcttctccaaaaagtggatggcttactacaacccatctgaagacatccaaaagatctacaagaacggtactttcaaaaagggtgacatgttcaacttaaacgactgtcacaagttgatcgacttcttcaaggactccatctctagatacccaaaatggtccaacgcttacgatttcaacttctctgaaactgaaaaatacaaggatattgctggtttctaccgtgaagtcgaggaacaaggttataaggtttctttcgaatccgcttctaagaaagaagttgacaaattagtcgaagaaggtaagttgtacatgttccaaatctacaacaaagatttctccgacaagtctcacggtactccaaacttgcacaccatgtacttcaagttgctattcgatgaaaacaaccacggtcaaatcagattgtctggtggtgctgaattgttcatgagacgtgcttctctaaagaaggaagaattagtcgtccacccagctaactctccaattgccaacaagaacccagacaaccctaagaagaccaccactttgtcctacgacgtttacaaggacaagagattctccgaagaccaatacgaattgcacattccaattgctatcaacaagtgtccaaagaacatcttcaagatcaacactgaagtcagagttttgttaaagcacgatgacaacccttacgttattggtatcgaccgtggtgaaagaaatttgttgtacattgttgttgttgacggtaagggtaacatcgttgaacaatactccttgaacgaaatcatcaacaacttcaacggtattagaatcaagactgattaccactctttgttggataagaaggaaaaggaacgttttgaagctcgtcaaaactggacctctattgaaaacatcaaagaattgaaggctggttacatcagtcaagttgtccacaagatctgtgaattggtcgagaagtacgatgccgttattgccttggaagatttgaactctggttttaagaactctcgtgtcaaggttgaaaagcaagtctaccaaaagttcgaaaagatgttaatcgacaaattgaactacatggttgacaagaaatccaacccatgtgctaccggtggtgctttgaaaggttaccaaatcaccaacaaattcgaatctttcaaatctatgtccactcaaaacgggttcatcttctacattccagcttggttgacctccaagatcgacccatctaccggtttcgttaacttgttgaagaccaagtacacttccattgctgattccaagaagttcatctcttctttcgacagaatcatgtacgttccagaagaagacttgttcgaattcgccttggactataagaacttctccagaaccgatgctgactacattaagaaatggaaattgtactcctacggtaacagaatcagaattttcagaaacccaaagaaaaacaacgttttcgattgggaagaagtttgtttgacttctgcctacaaggaattattcaacaaatacggtatcaactaccaacaaggtgatatcagagctttgttgtgtgaacaatctgacaaggctttctactcttccttcatggctttgatgtccttgatgttgcaaatgagaaactccatcactggtagaactgatgtcgacttcctcatttctccagttaagaattctgacggtattttctacgactctagaaattacgaagctcaagaaaacgctattttgccaaagaacgctgatgctaacggtgcttacaatattgctagaaaggttttgtgggctatcggtcaattcaagaaggctgaagacgaaaagctagacaaggtcaagattgctatttctaacaaggaatggttggaatacgctcaaacctccgtcaagcactccagagctgatccaaagaagaagagaaaggtataaaggagttaaaggcaaagttttcttttctagagccgttcccacaaataattatacgtatatgcttcttttcgtttactatatatctatatttacaagcctttattcactgatgcaatttgtttccaaatacttttttggagatctcataactagatatcatgatggcgcaacttggcgctatcttaattactctggctgccaggcccgtgtagagggccgcaagaccttctgtacgccatatagtctctaagaacttgaacaagtttctagacctattgccgcctttcggatcgctattgttgcggccgccagctgaagcttcgtacgctgcaggtcgacgaattctaccgttcgtataatgtatgctatacgaagttatagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcataacttcgtataatgtatgctatacgaacggtagaattcgaatcagatccactagtggcctatgcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 52 - DNA, 21 bp, Artificial Sequence
  Nucleotide sequence of direct repeat part of crRNA cassette of LbCpf1
taatttctactaagtgtagat

SEQ ID NO: 53 - DNA, 20 bp, Artificial Sequence
  Nucleotide sequence of guide sequence (genomic target sequence) of INT1 for LbCpf1
ctggtgggagagaaagctta

SEQ ID NO: 54 - DNA, 430 bp, Artificial Sequence
  Nucleotide sequence of the complete guide RNA cassette for targeting LbCpf1 to the INT1 locus in the genome that contains homology to vector backbone pRN1120 for homologous recombination
cggagctagcatgcggccgctctagaactagtggatcccccgggctgcagtctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatctggtgggagagaaagcttatttttttgttttttatgtctggggggcccggtacccagcttttgttccctttagtgagggttaattccga

SEQ ID NO: 55 - DNA, 439 bp, Artificial Sequence
  Nucleotide sequence of CTEC-7 comprising a guide RNA cassette (crRNA) for targeting LbCpf1 to the YFP gene and donor DNA on the 3′ side
tctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatcaatgttttgctagataccctttttttgttttttatgtctatttgtactactggtaaattgccagttccatggccaaccttagtcactactttaggttatggtgcaatgttttgctagatacccagatcatatgaaacaacatgacttt

SEQ ID NO: 56 - DNA, 489 bp, Artificial Sequence
  Nucleotide sequence of CTEC-8 comprising a guide RNA cassette (crRNA) for targeting LbCpf1 to the YFP gene, connector A and donor DNA on the 3′ side
tctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatcaatgttttgctagataccctttttttgttttttatgtctttgcccatcgaacgtacaagtactcctctgttctctccttcctttgctttatttgtactactggtaaattgccagttccatggccaaccttagtcactactttaggttatggtgcaatgttttgctagatacccagatcatatgaaacaacatgacttt

SEQ ID NO: 57 - DNA, 439 bp, Artificial Sequence
  Nucleotide sequence of CTEC-9 comprising a guide RNA cassette (crRNA) for targeting LbCpf1 to the YFP gene and donor DNA on the 5′ side
atttgtactactggtaaattgccagttccatggccaaccttagtcactactttaggttatggtgcaatgttttgctagatacccagatcatatgaaacaacatgacttttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatcaatgttttgctagataccctttttttgttttttatgtct

SEQ ID NO: 58 - DNA, 489 bp, Artificial Sequence
  Nucleotide sequence of CTEC-10 comprising a guide RNA cassette (crRNA) for targeting LbCpf1 to the YFP gene, connector A and donor DNA on the 5′ side
atttgtactactggtaaattgccagttccatggccaaccttagtcactactttaggttatggtgcaatgttttgctagatacccagatcatatgaaacaacatgactttttgcccatcgaacgtacaagtactcctctgttctctccttcctttgcttttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatcaatgttttgctagataccctttttttgttttttatgtct

SEQ ID NO: 59 - DNA, 459 bp, Artificial Sequence
  Nucleotide sequence of CTEC-11 comprising a guide RNA cassette (crRNA) for targeting LbCpf1 to the YFP gene, PAM and guide target sequence and donor DNA on the 3′ side (2 x 18 bp guide)
tctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatcaatgttttgctagatactttttttgttttttatgtcttttgcaatgttttgctagatacatttgtactactggtaaattgccagttccatggccaaccttagtcactactttaggttatggtgcaatgttttgctagatacccagatcatatgaaacaacatgacttt

SEQ ID NO: 60 - DNA, 463 bp, Artificial Sequence
  Nucleotide sequence of CTEC-11 comprising a guide RNA cassette (crRNA) for targeting LbCpf1 to the YFP gene, PAM and guide target sequence and donor DNA on the 3′ side (2 x 20 bp guide)
tctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatcaatgttttgctagataccctttttttgttttttatgtcttttgcaatgttttgctagatacccatttgtactactggtaaattgccagttccatggccaaccttagtcactactttaggttatggtgcaatgttttgctagatacccagatcatatgaaacaacatgacttt

SEQ ID NO: 61 - DNA, 459 bp, Artificial Sequence
  Nucleotide sequence of CTEC-12 comprising a guide RNA cassette (crRNA) for targeting LbCpf1 to the YFP gene, PAM and guide target sequence and donor DNA on the 5′ side (2 x 18 bp guide)
atttgtactactggtaaattgccagttccatggccaaccttagtcactactttaggttatggtgcaatgttttgctagatacccagatcatatgaaacaacatgacttttttgcaatgttttgctagatactctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatcaatgttttgctagatactttttttgttttttatgtct

SEQ ID NO: 62 - DNA, 463 bp, Artificial Sequence
  Nucleotide sequence of CTEC-12 comprising a guide RNA cassette (crRNA) for targeting LbCpf1 to the YFP gene, PAM and guide target sequence and donor DNA on the 5′ side (2 x 20 bp guide)
atttgtactactggtaaattgccagttccatggccaaccttagtcactactttaggttatggtgcaatgttttgctagatacccagatcatatgaaacaacatgacttttttgcaatgttttgctagataccctctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatcaatgttttgctagataccctttttttgttttttatgtct

SEQ ID NO: 63 - DNA, 430 bp, Artificial Sequence
  Nucleotide sequence of CTEC-7 comprising a guide RNA cassette (crRNA) for targeting LbCpf1 to INT1 and donor DNA on the 3′ side
tctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatctggtgggagagaaagcttatttttttgttttttatgtctgtttcttttggaaaacttcagtcgctcattattagaaccagggaggtccaggcccggctggtgggagagaaagcttatgaagctggggttgcagatttgt

SEQ ID NO: 64 - DNA, 480 bp, Artificial Sequence
  Nucleotide sequence of CTEC-8 comprising a guide RNA cassette (crRNA) for targeting LbCpf1 to INT1, connector A and donor DNA on the 3′
tctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatctggtgggagagaaagcttatttttttgttttttatgtctttgcccatcgaacgtacaagtactcctctgttctctccttcctttgctttgtttcttttggaaaacttcagtcgctcattattagaaccagggaggtccaggcccggctggtgggagagaaagcttatgaagctggggttgcagatttgt

SEQ ID NO: 65-66 - skipped (2 entries)

SEQ ID NO: 67 - DNA, 452 bp, Artificial Sequence
  Nucleotide sequence of CTEC-11 comprising a guide RNA cassette (crRNA) for targeting LbCpf1 to INT1, PAM and guide target sequence and donor DNA on the 3′ side (1 x 20 bp, 1x 18 bp guide)
tctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatctggtgggagagaaagcttatttttttgttttttatgtcttttgctggtgggagagaaagctgtttcttttggaaaacttcagtcgctcattattagaaccagggaggtccaggcccggctggtgggagagaaagcttatgaagctggggttgcagatttgt

SEQ ID NO: 68 - DNA, 454 bp, Artificial Sequence
  Nucleotide sequence of CTEC-11 comprising a guide RNA cassette (crRNA) for targeting LbCpf1 to INT1, PAM and guide target sequence and donor DNA on the 3′ side (2 x 20 bp guide)
tctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatctggtgggagagaaagcttatttttttgttttttatgtcttttgctggtgggagagaaagcttagtttcttttggaaaacttcagtcgctcattattagaaccagggaggtccaggcccggctggtgggagagaaagcttatgaagctggggttgcagatttgt

SEQ ID NO: 69 - DNA, 20 bp, Artificial Sequence
  Nucleotide sequence of the guide sequence (genomic target) of the CTEC fragments targeting YFP by LbCpf1 in strain CSN010
caatgttttgctagataccc

SEQ ID NO: 70 - DNA, 20 bp, Artificial Sequence
  Nucleotide sequence of the guide sequence (genomic target) of the CTEC fragments targeting INT1 by LbCpf1 in strain CSN004
ctggtgggagagaaagctta

SEQ ID NO: 71 - DNA, 109 bp, Artificial Sequence
  Nucleotide sequence of YFP donor DNA that is part of CTEC fragments for LbCpf1 mediated editing in strain CSN010
atttgtactactggtaaattgccagttccatggccaaccttagtcactactttaggttatggtgcaatgttttgctagatacccagatcatatgaaacaacatgacttt

SEQ ID NO: 72 - DNA, 100 bp, Artificial Sequence
  Nucleotide sequence of INT donor DNA that is part of CTEC fragments for LbCpf1 mediated editing in strain CSN004
gtttcttttggaaaacttcagtcgctcattattagaaccagggaggtccaggcccggctggtgggagagaaagcttatgaagctggggttgcagatttgt

SEQ ID NO: 73 - DNA, 330 bp, Artificial Sequence
  Nucleotide sequence of complete guide RNA expression cassette for targeting LbCpf1 to the INT1 locus in the genome of CSN004
tctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatctggtgggagagaaagcttatttttttgttttttatgtct

SEQ ID NO: 74 - DNA, 330 bp, Artificial Sequence
  Nucleotide sequence of complete guide RNA expression cassette for targeting LbCpf1 to the YFP expression cassette in the genome of CSN010
tctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatcaatgttttgctagataccctttttttgttttttatgtct

SEQ ID NO: 75 - DNA, 18 bp, Artificial Sequence
  Nucleotide sequence of the 18 bp guide sequence (genomic target sequence) for digestion of the CTEC fragment by LbCpf1 thereby separating the INT1 donor DNA from the guide RNA expression cassette
ctggtgggagagaaagct

SEQ ID NO: 76 - DNA, 18 bp, Artificial Sequence
  Nucleotide sequence of the 18 bp guide sequence (genomic target sequence) for digestion of the CTEC fragment by LbCpf1 thereby separating the YFP donor DNA from the guide RNA expression cassette
caatgttttgctagatac

SEQ ID NO: 77 - DNA, 20 bp, Artificial Sequence
  Nucleotide sequence of the 20 bp guide sequence (genomic target sequence) for digestion of the CTEC fragment by LbCpf1 thereby separating the INT1 donor DNA from the guide RNA expression cassette
ctggtgggagagaaagctta

SEQ ID NO: 78 - DNA, 20 bp, Artificial Sequence
  Nucleotide sequence of the 20 bp guide sequence (genomic target sequence) for digestion of the CTEC fragment by LbCpf1 thereby separating the YFP donor DNA from the guide RNA expression cassette
caatgttttgctagataccc

SEQ ID NO: 79 - DNA, 22 bp, Artificial Sequence
  Nucleotide sequence of the 18 bp guide sequence (genomic target sequence) including the PAM sequence for digestion of the CTEC fragment by LbCpf1 thereby separating the INT1 donor DNA from the guide RNA expression cassette
tttgctggtgggagagaaagct

SEQ ID NO: 80 - DNA, 24 bp, Artificial Sequence
  Nucleotide sequence of the 20 bp guide sequence (genomic target sequence) including the PAM sequence for digestion of the CTEC fragment by LbCpf1 thereby separating the INT1 donor DNA from the guide RNA expression cassette
tttgctggtgggagagaaagctta

SEQ ID NO: 81 - DNA, 22 bp, Artificial Sequence
  Nucleotide sequence of the 18 bp guide sequence (genomic target sequence) including the PAM for digestion of the CTEC fragment by LbCpf1 thereby separating the YFP donor DNA from the guide RNA expression cassette
tttgcaatgttttgctagatac

SEQ ID NO: 82 - DNA, 24 bp, Artificial Sequence
  Nucleotide sequence of the 20 bp guide sequence (genomic target sequence) including the PAM sequence for digestion of the CTEC fragment by LbCpf1 thereby separating the YFP donor DNA from the guide RNA expression cassette
tttgcaatgttttgctagataccc

SEQ ID NO: 83 - DNA, 30 bp, Artificial Sequence
  Nucleotide sequence of the reverse primer to amplify CTEC fragments having the YFP donor on the 5′ side and a 20 bp guide sequence for LbCpf1
agacataaaaaacaaaaaaagggtatctag

SEQ ID NO: 84 - DNA, 28 bp, Artificial Sequence
  Nucleotide sequence of the reverse primer to amplify CTEC fragments having the YFP donor on the 5′ side and a 18 bp guide sequence for LbCpf1
agacataaaaaacaaaaaaagtatctag

SEQ ID NO: 85 - DNA, 25 bp, Artificial Sequence
  Nucleotide sequence of the forward primer to amplify CTEC fragments having the INT1 donor on the 5′ side for LbCpf1 editing
gtttcttttggaaaacttcagtcgc

SEQ ID NO: 86 - DNA, 22 bp, Artificial Sequence
  Nucleotide sequence of the reverse primer to amplify CTEC fragments having the INT1 donor on the 3′ side for LbCpf1 editing
acaaatctgcaaccccagcttc

SEQ ID NO: 87 - DNA, 539 bp, Artificial Sequence
  Nucleotide sequence of CTEC-7 comprising a guide RNA cassette (crRNA) for targeting LbCpf1 to the YFP gene and donor DNA on the 3′ side, flanked by connector 5 sequence on the 5′ side and connector 3 on the 3′ side
aagcgacttccaatcgctttgcatatccagtaccacacccacaggcgttttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatcaatgttttgctagataccctttttttgttttttatgtctatttgtactactggtaaattgccagttccatggccaaccttagtcactactttaggttatggtgcaatgttttgctagatacccagatcatatgaaacaacatgactttagaaagcctgtatgcgaagccacaatcctttccaacagaccatactaagt

SEQ ID NO: 88 - DNA, 589 bp, Artificial Sequence
  Nucleotide sequence of CTEC-8 comprising a guide RNA cassette (crRNA) for targeting LbCpf1 to the YFP gene, connector A and donor DNA on the 3′ side, flanked by connector 5 sequence on the 5′ side and connector 3 on the 3′ side
aagcgacttccaatcgctttgcatatccagtaccacacccacaggcgttttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatcaatgttttgctagataccctttttttgttttttatgtctttgcccatcgaacgtacaagtactcctctgttctctccttcctttgctttatttgtactactggtaaattgccagttccatggccaaccttagtcactactttaggttatggtgcaatgttttgctagatacccagatcatatgaaacaacatgactttagaaagcctgtatgcgaagccacaatcctttccaacagaccatactaagt

SEQ ID NO: 89 - DNA, 539 bp, Artificial Sequence
  Nucleotide sequence of CTEC-9 comprising a guide RNA cassette (crRNA) for targeting LbCpf1 to the YFP gene and donor DNA on the 5′ side, flanked by connector 5 sequence on the 5′ side and connector 3 on the 3′ side
aagcgacttccaatcgctttgcatatccagtaccacacccacaggcgtttatttgtactactggtaaattgccagttccatggccaaccttagtcactactttaggttatggtgcaatgttttgctagatacccagatcatatgaaacaacatgacttttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatcaatgttttgctagataccctttttttgttttttatgtctagaaagcctgtatgcgaagccacaatcctttccaacagaccatactaagt

SEQ ID NO: 90 - DNA, 589 bp, Artificial Sequence
  Nucleotide sequence of CTEC-10 comprising a guide RNA cassette (crRNA) for targeting LbCpf1 to the YFP gene, connector A and donor DNA on the 5′ side, flanked by connector 5 sequence on the 5′ side and connector 3 on the 3′ side
aagcgacttccaatcgctttgcatatccagtaccacacccacaggcgtttatttgtactactggtaaattgccagttccatggccaaccttagtcactactttaggttatggtgcaatgttttgctagatacccagatcatatgaaacaacatgactttttgcccatcgaacgtacaagtactcctctgttctctccttcctttgcttttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatcaatgttttgctagataccctttttttgttttttatgtctagaaagcctgtatgcgaagccacaatcctttccaacagaccatactaagt

SEQ ID NO: 91 - DNA, 559 bp, Artificial Sequence
  Nucleotide sequence of CTEC-11 comprising a guide RNA cassette (crRNA) for targeting LbCpf1 to the YFP gene, PAM and guide target sequence and donor DNA on the 3′ side (2 x 18 bp guide), flanked by connector 5 sequence on the 5′ side and connector 3 on
aagcgacttccaatcgctttgcatatccagtaccacacccacaggcgttttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatcaatgttttgctagatactttttttgttttttatgtcttttgcaatgttttgctagatacatttgtactactggtaaattgccagttccatggccaaccttagtcactactttaggttatggtgcaatgttttgctagatacccagatcatatgaaacaacatgactttagaaagcctgtatgcgaagccacaatcctttccaacagaccatactaagt

SEQ ID NO: 92 - DNA, 563 bp, Artificial Sequence
  Nucleotide sequence of CTEC-11 comprising a guide RNA cassette (crRNA) for targeting LbCpf1 to the YFP gene, PAM and guide target sequence and donor DNA on the 3′ side (2 x  20 bp guide), flanked by connector 5 sequence on the 5′ side and connector 3 on
aagcgacttccaatcgctttgcatatccagtaccacacccacaggcgttttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatcaatgttttgctagataccctttttttgttttttatgtcttttgcaatgttttgctagatacccatttgtactactggtaaattgccagttccatggccaaccttagtcactactttaggttatggtgcaatgttttgctagatacccagatcatatgaaacaacatgactttagaaagcctgtatgcgaagccacaatcctttccaacagaccatactaagt

SEQ ID NO: 93 - DNA, 559 bp, Artificial Sequence
  Nucleotide sequence of CTEC-12 comprising a guide RNA cassette (crRNA) for targeting LbCpf1 to the YFP gene, PAM and guide target sequence and donor DNA on the 5′ side (2 x 18 bp guide), flanked by connector 5 on the 5′ side and connector 3 on the 3′
aagcgacttccaatcgctttgcatatccagtaccacacccacaggcgtttatttgtactactggtaaattgccagttccatggccaaccttagtcactactttaggttatggtgcaatgttttgctagatacccagatcatatgaaacaacatgacttttttgcaatgttttgctagatactctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatcaatgttttgctagatactttttttgttttttatgtctagaaagcctgtatgcgaagccacaatcctttccaacagaccatactaagt

SEQ ID NO: 94 - DNA, 563 bp, Artificial Sequence
  Nucleotide sequence of CTEC-12 comprising a guide RNA cassette (crRNA) for targeting LbCpf1 to the YFP gene, PAM and guide target sequence and donor DNA on the 5′ side (2 x 20 bp guide), flanked by connector 5 on the 5′ side and connector 3 on the 3′
aagcgacttccaatcgctttgcatatccagtaccacacccacaggcgtttatttgtactactggtaaattgccagttccatggccaaccttagtcactactttaggttatggtgcaatgttttgctagatacccagatcatatgaaacaacatgacttttttgcaatgttttgctagataccctctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatcaatgttttgctagataccctttttttgttttttatgtctagaaagcctgtatgcgaagccacaatcctttccaacagaccatactaagt

SEQ ID NO: 95 - DNA, 27 bp, Artificial Sequence
  Nucleotide sequence of the forward primer to amplify CTEC fragments with connector 5 on the 5′ side
aagcgacttccaatcgctttgcatatc

SEQ ID NO: 96 - DNA, 27 bp, Artificial Sequence
  Nucleotide sequence of the reverse primer to amplify CTEC fragments with connector 3 on the 3′ side
cttagtatggtctgttggaaaggattg

SEQ ID NO: 97 - DNA, 50 bp, Artificial Sequence
  Nucleotide sequence of connector 5
aagcgacttccaatcgctttgcatatccagtaccacacccacaggcgttt

SEQ ID NO: 98 - DNA, 50 bp, Artificial Sequence
  Nucleotide sequence of connector 3
agaaagcctgtatgcgaagccacaatcctttccaacagaccatactaagt

SEQ ID NO: 99 - DNA, 489 bp, Artificial Sequence
  Nucleotide sequence of CTEC-7 comprising a guide RNA cassette (crRNA) for targeting LbCpf1 to the YFP gene and donor DNA on the 3′ side, flanked by connector 5 sequence on the 5′ side
aagcgacttccaatcgctttgcatatccagtaccacacccacaggcgttttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatcaatgttttgctagataccctttttttgttttttatgtctatttgtactactggtaaattgccagttccatggccaaccttagtcactactttaggttatggtgcaatgttttgctagatacccagatcatatgaaacaacatgacttt

SEQ ID NO: 100 - DNA, 539 bp, Artificial Sequence
  Nucleotide sequence of CTEC-8 comprising a guide RNA cassette (crRNA) for targeting LbCpf1 to the YFP gene, connector A and donor DNA on the 3′ side, flanked by connector 5 sequence on the 5′ side
aagcgacttccaatcgctttgcatatccagtaccacacccacaggcgttttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatcaatgttttgctagataccctttttttgttttttatgtctttgcccatcgaacgtacaagtactcctctgttctctccttcctttgctttatttgtactactggtaaattgccagttccatggccaaccttagtcactactttaggttatggtgcaatgttttgctagatacccagatcatatgaaacaacatgacttt

SEQ ID NO: 101 - DNA, 489 bp, Artificial Sequence
  Nucleotide sequence of CTEC-9 comprising a guide RNA cassette (crRNA) for targeting LbCpf1 to the YFP gene and donor DNA on the 5′ side, flanked by connector 5 sequence on the 5′ side
aagcgacttccaatcgctttgcatatccagtaccacacccacaggcgtttatttgtactactggtaaattgccagttccatggccaaccttagtcactactttaggttatggtgcaatgttttgctagatacccagatcatatgaaacaacatgacttttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatcaatgttttgctagataccctttttttgttttttatgtct

SEQ ID NO: 102 - DNA, 539 bp, Artificial Sequence
  Nucleotide sequence of CTEC-10 comprising a guide RNA cassette (crRNA) for targeting LbCpf1 to the YFP gene, connector A and donor DNA on the 5′ side, flanked by connector 5 sequence on the 5′ side
aagcgacttccaatcgctttgcatatccagtaccacacccacaggcgtttatttgtactactggtaaattgccagttccatggccaaccttagtcactactttaggttatggtgcaatgttttgctagatacccagatcatatgaaacaacatgactttttgcccatcgaacgtacaagtactcctctgttctctccttcctttgcttttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatcaatgttttgctagataccctttttttgttttttatgtct

SEQ ID NO: 103 - DNA, 509 bp, Artificial Sequence
  Nucleotide sequence of CTEC-11 comprising a guide RNA cassette (crRNA) for targeting LbCpf1 to the YFP gene, PAM and guide target sequence and donor DNA on the 3′ side (2 x 18 bp guide), flanked by connector 5 sequence on the 5′ side
aagcgacttccaatcgctttgcatatccagtaccacacccacaggcgttttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatcaatgttttgctagatactttttttgttttttatgtcttttgcaatgttttgctagatacatttgtactactggtaaattgccagttccatggccaaccttagtcactactttaggttatggtgcaatgttttgctagatacccagatcatatgaaacaacatgacttt

SEQ ID NO: 104 - DNA, 513 bp, Artificial Sequence
  Nucleotide sequence of CTEC-11 comprising a guide RNA cassette (crRNA) for targeting LbCpf1 to the YFP gene, PAM and guide target sequence and donor DNA on the 3′ side (2 x  20 bp guide), flanked by connector 5 sequence on the 5′ side
aagcgacttccaatcgctttgcatatccagtaccacacccacaggcgttttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatcaatgttttgctagataccctttttttgttttttatgtcttttgcaatgttttgctagatacccatttgtactactggtaaattgccagttccatggccaaccttagtcactactttaggttatggtgcaatgttttgctagatacccagatcatatgaaacaacatgacttt

SEQ ID NO: 105 - DNA, 509 bp, Artificial Sequence
  Nucleotide sequence of CTEC-12 comprising a guide RNA cassette (crRNA) for targeting LbCpf1 to the YFP gene, PAM and guide target sequence and donor DNA on the 5′ side (2 x 18 bp guide), flanked by connector 5 sequence on the 5′ side
aagcgacttccaatcgctttgcatatccagtaccacacccacaggcgtttatttgtactactggtaaattgccagttccatggccaaccttagtcactactttaggttatggtgcaatgttttgctagatacccagatcatatgaaacaacatgacttttttgcaatgttttgctagatactctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatcaatgttttgctagatactttttttgttttttatgtct

SEQ ID NO: 106 - DNA, 513 bp, Artificial Sequence
  Nucleotide sequence of CTEC-12 comprising a guide RNA cassette (crRNA) for targeting LbCpf1 to the YFP gene, PAM and guide target sequence and donor DNA on the 5′ side (2 x 20 bp guide), flanked by connector 5 sequence on the 5′ side
aagcgacttccaatcgctttgcatatccagtaccacacccacaggcgtttatttgtactactggtaaattgccagttccatggccaaccttagtcactactttaggttatggtgcaatgttttgctagatacccagatcatatgaaacaacatgacttttttgcaatgttttgctagataccctctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatcaatgttttgctagataccctttttttgttttttatgtct

SEQ ID NO: 107 - DNA, 489 bp, Artificial Sequence
  Nucleotide sequence of CTEC-7 comprising a guide RNA cassette (crRNA) for targeting LbCpf1 to the YFP gene and donor DNA on the 3′ side, flanked by connector 3 sequence on the 3′ side
tctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatcaatgttttgctagataccctttttttgttttttatgtctatttgtactactggtaaattgccagttccatggccaaccttagtcactactttaggttatggtgcaatgttttgctagatacccagatcatatgaaacaacatgactttagaaagcctgtatgcgaagccacaatcctttccaacagaccatactaagt

SEQ ID NO: 108 - DNA, 539 bp, Artificial Sequence
  Nucleotide sequence of CTEC-8 comprising a guide RNA cassette (crRNA) for targeting LbCpf1 to the YFP gene, connector A and donor DNA on the 3′ side, flanked by connector 3 sequence on the 3′ side
tctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatcaatgttttgctagataccctttttttgttttttatgtctttgcccatcgaacgtacaagtactcctctgttctctccttcctttgctttatttgtactactggtaaattgccagttccatggccaaccttagtcactactttaggttatggtgcaatgttttgctagatacccagatcatatgaaacaacatgactttagaaagcctgtatgcgaagccacaatcctttccaacagaccatactaagt

SEQ ID NO: 109 - DNA, 489 bp, Artificial Sequence
  Nucleotide sequence of CTEC-9 comprising a guide RNA cassette (crRNA) for targeting LbCpf1 to the YFP gene and donor DNA on the 5′ side, flanked by connector 3 sequence on the 3′ side
atttgtactactggtaaattgccagttccatggccaaccttagtcactactttaggttatggtgcaatgttttgctagatacccagatcatatgaaacaacatgacttttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatcaatgttttgctagataccctttttttgttttttatgtctagaaagcctgtatgcgaagccacaatcctttccaacagaccatactaagt

SEQ ID NO: 110 - DNA, 539 bp, Artificial Sequence
  Nucleotide sequence of CTEC-10 comprising a guide RNA cassette (crRNA) for targeting LbCpf1 to the YFP gene, connector A and donor DNA on the 5′ side, flanked by connector 3 sequence on the 3′ side
atttgtactactggtaaattgccagttccatggccaaccttagtcactactttaggttatggtgcaatgttttgctagatacccagatcatatgaaacaacatgactttttgcccatcgaacgtacaagtactcctctgttctctccttcctttgcttttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatcaatgttttgctagataccctttttttgttttttatgtctagaaagcctgtatgcgaagccacaatcctttccaacagaccatactaagt

SEQ ID NO: 111 - DNA, 509 bp, Artificial Sequence
  Nucleotide sequence of CTEC-11 comprising a guide RNA cassette (crRNA) for targeting LbCpf1 to the YFP gene, PAM and guide target sequence and donor DNA on the 3′ side (2 x 18 bp guide), flanked by connector 3 sequence on the 3′ side
tctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatcaatgttttgctagatactttttttgttttttatgtcttttgcaatgttttgctagatacatttgtactactggtaaattgccagttccatggccaaccttagtcactactttaggttatggtgcaatgttttgctagatacccagatcatatgaaacaacatgactttagaaagcctgtatgcgaagccacaatcctttccaacagaccatactaagt

SEQ ID NO: 112 - DNA, 509 bp, Artificial Sequence; identical to SEQ ID NO: 111
  Nucleotide sequence of CTEC-11 comprising a guide RNA cassette (crRNA) for targeting LbCpf1 to the YFP gene, PAM and guide target sequence and donor DNA on the 3′ side (2 x  20 bp guide), flanked by connector 3 sequence on the 3′ side

SEQ ID NO: 113 - DNA, 513 bp, Artificial Sequence
  Nucleotide sequence of CTEC-12 comprising a guide RNA cassette (crRNA) for targeting LbCpf1 to the YFP gene, PAM and guide target sequence and donor DNA on the 5′ side (2 x 18 bp guide), flanked by connector 3 sequence on the 3′ side
tctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatcaatgttttgctagataccctttttttgttttttatgtcttttgcaatgttttgctagatacccatttgtactactggtaaattgccagttccatggccaaccttagtcactactttaggttatggtgcaatgttttgctagatacccagatcatatgaaacaacatgactttagaaagcctgtatgcgaagccacaatcctttccaacagaccatactaagt

SEQ ID NO: 114 - DNA, 513 bp, Artificial Sequence
  Nucleotide sequence of CTEC-12 comprising a guide RNA cassette (crRNA) for targeting LbCpf1 to the YFP gene, PAM and guide target sequence and donor DNA on the 5′ side (2 x 20 bp guide), flanked by connector 3 sequence on the 3′ side
atttgtactactggtaaattgccagttccatggccaaccttagtcactactttaggttatggtgcaatgttttgctagatacccagatcatatgaaacaacatgacttttttgcaatgttttgctagataccctctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctaatttctactaagtgtagatcaatgttttgctagataccctttttttgttttttatgtctagaaagcctgtatgcgaagccacaatcctttccaacagaccatactaagt

SEQ ID NO: 115 - DNA, 598 bp, Artificial Sequence
  Nucleotide sequence of CTEC-1 comprising a guide RNA cassette (sgRNA) for targeting Cas9 to the YFP gene and donor DNA of 60 bp, which encodes a frameshift, on the 3′ side
cggagctagcatgcggccgctctagaactagtggatcccccgggctgcagtctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcttagtcactactttaggttagttttagagctagaaatagcaagttaaaataaggctagtccgttatcaacttgaaaaagtggcaccgagtcggtggtgctttttttgttttttatgtctttccatggccaaccttagtcactactttagttatggtttgcaatgttttgctagatacccgaaaccttcgaatccagccagcatgtcgacacccacaagatgtagtgcacggggggcccggtacccagcttttgttccctttagtgagggttaattccga

SEQ ID NO: 116 - DNA, 618 bp, Artificial Sequence
  Nucleotide sequence of CTEC-1 comprising a guide RNA cassette (sgRNA) for targeting Cas9 to the YFP gene and donor DNA of 80 bp, which encodes a frameshift, on the 3′ side
cggagctagcatgcggccgctctagaactagtggatcccccgggctgcagtctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcttagtcactactttaggttagttttagagctagaaatagcaagttaaaataaggctagtccgttatcaacttgaaaaagtggcaccgagtcggtggtgctttttttgttttttatgtctaaattgccagttccatggccaaccttagtcactactttagttatggtttgcaatgttttgctagatacccagatcatatggaaaccttcgaatccagccagcatgtcgacacccacaagatgtagtgcacggggggcccggtacccagcttttgttccctttagtgagggttaattccga

SEQ ID NO: 117 - DNA, 638 bp, Artificial Sequence
  Nucleotide sequence of CTEC-1 comprising a guide RNA cassette (sgRNA) for targeting Cas9 to the YFP gene and donor DNA of 100 bp, which encodes a frameshift, on the 3′ side
cggagctagcatgcggccgctctagaactagtggatcccccgggctgcagtctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcttagtcactactttaggttagttttagagctagaaatagcaagttaaaataaggctagtccgttatcaacttgaaaaagtggcaccgagtcggtggtgctttttttgttttttatgtcttactactggtaaattgccagttccatggccaaccttagtcactactttagttatggtttgcaatgttttgctagatacccagatcatatgaaacaacatggaaaccttcgaatccagccagcatgtcgacacccacaagatgtagtgcacggggggcccggtacccagcttttgttccctttagtgagggttaattccga

SEQ ID NO: 118 - DNA, 598 bp, Artificial Sequence
  Nucleotide sequence of CTEC-1 comprising a guide RNA cassette (sgRNA) for targeting Cas9 to the YFP gene and donor DNA of 60 bp, which encodes the full knock out of the YFP expression cassette, on the 3′ side
cggagctagcatgcggccgctctagaactagtggatcccccgggctgcagtctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcttagtcactactttaggttagttttagagctagaaatagcaagttaaaataaggctagtccgttatcaacttgaaaaagtggcaccgagtcggtggtgctttttttgttttttatgtctcgtgctgagctcaacagtgcccaacccttgattctttgtcatcagacaacttgttgagtggaaaccttcgaatccagccagcatgtcgacacccacaagatgtagtgcacggggggcccggtacccagcttttgttccctttagtgagggttaattccga

SEQ ID NO: 119 - DNA, 618 bp, Artificial Sequence
  Nucleotide sequence of CTEC-1 comprising a guide RNA cassette (sgRNA) for targeting Cas9 to the YFP gene and donor DNA of 80 bp, which encodes the full knock out of the YFP expression cassette, on the 3′ side
cggagctagcatgcggccgctctagaactagtggatcccccgggctgcagtctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcttagtcactactttaggttagttttagagctagaaatagcaagttaaaataaggctagtccgttatcaacttgaaaaagtggcaccgagtcggtggtgctttttttgttttttatgtctgcaatagttgcgtgctgagctcaacagtgcccaacccttgattctttgtcatcagacaacttgttgagtggtactaaagggaaaccttcgaatccagccagcatgtcgacacccacaagatgtagtgcacggggggcccggtacccagcttttgttccctttagtgagggttaattccga

SEQ ID NO: 120 - DNA, 638 bp, Artificial Sequence
  Nucleotide sequence of CTEC-1 comprising a guide RNA cassette (sgRNA) for targeting Cas9 to the YFP gene and donor DNA of 100 bp, which encodes the full knock out of the YFP expression cassette, on the 3′ side
cggagctagcatgcggccgctctagaactagtggatcccccgggctgcagtctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcttagtcactactttaggttagttttagagctagaaatagcaagttaaaataaggctagtccgttatcaacttgaaaaagtggcaccgagtcggtggtgctttttttgttttttatgtctcttcatgccagcaatagttgcgtgctgagctcaacagtgcccaacccttgattctttgtcatcagacaacttgttgagtggtactaaaggagtgcttttcgaaaccttcgaatccagccagcatgtcgacacccacaagatgtagtgcacggggggcccggtacccagcttttgttccctttagtgagggttaattccga

SEQ ID NO: 121 - DNA, 438 bp, Artificial Sequence
  Nucleotide sequence of the complete guide RNA expression cassette (sgRNA) for targeting Cas9 to the YFP expression cassette in the genome of CSN009
cggagctagcatgcggccgctctagaactagtggatcccccgggctgcagtctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcttagtcactactttaggttagttttagagctagaaatagcaagttaaaataaggctagtccgttatcaacttgaaaaagtggcaccgagtcggtggtgctttttttgttttttatgtct

SEQ ID NO: 122 - DNA, 20 bp, Artificial Sequence
  Nucleotide sequence of the guide sequence (genomic target) of the CTEC fragments targeting YFP by Cas9 in strain CSN009
ttagtcactactttaggtta

SEQ ID NO: 123 - DNA, 60 bp, Artificial Sequence
  Nucleotide sequence of the donor DNA encoding a frameshift in the YFP gene, 60 bp
ttccatggccaaccttagtcactactttagttatggtttgcaatgttttgctagataccc

SEQ ID NO: 124 - DNA, 80 bp, Artificial Sequence
  Nucleotide sequence of the donor DNA encoding a frameshift in the YFP gene, 80 bp
aaattgccagttccatggccaaccttagtcactactttagttatggtttgcaatgttttgctagatacccagatcatatg

SEQ ID NO: 125 - DNA, 100 bp, Artificial Sequence
  Nucleotide sequence of the donor DNA encoding a frameshift in the YFP gene, 100 bp
tactactggtaaattgccagttccatggccaaccttagtcactactttagttatggtttgcaatgttttgctagatacccagatcatatgaaacaacatg

SEQ ID NO: 126 - DNA, 60 bp, Artificial Sequence
  Nucleotide sequence of the donor DNA encoding the knock out of the YFP expression cassette, 60 bp
cgtgctgagctcaacagtgcccaacccttgattctttgtcatcagacaacttgttgagtg

SEQ ID NO: 127 - DNA, 80 bp, Artificial Sequence
  Nucleotide sequence of the donor DNA encoding the knock out of the YFP expression cassette, 80 bp
gcaatagttgcgtgctgagctcaacagtgcccaacccttgattctttgtcatcagacaacttgttgagtggtactaaagg

SEQ ID NO: 128 - DNA, 100 bp, Artificial Sequence
  Nucleotide sequence of the donor DNA encoding the knock out of the YFP expression cassette, 100 bp
cttcatgccagcaatagttgcgtgctgagctcaacagtgcccaacccttgattctttgtcatcagacaacttgttgagtggtactaaaggagtgcttttc

SEQ ID NO: 129 - DNA, 19 bp, Artificial Sequence
  Nucleotide sequence of the forward primer for amplification of CTEC fragments (SEQ ID NO′s: 115, 116, 117, 118, 119 and 120) that are flanked by 50 bp sequences homologous to the linearized pRN1120 vector backbone fragment (EcoRI and XhoI digested)
cggagctagcatgcggccg

SEQ ID NO: 130 - DNA, 23 bp, Artificial Sequence
  Nucleotide sequence of the reverse primer for amplification of CTEC fragments (SEQ ID NO′s: 115, 116, 117, 118, 119 and 120) that are flanked by 50 bp sequences homologous to the linearized pRN1120 vector backbone fragment (EcoRI and XhoI digested)
tcggaattaaccctcactaaagg

SEQ ID NO: 131 - DNA, 50 bp, Artificial Sequence
  Nucleotide sequence of connector F (CONF)
gaaaccttcgaatccagccagcatgtcgacacccacaagatgtagtgcac

SEQ ID NO: 132 - DNA, 20 bp, Artificial Sequence
  Nucleotide sequence of the wild-type genomic target (example 4)
ttagtcactactttaggtta

SEQ ID NO: 133 - DNA, 19 bp, Artificial Sequence
  Nucleotide sequence of the modified genomic target (example 4)
ttagtcactactttagtta

SEQ ID NO: 134 - DNA, 1587 bp, Artificial Sequence
  Nucleotide sequence of CTEC DNA fragment 3, comprising a guide RNA expression cassette (sgRNA) for targeting Cas9 to the GFP gene and donor DNA of 100-bp, which encodes a 2 base modification in the PAM sequence, changing it from CGG to TAG, on the 3′ side
acgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatgcattggggatagcacagggttggggtgtcttgtggactcaatgggtgaaaggagatggaaaagggcggtgaaaagtggtagaatcgaaatccctgacgtcaatttataaagtaaaatgcgtttctgccattttgctcccctccttctttcgcaatcgcctccccaaaagttgtcgtggcagtacacatgcttgcatacaatgaagctaatccggcttgctcagtagttgctatatccaggcatggtgtgaaacccctcaaagtatatataggagcggtgagccccagtctggggtcttttctctccatctcaaaactactttctcacaatggtattgctgatgagtccgtgaggacgaaacgagtaagctcgtccaatacccttaagctcgattgttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggactaaacttcgagctaatccagtagcttacgttacccaggggcaggtcaactggctagccacgagtctgtcccaggtcgcaatttagtgtaataaacaatatatatattgagtctaaagggaattgtagctattgtgattgtgtgattttcgtcttgctggttcttattgtgtcccattcgtttcatcctgatgaggacccctggaaccggtgttttcttagtctctgcaatcgctagtcttgttgctatgacagttgcgtcgacactattcaggtcatctatcggttattctgatattataatatccagcttgtgaccgagaatgttaccatcctccttgaaatcaatacccttaagctcgatttagttaacgagggtatcaccctcaaacttaacctcagctc

SEQ ID NO: 135 - DNA, 1587 bp, Artificial Sequence
  Nucleotide sequence of CTEC DNA fragment 4, comprising a guide RNA expression cassette (sgRNA) for targeting Cas9 to the GFP gene and donor DNA of 100-bp, which encodes a silent mutation in the GFP gene by changing the PAM sequence from CGG to CGA
acgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatgcattggggatagcacagggttggggtgtcttgtggactcaatgggtgaaaggagatggaaaagggcggtgaaaagtggtagaatcgaaatccctgacgtcaatttataaagtaaaatgcgtttctgccattttgctcccctccttctttcgcaatcgcctccccaaaagttgtcgtggcagtacacatgcttgcatacaatgaagctaatccggcttgctcagtagttgctatatccaggcatggtgtgaaacccctcaaagtatatataggagcggtgagccccagtctggggtcttttctctccatctcaaaactactttctcacaatggtattgctgatgagtccgtgaggacgaaacgagtaagctcgtccaatacccttaagctcgattgttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggactaaacttcgagctaatccagtagcttacgttacccaggggcaggtcaactggctagccacgagtctgtcccaggtcgcaatttagtgtaataaacaatatatatattgagtctaaagggaattgtagctattgtgattgtgtgattttcgtcttgctggttcttattgtgtcccattcgtttcatcctgatgaggacccctggaaccggtgttttcttagtctctgcaatcgctagtcttgttgctatgacagttgcgtcgacactattcaggtcatctatcggttattctgatattataatactccagcttgtgaccgagaatgttaccatcctcctagaaatcaatacccttaagctcgattcgattaacgagggtatcaccctcaaacttaacctcagct

SEQ ID NO: 136 - DNA, 973 bp, Artificial Sequence
  Nucleotide sequence of Yarrowia Yl_HYPO promoter
acgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatgcattggggatagcacagggttggggtgtcttgtggactcaatgggtgaaaggagatggaaaagggcggtgaaaagtggtagaatcgaaatccctgacgtcaatttataaagtaaaatgcgtttctgccattttgctcccctccttctttcgcaatcgcctccccaaaagttgtcgtggcagtacacatgcttgcatacaatgaagctaatccggcttgctcagtagttgctatatccaggcatggtgtgaaacccctcaaagtatatataggagcggtgagccccagtctggggtcttttctctccatctcaaaactactttctcacaatg

SEQ ID NO: 137 - DNA, 6 bp, Artificial Sequence
  Nucleotide sequence of the 6-bp inverted repeat of the guide sequence of the GFP gene
gtattg

SEQ ID NO: 138 - DNA, 37 bp, Artificial Sequence
  Nucleotide sequence of the HH ribozyme
ctgatgagtccgtgaggacgaaacgagtaagctcgtc

SEQ ID NO: 139 - DNA, 148 bp, Artificial Sequence
  Nucleotide sequence of the HDV ribozyme
gttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggac

SEQ ID NO: 140 - DNA, 20 bp, Artificial Sequence
  Nucleotide sequence of the 20-bp genomic target sequence of the GFP gene
caatacccttaagctcgatt

SEQ ID NO: 141 - DNA, 303 bp, Artificial Sequence
  Nucleotide sequence of the Yarrowia Yl_PGM terminator
taaacttcgagctaatccagtagcttacgttacccaggggcaggtcaactggctagccacgagtctgtcccaggtcgcaatttagtgtaataaacaatatatatattgagtctaaagggaattgtagctattgtgattgtgtgattttcgtcttgctggttcttattgtgtcccattcgtttcatcctgatgaggacccctggaaccggtgttttcttagtctctgcaatcgctagtcttgttgctatgacagttgcgtcgacactattcaggtcatctatcggttattctgatattataata

SEQ ID NO: 142 - DNA, 1487 bp, Artificial Sequence
  Nucleotide sequence of guide-RNA expression cassette (sgRNA) targeting the GFP gene
acgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatgcattggggatagcacagggttggggtgtcttgtggactcaatgggtgaaaggagatggaaaagggcggtgaaaagtggtagaatcgaaatccctgacgtcaatttataaagtaaaatgcgtttctgccattttgctcccctccttctttcgcaatcgcctccccaaaagttgtcgtggcagtacacatgcttgcatacaatgaagctaatccggcttgctcagtagttgctatatccaggcatggtgtgaaacccctcaaagtatatataggagcggtgagccccagtctggggtcttttctctccatctcaaaactactttctcacaatggtattgctgatgagtccgtgaggacgaaacgagtaagctcgtccaatacccttaagctcgattgttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggactaaacttcgagctaatccagtagcttacgttacccaggggcaggtcaactggctagccacgagtctgtcccaggtcgcaatttagtgtaataaacaatatatatattgagtctaaagggaattgtagctattgtgattgtgtgattttcgtcttgctggttcttattgtgtcccattcgtttcatcctgatgaggacccctggaaccggtgttttcttagtctctgcaatcgctagtcttgttgctatgacagttgcgtcgacactattcaggtcatctatcggttattctgatattataata

SEQ ID NO: 143 - DNA, 100 bp, Artificial Sequence
  Nucleotide sequence of 100-bp donor DNA of CTEC DNA fragment 1
gggaaacatgtcctggacttacaacttgcttcgctcttgatcttcggatagtagtataagtgtgtgtgttggtgctaataatccgtcctctccacccctt

SEQ ID NO: 144 - DNA, 100 bp, Artificial Sequence
  Nucleotide sequence of 100-bp donor DNA of CTEC DNA fragment 2
tccagcttgtgaccgagaatgttaccatcctccttgaaatcaatacccttaagctcgattcgttaacgagggtatcaccctcaaacttaacctcagctcg

SEQ ID NO: 145 - DNA, 100 bp, Artificial Sequence
  Nucleotide sequence of 100-bp donor DNA of CTEC DNA fragment 3
tccagcttgtgaccgagaatgttaccatcctccttgaaatcaatacccttaagctcgatttagttaacgagggtatcaccctcaaacttaacctcagctc

SEQ ID NO: 146 - DNA, 100 bp, Artificial Sequence
  Nucleotide sequence of 100-bp donor DNA of CTEC DNA fragment 4
ctccagcttgtgaccgagaatgttaccatcctcctagaaatcaatacccttaagctcgattcgattaacgagggtatcaccctcaaacttaacctcagct

SEQ ID NO: 147 - DNA, 11606 bp, Artificial Sequence
  Nucleotide sequence of plasmid MB7452
cgcgtggatcgccggtgcgttgacgttggtgacctccagccagaggtgcccggcgccccgctcgccggcgaactccgtcgcgagccccatcaacgcgcgcccgaccccgtgcccccggtgctccggggcgacctcgatgtcctcgacggtcagccggcggttccacgccgagtacgagatgaccacgaagcccgccaggtcgccgtcgtccccgtacgcgacgaacgtccgggagtccgggtcgccgtcctccccgtcgtccgattcgtcgtccgattcgtcgtcggggaacaccttggtcaggggcgggtccaccggcacctcccgcagggtgaagccgtccccggtggcggtgacgcggaagacggtgtcggtggtgaaggacccatccagtgcctcgatggcctcggcgtcccccgggacactggtgcggtaccggtaagccgtgtcgtcaagagtggtcatttttgtgtctaggtgtttgtgtttggactgcgatcagtgaagaaaagaagaggaaaaattgtgcaagaaattttgctttcaagacttggctgatgcagcagggtaactctgggacacagacctatgtttgtggttaaactcaatgcacgtggtacgtgcgtggagcgcttacccatccaagggtgtggacatggaaccgacggtccgtggagttgtgtaatgtcattttggcgactcttgaagcaaggctataaaaaaattgtgtggcttgagtcttatcgagctcggtcactacaagagttaatcttcctgtctcaggcagacaggtcaggcagggttacttttgggtgtgctgtaactcactgtatggccgttagtgcgcatagacgttgtacatactggaccgaattgtagcgtgctcaatagggccaataaagctattgtagggatccgaattttcagaacctaatttatctgttacccggcctgtggctcgcacagcttaaaaatggtcaaactttccccttcttgtctttttttcctcacattcatcaggttcttgtcttgatctttcaagtgagtattaattaccgaccttggttcttcattgggagagcattggaagccgtggtgcagcaaccacaaaacggttcttccccttcgataccttcttgcctgcctttcaatacaagtcggctcgattagcggtggtcgcccccgccagcggagaacatggaactaacccagaatgagagctaagtggagaaagaagagagtcagacgactcaagcgaaagcgccgcaaggtccgagctcgatccaaataagcggtttttaacggagatttaacactaaatcgaagaacttttcccgtttcatttgcgaatgagctcgttaacaaaatcccccagtttttttatccagctgtaaggattgacattagtaatgaattattgtttggtatatttaaatctgtagttcctttctgtccgtgtcggcaactgtcgtactcgtgatttacttgtattgacgaatacttactgtagcgcactctgctgctactggtcgtaaggatgtgctatttcggtgtatggtgggttttttgggggtcggaaccgaagactgttacacgggcacggctcgttgtgtacacgcacagagctcttgcgagtcatgttgtagctagctcgtcgtgttcaggaactgttcgatggttcggagagagtcgccgcccagaacatacgcgcaccgatgtcagcagacagccttattacaagtatattcaagcaagtatatccgtagggtgcgggtgatttggatctaaggttcgtactcaacactcacgagcagcttgcctatgttacatccttttatcagacataacataattggagtttacttacacacggggtgtacctgtatgagcaccacctacaattgtagcactggtacttgtacaaagaatttattcgtacgaatcacagggacggccgccctcaccgaaccagcgaatacctcagcggtcccctgcagtgactcaacaaagcgatatgaacatcttgcgatggtatcctgctgatagtttttactgtacaaacacctgtgtagctccttctagcatttttaagttattcacacctcaaggggagggataaattaaataaattccaaaagcgaagatcgagaaactaaattaaaattccaaaaacgaagttggaacacaaccccccgaaaaaaaacaacaaacaaaaaacccaacaaaataaacaaaaacaaaataaatatataactaccagtatctgactaaaagttcaaatactcgtacttacaacaaatagaaatgagccggccaaaattctgcagaaaaaaatttcaaacaagtactggtataattaaattaaaaaacacatcaaagtatcataacgttagttattttattttatttaataaaagaaaacaacaagatgggctcaaaactttcaacttatacgatacataccaaataacaatttagtatttatctaagtgcttttcgtagataatggaatacaaatggatatccagagtatacacatggatagtatacactgacacgacaattctgtatctctttatgttaactactgtgaggcattaaatagagcttgatatataaaatgttacatttcacagtctgaacttttgcagattacctaatttggtaagatattaattatgaactgaaagttgatggcatccctaaatttgatgaaagatgaaattgtaaatgaggtggtaaaagagctacagtcgttttgttttgagataccatcatctctaacgaaatatctattaaaaatctcagtgtgatcatgagtcattgccatcctggaaaatgtcatcatggctgatatttctaactgtttacttgagataaatatatatttacaagaacttcccttgaaattaatttagatataaaatgtttgcgggcaagttactacgaggaataaattatatctagaggttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctggcctaggaagcgacttccaatcgctttgcatatccagtaccacacccacaggcgtttgtgctactctactgatagcaatagatgcgtcataattggttggcccgctgagcctccacaggatactattgcacataccctggtcatgtgcagatcagctcatttgtggagactctggagtaacttagacgacgcctggttcaattgccgcaatgtgcgcccacgcagataatgtattgaggggtggagcgcctcttggggacttgctgtacttgtacgggatattaaacgcactcagcaagaccatgacgtaaaacacacctactgtacgatacgtactgtaggtattgtactcgtacccggtactacaaatagtacgatactatacggagtgtatttgtaccttgatatacgactggcggagtgaagagaaggagttgaacaagaccagatggggatatcagccccagtgctttgtattacaagtacgagtacttaatagatactgtaaggctattgatacggatggcagtaagtcattgagtaagcaattgtggcccagcatctcccctacgtacttgtaccataccccatggagacaccaatggtctttcacgcacactgtcgtgtgctgtatcgcagaatcgggtgtccaaccaaatgccgttacccccacgtcacagccgatagacagatacaccatcaataccagcaggttgtatcatgcggttggctgaaggtaagctgattggtctaaaaactgtagctgtcctaattcaacgagcgctatttggggccaaccacctcggccaagcggcctttaatctgcgtgccccagaggcgtctaatgaggctctggccgccactgtaggagtgtttctctgtgcgcacacgcagttttgagtttgggcgactttccctttttcccaattgcgtacacacacagctccgagctaagcgctgtccttgaaccttctccctcttttccctctttttctcttccccttcccctcctccacattaaggccaaatcctgaattgcaccaactagtacaacgacaacaatggacaagaagtactccatcggtttggacattggtactaactctgtcggctgggccgtcatcaccgacgagtacaaggttccctccaagaagttcaaggtccttggcaacaccgaccgacactctatcaagaagaacctgatcggtgctctgctgttcgactctggcgagactgccgaggccacccgactgaagcgaaccgctcgacgccgatacacccgacgaaagaaccgaatctgttacctccaggagatcttcagcaacgagatggctaaggtcgacgactccttcttccaccgactcgaggagtctttcctggtcgaagaggataagaagcacgagcgacaccccatcttcggcaacattgttgatgaggttgcctaccatgagaagtaccccaccatctaccacctccgaaagaagctcgtcgactccactgacaaggctgacctccgactcatctaccttgctctcgcccacatgatcaagttccgaggtcacttcctcattgagggtgatctcaaccccgacaactccgacgttgacaagctgttcatccagctcgtccagacctacaaccagctctttgaggagaaccctatcaacgcttctggtgttgacgccaaggccattctctccgcccgactctctaagtcccgacgactcgagaacctcattgcccagctgcccggcgagaagaagaacggcctcttcggtaacctgattgctctctctcttggtctgacccccaacttcaagtccaactttgacctcgccgaggacgccaagctccagctgtccaaggacacctacgatgacgatctggacaacctcctggcccagatcggtgaccagtacgccgatctcttccttgccgccaagaacctctccgacgccatcctgctctccgacatcctccgagtcaacaccgagattaccaaggctcctctgtctgcctctatgatcaagcgatacgacgagcaccaccaggatctcactcttctcaaggctctcgtccgacagcagctccccgagaagtacaaggagattttctttgaccagtccaagaacggttacgctggctacattgacggtggtgcttcccaggaagagttttacaagttcatcaagcctattctggagaagatggacggtaccgaggagctgctcgtcaagctcaaccgagaggacctccttcgaaagcagcgaaccttcgataacggctccatcccccaccagatccacctgggtgagctccacgccattctccgaagacaagaggacttctaccccttcctaaaggataaccgagagaagatcgagaagattctcaccttccgaatcccctactacgtcggtcccctcgctcgaggtaactcccgatttgcttggatgacccgaaagtccgaggagactatcaccccctggaactttgaagaggtagtcgacaagggtgcctccgcccagtctttcattgagcggatgaccaacttcgataagaacctccccaacgagaaggtccttcccaagcactctctcctctacgagtacttcaccgtctacaacgagctgaccaaggtcaagtacgttaccgagggcatgcgaaagcccgctttcctctctggtgagcagaagaaggccattgtcgacctcctgttcaagactaaccgaaaagtcaccgtcaagcagctcaaggaagactacttcaagaagattgagtgcttcgactccgtcgagatttccggtgtcgaggaccgattcaacgcctccctcggcacctaccacgatcttctgaagatcatcaaggacaaggactttcttgataacgaggagaacgaggacattctcgaggacatcgtcctcaccctcacccttttcgaggatcgagagatgatcgaggagcgactcaagacctacgcccatctcttcgacgacaaggtcatgaagcaactcaagcgacgacgatacactggctggggccgactttcccgaaagctcatcaacggcatccgagacaagcagtctggcaagaccatcctggacttcctgaagtccgacggtttcgccaaccgaaacttcatgcagctcatccacgacgactctcttaccttcaaagaggatatccagaaggcccaggtttctggccagggcgactccctccacgagcacattgccaacctcgccggatcccccgccatcaaaaagggtatcctccagaccgtcaaggttgtcgacgaactcgtgaaggtcatgggccgacacaagcccgagaacatcgttatcgagatggcccgagagaaccagaccacccagaagggtcagaagaactcccgagagcgaatgaagcgaatcgaagagggtatcaaggagctcggttcccagattctcaaggagcaccccgtcgagaacacccagctccagaacgagaaactctacctgtactacctccagaatggccgagacatgtacgttgaccaggagctcgacatcaaccgactctccgactacgacgtcgaccacattgttcctcagtccttcctcaaggacgactccatcgacaacaaggttctgacccgatctgacaagaaccgaggtaagtccgacaacgttccctccgaagaggtcgttaagaagatgaagaactactggcgacagcttctcaacgccaaactgatcacccagcgaaagtttgacaacctcaccaaggccgagcgaggtggtctgtccgagctggacaaggccggcttcattaagcgacagctggtcgagactcgacagatcaccaagcacgtcgcccagatcctcgactcccgaatgaacaccaagtacgacgagaacgacaagctcatccgggaggtcaaggtcatcaccctgaagtctaagcttgtctccgacttccgaaaggacttccagttctacaaggtccgagagatcaacaactaccaccacgcccacgacgcctacctcaacgccgttgttggtaccgccctcatcaagaagtatcccaagctcgagtccgagttcgtttacggcgactacaaggtttacgatgtccgaaagatgattgccaagtccgagcaggagatcggtaaggccaccgccaagtactttttctactccaacatcatgaatttcttcaagaccgagatcactctcgccaacggtgagattcgaaagcgacccctgattgagactaatggtgagactggtgagatcgtctgggataagggccgagacttcgccaccgtccgaaaggtcctgtccatgccccaggtcaacattgtcaagaagaccgaggtccagaccggtggcttctccaaggagtccattctccccaagcgaaactccgacaaactcatcgcccgtaagaaggactgggatccgaagaagtacggtggtttcgattctcccaccgttgcctactccgtcctcgttgttgctaaagtcgagaagggtaagtctaagaaactcaagtccgtgaaggagctactcggtatcaccatcatggagcgatcttcttttgagaagaaccccattgacttcctcgaggccaagggttacaaagaggtcaagaaggacctgattatcaagctgcccaagtactccctctttgagctcgagaacggccgaaagcgaatgctggcttccgctggtgagctgcagaagggcaacgagctcgctctgccctccaagtacgtcaacttcctctacctggcctcccactacgagaagctcaagggctcccccgaggacaacgagcagaagcagctgttcgttgagcagcacaagcactacctcgacgagatcatcgagcagatctccgagttctccaagcgagtcatcctcgctgacgccaaccttgataaggttctctctgcttacaacaagcaccgggacaagcccatccgagagcaggccgagaatatcatccacctcttcactctcaccaacctcggcgctcctgctgccttcaagtacttcgacaccaccattgaccgaaagaggtacacctccaccaaggaagtcctcgacgccaccctgatccaccagtccatcaccggcctctacgaaacccgaatcgacctctcccagctcggcggtgactctcgagccgaccccaagaagaagcgaaaagtctaaatatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatttgatatatatactaattacaatagtacatattagaacatacaatagttagtgccgtgaagtggcttaaaataccgcgagtcgattacgtaatattattacctcttgcccatcgaacgtacaagtactcctctgttctctccttcctttgctttgtgcacgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatggccgccagtgtgctggaattgaatatttaccgttcgtataatgtatgctatacgaagttataccggtctcgtagtgttcacgttcagttcacggtgagcttaaaactatcttcaagaagagatttgagacctgatttatacttgcagcaatgtttacttcttatcgcgatacacgaatgtgatacggatcaaagtaagcaggactacgataagataacgaatgcggtgcagtccatgtcgattaggtatagatacatttattttgtgttatgttacattttggggggatactgtcctacttgtagtacctacttgtagtggcgcgttaggggcagggcatgctcatgtagagcgcctgccgctcgccgtccgaggcggtgccgtcgtacagggcggtgtccaggccgcagagggtgaaccccatccgccggta

SEQ ID NO: 148 - DNA, 5444 bp, Artificial Sequence
  Nucleotide sequence of Cas9, including a C-terminal SV40 nuclear localization signal, codon optimized for expression in Yarrowia lipolytica
gtgctactctactgatagcaatagatgcgtcataattggttggcccgctgagcctccacaggatactattgcacataccctggtcatgtgcagatcagctcatttgtggagactctggagtaacttagacgacgcctggttcaattgccgcaatgtgcgcccacgcagataatgtattgaggggtggagcgcctcttggggacttgctgtacttgtacgggatattaaacgcactcagcaagaccatgacgtaaaacacacctactgtacgatacgtactgtaggtattgtactcgtacccggtactacaaatagtacgatactatacggagtgtatttgtaccttgatatacgactggcggagtgaagagaaggagttgaacaagaccagatggggatatcagccccagtgctttgtattacaagtacgagtacttaatagatactgtaaggctattgatacggatggcagtaagtcattgagtaagcaattgtggcccagcatctcccctacgtacttgtaccataccccatggagacaccaatggtctttcacgcacactgtcgtgtgctgtatcgcagaatcgggtgtccaaccaaatgccgttacccccacgtcacagccgatagacagatacaccatcaataccagcaggttgtatcatgcggttggctgaaggtaagctgattggtctaaaaactgtagctgtcctaattcaacgagcgctatttggggccaaccacctcggccaagcggcctttaatctgcgtgccccagaggcgtctaatgaggctctggccgccactgtaggagtgtttctctgtgcgcacacgcagttttgagtttgggcgactttccctttttcccaattgcgtacacacacagctccgagctaagcgctgtccttgaaccttctccctcttttccctctttttctcttccccttcccctcctccacattaaggccaaatcctgaattgcaccaactagtacaacgacaacaatggacaagaagtactccatcggtttggacattggtactaactctgtcggctgggccgtcatcaccgacgagtacaaggttccctccaagaagttcaaggtccttggcaacaccgaccgacactctatcaagaagaacctgatcggtgctctgctgttcgactctggcgagactgccgaggccacccgactgaagcgaaccgctcgacgccgatacacccgacgaaagaaccgaatctgttacctccaggagatcttcagcaacgagatggctaaggtcgacgactccttcttccaccgactcgaggagtctttcctggtcgaagaggataagaagcacgagcgacaccccatcttcggcaacattgttgatgaggttgcctaccatgagaagtaccccaccatctaccacctccgaaagaagctcgtcgactccactgacaaggctgacctccgactcatctaccttgctctcgcccacatgatcaagttccgaggtcacttcctcattgagggtgatctcaaccccgacaactccgacgttgacaagctgttcatccagctcgtccagacctacaaccagctctttgaggagaaccctatcaacgcttctggtgttgacgccaaggccattctctccgcccgactctctaagtcccgacgactcgagaacctcattgcccagctgcccggcgagaagaagaacggcctcttcggtaacctgattgctctctctcttggtctgacccccaacttcaagtccaactttgacctcgccgaggacgccaagctccagctgtccaaggacacctacgatgacgatctggacaacctcctggcccagatcggtgaccagtacgccgatctcttccttgccgccaagaacctctccgacgccatcctgctctccgacatcctccgagtcaacaccgagattaccaaggctcctctgtctgcctctatgatcaagcgatacgacgagcaccaccaggatctcactcttctcaaggctctcgtccgacagcagctccccgagaagtacaaggagattttctttgaccagtccaagaacggttacgctggctacattgacggtggtgcttcccaggaagagttttacaagttcatcaagcctattctggagaagatggacggtaccgaggagctgctcgtcaagctcaaccgagaggacctccttcgaaagcagcgaaccttcgataacggctccatcccccaccagatccacctgggtgagctccacgccattctccgaagacaagaggacttctaccccttcctaaaggataaccgagagaagatcgagaagattctcaccttccgaatcccctactacgtcggtcccctcgctcgaggtaactcccgatttgcttggatgacccgaaagtccgaggagactatcaccccctggaactttgaagaggtagtcgacaagggtgcctccgcccagtctttcattgagcggatgaccaacttcgataagaacctccccaacgagaaggtccttcccaagcactctctcctctacgagtacttcaccgtctacaacgagctgaccaaggtcaagtacgttaccgagggcatgcgaaagcccgctttcctctctggtgagcagaagaaggccattgtcgacctcctgttcaagactaaccgaaaagtcaccgtcaagcagctcaaggaagactacttcaagaagattgagtgcttcgactccgtcgagatttccggtgtcgaggaccgattcaacgcctccctcggcacctaccacgatcttctgaagatcatcaaggacaaggactttcttgataacgaggagaacgaggacattctcgaggacatcgtcctcaccctcacccttttcgaggatcgagagatgatcgaggagcgactcaagacctacgcccatctcttcgacgacaaggtcatgaagcaactcaagcgacgacgatacactggctggggccgactttcccgaaagctcatcaacggcatccgagacaagcagtctggcaagaccatcctggacttcctgaagtccgacggtttcgccaaccgaaacttcatgcagctcatccacgacgactctcttaccttcaaagaggatatccagaaggcccaggtttctggccagggcgactccctccacgagcacattgccaacctcgccggatcccccgccatcaaaaagggtatcctccagaccgtcaaggttgtcgacgaactcgtgaaggtcatgggccgacacaagcccgagaacatcgttatcgagatggcccgagagaaccagaccacccagaagggtcagaagaactcccgagagcgaatgaagcgaatcgaagagggtatcaaggagctcggttcccagattctcaaggagcaccccgtcgagaacacccagctccagaacgagaaactctacctgtactacctccagaatggccgagacatgtacgttgaccaggagctcgacatcaaccgactctccgactacgacgtcgaccacattgttcctcagtccttcctcaaggacgactccatcgacaacaaggttctgacccgatctgacaagaaccgaggtaagtccgacaacgttccctccgaagaggtcgttaagaagatgaagaactactggcgacagcttctcaacgccaaactgatcacccagcgaaagtttgacaacctcaccaaggccgagcgaggtggtctgtccgagctggacaaggccggcttcattaagcgacagctggtcgagactcgacagatcaccaagcacgtcgcccagatcctcgactcccgaatgaacaccaagtacgacgagaacgacaagctcatccgggaggtcaaggtcatcaccctgaagtctaagcttgtctccgacttccgaaaggacttccagttctacaaggtccgagagatcaacaactaccaccacgcccacgacgcctacctcaacgccgttgttggtaccgccctcatcaagaagtatcccaagctcgagtccgagttcgtttacggcgactacaaggtttacgatgtccgaaagatgattgccaagtccgagcaggagatcggtaaggccaccgccaagtactttttctactccaacatcatgaatttcttcaagaccgagatcactctcgccaacggtgagattcgaaagcgacccctgattgagactaatggtgagactggtgagatcgtctgggataagggccgagacttcgccaccgtccgaaaggtcctgtccatgccccaggtcaacattgtcaagaagaccgaggtccagaccggtggcttctccaaggagtccattctccccaagcgaaactccgacaaactcatcgcccgtaagaaggactgggatccgaagaagtacggtggtttcgattctcccaccgttgcctactccgtcctcgttgttgctaaagtcgagaagggtaagtctaagaaactcaagtccgtgaaggagctactcggtatcaccatcatggagcgatcttcttttgagaagaaccccattgacttcctcgaggccaagggttacaaagaggtcaagaaggacctgattatcaagctgcccaagtactccctctttgagctcgagaacggccgaaagcgaatgctggcttccgctggtgagctgcagaagggcaacgagctcgctctgccctccaagtacgtcaacttcctctacctggcctcccactacgagaagctcaagggctcccccgaggacaacgagcagaagcagctgttcgttgagcagcacaagcactacctcgacgagatcatcgagcagatctccgagttctccaagcgagtcatcctcgctgacgccaaccttgataaggttctctctgcttacaacaagcaccgggacaagcccatccgagagcaggccgagaatatcatccacctcttcactctcaccaacctcggcgctcctgctgccttcaagtacttcgacaccaccattgaccgaaagaggtacacctccaccaaggaagtcctcgacgccaccctgatccaccagtccatcaccggcctctacgaaacccgaatcgacctctcccagctcggcggtgactctcgagccgaccccaagaagaagcgaaaagtctaaatatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatttgatatatatactaattacaatagtacatattagaacatacaatagttagtgccgtgaagtggcttaaaataccgcgagtcgattacgtaatattatta

SEQ ID NO: 149 - DNA, 1004 bp, Artificial Sequence
  Nucleotide sequence of Yarrowia Yl_007 promoter
gtgctactctactgatagcaatagatgcgtcataattggttggcccgctgagcctccacaggatactattgcacataccctggtcatgtgcagatcagctcatttgtggagactctggagtaacttagacgacgcctggttcaattgccgcaatgtgcgcccacgcagataatgtattgaggggtggagcgcctcttggggacttgctgtacttgtacgggatattaaacgcactcagcaagaccatgacgtaaaacacacctactgtacgatacgtactgtaggtattgtactcgtacccggtactacaaatagtacgatactatacggagtgtatttgtaccttgatatacgactggcggagtgaagagaaggagttgaacaagaccagatggggatatcagccccagtgctttgtattacaagtacgagtacttaatagatactgtaaggctattgatacggatggcagtaagtcattgagtaagcaattgtggcccagcatctcccctacgtacttgtaccataccccatggagacaccaatggtctttcacgcacactgtcgtgtgctgtatcgcagaatcgggtgtccaaccaaatgccgttacccccacgtcacagccgatagacagatacaccatcaataccagcaggttgtatcatgcggttggctgaaggtaagctgattggtctaaaaactgtagctgtcctaattcaacgagcgctatttggggccaaccacctcggccaagcggcctttaatctgcgtgccccagaggcgtctaatgaggctctggccgccactgtaggagtgtttctctgtgcgcacacgcagttttgagtttgggcgactttccctttttcccaattgcgtacacacacagctccgagctaagcgctgtccttgaaccttctccctcttttccctctttttctcttccccttcccctcctccacattaaggccaaatcctgaattgcaccaactagtacaacgacaaca

SEQ ID NO: 150 - DNA, 300 bp, Artificial Sequence
  Nucleotide sequence of Yarrowia Yl_GPD terminator
atatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatttgatatatatactaattacaatagtacatattagaacatacaatagttagtgccgtgaagtggcttaaaataccgcgagtcgattacgtaatattatta

SEQ ID NO: 151 - DNA, 12810 bp, Artificial Sequence
  Nucleotide sequence of pSTV089
ggttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctggcctaggaagcgacttccaatcgctttgcatatccagtaccacacccacaggcgtttgtgctactctactgatagcaatagatgcgtcataattggttggcccgctgagcctccacaggatactattgcacataccctggtcatgtgcagatcagctcatttgtggagactctggagtaacttagacgacgcctggttcaattgccgcaatgtgcgcccacgcagataatgtattgaggggtggagcgcctcttggggacttgctgtacttgtacgggatattaaacgcactcagcaagaccatgacgtaaaacacacctactgtacgatacgtactgtaggtattgtactcgtacccggtactacaaatagtacgatactatacggagtgtatttgtaccttgatatacgactggcggagtgaagagaaggagttgaacaagaccagatggggatatcagccccagtgctttgtattacaagtacgagtacttaatagatactgtaaggctattgatacggatggcagtaagtcattgagtaagcaattgtggcccagcatctcccctacgtacttgtaccataccccatggagacaccaatggtctttcacgcacactgtcgtgtgctgtatcgcagaatcgggtgtccaaccaaatgccgttacccccacgtcacagccgatagacagatacaccatcaataccagcaggttgtatcatgcggttggctgaaggtaagctgattggtctaaaaactgtagctgtcctaattcaacgagcgctatttggggccaaccacctcggccaagcggcctttaatctgcgtgccccagaggcgtctaatgaggctctggccgccactgtaggagtgtttctctgtgcgcacacgcagttttgagtttgggcgactttccctttttcccaattgcgtacacacacagctccgagctaagcgctgtccttgaaccttctccctcttttccctctttttctcttccccttcccctcctccacattaaggccaaatcctgaattgcaccaactagtacaacgacaacaatggacaagaagtactccatcggtttggacattggtactaactctgtcggctgggccgtcatcaccgacgagtacaaggttccctccaagaagttcaaggtccttggcaacaccgaccgacactctatcaagaagaacctgatcggtgctctgctgttcgactctggcgagactgccgaggccacccgactgaagcgaaccgctcgacgccgatacacccgacgaaagaaccgaatctgttacctccaggagatcttcagcaacgagatggctaaggtcgacgactccttcttccaccgactcgaggagtctttcctggtcgaagaggataagaagcacgagcgacaccccatcttcggcaacattgttgatgaggttgcctaccatgagaagtaccccaccatctaccacctccgaaagaagctcgtcgactccactgacaaggctgacctccgactcatctaccttgctctcgcccacatgatcaagttccgaggtcacttcctcattgagggtgatctcaaccccgacaactccgacgttgacaagctgttcatccagctcgtccagacctacaaccagctctttgaggagaaccctatcaacgcttctggtgttgacgccaaggccattctctccgcccgactctctaagtcccgacgactcgagaacctcattgcccagctgcccggcgagaagaagaacggcctcttcggtaacctgattgctctctctcttggtctgacccccaacttcaagtccaactttgacctcgccgaggacgccaagctccagctgtccaaggacacctacgatgacgatctggacaacctcctggcccagatcggtgaccagtacgccgatctcttccttgccgccaagaacctctccgacgccatcctgctctccgacatcctccgagtcaacaccgagattaccaaggctcctctgtctgcctctatgatcaagcgatacgacgagcaccaccaggatctcactcttctcaaggctctcgtccgacagcagctccccgagaagtacaaggagattttctttgaccagtccaagaacggttacgctggctacattgacggtggtgcttcccaggaagagttttacaagttcatcaagcctattctggagaagatggacggtaccgaggagctgctcgtcaagctcaaccgagaggacctccttcgaaagcagcgaaccttcgataacggctccatcccccaccagatccacctgggtgagctccacgccattctccgaagacaagaggacttctaccccttcctaaaggataaccgagagaagatcgagaagattctcaccttccgaatcccctactacgtcggtcccctcgctcgaggtaactcccgatttgcttggatgacccgaaagtccgaggagactatcaccccctggaactttgaagaggtagtcgacaagggtgcctccgcccagtctttcattgagcggatgaccaacttcgataagaacctccccaacgagaaggtccttcccaagcactctctcctctacgagtacttcaccgtctacaacgagctgaccaaggtcaagtacgttaccgagggcatgcgaaagcccgctttcctctctggtgagcagaagaaggccattgtcgacctcctgttcaagactaaccgaaaagtcaccgtcaagcagctcaaggaagactacttcaagaagattgagtgcttcgactccgtcgagatttccggtgtcgaggaccgattcaacgcctccctcggcacctaccacgatcttctgaagatcatcaaggacaaggactttcttgataacgaggagaacgaggacattctcgaggacatcgtcctcaccctcacccttttcgaggatcgagagatgatcgaggagcgactcaagacctacgcccatctcttcgacgacaaggtcatgaagcaactcaagcgacgacgatacactggctggggccgactttcccgaaagctcatcaacggcatccgagacaagcagtctggcaagaccatcctggacttcctgaagtccgacggtttcgccaaccgaaacttcatgcagctcatccacgacgactctcttaccttcaaagaggatatccagaaggcccaggtttctggccagggcgactccctccacgagcacattgccaacctcgccggatcccccgccatcaaaaagggtatcctccagaccgtcaaggttgtcgacgaactcgtgaaggtcatgggccgacacaagcccgagaacatcgttatcgagatggcccgagagaaccagaccacccagaagggtcagaagaactcccgagagcgaatgaagcgaatcgaagagggtatcaaggagctcggttcccagattctcaaggagcaccccgtcgagaacacccagctccagaacgagaaactctacctgtactacctccagaatggccgagacatgtacgttgaccaggagctcgacatcaaccgactctccgactacgacgtcgaccacattgttcctcagtccttcctcaaggacgactccatcgacaacaaggttctgacccgatctgacaagaaccgaggtaagtccgacaacgttccctccgaagaggtcgttaagaagatgaagaactactggcgacagcttctcaacgccaaactgatcacccagcgaaagtttgacaacctcaccaaggccgagcgaggtggtctgtccgagctggacaaggccggcttcattaagcgacagctggtcgagactcgacagatcaccaagcacgtcgcccagatcctcgactcccgaatgaacaccaagtacgacgagaacgacaagctcatccgggaggtcaaggtcatcaccctgaagtctaagcttgtctccgacttccgaaaggacttccagttctacaaggtccgagagatcaacaactaccaccacgcccacgacgcctacctcaacgccgttgttggtaccgccctcatcaagaagtatcccaagctcgagtccgagttcgtttacggcgactacaaggtttacgatgtccgaaagatgattgccaagtccgagcaggagatcggtaaggccaccgccaagtactttttctactccaacatcatgaatttcttcaagaccgagatcactctcgccaacggtgagattcgaaagcgacccctgattgagactaatggtgagactggtgagatcgtctgggataagggccgagacttcgccaccgtccgaaaggtcctgtccatgccccaggtcaacattgtcaagaagaccgaggtccagaccggtggcttctccaaggagtccattctccccaagcgaaactccgacaaactcatcgcccgtaagaaggactgggatccgaagaagtacggtggtttcgattctcccaccgttgcctactccgtcctcgttgttgctaaagtcgagaagggtaagtctaagaaactcaagtccgtgaaggagctactcggtatcaccatcatggagcgatcttcttttgagaagaaccccattgacttcctcgaggccaagggttacaaagaggtcaagaaggacctgattatcaagctgcccaagtactccctctttgagctcgagaacggccgaaagcgaatgctggcttccgctggtgagctgcagaagggcaacgagctcgctctgccctccaagtacgtcaacttcctctacctggcctcccactacgagaagctcaagggctcccccgaggacaacgagcagaagcagctgttcgttgagcagcacaagcactacctcgacgagatcatcgagcagatctccgagttctccaagcgagtcatcctcgctgacgccaaccttgataaggttctctctgcttacaacaagcaccgggacaagcccatccgagagcaggccgagaatatcatccacctcttcactctcaccaacctcggcgctcctgctgccttcaagtacttcgacaccaccattgaccgaaagaggtacacctccaccaaggaagtcctcgacgccaccctgatccaccagtccatcaccggcctctacgaaacccgaatcgacctctcccagctcggcggtgactctcgagccgaccccaagaagaagcgaaaagtctaaatatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatttgatatatatactaattacaatagtacatattagaacatacaatagttagtgccgtgaagtggcttaaaataccgcgagtcgattacgtaatattattacctcttgcccatcgaacgtacaagtactcctctgttctctccttcctttgctttgtgcacgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatgcattggggatagcacagggttggggtgtcttgtggactcaatgggtgaaaggagatggaaaagggcggtgaaaagtggtagaatcgaaatccctgacgtcaatttataaagtaaaatgcgtttctgccattttgctcccctccttctttcgcaatcgcctccccaaaagttgtcgtggcagtacacatgcttgcatacaatgaagctaatccggcttgctcagtagttgctatatccaggcatggtgtgaaacccctcaaagtatatataggagcggtgagccccagtctggggtcttttctctccatctcaaaactactttctcacaatgcgatatctgatgagtccgtgaggacgaaacgagtaagctcgtcatatcgccgcaagattacacgttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggactaaacttcgagctaatccagtagcttacgttacccaggggcaggtcaactggctagccacgagtctgtcccaggtcgcaatttagtgtaataaacaatatatatattgagtctaaagggaattgtagctattgtgattgtgtgattttcgtcttgctggttcttattgtgtcccattcgtttcatcctgatgaggacccctggaaccggtgttttcttagtctctgcaatcgctagtcttgttgctatgacagttgcgtcgacactattcaggtcatctatcggttattctgatattataatacctccggatcgatgtacctgatttatacttgcagcaatgtttacttcttatcgcgatacacgaatgtgatacggatcaaagtaagcaggactacgataagataacgaatgcggtgcagtccatgtcgattaggtatagatacatttattttgtgttatgttacattttggggggatactgtcctacttgtagtacctacttgtagtggcgcgtctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatgggtacctgagaacatttttgtgtctaggtgtttgtgtttggactgcgatcagtgaagaaaagaagaggaaaaattgtgcaagaaattttgctttcaagacttggctgatgcagcagggtaactctgggacacagacctatgtttgtggttaaactcaatgcacgtggtacgtgcgtggagcgcttacccatccaagggtgtggacatggaaccgacggtccgtggagttgtgtaatgtcattttggcgactcttgaagcaaggctataaaaaaattgtgtggcttgagtcttatcgagctcggtcactacaagagttaatcttcctgtctcaggcagacaggtcaggcagggttacttttgggtgtgctgtaactcactgtatggccgttagtgcgcatagacgttgtacatactggaccgaattgtagcgtgctcaatagggccaataaagctattgtagggatccgaattttcagaacctaatttatctgttacccggcctgtggctcgcacagcttaaaaatggtcaaactttccccttcttgtctttttttcctcacattcatcaggttcttgtcttgatctttcaagtgagtattaattaccgaccttggttcttcattgggagagcattggaagccgtggtgcagcaaccacaaaacggttcttccccttcgataccttcttgcctgcctttcaatacaagtcggctcgattagcggtggtcgcccccgccagcggagaacatggaactaacccagaatgagagctaagtggagaaagaagagagtcagacgactcaagcgaaagcgccgcaaggtccgagctcgatccaaataagcggtttttaacggagatttaacactaaatcgaagaacttttcccgtttcatttgcgaatgagctcgttaacaaaatcccccagtttttttatccagctgtaaggattgacattagtaatgaattattgtttggtatatttaaatctgtagttcctttctgtccgtgtcggcaactgtcgtactcgtgatttacttgtattgacgaatacttactgtagcgcactctgctgctactggtcgtaaggatgtgctatttcggtgtatggtgggttttttgggggtcggaaccgaagactgttacacgggcacggctcgttgtgtacacgcacagagctcttgcgagtcatgttgtagctagctcgtcgtgttcaggaactgttcgatggttcggagagagtcgccgcccagaacatacgcgcaccgatgtcagcagacagccttattacaagtatattcaagcaagtatatccgtagggtgcgggtgatttggatctaaggttcgtactcaacactcacgagcagcttgcctatgttacatccttttatcagacataacataattggagtttacttacacacggggtgtacctgtatgagcaccacctacaattgtagcactggtacttgtacaaagaatttattcgtacgaatcacagggacggccgccctcaccgaaccagcgaatacctcagcggtcccctgcagtgactcaacaaagcgatatgaacatcttgcgatggtatcctgctgatagtttttactgtacaaacacctgtgtagctccttctagcatttttaagttattcacacctcaaggggagggataaattaaataaattccaaaagcgaagatcgagaaactaaattaaaattccaaaaacgaagttggaacacaaccccccgaaaaaaaacaacaaacaaaaaacccaacaaaataaacaaaaacaaaataaatatataactaccagtatctgactaaaagttcaaatactcgtacttacaacaaatagaaatgagccggccaaaattctgcagaaaaaaatttcaaacaagtactggtataattaaattaaaaaacacatcaaagtatcataacgttagttattttattttatttaataaaagaaaacaacaagatgggctcaaaactttcaacttatacgatacataccaaataacaatttagtatttatctaagtgcttttcgtagataatggaatacaaatggatatccagagtatacacatggatagtatacactgacacgacaattctgtatctctttatgttaactactgtgaggcattaaatagagcttgatatataaaatgttacatttcacagtctgaacttttgcagattacctaatttggtaagatattaattatgaactgaaagttgatggcatccctaaatttgatgaaagatgaaattgtaaatgaggtggtaaaagagctacagtcgttttgttttgagataccatcatctctaacgaaatatctattaaaaatctcagtgtgatcatgagtcattgccatcctggaaaatgtcatcatggctgatatttctaactgtttacttgagataaatatatatttacaagaacttcccttgaaattaatttagatataaaatgtttgcgggcaagttactacgaggaataaattatatctaga

SEQ ID NO: 152 - DNA, 20 bp, Artificial Sequence
  Nucleotide sequence of the 20-bp genomic target of the KU70 gene
atatcgccgcaagattacac

SEQ ID NO: 153 - DNA, 100 bp, Artificial Sequence
  Nucleotide sequence of the 100-bp donor DNA fragment used for knocking out the KU70 gene in the Yarrowia genome
catcgcgacacgaacacgaaacacgaaccacgaaccgccgctttttgaaactagggaggcacatctaaacgaataacgaatattaatgataccatcatat

SEQ ID NO: 154 - DNA, 20 bp, Artificial Sequence
  Nucleotide sequence of the forward primer to confirm knock out of KU70 gene in the Yarrowia genome
gagcacgtcacgtgttctcc

SEQ ID NO: 155 - DNA, 20 bp, Artificial Sequence
  Nucleotide sequence of the reverse primer to confirm knock out of KU70 gene in the Yarrowia genome
agtggtaccgctactttgtg

SEQ ID NO: 156 - DNA, 2042 bp, Artificial Sequence
  Nucleotide sequence of the GFP expression cassette (Yl_HSP.pro - A.vic_eGFP ORF - Yl_GPD.ter)
gtgcaatcacatgttgctactgtacctgctgtggaccacgcacggcggaacgtaccgtacaaatattttcttgctcacatgactctctctcggccgcgcacgccggtggcaaattgctcttgcattggctctgtctctagacgtccaaaccgtccaaagtggcagggtgacgtgatgcgacgcacgaaggagatggcccggtggcgaggaaccggacacggcgagccggcgggaaaaaaggcggaaaacgaaaagcgaagggcacaatctgacggtgcggctgccaccaacccaaggaggctattttgggtcgctttccatttcacattcgccctcaatggccactttgcggtggtgaacatggtttctgaaacaaccccccagaattagagtatattgatgtgtttaagattgggttgctatttggccattgtgggggagggtagcgacgtggaggacattccagggcgaattgagcctagaaagtggtagcattccaaccgtctaagtcgtccgaattgatcgctataactatcacctctctcacatgtctacttccccaaccaacatccccaacctcccccacactaaagttcacgccaataatgtaggcactctttctgggtgtgggacagcagagcaatacggaggggagattacacaacgagccacaattggggagatggtagccatctcactcgacccgtcgacttttggcaacgctcaattacccaccaaatttgggctggagttgaggggaccgtgttccagcgctgtaggaccagcaacacacacggtatcaacagcaaccaacgcccccgctaatgcacccagtactgcgcaggtgtgggccaggtgcgttccagatgcgagttggcgaaccctaagccgacagtgtactttttgggacgggcagtagcaatcgtgggcggaaaccccggtgtatataaaggggtggagaggacggattattagcaccaacacacacacttatactacaatggagggtgatattcacggtgcttctaagggtgaggagcttttcaccggcgttgtccctattctcgttgagcttgatggtgatgtcaacggtcacaagttttctgtctctggtgagggtgagggtgatgctacttacggtaagctcaccctcaagtttatttgtaccaccggcaagctccccgttccctggcctaccctcgttaccactctcacctacggtgttcagtgtttttcccgataccccgatcacatgaagcgacacgactttttcaagtctgccatgcccgagggttacgttcaggagcgaactatttctttcaaggatgacggtaactacaagacccgagctgaggttaagtttgagggtgataccctcgttaaccgaatcgagcttaagggtattgatttcaaggaggatggtaacattctcggtcacaagctggagtacaactacaactctcacaacgtttacatcaccgctgacaagcagaagaacggtatcaaggctaacttcaagattcgacacaacattgaggatggttccgttcagcttgctgaccactaccagcagaacactcccattggcgatggccctgtcctcctccccgacaaccactacctctctacccagtctgccctttctaaggaccccaacgagaagcgagatcacatggtcctccttgagttcgttaccgctgctggtattactcacggtatggatgagctctacaagtaaataaatatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatttgatatatatactaattacaatagtacatattagaacatacaatagttagtgccgtgaagtggcttaaaataccgcgagtcgattacgtaatattatta

SEQ ID NO: 157 - DNA, 12810 bp, Artificial Sequence
  Nucleotide sequence of plasmid pSTV086
ggttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctggcctaggaagcgacttccaatcgctttgcatatccagtaccacacccacaggcgtttgtgctactctactgatagcaatagatgcgtcataattggttggcccgctgagcctccacaggatactattgcacataccctggtcatgtgcagatcagctcatttgtggagactctggagtaacttagacgacgcctggttcaattgccgcaatgtgcgcccacgcagataatgtattgaggggtggagcgcctcttggggacttgctgtacttgtacgggatattaaacgcactcagcaagaccatgacgtaaaacacacctactgtacgatacgtactgtaggtattgtactcgtacccggtactacaaatagtacgatactatacggagtgtatttgtaccttgatatacgactggcggagtgaagagaaggagttgaacaagaccagatggggatatcagccccagtgctttgtattacaagtacgagtacttaatagatactgtaaggctattgatacggatggcagtaagtcattgagtaagcaattgtggcccagcatctcccctacgtacttgtaccataccccatggagacaccaatggtctttcacgcacactgtcgtgtgctgtatcgcagaatcgggtgtccaaccaaatgccgttacccccacgtcacagccgatagacagatacaccatcaataccagcaggttgtatcatgcggttggctgaaggtaagctgattggtctaaaaactgtagctgtcctaattcaacgagcgctatttggggccaaccacctcggccaagcggcctttaatctgcgtgccccagaggcgtctaatgaggctctggccgccactgtaggagtgtttctctgtgcgcacacgcagttttgagtttgggcgactttccctttttcccaattgcgtacacacacagctccgagctaagcgctgtccttgaaccttctccctcttttccctctttttctcttccccttcccctcctccacattaaggccaaatcctgaattgcaccaactagtacaacgacaacaatggacaagaagtactccatcggtttggacattggtactaactctgtcggctgggccgtcatcaccgacgagtacaaggttccctccaagaagttcaaggtccttggcaacaccgaccgacactctatcaagaagaacctgatcggtgctctgctgttcgactctggcgagactgccgaggccacccgactgaagcgaaccgctcgacgccgatacacccgacgaaagaaccgaatctgttacctccaggagatcttcagcaacgagatggctaaggtcgacgactccttcttccaccgactcgaggagtctttcctggtcgaagaggataagaagcacgagcgacaccccatcttcggcaacattgttgatgaggttgcctaccatgagaagtaccccaccatctaccacctccgaaagaagctcgtcgactccactgacaaggctgacctccgactcatctaccttgctctcgcccacatgatcaagttccgaggtcacttcctcattgagggtgatctcaaccccgacaactccgacgttgacaagctgttcatccagctcgtccagacctacaaccagctctttgaggagaaccctatcaacgcttctggtgttgacgccaaggccattctctccgcccgactctctaagtcccgacgactcgagaacctcattgcccagctgcccggcgagaagaagaacggcctcttcggtaacctgattgctctctctcttggtctgacccccaacttcaagtccaactttgacctcgccgaggacgccaagctccagctgtccaaggacacctacgatgacgatctggacaacctcctggcccagatcggtgaccagtacgccgatctcttccttgccgccaagaacctctccgacgccatcctgctctccgacatcctccgagtcaacaccgagattaccaaggctcctctgtctgcctctatgatcaagcgatacgacgagcaccaccaggatctcactcttctcaaggctctcgtccgacagcagctccccgagaagtacaaggagattttctttgaccagtccaagaacggttacgctggctacattgacggtggtgcttcccaggaagagttttacaagttcatcaagcctattctggagaagatggacggtaccgaggagctgctcgtcaagctcaaccgagaggacctccttcgaaagcagcgaaccttcgataacggctccatcccccaccagatccacctgggtgagctccacgccattctccgaagacaagaggacttctaccccttcctaaaggataaccgagagaagatcgagaagattctcaccttccgaatcccctactacgtcggtcccctcgctcgaggtaactcccgatttgcttggatgacccgaaagtccgaggagactatcaccccctggaactttgaagaggtagtcgacaagggtgcctccgcccagtctttcattgagcggatgaccaacttcgataagaacctccccaacgagaaggtccttcccaagcactctctcctctacgagtacttcaccgtctacaacgagctgaccaaggtcaagtacgttaccgagggcatgcgaaagcccgctttcctctctggtgagcagaagaaggccattgtcgacctcctgttcaagactaaccgaaaagtcaccgtcaagcagctcaaggaagactacttcaagaagattgagtgcttcgactccgtcgagatttccggtgtcgaggaccgattcaacgcctccctcggcacctaccacgatcttctgaagatcatcaaggacaaggactttcttgataacgaggagaacgaggacattctcgaggacatcgtcctcaccctcacccttttcgaggatcgagagatgatcgaggagcgactcaagacctacgcccatctcttcgacgacaaggtcatgaagcaactcaagcgacgacgatacactggctggggccgactttcccgaaagctcatcaacggcatccgagacaagcagtctggcaagaccatcctggacttcctgaagtccgacggtttcgccaaccgaaacttcatgcagctcatccacgacgactctcttaccttcaaagaggatatccagaaggcccaggtttctggccagggcgactccctccacgagcacattgccaacctcgccggatcccccgccatcaaaaagggtatcctccagaccgtcaaggttgtcgacgaactcgtgaaggtcatgggccgacacaagcccgagaacatcgttatcgagatggcccgagagaaccagaccacccagaagggtcagaagaactcccgagagcgaatgaagcgaatcgaagagggtatcaaggagctcggttcccagattctcaaggagcaccccgtcgagaacacccagctccagaacgagaaactctacctgtactacctccagaatggccgagacatgtacgttgaccaggagctcgacatcaaccgactctccgactacgacgtcgaccacattgttcctcagtccttcctcaaggacgactccatcgacaacaaggttctgacccgatctgacaagaaccgaggtaagtccgacaacgttccctccgaagaggtcgttaagaagatgaagaactactggcgacagcttctcaacgccaaactgatcacccagcgaaagtttgacaacctcaccaaggccgagcgaggtggtctgtccgagctggacaaggccggcttcattaagcgacagctggtcgagactcgacagatcaccaagcacgtcgcccagatcctcgactcccgaatgaacaccaagtacgacgagaacgacaagctcatccgggaggtcaaggtcatcaccctgaagtctaagcttgtctccgacttccgaaaggacttccagttctacaaggtccgagagatcaacaactaccaccacgcccacgacgcctacctcaacgccgttgttggtaccgccctcatcaagaagtatcccaagctcgagtccgagttcgtttacggcgactacaaggtttacgatgtccgaaagatgattgccaagtccgagcaggagatcggtaaggccaccgccaagtactttttctactccaacatcatgaatttcttcaagaccgagatcactctcgccaacggtgagattcgaaagcgacccctgattgagactaatggtgagactggtgagatcgtctgggataagggccgagacttcgccaccgtccgaaaggtcctgtccatgccccaggtcaacattgtcaagaagaccgaggtccagaccggtggcttctccaaggagtccattctccccaagcgaaactccgacaaactcatcgcccgtaagaaggactgggatccgaagaagtacggtggtttcgattctcccaccgttgcctactccgtcctcgttgttgctaaagtcgagaagggtaagtctaagaaactcaagtccgtgaaggagctactcggtatcaccatcatggagcgatcttcttttgagaagaaccccattgacttcctcgaggccaagggttacaaagaggtcaagaaggacctgattatcaagctgcccaagtactccctctttgagctcgagaacggccgaaagcgaatgctggcttccgctggtgagctgcagaagggcaacgagctcgctctgccctccaagtacgtcaacttcctctacctggcctcccactacgagaagctcaagggctcccccgaggacaacgagcagaagcagctgttcgttgagcagcacaagcactacctcgacgagatcatcgagcagatctccgagttctccaagcgagtcatcctcgctgacgccaaccttgataaggttctctctgcttacaacaagcaccgggacaagcccatccgagagcaggccgagaatatcatccacctcttcactctcaccaacctcggcgctcctgctgccttcaagtacttcgacaccaccattgaccgaaagaggtacacctccaccaaggaagtcctcgacgccaccctgatccaccagtccatcaccggcctctacgaaacccgaatcgacctctcccagctcggcggtgactctcgagccgaccccaagaagaagcgaaaagtctaaatatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatttgatatatatactaattacaatagtacatattagaacatacaatagttagtgccgtgaagtggcttaaaataccgcgagtcgattacgtaatattattacctcttgcccatcgaacgtacaagtactcctctgttctctccttcctttgctttgtgcacgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatgcattggggatagcacagggttggggtgtcttgtggactcaatgggtgaaaggagatggaaaagggcggtgaaaagtggtagaatcgaaatccctgacgtcaatttataaagtaaaatgcgtttctgccattttgctcccctccttctttcgcaatcgcctccccaaaagttgtcgtggcagtacacatgcttgcatacaatgaagctaatccggcttgctcagtagttgctatatccaggcatggtgtgaaacccctcaaagtatatataggagcggtgagccccagtctggggtcttttctctccatctcaaaactactttctcacaatgaggccactgatgagtccgtgaggacgaaacgagtaagctcgtctggcctgttgagtcaacccggttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggactaaacttcgagctaatccagtagcttacgttacccaggggcaggtcaactggctagccacgagtctgtcccaggtcgcaatttagtgtaataaacaatatatatattgagtctaaagggaattgtagctattgtgattgtgtgattttcgtcttgctggttcttattgtgtcccattcgtttcatcctgatgaggacccctggaaccggtgttttcttagtctctgcaatcgctagtcttgttgctatgacagttgcgtcgacactattcaggtcatctatcggttattctgatattataatacctccggatcgatgtacctgatttatacttgcagcaatgtttacttcttatcgcgatacacgaatgtgatacggatcaaagtaagcaggactacgataagataacgaatgcggtgcagtccatgtcgattaggtatagatacatttattttgtgttatgttacattttggggggatactgtcctacttgtagtacctacttgtagtggcgcgtctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatgggtacctgagaacatttttgtgtctaggtgtttgtgtttggactgcgatcagtgaagaaaagaagaggaaaaattgtgcaagaaattttgctttcaagacttggctgatgcagcagggtaactctgggacacagacctatgtttgtggttaaactcaatgcacgtggtacgtgcgtggagcgcttacccatccaagggtgtggacatggaaccgacggtccgtggagttgtgtaatgtcattttggcgactcttgaagcaaggctataaaaaaattgtgtggcttgagtcttatcgagctcggtcactacaagagttaatcttcctgtctcaggcagacaggtcaggcagggttacttttgggtgtgctgtaactcactgtatggccgttagtgcgcatagacgttgtacatactggaccgaattgtagcgtgctcaatagggccaataaagctattgtagggatccgaattttcagaacctaatttatctgttacccggcctgtggctcgcacagcttaaaaatggtcaaactttccccttcttgtctttttttcctcacattcatcaggttcttgtcttgatctttcaagtgagtattaattaccgaccttggttcttcattgggagagcattggaagccgtggtgcagcaaccacaaaacggttcttccccttcgataccttcttgcctgcctttcaatacaagtcggctcgattagcggtggtcgcccccgccagcggagaacatggaactaacccagaatgagagctaagtggagaaagaagagagtcagacgactcaagcgaaagcgccgcaaggtccgagctcgatccaaataagcggtttttaacggagatttaacactaaatcgaagaacttttcccgtttcatttgcgaatgagctcgttaacaaaatcccccagtttttttatccagctgtaaggattgacattagtaatgaattattgtttggtatatttaaatctgtagttcctttctgtccgtgtcggcaactgtcgtactcgtgatttacttgtattgacgaatacttactgtagcgcactctgctgctactggtcgtaaggatgtgctatttcggtgtatggtgggttttttgggggtcggaaccgaagactgttacacgggcacggctcgttgtgtacacgcacagagctcttgcgagtcatgttgtagctagctcgtcgtgttcaggaactgttcgatggttcggagagagtcgccgcccagaacatacgcgcaccgatgtcagcagacagccttattacaagtatattcaagcaagtatatccgtagggtgcgggtgatttggatctaaggttcgtactcaacactcacgagcagcttgcctatgttacatccttttatcagacataacataattggagtttacttacacacggggtgtacctgtatgagcaccacctacaattgtagcactggtacttgtacaaagaatttattcgtacgaatcacagggacggccgccctcaccgaaccagcgaatacctcagcggtcccctgcagtgactcaacaaagcgatatgaacatcttgcgatggtatcctgctgatagtttttactgtacaaacacctgtgtagctccttctagcatttttaagttattcacacctcaaggggagggataaattaaataaattccaaaagcgaagatcgagaaactaaattaaaattccaaaaacgaagttggaacacaaccccccgaaaaaaaacaacaaacaaaaaacccaacaaaataaacaaaaacaaaataaatatataactaccagtatctgactaaaagttcaaatactcgtacttacaacaaatagaaatgagccggccaaaattctgcagaaaaaaatttcaaacaagtactggtataattaaattaaaaaacacatcaaagtatcataacgttagttattttattttatttaataaaagaaaacaacaagatgggctcaaaactttcaacttatacgatacataccaaataacaatttagtatttatctaagtgcttttcgtagataatggaatacaaatggatatccagagtatacacatggatagtatacactgacacgacaattctgtatctctttatgttaactactgtgaggcattaaatagagcttgatatataaaatgttacatttcacagtctgaacttttgcagattacctaatttggtaagatattaattatgaactgaaagttgatggcatccctaaatttgatgaaagatgaaattgtaaatgaggtggtaaaagagctacagtcgttttgttttgagataccatcatctctaacgaaatatctattaaaaatctcagtgtgatcatgagtcattgccatcctggaaaatgtcatcatggctgatatttctaactgtttacttgagataaatatatatttacaagaacttcccttgaaattaatttagatataaaatgtttgcgggcaagttactacgaggaataaattatatctaga

SEQ ID NO: 158 - DNA, 2142 bp, Artificial Sequence
  Nucleotide sequence of the GFP expression cassette (Yl_HSP.pro - A.vic_eGFP ORF - Yl_GPD.ter) flanked by 50-bp genomic DNA sequences on either side for targeted integration in the INT05 locus
tgtgaccttgtaaacaacaacccgactttacagtaacgcttggacacactgtgcaatcacatgttgctactgtacctgctgtggaccacgcacggcggaacgtaccgtacaaatattttcttgctcacatgactctctctcggccgcgcacgccggtggcaaattgctcttgcattggctctgtctctagacgtccaaaccgtccaaagtggcagggtgacgtgatgcgacgcacgaaggagatggcccggtggcgaggaaccggacacggcgagccggcgggaaaaaaggcggaaaacgaaaagcgaagggcacaatctgacggtgcggctgccaccaacccaaggaggctattttgggtcgctttccatttcacattcgccctcaatggccactttgcggtggtgaacatggtttctgaaacaaccccccagaattagagtatattgatgtgtttaagattgggttgctatttggccattgtgggggagggtagcgacgtggaggacattccagggcgaattgagcctagaaagtggtagcattccaaccgtctaagtcgtccgaattgatcgctataactatcacctctctcacatgtctacttccccaaccaacatccccaacctcccccacactaaagttcacgccaataatgtaggcactctttctgggtgtgggacagcagagcaatacggaggggagattacacaacgagccacaattggggagatggtagccatctcactcgacccgtcgacttttggcaacgctcaattacccaccaaatttgggctggagttgaggggaccgtgttccagcgctgtaggaccagcaacacacacggtatcaacagcaaccaacgcccccgctaatgcacccagtactgcgcaggtgtgggccaggtgcgttccagatgcgagttggcgaaccctaagccgacagtgtactttttgggacgggcagtagcaatcgtgggcggaaaccccggtgtatataaaggggtggagaggacggattattagcaccaacacacacacttatactacaatggagggtgatattcacggtgcttctaagggtgaggagcttttcaccggcgttgtccctattctcgttgagcttgatggtgatgtcaacggtcacaagttttctgtctctggtgagggtgagggtgatgctacttacggtaagctcaccctcaagtttatttgtaccaccggcaagctccccgttccctggcctaccctcgttaccactctcacctacggtgttcagtgtttttcccgataccccgatcacatgaagcgacacgactttttcaagtctgccatgcccgagggttacgttcaggagcgaactatttctttcaaggatgacggtaactacaagacccgagctgaggttaagtttgagggtgataccctcgttaaccgaatcgagcttaagggtattgatttcaaggaggatggtaacattctcggtcacaagctggagtacaactacaactctcacaacgtttacatcaccgctgacaagcagaagaacggtatcaaggctaacttcaagattcgacacaacattgaggatggttccgttcagcttgctgaccactaccagcagaacactcccattggcgatggccctgtcctcctccccgacaaccactacctctctacccagtctgccctttctaaggaccccaacgagaagcgagatcacatggtcctccttgagttcgttaccgctgctggtattactcacggtatggatgagctctacaagtaaataaatatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatttgatatatatactaattacaatagtacatattagaacatacaatagttagtgccgtgaagtggcttaaaataccgcgagtcgattacgtaatattattacatcaatatatcggaccaatatatcggaccacggtcgccgccgaatcgcc

SEQ ID NO: 159 - DNA, 24 bp, Artificial Sequence
  Nucleotide sequence of the forward primer to confirm integration of the GFP expression cassette in the INT05 locus in the Yarrowia genome
cattgatcttactgtcgaatgtac

SEQ ID NO: 160 - DNA, 21 bp, Artificial Sequence
  Nucleotide sequence of the reverse primer to confirm integration of the GFP expression cassette in the INT05 locus in the Yarrowia genome
ttggcgtaggtgccgatgtag

SEQ ID NO: 161 - DNA, 5844 bp, Artificial Sequence
  Nucleotide sequence of plasmid pSTV077
agatcttaattaagcttggcgcgcctccggatcgatgtacctgatttatacttgcagcaatgtttacttcttatcgcgatacacgaatgtgatacggatcaaagtaagcaggactacgataagataacgaatgcggtgcagtccatgtcgattaggtatagatacatttattttgtgttatgttacattttggggggatactgtcctacttgtagtacctacttgtagtggcgcgtctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatgggtacctgagaacatttttgtgtctaggtgtttgtgtttggactgcgatcagtgaagaaaagaagaggaaaaattgtgcaagaaattttgctttcaagacttggctgatgcagcagggtaactctgggacacagacctatgtttgtggttaaactcaatgcacgtggtacgtgcgtggagcgcttacccatccaagggtgtggacatggaaccgacggtccgtggagttgtgtaatgtcattttggcgactcttgaagcaaggctataaaaaaattgtgtggcttgagtcttatcgagctcggtcactacaagagttaatcttcctgtctcaggcagacaggtcaggcagggttacttttgggtgtgctgtaactcactgtatggccgttagtgcgcatagacgttgtacatactggaccgaattgtagcgtgctcaatagggccaataaagctattgtagggatccgaattttcagaacctaatttatctgttacccggcctgtggctcgcacagcttaaaaatggtcaaactttccccttcttgtctttttttcctcacattcatcaggttcttgtcttgatctttcaagtgagtattaattaccgaccttggttcttcattgggagagcattggaagccgtggtgcagcaaccacaaaacggttcttccccttcgataccttcttgcctgcctttcaatacaagtcggctcgattagcggtggtcgcccccgccagcggagaacatggaactaacccagaatgagagctaagtggagaaagaagagagtcagacgactcaagcgaaagcgccgcaaggtccgagctcgatccaaataagcggtttttaacggagatttaacactaaatcgaagaacttttcccgtttcatttgcgaatgagctcgttaacaaaatcccccagtttttttatccagctgtaaggattgacattagtaatgaattattgtttggtatatttaaatctgtagttcctttctgtccgtgtcggcaactgtcgtactcgtgatttacttgtattgacgaatacttactgtagcgcactctgctgctactggtcgtaaggatgtgctatttcggtgtatggtgggttttttgggggtcggaaccgaagactgttacacgggcacggctcgttgtgtacacgcacagagctcttgcgagtcatgttgtagctagctcgtcgtgttcaggaactgttcgatggttcggagagagtcgccgcccagaacatacgcgcaccgatgtcagcagacagccttattacaagtatattcaagcaagtatatccgtagggtgcgggtgatttggatctaaggttcgtactcaacactcacgagcagcttgcctatgttacatccttttatcagacataacataattggagtttacttacacacggggtgtacctgtatgagcaccacctacaattgtagcactggtacttgtacaaagaatttattcgtacgaatcacagggacggccgccctcaccgaaccagcgaatacctcagcggtcccctgcagtgactcaacaaagcgatatgaacatcttgcgatggtatcctgctgatagtttttactgtacaaacacctgtgtagctccttctagcatttttaagttattcacacctcaaggggagggataaattaaataaattccaaaagcgaagatcgagaaactaaattaaaattccaaaaacgaagttggaacacaaccccccgaaaaaaaacaacaaacaaaaaacccaacaaaataaacaaaaacaaaataaatatataactaccagtatctgactaaaagttcaaatactcgtacttacaacaaatagaaatgagccggccaaaattctgcagaaaaaaatttcaaacaagtactggtataattaaattaaaaaacacatcaaagtatcataacgttagttattttattttatttaataaaagaaaacaacaagatgggctcaaaactttcaacttatacgatacataccaaataacaatttagtatttatctaagtgcttttcgtagataatggaatacaaatggatatccagagtatacacatggatagtatacactgacacgacaattctgtatctctttatgttaactactgtgaggcattaaatagagcttgatatataaaatgttacatttcacagtctgaacttttgcagattacctaatttggtaagatattaattatgaactgaaagttgatggcatccctaaatttgatgaaagatgaaattgtaaatgaggtggtaaaagagctacagtcgttttgttttgagataccatcatctctaacgaaatatctattaaaaatctcagtgtgatcatgagtcattgccatcctggaaaatgtcatcatggctgatatttctaactgtttacttgagataaatatatatttacaagaacttcccttgaaattaatttagatataaaatgtttgcgggcaagttactacgaggaataaattatatctagaggttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctggcctaggaagcgacttccaatcgctttgcatatccagtaccacacccacaggcgttt

SEQ ID NO: 162 - DNA, 1000 bp, Artificial Sequence
  Nucleotide sequence of Yarrowia Yl_HSP promoter
gtgcaatcacatgttgctactgtacctgctgtggaccacgcacggcggaacgtaccgtacaaatattttcttgctcacatgactctctctcggccgcgcacgccggtggcaaattgctcttgcattggctctgtctctagacgtccaaaccgtccaaagtggcagggtgacgtgatgcgacgcacgaaggagatggcccggtggcgaggaaccggacacggcgagccggcgggaaaaaaggcggaaaacgaaaagcgaagggcacaatctgacggtgcggctgccaccaacccaaggaggctattttgggtcgctttccatttcacattcgccctcaatggccactttgcggtggtgaacatggtttctgaaacaaccccccagaattagagtatattgatgtgtttaagattgggttgctatttggccattgtgggggagggtagcgacgtggaggacattccagggcgaattgagcctagaaagtggtagcattccaaccgtctaagtcgtccgaattgatcgctataactatcacctctctcacatgtctacttccccaaccaacatccccaacctcccccacactaaagttcacgccaataatgtaggcactctttctgggtgtgggacagcagagcaatacggaggggagattacacaacgagccacaattggggagatggtagccatctcactcgacccgtcgacttttggcaacgctcaattacccaccaaatttgggctggagttgaggggaccgtgttccagcgctgtaggaccagcaacacacacggtatcaacagcaaccaacgcccccgctaatgcacccagtactgcgcaggtgtgggccaggtgcgttccagatgcgagttggcgaaccctaagccgacagtgtactttttgggacgggcagtagcaatcgtgggcggaaaccccggtgtatataaaggggtggagaggacggattattagcaccaacacacacacttatactaca

SEQ ID NO: 163 - DNA, 742 bp, Artificial Sequence
  Nucleotide sequence of Aequorea victoria eGFP gene (A. vic_eGFP ORF)
atggagggtgatattcacggtgcttctaagggtgaggagcttttcaccggcgttgtccctattctcgttgagcttgatggtgatgtcaacggtcacaagttttctgtctctggtgagggtgagggtgatgctacttacggtaagctcaccctcaagtttatttgtaccaccggcaagctccccgttccctggcctaccctcgttaccactctcacctacggtgttcagtgtttttcccgataccccgatcacatgaagcgacacgactttttcaagtctgccatgcccgagggttacgttcaggagcgaactatttctttcaaggatgacggtaactacaagacccgagctgaggttaagtttgagggtgataccctcgttaaccgaatcgagcttaagggtattgatttcaaggaggatggtaacattctcggtcacaagctggagtacaactacaactctcacaacgtttacatcaccgctgacaagcagaagaacggtatcaaggctaacttcaagattcgacacaacattgaggatggttccgttcagcttgctgaccactaccagcagaacactcccattggcgatggccctgtcctcctccccgacaaccactacctctctacccagtctgccctttctaaggaccccaacgagaagcgagatcacatggtcctccttgagttcgttaccgctgctggtattactcacggtatggatgagctctacaagtaaataa

SEQ ID NO: 164 - DNA, 300 bp, Artificial Sequence; identical to SEQ ID NO: 150
  Nucleotide sequence of Yarrowia Yl_GPD terminator

SEQ ID NO: 165 - DNA, 22 bp, Artificial Sequence
  Nucleotide sequence of the forward primer to amplify the edited GFP ORF from the Yarrowia genome
atggagggtgatattcacggtg

SEQ ID NO: 166 - DNA, 23 bp, Artificial Sequence
  Nucleotide sequence of the reverse primer to amplify the edited GFP ORF from the Yarrowia genome
ttacttgtagagctcatccatac

SEQ ID NO: 167 - DNA, 6 bp, Artificial Sequence
  Nucleotide sequence of 6 bp inverted repeat of the KU70 genomic target
cgatat

SEQ ID NO: 168 - DNA, 6 bp, Artificial Sequence
  Nucleotide sequence of 6 bp inverted repeat of the INT05 genomic target
aggcca

SEQ ID NO: 169 - DNA, 20 bp, Artificial Sequence
  Nucleotide sequence of the 20-bp genomic target sequence of the INT05 locus
tggcctgttgagtcaacccg

SEQ ID NO: 170 - DNA, 1587 bp, Artificial Sequence
  Nucleotide sequence of CTEC DNA fragment 1, comprising a guide RNA expression cassette (sgRNA) for targeting Cas9 to the GFP gene and donor DNA of 100-bp, which encodes for the full knock out of the GFP ORF, on the 3′ side
acgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatgcattggggatagcacagggttggggtgtcttgtggactcaatgggtgaaaggagatggaaaagggcggtgaaaagtggtagaatcgaaatccctgacgtcaatttataaagtaaaatgcgtttctgccattttgctcccctccttctttcgcaatcgcctccccaaaagttgtcgtggcagtacacatgcttgcatacaatgaagctaatccggcttgctcagtagttgctatatccaggcatggtgtgaaacccctcaaagtatatataggagcggtgagccccagtctggggtcttttctctccatctcaaaactactttctcacaatggtattgctgatgagtccgtgaggacgaaacgagtaagctcgtccaatacccttaagctcgattgttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggactaaacttcgagctaatccagtagcttacgttacccaggggcaggtcaactggctagccacgagtctgtcccaggtcgcaatttagtgtaataaacaatatatatattgagtctaaagggaattgtagctattgtgattgtgtgattttcgtcttgctggttcttattgtgtcccattcgtttcatcctgatgaggacccctggaaccggtgttttcttagtctctgcaatcgctagtcttgttgctatgacagttgcgtcgacactattcaggtcatctatcggttattctgatattataatagggaaacatgtcctggacttacaacttgcttcgctcttgatcttcggatagtagtataagtgtgtgtgttggtgctaataatccgtcctctccacccctt

SEQ ID NO: 171 - DNA, 1587 bp, Artificial Sequence
  Nucleotide sequence of CTEC DNA fragment 2, comprising a guide RNA expression cassette (sgRNA) for targeting Cas9 to the GFP gene and donor DNA of 100-bp, which encodes a base deletion in the PAM sequence, changing it from CGG to CG, on the 3′ side
acgaagaactgcggtcaggtgacacaactttttccatctcagggtgtgtcgcgtgtgcttcatccaaactttagttggggttcgggttcgcgcgagatgatcacgtgccctgatttggtgtcgtcccccgtcgcgctgcgcacgtgatttatttatttccggtggctgctgtctacgcggggccttctctgcccttctgtttcaaccttcgggcggttctcgtaaccagcagtagcaatccatttcgaaactcaaagagctaaaaacgttaaacctcagcagtcgctcgacgaatgggctgcggttgggaagcccacgaggcctatagccagagcctcgagttgacaggagcccagacgccttttccaacggcaacttttatataaaatggcaatgtattcatgcaattgcggccgtgtcaggttggagacactggaccacactctccattgcttcctgaggagatggatcattgctagtgcatctacgcgcagcaatcccgcaagctcgacaaccgtagatgggctttggtgggccaatcaattacgcaacccgcacgttaaattgtatgaggaaggaaggccacggtacaaagtgggtggtcttcacccagtggttgttggtggcgtcatgcagaccatgcattggggatagcacagggttggggtgtcttgtggactcaatgggtgaaaggagatggaaaagggcggtgaaaagtggtagaatcgaaatccctgacgtcaatttataaagtaaaatgcgtttctgccattttgctcccctccttctttcgcaatcgcctccccaaaagttgtcgtggcagtacacatgcttgcatacaatgaagctaatccggcttgctcagtagttgctatatccaggcatggtgtgaaacccctcaaagtatatataggagcggtgagccccagtctggggtcttttctctccatctcaaaactactttctcacaatggtattgctgatgagtccgtgaggacgaaacgagtaagctcgtccaatacccttaagctcgattgttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggactaaacttcgagctaatccagtagcttacgttacccaggggcaggtcaactggctagccacgagtctgtcccaggtcgcaatttagtgtaataaacaatatatatattgagtctaaagggaattgtagctattgtgattgtgtgattttcgtcttgctggttcttattgtgtcccattcgtttcatcctgatgaggacccctggaaccggtgttttcttagtctctgcaatcgctagtcttgttgctatgacagttgcgtcgacactattcaggtcatctatcggttattctgatattataatatccagcttgtgaccgagaatgttaccatcctccttgaaatcaatacccttaagctcgattcgttaacgagggtatcaccctcaaacttaacctcagctcg

(19) United States
(12) Patent Application Publication
(10) Pub. No.: US 2021/0071174 A1
(43) Pub. Date: Mar. 11, 2021

(54) CRISPR TRANSIENT EXPRESSION CONSTRUCT (CTEC)

(71) Applicant: DSM IP ASSETS B.V., Heerlen (NL)

(72) Inventors: Johannes Andries ROUBOS, Echt (NL); Rene VERWAAL, Echt (NL); Brenda VONK, Echt (NL)

(21) Appl. No.: 17/053,265

(22) PCT Filed: May 6, 2019
(86) PCT No.: PCT/EP2019/061587

§ 371 Date: Nov. 5, 2020

(30) Foreign Application Priority Data
May 9, 2018 (EP) 18171496.5
Jul. 18, 2018 (EP) 18184210.5

(51) Int. Cl. C12N 15/11; C12N 9/22; C12N 15/81

(52) U.S. Cl. CPC C12N 15/11; C12N 9/22; C12N 2330/51; C12N 2310/20; C12N 15/81

(57) ABSTRACT

The present invention relates to the field of molecular biology and cell biology. More specifically, the present invention relates to a CRISPR transient expression construct (CTEC) for a genome editing system.

### FIELD

The present invention relates to the field of molecular biology and cell biology. More specifically, the present invention relates to a CRISPR transient expression construct for a genome editing system.

### BACKGROUND

A polynucleotide-guided nuclease system, also referred to as polynucleotide-guided genome editing system, from which the best known are the CRISPR/Cas9 and CRISPR/Cpf1 systems, is a powerful tool that has been leveraged for genome editing and gene regulation, e.g. to generate within a host cell a targeted mutation, a targeted insertion or a targeted deletion/knock-out. This tool requires at least a polynucleotide-guided nuclease such as Cas9 and Cpf1 and a guide-polynucleotide such as a guide-RNA that enables the genome editing enzyme to target a specific sequence of DNA. In addition, for editing of the genome in a precise way, a donor polynucleotide such as a donor DNA is mostly required, especially when relying on homologous recombination for editing precisely at a desired spot in the genome instead of relying on repair by a random repair process, such as non-homologous end joining. For each target site, a donor polynucleotide needs to be designed and synthesized. In addition, a guide-polynucleotide specific for a target site in the genome needs to be designed and needs to be expressed within the cell or needs to be expressed in vitro and introduced into the cell. For targeted modification with a polynucleotide-guided genome editing system, a combination of a guide-polynucleotide and a donor polynucleotide which are specific for a target need to be used. Especially for multiplex approaches such as when screening, e.g., a knock-out library, a knock-down library or a promoter-replacement library, the experimental work is quite laborious since matching compositions comprising a guide-polynucleotide or guide-polynucleotide expression construct and a matching donor polynucleotide will have to be transformed together. For screening multiple targets and/or multiple modifications in one experiment, the state of the art set-up requires a multitude of polynucleotides to be added and used and an even higher amount of screenings for a cell comprising the desired properties. Accordingly, there is a continuing urge to develop improved and simplified guide-polynucleotide and donor polynucleotide tools.

### BRIEF DESCRIPTION OF THE DRAWINGS

FIG. 1 depicts the vector map of single copy (CEN/ARS) vector pCSNO61 encoding Cas9 codon-pair optimized (CPO) for expression in S. cerevisiae. CPO Cas9 is expressed from the Kluyveromyces lactis KLLA0F20031g promoter and the S. cerevisiae GND2 terminator. A KanMX marker cassette is present on the vector, which confers resistance against G418 to allow selection of transformants on plate or in liquid cultures. The TRP1 marker allows selection of the plasmid in yeast strains with a trp1 auxotrophy.

FIG. 2 depicts the vector map of multi-copy (2 micron) vector pRN1120. A NatMX marker cassette is present on the vector, which confers resistance against nourseothricin to allow selection of transformants on plate or in liquid cultures. The vector is used for used for in vivo (within a cell) recombination with an sgRNA expression cassette after linearization using EcoRI and XhoI.

FIG. 3 depicts designs of CTEC DNA fragments for Cas9 editing. The CTEC DNA fragments consist of the sgRNA expression cassette which comprises the SNR52p RNA polymerase III promoter, a guide-sequence (also referred to as genomic target sequence; targeting either the INT1 genomic locus or the YFP gene), the gRNA structural component and the SUP4 3′ flanking region as described in DiCarlo et al., 2013, and the donor DNA that encodes a DNA base substitution (INT1) or DNA base deletion causing a frameshift (YFP).

FIG. 4 depicts designs of CTEC DNA fragments for Cpf1 editing. The CTEC DNA fragments consist of the crRNA expression cassette which comprises the SNR52p RNA polymerase III promoter, a guide-RNA sequence consisting of the direct repeat and the genomic target sequence, targeting either the INT1 genomic locus or the YFP gene, followed by the SUP4 terminator as described in Zetsche et al., 2015., and the donor DNA that encodes 3 bp substitution (INT1) or 2 base pair deletion causing a frameshift (YFP).

FIG. 5 depicts the vector map of single copy (CEN/ARS) vector pCSN067 expressing LbCpf1 (from Lachnospiraceae bacterium ND2006). A KanMX marker is present on the vector.

FIG. 6 depicts designs of the CTEC DNA fragments for Cpf1 editing. The CTEC DNA fragments consist of the crRNA expression cassette which comprises the SNR52p RNA polymerase III promoter, a guide-RNA sequence consisting of the direct repeat and the genomic target sequence, targeting the YFP gene, followed by the SUP4 terminator as described in Zetsche et al., 2015., and the donor DNA that encodes a 2 base pair deletion causing a frameshift in the YFP gene. To be able to amplify different CTEC fragments with the same primer set, connector 5 and/or connector 3 are attached to the CTEC fragments.

FIG. 7 depicts designs of the CTEC DNA fragments for Cpf1 editing. The CTEC DNA fragments consist of the crRNA expression cassette which comprises the SNR52p RNA polymerase III promoter, a guide-RNA sequence consisting of the direct repeat and the genomic target sequence, targeting the YFP gene, followed by the SUP4 terminator as described in Zetsche et al., 2015., and the donor DNA. Donor DNA encodes a 2 base pair deletion causing a frameshift in the YFP gene (CTEC-31, CTEC-32 and CTEC-33) or encodes flanking regions just outside the YFP expression cassette (CTEC-34, CTEC-35 and CTEC-36).

FIG. 8 depicts ex vivo use of a CRISPR transient expression construct (CTEC) according to the invention.

In 8A, the CTEC is applied in a transformation together with an autonomous replicating plasmid with a selection marker on it and used in a cell that pre-expresses a Cas protein (e.g. Cas9, Cpf, a variant of these or other Cas protein).

In 8B, the CTEC is applied in a transformation together with an autonomous replicating plasmid with a selection marker and an expression cassette for Cas protein on it (e.g. Cas9, Cpf, a variant of these or other Cas protein).

In 8C, the CTEC is applied in a transformation together with an autonomous replicating plasmid with a selection marker and together with a CAS protein (e.g. Cas9, Cpf, a variant of these or other Cas protein).

FIG. 9 depicts a CRISPR transient expression construct (CTEC) according to the invention.

In 9A, the CTEC is one double-stranded DNA fragment.

In 9B, the CTEC fragment recombines in the cell based on two or more fragments provided, here depicted with an in-vivo assembly using a homology stretch of DNA on the additional polynucleotide element that encodes for the donor DNA (that encodes for example for a targeted SNP, InDel, knock-out or insertion of DNA at the chromosome).

In 9c, the CTEC fragment recombines in the cell based on 2 or more fragments provided, here depicted with an in-vivo assembly using a homology stretch of DNA on the guide-RNA expression cassette.

In 9D, two (or more) CTEC are provided to generate two (or more) multiple events at the chromosome.

In 9E, two (or more) split CTEC are provided to generate one (or more) events at the chromosome, here with multiple guide-RNA expression cassettes that can recombine at a CTEC, for example to have two or more RNA guides act at one or more sites on a chromosome.

In 9F, a variant of 9E is depicted, where two (or more) split CTEC are provided to generate one (or more) events at the chromosome, here with multiple guide-RNA expression cassettes that can recombine at a CTEC, for example to have two or more RNA guides act at one or more sites on a chromosome.

In 9G, two (or more) split CTEC are provided to generate one (or more) events at the chromosome, here with a guide-RNA expression cassettes that can recombine with multiple variants of the additional polynucleotide element that encodes for the donor DNA (that encodes for example for a targeted SNP, InDel, knock-out or insertion of DNA at the chromosome).

FIG. 10 depicts ex vivo use of a CRISPR transient expression construct (CTEC) according to the invention.

In 10A, a guide-RNA expression cassette, and an additional polynucleotide element are depicted, where the additional polynucleotide element are encoded next to each other from right to left.

In 10B, a guide-RNA expression cassette, and an additional polynucleotide element are depicted, where the additional polynucleotide element is connected to a guide-RNA expression cassette by a linker that encodes a guide-RNA target sequence that is recognized by the guide-RNA encoded on the expression cassette, and by that the CTEC might be split in the ex vivo.

In 10C, a variant of 10A is shown where the elements are in different order at the CTEC. In 10D, a variant of 10B is shown where the elements are in different order at the CTEC.

FIG. 11 depicts ex vivo use of a CRISPR transient expression construct (CTEC) according to the invention.

In 11A-H, variants of CTEC are shown with and without a linker sequence, where in the CTEC a left (LF) and right (RF) homology flank are indicated, that can be used to make DNA knock-out, for example using 50-bp left and right homology flanks, with a RNA-targeted cut in between at the chromosome, or, for example, when a linker encodes for a promoter sequence, make a targeted insertion of that promoter, or insert another sequence encoded by the linker on the genome using RNA-guided DNA editing with a CTEC.

FIG. 12 depicts variants of constructs as depicted in FIG. 10. Here, flank DNA sequence are added at the 5′ and 3′ of the CTEC. These can be applied to have generic flanks, for example, to facilitate simple PCR, or PCR from a library (mix) of CTEC cassettes.

FIG. 13 depicts variants of constructs as depicted in FIG. 11. Here flank DNA sequence are added at the 5′ and 3′ of the CTEC. These can be applied to have generic flanks, for example, to facilitate simple PCR, or PCR from a library (mix) of CTEC cassettes.

FIG. 14 depicts ex vivo use of a CRISPR transient expression construct (CTEC) according to the invention.

In 14A, the CTEC is applied in a transformation together with a linearized (or linear part of) an autonomous replicating plasmid with a selection marker on it. A CTEC will in the cell recombine with the linearized (or linear part of) an autonomous replicating plasmid with a selection marker on it. The use of this will facilitate the genome-editing by selecting for cells that are capable of homologous recombination (for example due to cell cycle stage), and by that facilitate the genome editing process.

In 14B, a variant use of 14A is depicted, with multiple CTEC integrating in one vector, as their linkers overlap with each-other, to further facilitate multiplex editing.

FIG. 15 depicts the genome editing by ex vivo use of a CRISPR transient expression construct (CTEC) according to the invention. The CTEC is introduced into a cell that expresses an RNA-guided genome editing enzyme (e.g. Cas9, Cpf, a variant of these or other Cas-like protein) e.g. by transformation together with an autonomous replicating plasmid comprising a selection marker and an expression cassette for Cas9 or Cpf1 or by transformation together with an autonomous replicating plasmid with a selection marker and with Cas9 or Cpf1 protein.

FIG. 16 depicts the genome editing by ex vivo use of a CRISPR transient expression construct (CTEC) according to the invention. The CTEC is introduced into a cell that pre-expresses an RNA-guided genome editing enzyme (e.g. Cas9, Cpf, a variant of these or other Cas-like protein) e.g. by transformation together with an autonomous replicating plasmid comprising a selection marker and an expression cassette for Cas9 or Cpf1 or by transformation together with an autonomous replicating plasmid with a selection marker together with Cas9 protein or Cpf1 protein.

FIG. 17 depicts designs of the CTEC DNA fragments for Cas9 editing. The CTEC DNA fragments consist of the sgRNA expression cassette which comprises the SNR52p RNA polymerase III promoter, a guide-sequence (also referred to as genomic target sequence), targeting the YFP gene, followed by the gRNA structural component and the SUP4 3′ flanking region as described in DiCarlo et al., 2013, and the donor DNA. The donor encodes either a frameshift, 1 DNA base deletion or encodes 2 flanking regions just outside the YFP expression cassette that are adjacent to one another in the donor DNA resulting in the full knockout of the YFP expression cassette. The length of the donor DNA varies from 60 to 100 bp in size, for complete knock out of the YFP gene as well as introduction of a frameshift, in both cases when the donor DNA is incorporated the YFP fluorescence is lost. The CTEC fragments used have a 50 bp sequence homologous to linearized pRN1120 vector backbone (digested by EcoRI and XhoI) on either side for in-vivo circularization of the pRN1120 plasmid containing the CTEC fragment. On the 3′ side connector F (CONF) is included in between the donor DNA and the 50 bp sequence homologous to the linearized pRN1120 fragment.

FIG. 18 depicts the vector map of the single copy (CEN/ARS) vector MB7452 encoding Cas9 codon optimized for expression in Yarrowia lipolytica. Codon optimized Cas9 is expressed from the Yarrowia lipolytica 007 promoter and the Yarrowia lipolytica GPD terminator. A NatMX marker cassette is present on the vector, which confers resistance against nourseothricin to allow selection of transformants on agar plate or in liquid cultures. The beta lactamase marker allows for selection of the plasmid in E. coli.

FIG. 19 depicts the vector map of vector pSTV089. A HygB marker cassette is present on the vector, which confers resistance against hygromycin B to allow selection of transformants on agar plate or in liquid cultures. The vector expresses Cas9 (codon optimized for expression in Yarrowia lipolytica) as well as the sgRNA expression cassette targeting the Yarrowia KU70 gene. The sgRNA expression cassette comprises the Yarrowia YI_HYPO promoter, 6 bp inverted repeat of the KU70 genomic target, HH ribozyme, KU70 genomic target, HDV ribozyme and Yarrowia PGM terminator.

FIG. 20 depicts the vector map of vector pSTV086. A HygB marker cassette is present on the vector, which confers resistance against hygromycin B to allow selection of transformants on agar plate or in liquid cultures. The vector expresses Cas9 (codon optimized for expression in Yarrowia lipolytica) as well as the sgRNA expression cassette targeting the INT05 locus in the Yarrowia genome. The sgRNA expression cassette comprises the Yarrowia YI_HYPO promoter, 6 bp inverted repeat of the INT05 genomic target, HH ribozyme, INT05 genomic target, HDV ribozyme and Yarrowia PGM terminator.

FIG. 21 depicts the vector map of vector pSTV077. A HygB marker cassette is present on the vector, which confers resistance against hygromycin B to allow selection of Yarrowia lipolytica transformants on agar plate or in liquid cultures. The beta lactamase marker allows for selection of the plasmid in E. coli.

### DESCRIPTION OF THE SEQUENCES

SEQ ID NO: 1 sets out the nucleotide sequence of Cas9, including a C-terminal SV40 nuclear localization signal, codon pair optimized for expression in Saccharomyces cerevisiae. The sequence includes the KI11 promoter (promoter of KLLA0F20031g) from Kluyveromyces lactis and the GND2 terminator sequence from Saccharomyces cerevisiae.

SEQ ID NO: 2 sets out the nucleotide sequence of vector pCSN061.

SEQ ID NO: 3 sets out the nucleotide sequence of vector pRN1120.

SEQ ID NO: 4 sets out the nucleotide sequence of the forward primer to obtain Pthd3-YFP-Tenol expression cassette.

SEQ ID NO: 5 sets out the nucleotide sequence of the reverse primer to obtain Pthd3-YFP-Tenol expression cassette.

SEQ ID NO: 6 sets out the nucleotide sequence of the forward primer to attach connector 5 to the Pthd3-YFP-Tenol expression cassette.

SEQ ID NO: 7 sets out the nucleotide sequence of the reverse primer to attach connector 3 to the Pthd3-YFP-Tenol expression cassette.

SEQ ID NO: 8 sets out the nucleotide sequence of the Pthd3-YFP-Tenol expression cassette flanked by connector 5 (CON5) and connector 3 (CON3); CON5-Pthd3-YFP-Tenol-CON3.

SEQ ID NO: 9 sets out the nucleotide sequence of the forward primer to attach a 50 bp genomic DNA flank to connector 5 of YFP expression cassette; CON5-Pthd3-YFP-Tenol-CON3.

SEQ ID NO: 10 sets out the nucleotide sequence of the reverse primer to attach a 50 bp genomic DNA flank to connector 3 of YFP expression cassette; CON5-Pthd3-YFP-Tenol-CON3.

SEQ ID NO: 11 sets out the nucleotide sequence of CON5-Pthd3-YFP-Tenol-CON3 expression cassette that contains 50 bp genomic DNA flanks at 5′ and 3′ side for integration in the genome.

SEQ ID NO: 12 sets out the nucleotide sequence of the guide sequence (genomic target sequence) of INT1 for Cas9.

SEQ ID NO: 13 sets out the nucleotide sequence of the complete guide RNA cassette for targeting CAS9 to INT1 locus in the genome that contains homology to vector backbone pRN1120 for homologous recombination.

SEQ ID NO: 14 sets out the nucleotide sequence of CTEC-1 comprising a guide RNA cassette (sgRNA) for Cas9 targeting to INT1 and donor DNA on the 3′ side.

SEQ ID NO: 15 sets out the nucleotide sequence of CTEC-2 comprising a guide RNA cassette (sgRNA) for Cas9 targeting to INT1, connector A and donor DNA on the 3′ side.

SEQ ID NO: 16 sets out the nucleotide sequence of CTEC-3 comprising a guide RNA cassette (sgRNA) for Cas9 targeting to INT1 and donor DNA on the 5′ side.

SEQ ID NO: 17 sets out the nucleotide sequence of CTEC-4 comprising a guide RNA cassette (sgRNA) for Cas9 targeting to INT1, connector A and donor DNA on the 5′ side.

SEQ ID NO: 18 sets out the nucleotide sequence of CTEC-5 comprising a guide RNA cassette (sgRNA) for Cas9 targeting to INT1, PAM and guide target sequence and donor DNA on the 5′ side.

SEQ ID NO: 19 sets out the nucleotide sequence of CTEC-6B comprising a guide RNA cassette (sgRNA) for Cas9 targeting to INT1, PAM and guide target sequence and donor DNA on the 3′ side.

SEQ ID NO: 20 sets out the nucleotide sequence of CTEC-1 comprising a guide RNA cassette (sgRNA) for Cas9 targeting to the YFP gene and donor DNA on the 3′ side.

SEQ ID NO: 21 sets out the nucleotide sequence of CTEC-2 comprising a guide RNA cassette (sgRNA) for Cas9 targeting to the YFP gene, connector A and donor DNA on the 3′ side.

SEQ ID NO: 22 sets out the nucleotide sequence of CTEC-3 comprising a guide RNA cassette (sgRNA) for Cas9 targeting to the YFP gene and donor DNA on the 5′ side.

SEQ ID NO: 23 sets out the nucleotide sequence of CTEC-4 comprising a guide RNA cassette (sgRNA) for Cas9 targeting to the YFP gene, connector A and donor DNA on the 5′ side.

SEQ ID NO: 24 sets out the nucleotide sequence of CTEC-5 comprising a guide RNA cassette (sgRNA) for Cas9 targeting to the YFP gene, PAM and guide target sequence and donor DNA on the 5′ side.

SEQ ID NO: 25 sets out the nucleotide sequence of CTEC-6A comprising a guide RNA cassette (sgRNA) for Cas9 targeting to the YFP gene, guide target and PAM sequence and donor DNA on the 3′ side.

SEQ ID NO: 26 sets out the nucleotide sequence of guide sequence (genomic target sequence) of INT1 for Cas9.

SEQ ID NO: 27 sets out the nucleotide sequence of guide sequence (genomic target sequence) of YFP for Cas9.

SEQ ID NO: 28 sets out the nucleotide sequence of connector A.

SEQ ID NO: 29 sets out the nucleotide sequence of the complete guide RNA expression cassette for targeting Cas9 to the YFP expression cassette in the genome of CSN009.

SEQ ID NO: 30 sets out the nucleotide sequence of the complete guide RNA expression cassette for targeting Cas9 to the INT1 locus in the genome of CSN001.

SEQ ID NO: 31 sets out the nucleotide sequence of the YFP donor DNA that is part of CTEC fragments for Cas9 editing.

SEQ ID NO: 32 sets out the nucleotide sequence of the INT1 donor DNA that is part of CTEC fragments for Cas9 editing.

SEQ ID NO: 33 sets out the nucleotide sequence of the forward primer to amplify CTEC fragments that contain donor DNA on the 3′ side.

SEQ ID NO: 34 sets out the nucleotide sequence of the forward primer to amplify CTEC fragments that contain the YFP donor DNA on the 5′ side.

SEQ ID NO: 35 sets out the nucleotide sequence of the reverse primer to amplify CTEC fragments that contain the YFP donor DNA on the 3′ side.

SEQ ID NO: 36 sets out the nucleotide sequence of the reverse primer to amplify CTEC fragments that contain donor DNA on the 5′ side.

SEQ ID NO: 37 sets out the nucleotide sequence of the forward primer to amplify CTEC fragments that contain the INT1 donor DNA on the 5′ side.

SEQ ID NO: 38 sets out the nucleotide sequence of the reverse primer to amplify CTEC fragments that contain the INT1 donor DNA on the 3′ side.

SEQ ID NO: 39 sets out the nucleotide sequence of the forward primer to amplify the YFP ORF.

SEQ ID NO: 40 sets out the nucleotide sequence of the reverse primer to amplify the YFP ORF.

SEQ ID NO: 41 sets out the nucleotide sequence of forward primer used for sequencing the YFP ORF.

SEQ ID NO: 42 sets out the nucleotide sequence of the forward primer to amplify part of the INT1 locus.

SEQ ID NO: 43 sets out the nucleotide sequence of the reverse primer to amplify part of the INT1 locus.

SEQ ID NO: 44 sets out the nucleotide sequence of the forward primer used for sequencing part of the INT1 locus.

SEQ ID NO: 45 sets out the nucleotide sequence of the forward primer to amplify the KI11p-pCSN061 backbone-GND2t PCR fragment.

SEQ ID NO: 46 sets out the nucleotide sequence of the reverse primer to amplify the KI11p-pCSN061 backbone-GND2t PCR fragment.

SEQ ID NO: 47 sets out the protein sequence of LbCpf1 (from Lachnospiraceae bacterium ND2006) including a C-terminal NLS.

SEQ ID NO: 48 sets out the nucleotide sequence CPO LbCpf1 including a C-terminal NLS.

SEQ ID NO: 49 sets out the nucleotide sequence of the forward primer to amplify LbCpf1 expression cassette.

SEQ ID NO: 50 sets out the nucleotide sequence of the reverse primer to amplify LbCpf1 expression cassette.

SEQ ID NO: 51 sets out the nucleotide sequence of vector pCSN067 encoding LbCpf1.

SEQ ID NO: 52 sets out the nucleotide sequence of direct repeat part of crRNA cassette of LbCpf1.

SEQ ID NO: 53 sets out the nucleotide sequence of guide sequence (genomic target sequence) of INT1 for LbCpf1.

SEQ ID NO: 54 sets out the nucleotide sequence of the complete guide RNA cassette for targeting LbCpf1 to the INT1 locus in the genome that contains homology to vector backbone pRN1120 for homologous recombination.

SEQ ID NO: 55 sets out the nucleotide sequence of CTEC-7 comprising a guide RNA cassette (crRNA) for targeting LbCpf1 to the YFP gene and donor DNA on the 3′ side.

SEQ ID NO: 56 sets out the nucleotide sequence of CTEC-8 comprising a guide RNA cassette (crRNA) for targeting LbCpf1 to the YFP gene, connector A and donor DNA on the 3′ side.

SEQ ID NO: 57 sets out the nucleotide sequence of CTEC-9 comprising a guide RNA cassette (crRNA) for targeting LbCpf1 to the YFP gene and donor DNA on the 5′ side.

SEQ ID NO: 58 sets out the nucleotide sequence of CTEC-10 comprising a guide RNA cassette (crRNA) for targeting LbCpf1 to the YFP gene, connector A and donor DNA on the 5′ side.

SEQ ID NO: 59 sets out the nucleotide sequence of CTEC-11 comprising a guide RNA cassette (crRNA) for targeting LbCpf1 to the YFP gene, PAM and guide target sequence and donor DNA on the 3′ side (2×18 bp guide).

SEQ ID NO: 60 sets out the nucleotide sequence of CTEC-11 comprising a guide RNA cassette (crRNA) for targeting LbCpf1 to the YFP gene, PAM and guide target sequence and donor DNA on the 3′ side (2×20 bp guide).

SEQ ID NO: 61 sets out the nucleotide sequence of CTEC-12 comprising a guide RNA cassette (crRNA) for targeting LbCpf1 to the YFP gene, PAM and guide target sequence and donor DNA on the 5′ side (2×18 bp guide).

SEQ ID NO: 62 sets out the nucleotide sequence of CTEC-12 comprising a guide RNA cassette (crRNA) for targeting LbCpf1 to the YFP gene, PAM and guide target sequence and donor DNA on the 5′ side (2×20 bp guide).

SEQ ID NO: 63 sets out the nucleotide sequence of CTEC-7 comprising a guide RNA cassette (crRNA) for targeting LbCpf1 to INT1 and donor DNA on the 3′ side.

SEQ ID NO: 64 sets out the nucleotide sequence of CTEC-8 comprising a guide RNA cassette (crRNA) for targeting LbCpf1 to INT1, connector A and donor DNA on the 3′.

SEQ ID NO: 67 sets out the nucleotide sequence of CTEC-11 comprising a guide RNA cassette (crRNA) for targeting LbCpf1 to INT1, PAM and guide target sequence and donor DNA on the 3′ side (1×20 bp, 1×18 bp guide).

SEQ ID NO: 68 sets out the nucleotide sequence of CTEC-11 comprising a guide RNA cassette (crRNA) for targeting LbCpf1 to INT1, PAM and guide target sequence and donor DNA on the 3′ side (2×20 bp guide).

SEQ ID NO: 69 sets out the nucleotide sequence of the guide sequence (genomic target) of the CTEC fragments targeting YFP by LbCpf1 in strain CSN010.

SEQ ID NO: 70 sets out the nucleotide sequence of the guide sequence (genomic target) of the CTEC fragments targeting INT1 by LbCpf1 in strain CSN004.

SEQ ID NO: 71 sets out the nucleotide sequence of YFP donor DNA that is part of CTEC fragments for LbCpf1 mediated editing in strain CSN010.

SEQ ID NO: 72 sets out the nucleotide sequence of INT donor DNA that is part of CTEC fragments for LbCpf1 mediated editing in strain CSN004.

SEQ ID NO: 73 sets out the nucleotide sequence of complete guide RNA expression cassette for targeting LbCpf1 to the INT1 locus in the genome of CSN004.

SEQ ID NO: 74 sets out the nucleotide sequence of complete guide RNA expression cassette for targeting LbCpf1 to the YFP expression cassette in the genome of CSN010.

SEQ ID NO: 75 sets out the nucleotide sequence of the 18 bp guide sequence (genomic target sequence) for digestion of the CTEC fragment by LbCpf1 thereby separating the INT1 donor DNA from the guide RNA expression cassette.

SEQ ID NO: 76 sets out the nucleotide sequence of the 18 bp guide sequence (genomic target sequence) for digestion of the CTEC fragment by LbCpf1 thereby separating the YFP donor DNA from the guide RNA expression cassette.

SEQ ID NO: 77 sets out the nucleotide sequence of the 20 bp guide sequence (genomic target sequence) for digestion of the CTEC fragment by LbCpf1 thereby separating the INT1 donor DNA from the guide RNA expression cassette.

SEQ ID NO: 78 sets out the nucleotide sequence of the 20 bp guide sequence (genomic target sequence) for digestion of the CTEC fragment by LbCpf1 thereby separating the YFP donor DNA from the guide RNA expression cassette.

SEQ ID NO: 79 sets out the nucleotide sequence of the 18 bp guide sequence (genomic target sequence) including the PAM sequence for digestion of the CTEC fragment by LbCpf1 thereby separating the INT1 donor DNA from the guide RNA expression cassette.

SEQ ID NO: 80 sets out the nucleotide sequence of the 20 bp guide sequence (genomic target sequence) including the PAM sequence for digestion of the CTEC fragment by LbCpf1 thereby separating the INT1 donor DNA from the guide RNA expression cassette.

SEQ ID NO: 81 sets out the nucleotide sequence of the 18 bp guide sequence (genomic target sequence) including the PAM for digestion of the CTEC fragment by LbCpf1 thereby separating the YFP donor DNA from the guide RNA expression cassette.

SEQ ID NO: 82 sets out the nucleotide sequence of the 20 bp guide sequence (genomic target sequence) including the PAM sequence for digestion of the CTEC fragment by LbCpf1 thereby separating the YFP donor DNA from the guide RNA expression cassette.

SEQ ID NO: 83 sets out the nucleotide sequence of the reverse primer to amplify CTEC fragments having the YFP donor on the 5′ side and a 20 bp guide sequence for LbCpf1.

SEQ ID NO: 84 sets out the nucleotide sequence of the reverse primer to amplify CTEC fragments having the YFP donor on the 5′ side and a 18 bp guide sequence for LbCpf1.

SEQ ID NO: 85 sets out the nucleotide sequence of the forward primer to amplify CTEC fragments having the INT1 donor on the 5′ side for LbCpf1 editing.

SEQ ID NO: 86 sets out the nucleotide sequence of the reverse primer to amplify CTEC fragments having the INT1 donor on the 3′ side for LbCpf1 editing.

SEQ ID NO: 87 sets out the nucleotide sequence of CTEC-7 comprising a guide RNA cassette (crRNA) for targeting LbCpf1 to the YFP gene and donor DNA on the 3′ side, flanked by connector 5 sequence on the 5′ side and connector 3 on the 3′ side.

SEQ ID NO: 88 sets out the nucleotide sequence of CTEC-8 comprising a guide RNA cassette (crRNA) for targeting LbCpf1 to the YFP gene, connector A and donor DNA on the 3′ side, flanked by connector 5 sequence on the 5′ side and connector 3 on the 3′ side.

SEQ ID NO: 89 sets out the nucleotide sequence of CTEC-9 comprising a guide RNA cassette (crRNA) for targeting LbCpf1 to the YFP gene and donor DNA on the 5′ side, flanked by connector 5 sequence on the 5′ side and connector 3 on the 3′ side.

SEQ ID NO: 90 sets out the nucleotide sequence of CTEC-10 comprising a guide RNA cassette (crRNA) for targeting LbCpf1 to the YFP gene, connector A and donor DNA on the 5′ side, flanked by connector 5 sequence on the 5′ side and connector 3 on the 3′ side.

SEQ ID NO: 91 sets out the nucleotide sequence of CTEC-11 comprising a guide RNA cassette (crRNA) for targeting LbCpf1 to the YFP gene, PAM and guide target sequence and donor DNA on the 3′ side (2×18 bp guide), flanked by connector 5 sequence on the 5′ side and connector 3 on the 3′ side.

SEQ ID NO: 92 sets out the nucleotide sequence of CTEC-11 comprising a guide RNA cassette (crRNA) for targeting LbCpf1 to the YFP gene, PAM and guide target sequence and donor DNA on the 3′ side (2×20 bp guide), flanked by connector 5 sequence on the 5′ side and connector 3 on the 3′ side.

SEQ ID NO: 93 sets out the nucleotide sequence of CTEC-12 comprising a guide RNA cassette (crRNA) for targeting LbCpf1 to the YFP gene, PAM and guide target sequence and donor DNA on the 5′ side (2×18 bp guide), flanked by connector 5 sequence on the 5′ side and connector 3 on the 3′ side.

SEQ ID NO: 94 sets out the nucleotide sequence of CTEC-12 comprising a guide RNA cassette (crRNA) for targeting LbCpf1 to the YFP gene, PAM and guide target sequence and donor DNA on the 5′ side (2×20 bp guide), flanked by connector 5 sequence on the 5′ side and connector 3 on the 3′ side.

SEQ ID NO: 95 sets out the nucleotide sequence of the forward primer to amplify CTEC fragments with connector 5 on the 5′ side.

SEQ ID NO: 96 sets out the nucleotide sequence of the reverse primer to amplify CTEC fragments with connector 3 on the 3′ side.

SEQ ID NO: 97 sets out the nucleotide sequence of connector 5.

SEQ ID NO: 98 sets out the nucleotide sequence of connector 3.

SEQ ID NO: 99 sets out the nucleotide sequence of CTEC-7 comprising a guide RNA cassette (crRNA) for targeting LbCpf1 to the YFP gene and donor DNA on the 3′ side, flanked by connector 5 sequence on the 5′ side.

SEQ ID NO: 100 sets out the nucleotide sequence of CTEC-8 comprising a guide RNA cassette (crRNA) for targeting LbCpf1 to the YFP gene, connector A and donor DNA on the 3′ side, flanked by connector 5 sequence on the 5′ side.

SEQ ID NO: 101 sets out the nucleotide sequence of CTEC-9 comprising a guide RNA cassette (crRNA) for targeting LbCpf1 to the YFP gene and donor DNA on the 5′ side, flanked by connector 5 sequence on the 5′ side.

SEQ ID NO: 102 sets out the nucleotide sequence of CTEC-10 comprising a guide RNA cassette (crRNA) for targeting LbCpf1 to the YFP gene, connector A and donor DNA on the 5′ side, flanked by connector 5 sequence on the 5′ side.

SEQ ID NO: 103 sets out the nucleotide sequence of CTEC-11 comprising a guide RNA cassette (crRNA) for targeting LbCpf1 to the YFP gene, PAM and guide target sequence and donor DNA on the 3′ side (2×18 bp guide), flanked by connector 5 sequence on the 5′ side.

SEQ ID NO: 104 sets out the nucleotide sequence of CTEC-11 comprising a guide RNA cassette (crRNA) for targeting LbCpf1 to the YFP gene, PAM and guide target sequence and donor DNA on the 3′ side (2×20 bp guide), flanked by connector 5 sequence on the 5′ side.

SEQ ID NO: 105 sets out the nucleotide sequence of CTEC-12 comprising a guide RNA cassette (crRNA) for targeting LbCpf1 to the YFP gene, PAM and guide target sequence and donor DNA on the 5′ side (2×18 bp guide), flanked by connector 5 sequence on the 5′ side.

SEQ ID NO: 106 sets out the nucleotide sequence of CTEC-12 comprising a guide RNA cassette (crRNA) for targeting LbCpf1 to the YFP gene, PAM and guide target sequence and donor DNA on the 5′ side (2×20 bp guide), flanked by connector 5 sequence on the 5′ side.

SEQ ID NO: 107 sets out the nucleotide sequence of CTEC-7 comprising a guide RNA cassette (crRNA) for targeting LbCpf1 to the YFP gene and donor DNA on the 3′ side, flanked by connector 3 sequence on the 3′ side.

SEQ ID NO: 108 sets out the nucleotide sequence of CTEC-8 comprising a guide RNA cassette (crRNA) for targeting LbCpf1 to the YFP gene, connector A and donor DNA on the 3′ side, flanked by connector 3 sequence on the 3′ side.

SEQ ID NO: 109 sets out the nucleotide sequence of CTEC-9 comprising a guide RNA cassette (crRNA) for targeting LbCpf1 to the YFP gene and donor DNA on the 5′ side, flanked by connector 3 sequence on the 3′ side.

SEQ ID NO: 110 sets out the nucleotide sequence of CTEC-10 comprising a guide RNA cassette (crRNA) for targeting LbCpf1 to the YFP gene, connector A and donor DNA on the 5′ side, flanked by connector 3 sequence on the 3′ side.

SEQ ID NO: 111 sets out the nucleotide sequence of CTEC-11 comprising a guide RNA cassette (crRNA) for targeting LbCpf1 to the YFP gene, PAM and guide target sequence and donor DNA on the 3′ side (2×18 bp guide), flanked by connector 3 sequence on the 3′ side.

SEQ ID NO: 112 sets out the nucleotide sequence of CTEC-11 comprising a guide RNA cassette (crRNA) for targeting LbCpf1 to the YFP gene, PAM and guide target sequence and donor DNA on the 3′ side (2×20 bp guide), flanked by connector 3 sequence on the 3′ side.

SEQ ID NO: 113 sets out the nucleotide sequence of CTEC-12 comprising a guide RNA cassette (crRNA) for targeting LbCpf1 to the YFP gene, PAM and guide target sequence and donor DNA on the 5′ side (2×18 bp guide), flanked by connector 3 sequence on the 3′ side.

SEQ ID NO: 114 sets out the nucleotide sequence of CTEC-12 comprising a guide RNA cassette (crRNA) for targeting LbCpf1 to the YFP gene, PAM and guide target sequence and donor DNA on the 5′ side (2×20 bp guide), flanked by connector 3 sequence on the 3′ side.

SEQ ID NO: 115 sets out the nucleotide sequence of CTEC-1 comprising a guide RNA cassette (sgRNA) for targeting Cas9 to the YFP gene and donor DNA of 60 bp, which encodes a frameshift, on the 3′ side. The CTEC fragment contains 50 bp homology on either side to the linearized pRN1120 vector fragment (EcoRI and XhoI digested) for in vivo circularization. On the 3′ side, connector F (CONF) is included in between the donor DNA and the 50 bp homology to linearized pRN1120 vector backbone fragment.

SEQ ID NO: 116 sets out the nucleotide sequence of CTEC-1 comprising a guide RNA cassette (sgRNA) for targeting Cas9 to the YFP gene and donor DNA of 80 bp, which encodes a frameshift, on the 3′ side. The CTEC fragment contains 50 bp homology on either side to the linearized pRN1120 vector fragment (EcoRI and XhoI digested) for in vivo circularization. On the 3′ side, connector F (CONF) is included in between the donor DNA and the 50 bp homology to linearized pRN1120 vector backbone fragment.

SEQ ID NO: 117 sets out the nucleotide sequence of CTEC-1 comprising a guide RNA cassette (sgRNA) for targeting Cas9 to the YFP gene and donor DNA of 100 bp, which encodes a frameshift, on the 3′ side. The CTEC fragment contains 50 bp homology on either side to the linearized pRN1120 vector fragment (EcoRI and XhoI digested) for in vivo circularization. On the 3′ side, connector F (CONF) is included in between the donor DNA and the 50 bp homology to linearized pRN1120 vector backbone fragment.

SEQ ID NO: 118 sets out the nucleotide sequence of CTEC-1 comprising a guide RNA cassette (sgRNA) for targeting Cas9 to the YFP gene and donor DNA of 60 bp, which encodes the full knock out of the YFP expression cassette, on the 3′ side. The CTEC fragment contains 50 bp homology on either side to the linearized pRN1120 vector fragment (EcoRI and XhoI digested) for in vivo circularization. On the 3′ side, connector F (CONF) is included in between the donor DNA and the 50 bp homology to linearized pRN1120 vector backbone fragment.

SEQ ID NO: 119 sets out the nucleotide sequence of CTEC-1 comprising a guide RNA cassette (sgRNA) for targeting Cas9 to the YFP gene and donor DNA of 80 bp, which encodes the full knock out of the YFP expression cassette, on the 3′ side. The CTEC fragment contains 50 bp homology on either side to the linearized pRN1120 vector fragment (EcoRI and XhoI digested) for in vivo circularization. On the 3′ side, connector F (CONF) is included in between the donor DNA and the 50 bp homology to linearized pRN1120 vector backbone fragment.

SEQ ID NO: 120 sets out the nucleotide sequence of CTEC-1 comprising a guide RNA cassette (sgRNA) for targeting Cas9 to the YFP gene and donor DNA of 100 bp, which encodes the full knock out of the YFP expression cassette, on the 3′ side. The CTEC fragment contains 50 bp homology on either side to the linearized pRN1120 vector fragment (EcoRI and XhoI digested) for in vivo circularization. On the 3′ side, connector F (CONF) is included in between the donor DNA and the 50 bp homology to linearized pRN1120 vector backbone fragment.

SEQ ID NO: 121 sets out the nucleotide sequence of the complete guide RNA expression cassette (sgRNA) for targeting Cas9 to the YFP expression cassette in the genome of CSN009.

SEQ ID NO: 122 sets out the nucleotide sequence of the guide sequence (genomic target) of the CTEC fragments targeting YFP by Cas9 in strain CSN009.

SEQ ID NO: 123 sets out the nucleotide sequence of the donor DNA encoding a frameshift in the YFP gene, 60 bp.

SEQ ID NO: 124 sets out the nucleotide sequence of the donor DNA encoding a frameshift in the YFP gene, 80 bp.

SEQ ID NO: 125 sets out the nucleotide sequence of the donor DNA encoding a frameshift in the YFP gene, 100 bp.

SEQ ID NO: 126 sets out the nucleotide sequence of the donor DNA encoding the knock out of the YFP expression cassette, 60 bp.

SEQ ID NO: 127 sets out the nucleotide sequence of the donor DNA encoding the knock out of the YFP expression cassette, 80 bp.

SEQ ID NO: 128 sets out the nucleotide sequence of the donor DNA encoding the knock out of the YFP expression cassette, 100 bp.

SEQ ID NO: 129 sets out the nucleotide sequence of the forward primer for amplification of CTEC fragments (SEQ ID NO's: 115, 116, 117, 118, 119 and 120) that are flanked by 50 bp sequences homologous to the linearized pRN1120 vector backbone fragment (EcoRI and XhoI digested).

SEQ ID NO: 130 sets out the nucleotide sequence of the reverse primer for amplification of CTEC fragments (SEQ ID NO's: 115, 116, 117, 118, 119 and 120) that are flanked by 50 bp sequences homologous to the linearized pRN1120 vector backbone fragment (EcoRI and XhoI digested).

SEQ ID NO: 131 sets out the nucleotide sequence of connector F (CONF).

SEQ ID NO: 132 sets out the nucleotide sequence of the wild-type genomic target (example 4)

SEQ ID NO: 133 sets out the nucleotide sequence of the modified genomic target (example 4)

SEQ ID NO: 134 sets out the nucleotide sequence of CTEC DNA fragment 3, comprising a guide RNA expression cassette (sgRNA) for targeting Cas9 to the GFP gene and donor DNA of 100-bp, which encodes a 2 base modification in the PAM sequence, changing it from CGG to TAG, on the 3′ side.

SEQ ID NO: 135 sets out the nucleotide sequence of CTEC DNA fragment 4, comprising a guide RNA expression cassette (sgRNA) for targeting Cas9 to the GFP gene and donor DNA of 100-bp, which encodes a silent mutation in the GFP gene by changing the PAM sequence from CGG to CGA. In addition to the PAM mutation, a base change from T to A is encoded in the donor DNA whereby a STOP codon is introduced. The donor DNA is located at the 3′ side of the CTEC DNA fragment 4.

SEQ ID NO: 136 sets out the nucleotide sequence of Yarrowia YI_HYPO promoter.

SEQ ID NO: 137 sets out the nucleotide sequence of the 6-bp inverted repeat of the guide sequence of the GFP gene.

SEQ ID NO: 138 sets out the nucleotide sequence of the HH ribozyme.

SEQ ID NO: 139 sets out the nucleotide sequence of the HDV ribozyme.

SEQ ID NO: 140 sets out the nucleotide sequence of the 20-bp genomic target sequence of the GFP gene.

SEQ ID NO: 141 sets out the nucleotide sequence of the Yarrowia YI_PGM terminator.

SEQ ID NO: 142 sets out the nucleotide sequence of guide-RNA expression cassette (sgRNA) targeting the GFP gene.

SEQ ID NO: 143 sets out the nucleotide sequence of 100-bp donor DNA of CTEC DNA fragment 1.

SEQ ID NO: 144 sets out the nucleotide sequence of 100-bp donor DNA of CTEC DNA fragment 2.

SEQ ID NO: 145 sets out the nucleotide sequence of 100-bp donor DNA of CTEC DNA fragment 3.

SEQ ID NO: 146 sets out the nucleotide sequence of 100-bp donor DNA of CTEC DNA fragment 4.

SEQ ID NO: 147 sets out the nucleotide sequence of plasmid MB7452.

SEQ ID NO: 148 sets out the nucleotide sequence of Cas9, including a C-terminal SV40 nuclear localization signal, codon optimized for expression in Yarrowia lipolytica. The sequence includes the 007 promoter sequence and the GPD terminator sequence, both from Yarrowia lipolytica.

SEQ ID NO: 149 sets out the nucleotide sequence of Yarrowia YI_007 promoter.

SEQ ID NO: 150 sets out the nucleotide sequence of Yarrowia YI_GPD terminator.

SEQ ID NO: 151 sets out the nucleotide sequence of pSTV089.

SEQ ID NO: 152 sets out the nucleotide sequence of the 20-bp genomic target of the KU70 gene.

SEQ ID NO: 153 sets out the nucleotide sequence of the 100-bp donor DNA fragment used for knocking out the KU70 gene in the Yarrowia genome.

SEQ ID NO: 154 sets out the nucleotide sequence of the forward primer to confirm knock out of KU70 gene in the Yarrowia genome

SEQ ID NO: 155 sets out the nucleotide sequence of the reverse primer to confirm knock out of KU70 gene in the Yarrowia genome.

SEQ ID NO: 156 sets out the nucleotide sequence of the GFP expression cassette (YI_HSP.pro-A.vic_eGFP ORF-YI_GPD.ter).

SEQ ID NO: 157 sets out the nucleotide sequence of plasmid pSTV086.

SEQ ID NO: 158 sets out the nucleotide sequence of the GFP expression cassette (YI_HSP.pro-A.vic_eGFP ORF-YI_GPD.ter) flanked by 50-bp genomic DNA sequences on either side for targeted integration in the INT05 locus.

SEQ ID NO: 159 sets out the nucleotide sequence of the forward primer to confirm integration of the GFP expression cassette in the INT05 locus in the Yarrowia genome.

SEQ ID NO: 160 sets out the nucleotide sequence of the reverse primer to confirm integration of the GFP expression cassette in the INT05 locus in the Yarrowia genome.

SEQ ID NO: 161 sets out the nucleotide sequence of plasmid pSTV077.

SEQ ID NO: 162 sets out the nucleotide sequence of Yarrowia YI_HSP promoter.

SEQ ID NO: 163 sets out the nucleotide sequence of Aequorea victoria eGFP gene (A.vic_eGFP ORF).

SEQ ID NO: 164 sets out the nucleotide sequence of Yarrowia YI_GPD terminator.

SEQ ID NO: 165 sets out the nucleotide sequence of the forward primer to amplify the edited GFP ORF from the Yarrowia genome.

SEQ ID NO: 166 sets out the nucleotide sequence of the reverse primer to amplify the edited GFP

ORF from the Yarrowia genome.

SEQ ID NO: 167 sets out the nucleotide sequence of 6 bp inverted repeat of the KU70 genomic target.

SEQ ID NO: 168 sets out the nucleotide sequence of 6 bp inverted repeat of the INT05 genomic target.

SEQ ID NO: 169 sets out the nucleotide sequence of the 20-bp genomic target sequence of the INT05 locus.

SEQ ID NO: 170 sets out the nucleotide sequence of CTEC DNA fragment 1, comprising a guide RNA expression cassette (sgRNA) for targeting Cas9 to the GFP gene and donor DNA of 100-bp, which encodes for the full knock out of the GFP ORF, on the 3′ side.

SEQ ID NO: 171 sets out the nucleotide sequence of CTEC DNA fragment 2, comprising a guide RNA expression cassette (sgRNA) for targeting Cas9 to the GFP gene and donor DNA of 100-bp, which encodes a base deletion in the PAM sequence, changing it from CGG to CG, on the 3′ side.

### DETAILED DESCRIPTION

The inventors have found that a CRISPR transient expression construct (CTEC) according to the invention provides a great improvement over the art. In this system, the guide-RNA is initially and transiently expressed from the CTEC. The expressed guide-RNA facilitates induction of a break into the target genome at the target sequence and subsequently the donor polynucleotide integrates into the target genome. This system can, e.g., conveniently be used using a library of CTECs where distinct additional functional or non-functional polynucleotide elements are present on the constructs which are linked to the guide-RNAs. The invention can conveniently be used to e.g. generate within a host cell a targeted mutation, a targeted insertion or a targeted deletion/knock-out. The CTEC as provided herein can be viewed as a donor polynucleotide in the sense as known in the art of e.g. CRISPR/Cas and CRISPR/Cpf1 gene editing, which contains its specific guide-RNA expression cassette. The specific lay-out of the CTEC according to the invention minimizes the chances of the guide-RNA part of the CTEC to integrate into the (edited) genome. This a substantial advantage over the art such as PCT/EP2018/058612 since it is no longer necessary to remove the guide-RNA cassette. In addition, it minimizes the risk of creating gene drives.

Using polynucleotide-guided nuclease/editing systems such as the CRISPR/Cas9 system, there is the possibility to develop gene drives capable of autonomously spreading genomic alterations by organisms via sexual replication, e.g. explained by DiCarlo et al., 2015. Neither the inventors, nor the applicant has intended, intends or will intend to create such gene drives or likewise autonomous gene editing tools (also known as mutagenic chain reaction or active genetics).

In a first aspect, there is provided for the ex vivo use of a CRISPR transient expression construct (CTEC) for expression in a host cell of a functional guide-RNA or part thereof that is specific for a target sequence in a target genome, wherein the CTEC is linear and comprises:
a guide-RNA expression cassette, and
an additional polynucleotide element, and,
   wherein the guide-RNA expression cassette is capable of expressing a functional guide-RNA, or a part thereof, that is specific for a target sequence in a target genome, and wherein the additional polynucleotide element has sequence identity with the target sequence in the target genome.

In the context of all embodiments of the invention, the CRISPR transient expression construct (CTEC) is a polynucleotide construct, which is not an autonomously replicating entity; it does not comprise an autonomously replicating sequence. The CTEC can be formed in vivo (within a cell) by recombination of two or more separate linear members. The term polynucleotide is defined in the “General Definitions” herein.

The target sequence in the target genome in a cell is the place where the complex of a functional polynucleotide-guided genome editing enzyme and a guide-RNA binds to and where, if applicable, a double-stranded break or single-stranded break (nick) is created (induced). Herein, the ‘target sequence’ is herein also referred to as ‘guide-RNA target’. The ‘guide-RNA expression cassette’ is herein also referred to as ‘crRNA cassette’.

The terms “targeted mutation”, “targeted insertion” and “targeted deletion/knock-out” in al embodiments of the invention mean that the mutation, insertion, deletion/knock-out is made in a pre-defined place in the genome of the host cell. A mutation can be a silent mutation or a mutation that results in an amino acid change. A mutation is not limited to mutation of a single nucleotide, two or more nucleotides may be mutated. An insertion means that at least one nucleotide is added to the target genome. An insertion can be combined with a mutation and/or a deletion as long the resulting genome is different from the target genome before CTEC editing. A deletion means that at least one nucleotide is deleted from the target genome. A deletion can be combined with a mutation and/or deletion as long as the resulting genome is different from the target genome before editing. An insertion may have any suitable length, such as at least one nucleotide, at least 10 nucleotides, at least 20, 30, 40, 50, 60, 70, 80, 90, 100, 110, 120, 130, 140, 150, 160, 170, 180, 190, 200, 250, 300, 400, 500, 600, 700, 800, 900, or at least 1000 nucleotides. An insertion may have at most 20, 30, 40, 50, 60, 70, 80, 90, 100, 110, 120, 130, 140, 150, 160, 170, 180, 190, 200, 250, 300, 400, 500, 600, 700, 800, 900, or at least 1000 nucleotides. An insertion may be within the range of 20-1000, 100-1000, 100-500, or 200-500 nucleotides. A deletion may have any suitable length, such as at least one, two, three, four, five, six, seven, eight, nine nucleotide(s), at least 10 nucleotides, at least 20, 30, 40, 50, 60, 70, 80, 90, 100, 110, 120, 130, 140, 150, 160, 170, 180, 190, 200, 250, 300, 400, 500, 600, 700, 800, 900, or at least 1000 nucleotides. A deletion may be at most 20, 30, 40, 50, 60, 70, 80, 90, 100, 110, 120, 130, 140, 150, 160, 170, 180, 190, 200, 250, 300, 400, 500, 600, 700, 800, 900, 1000, 2000, 3000, 4000 or 5000 nucleotides. An deletion may be within the range of 20-5000, 100-1000, 100-500, or 200-500 nucleotides. In all embodiments of the invention, the CRISPR transient expression construct (CTEC) is a linear CRISPR transient expression construct. Linear has the meaning as known in the art for a polynucleotide; it is to be construed that the polynucleotide is not circular, has two clearly defined ends, a 5′-end and a 3′-end, which ends are preferably both blunt ends. A CTEC according to the invention may be de novo synthesized, it may be generated by e.g. PCR or by digestion by a restriction enzyme from a vector, such as a plasmid, from a library or other system. A guide-RNA expression cassette according to the invention is a polynucleotide expression construct that comprises the components, except for the RNA polymerase, needed to express a functional guide-RNA or a part thereof, in vivo such as within a cell. The components include, but are not limited to, a promoter, a coding sequence encoding a guide-RNA or a part thereof and a terminator. Such components are known to the person skilled in the art and are preferably those as defined herein. The “part thereof” of the guide-RNA is preferably the part that comprises or consists of the guide-sequence. The guide-sequence is the recognition sequence, i.e. the sequence that is specific, i.e. substantially complementary, for the target sequence in the target genome and that allows targeting of a complex of a functional polynucleotide-guided genome editing enzyme and a functional guide-RNA to the target sequence in the target genome. The term “specific” in the context of the guide-sequence in the guide-RNA or part thereof, is to be construed that the guide-sequence is substantially complementary to the target sequence in the target genome, wherein “substantially complementary” means that there is sufficient complementarity (sequence identity) between target sequence and guide-sequence to allow hybridization under physiological conditions in a cell; in general one or two mismatches are allowed to still allow sufficient hybridization. The degree of complementarity (sequence identity), when optimally aligned using a suitable alignment algorithm, is preferably higher than 50%, 60%, 70%, 75%, 80%, 85%, 90%, 95%, 96%, 97%, 98% or higher than 99%. Different sequences can guide nucleases, like guide-RNA's for Cas9 (Mali et al., 2013; Cong et al., 2013) and guide-RNA's for Cpf1 (Zetsche et al., 2015) as known to the person skilled in the art. When the coding sequence in the CTEC does not encode a complete and functional guide-RNA, but encodes the part of the guide-RNA that comprises or consists of the guide-sequence, the other parts of the guide-RNA that together with the guide-sequence form a functional guide-RNA are encoded on a different construct or are present as such within the cell. The construct encoding the remaining components of the guide-RNA may be present in the genome or may be present on a vector or may be present as such in the cell or may be delivered as such to the cell. A functional polynucleotide-guided genome editing enzyme can be any system known to the person skilled in the art. Suitable functional genome editing systems for use in all embodiments of the invention include: RNA-guided endonucleases like CRISPR/Cas (Mali et al., 2013; Cong et al., 2013) or CRISPR/Cpf1 (Zetsche et al., 2015). The functional genome editing enzyme can be a native or a heterologous enzyme, and can be an enzyme such as a Cas enzyme, preferably Cas9 or Cas9 nickase; a Cpf1.

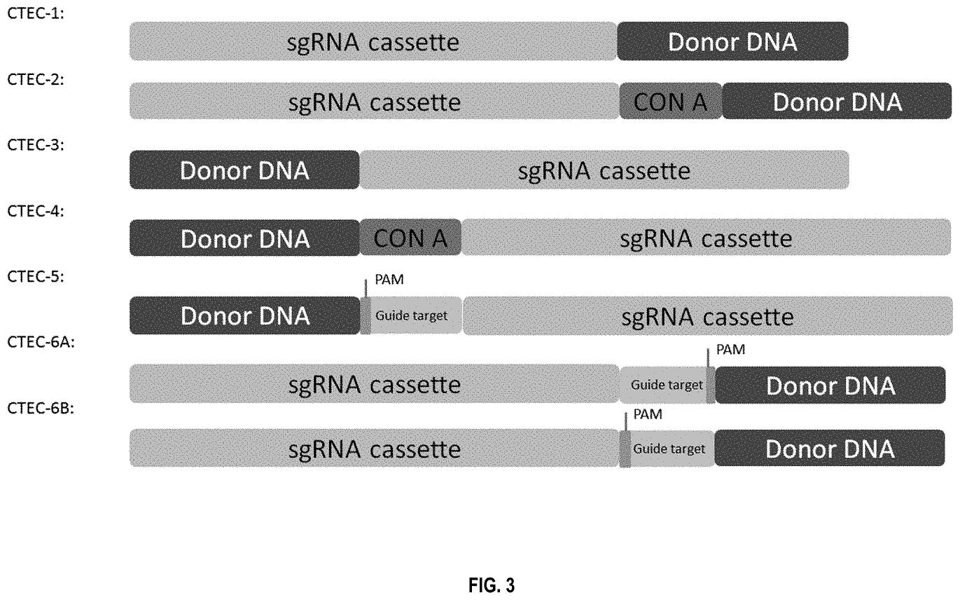

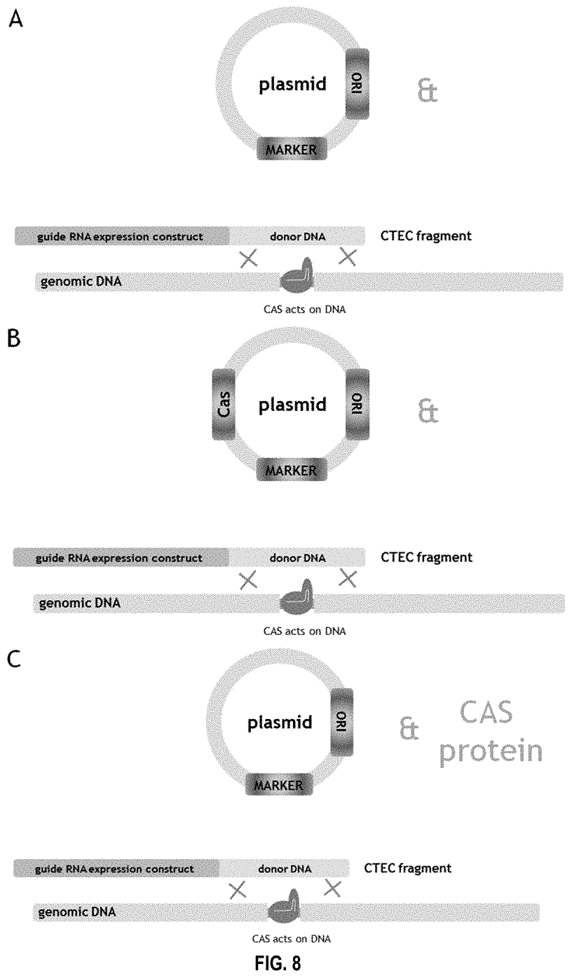

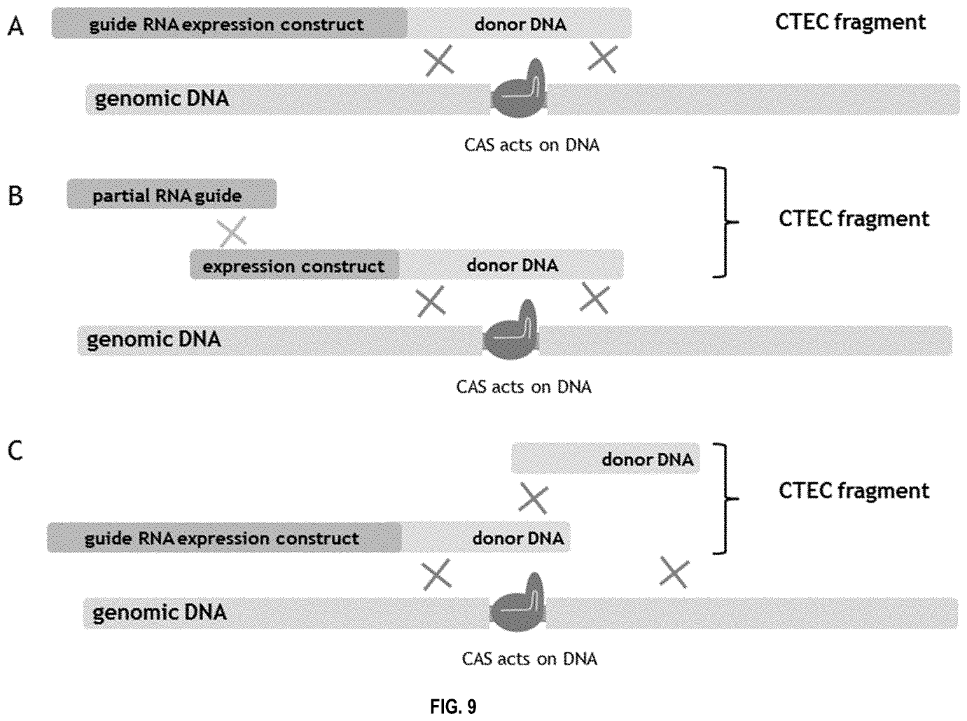

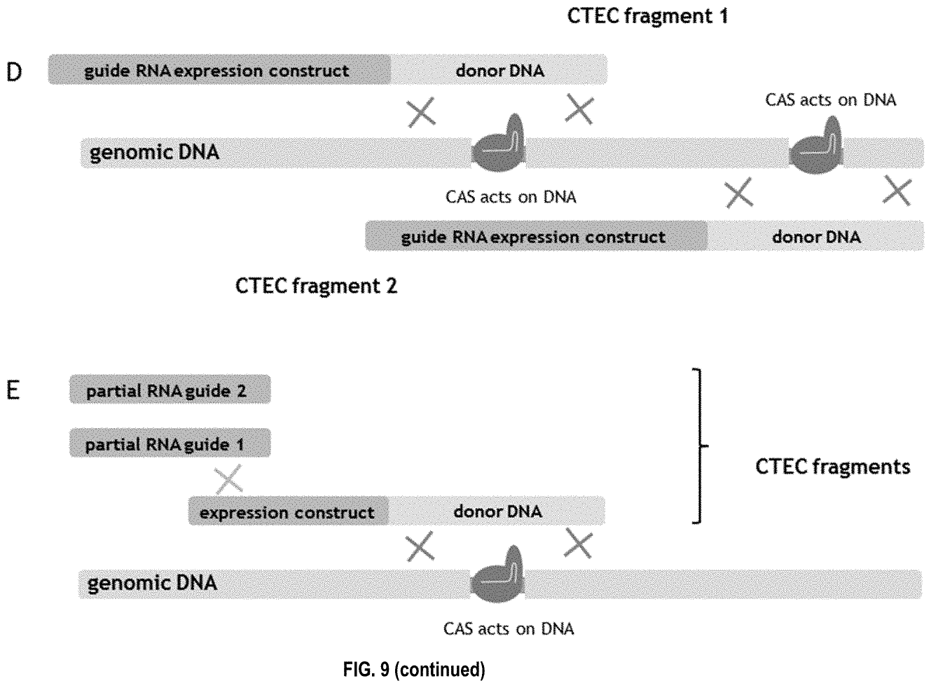

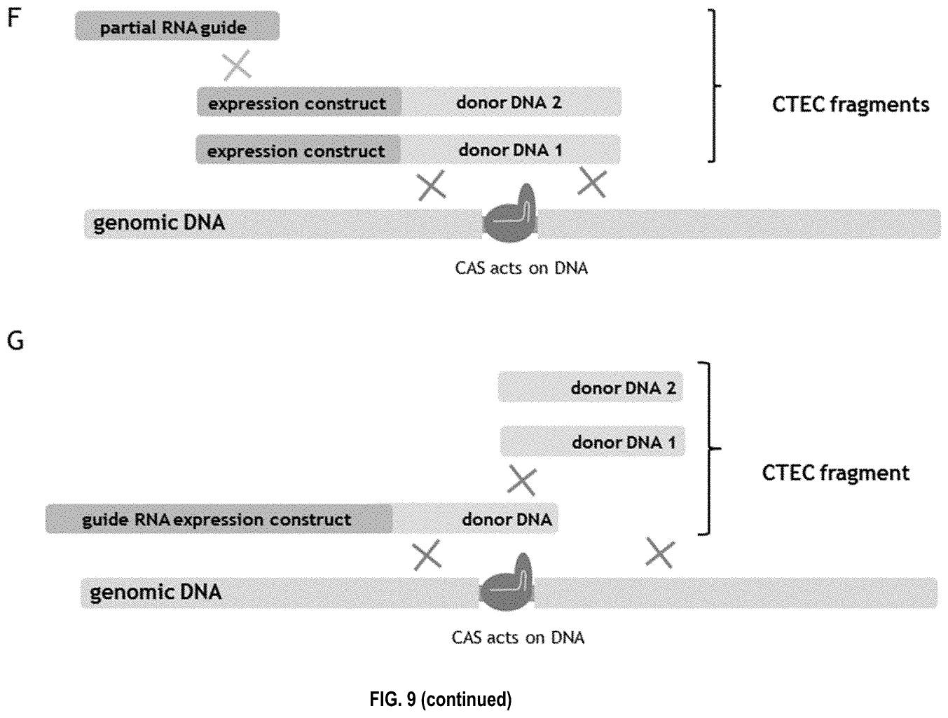

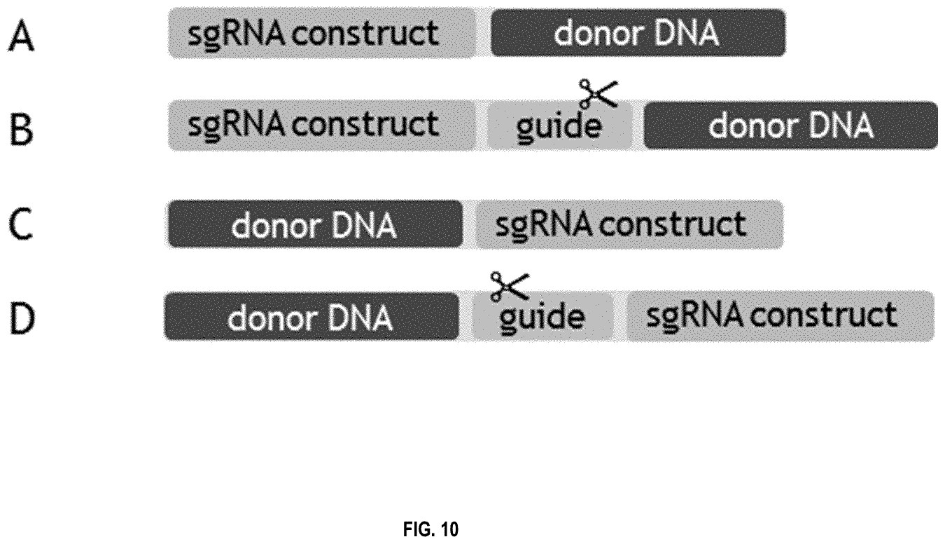

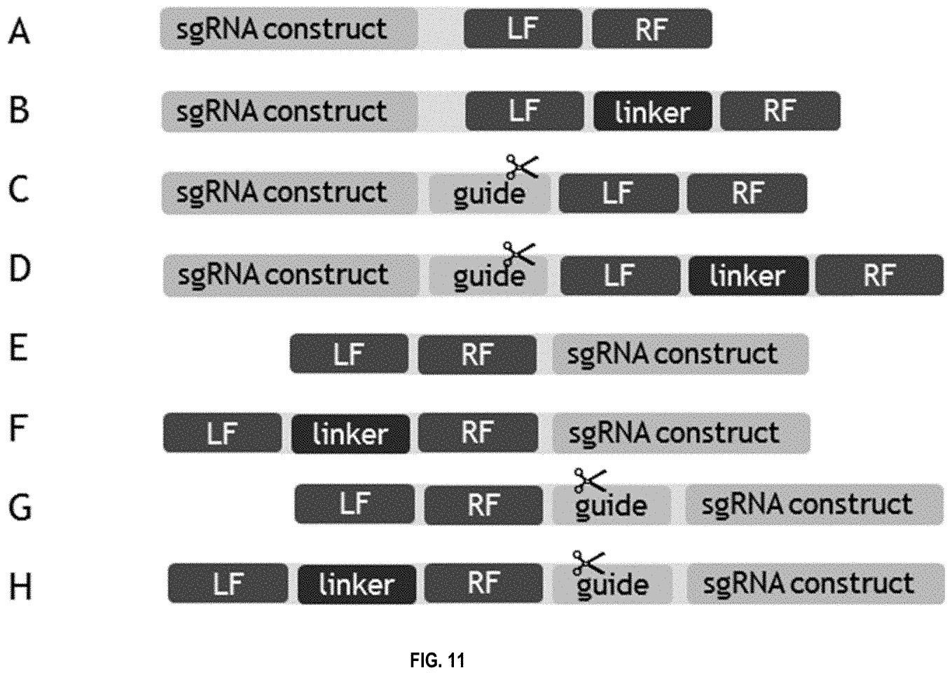

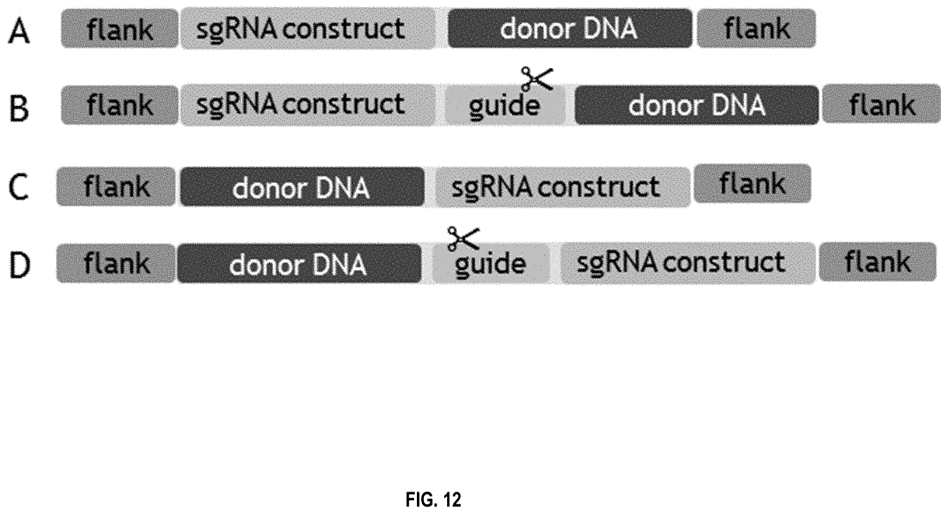

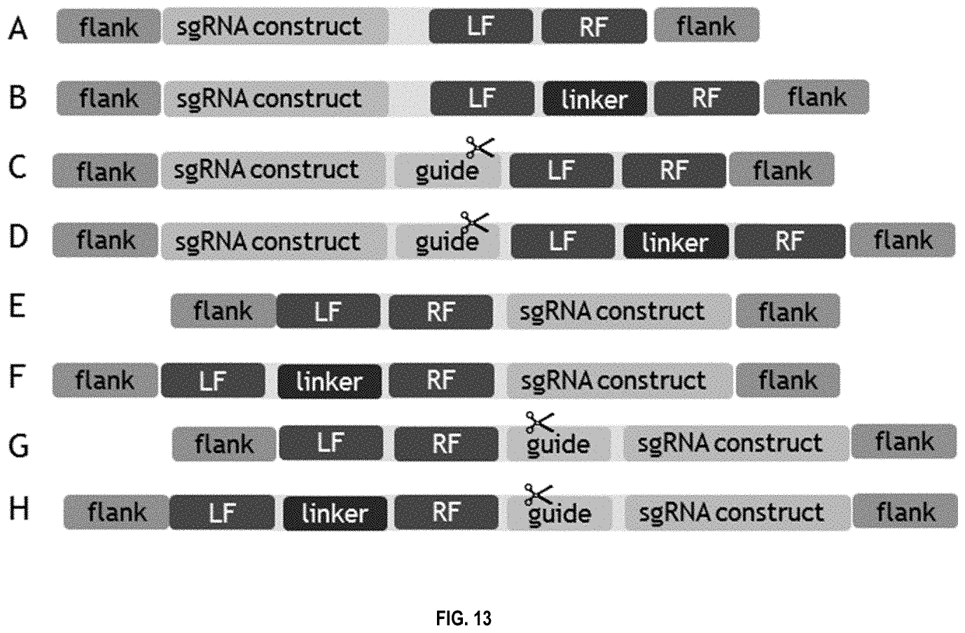

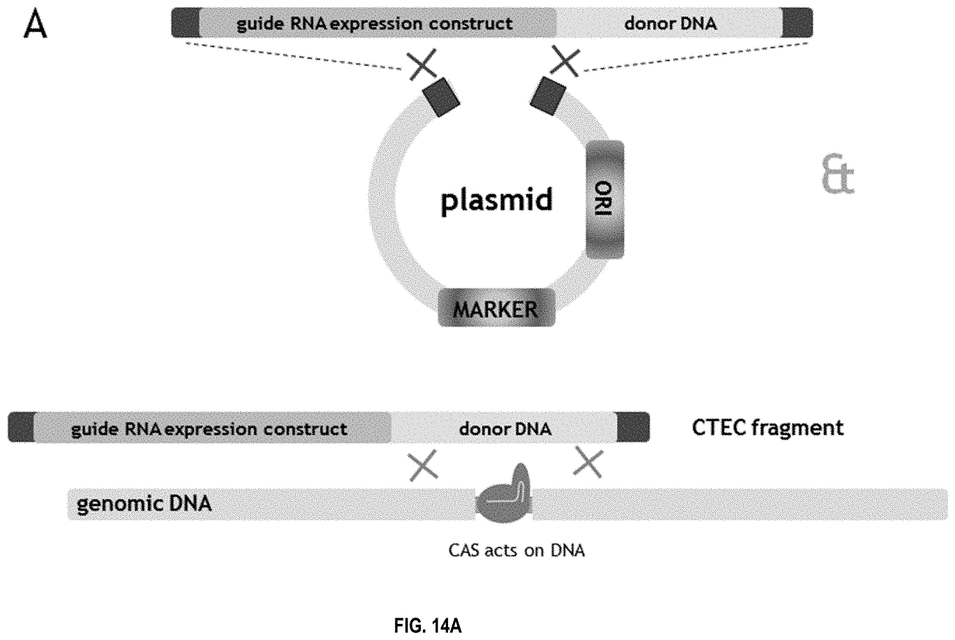

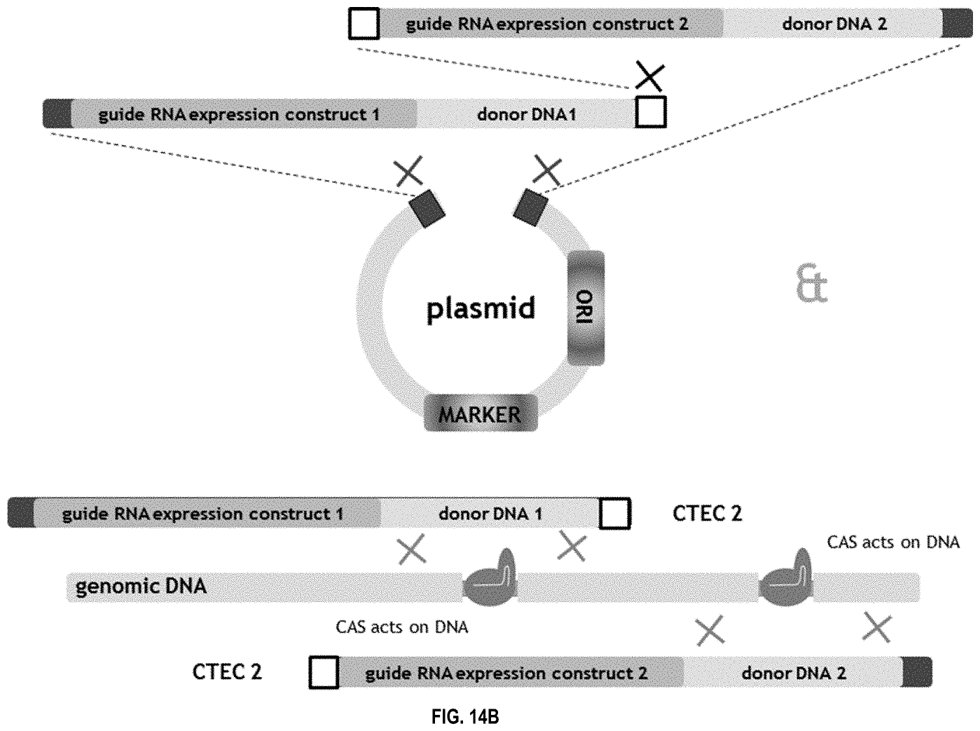

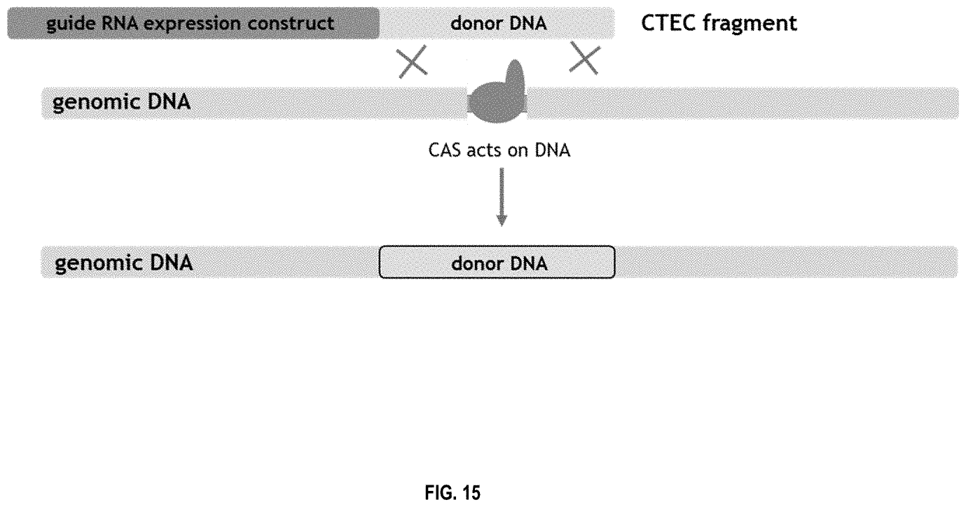

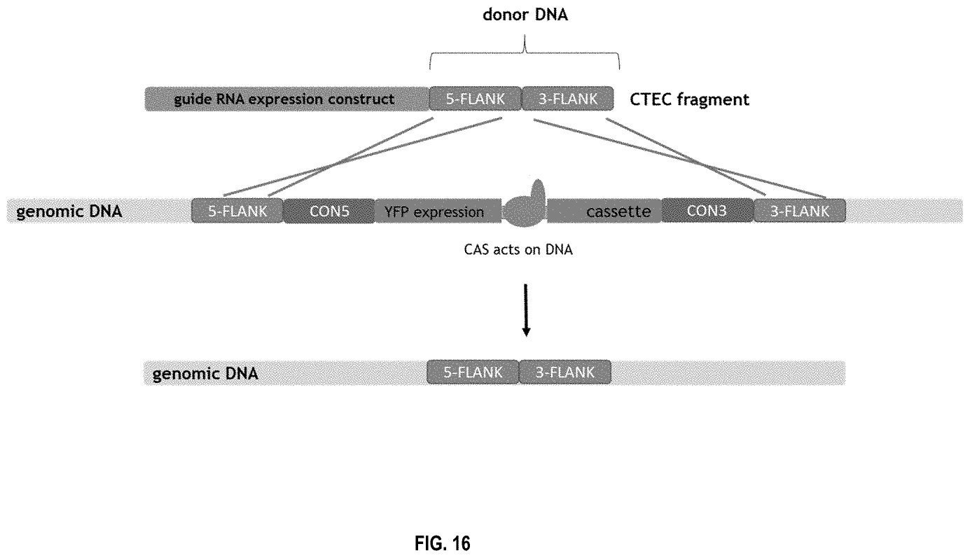

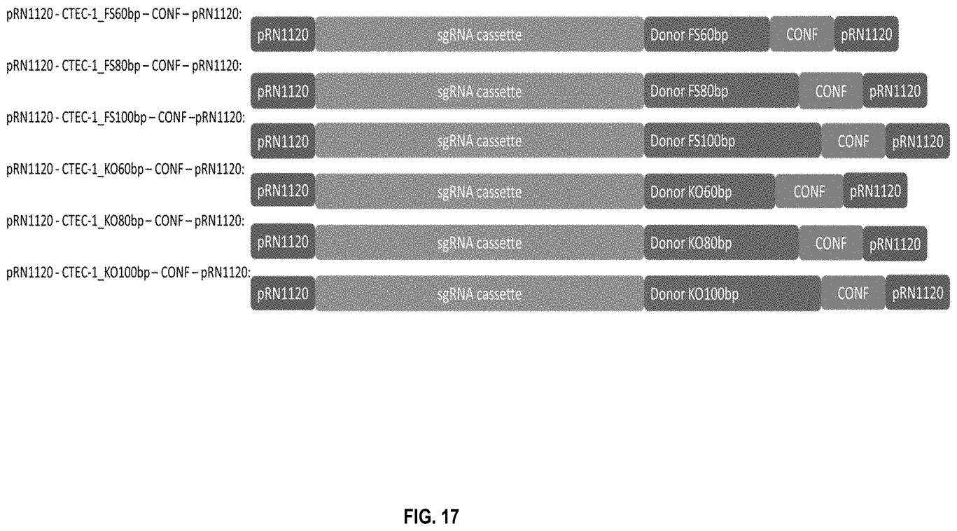

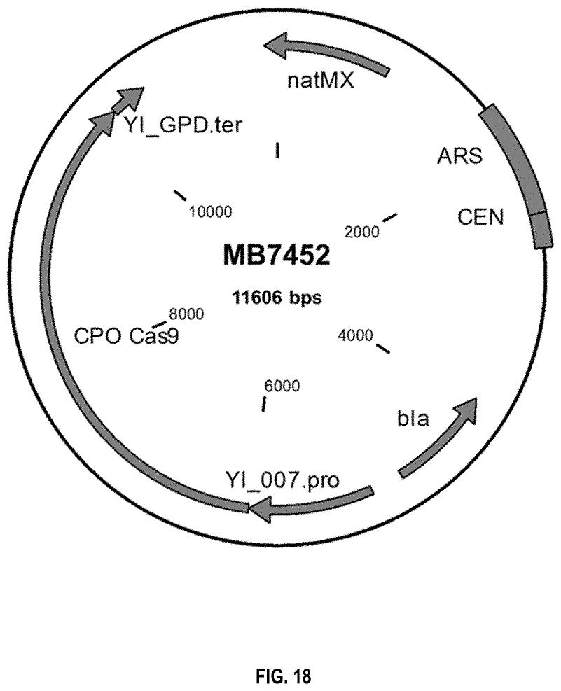

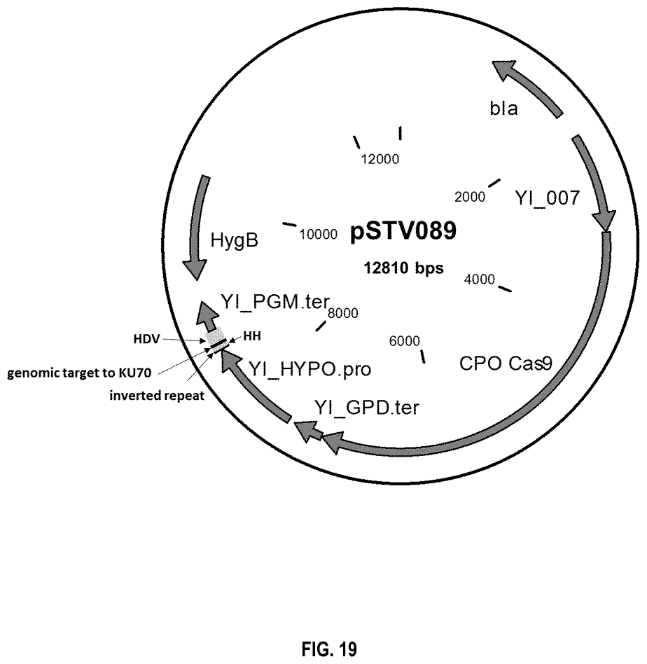

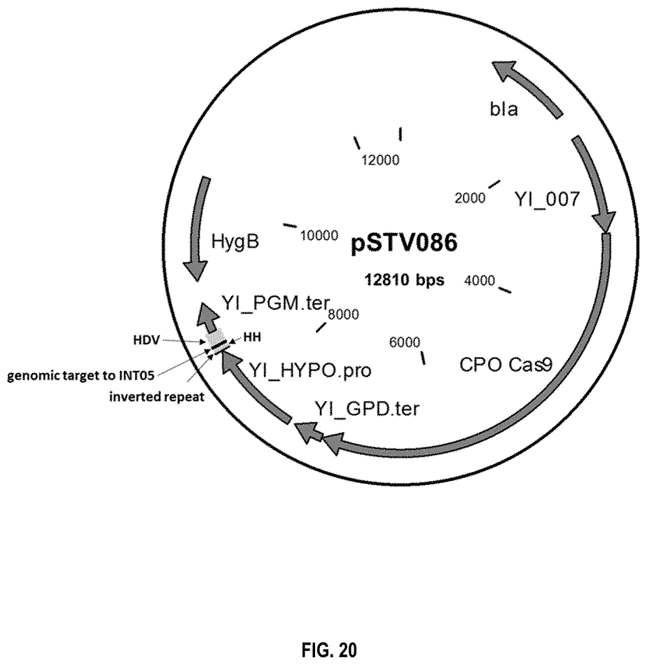

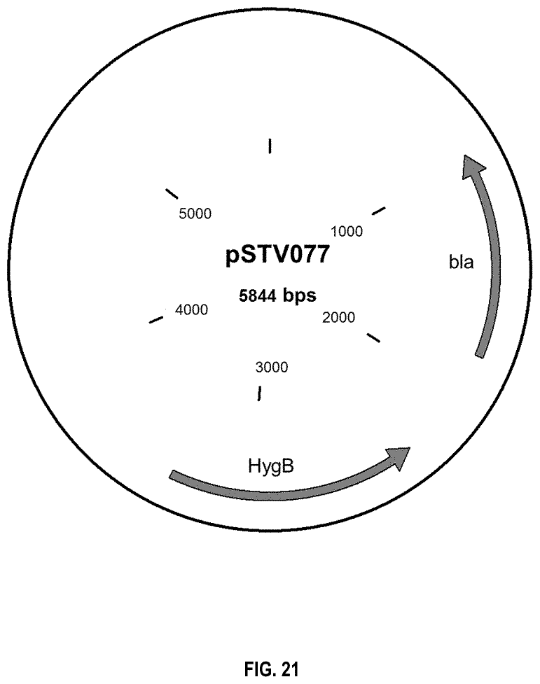

In the use according to the invention, in the CTEC, the additional polynucleotide element is located 3′-of the guide-RNA expression cassette or 5′-of the guide-RNA expression cassette; this means that the guide-RNA expression cassette is flanked at its 5′-end or at its 3′-end by the additional polynucleotide element that has sequence identity with the target sequence in the target genome. A non-limiting example of such construct is inter alia depicted in FIGS. 3, 4, 8 and 9.

Flanked at its 5′-end or at its 3′-end by an additional polynucleotide element is to be construed as that the additional polynucleotide element is located adjacent to the 5′-terminal side or to the 3′-terminal side of the guide-RNA expression cassette. For the avoidance of doubt, the CTEC is a single polynucleotide wherein the part: additional polynucleotide element-guide-RNA expression cassette or the guide-RNA expression cassette-additional polynucleotide element are recognizable but comprised of a single string of consecutive nucleotides. The ‘additional polynucleotide element’ is herein also referred to as ‘donor polynucleotide’ or ‘donor DNA’.

The additional polynucleotide element may be any suitable additional polynucleotide element, functional or non-functional, such as a control sequence, a marker, a gene of interest encoding a compound of interest as defined elsewhere herein, or a disruption construct. The control sequence may be any control sequence or combination of control sequences, such as a promotor, a KOZAK sequence, a signal sequence, a terminator, a pre-sequence, a pre-pro-sequence, a leader sequence, an activator sequence, a repressor sequence, a HIS-tag, a split-GFP tag or any other N-terminal tag. A preferred control sequence is a promoter sequence. This e.g. enables to insert a promoter or to replace an endogenous promoter, or a part thereof, by another promoter. The introduced promoter may be stronger or weaker than the endogenous promoter and/or may be an inducible promoter. Such promoters are known to the person skilled in the art. The marker may be any type of marker as long as it can be identified and thus serves as a marker. The marker may e.g. be a selection marker or may e.g. be an identifiable polynucleotide with known sequence to be used as a barcode or may be a tag such as a HIS-tag, GFP-tag, split GFP-tag, solubility tag. It should be noted that the self-guiding integration construct itself already provides a barcode marker due to its unique guide-sequence, which represents a barcode at the site of integration of the self-guiding integration construct. The gene of interest may be any gene of interest and is preferably one as defined in the section “General Definitions”. The gene of interest may be a complete expression construct comprising a promoter, a coding sequence and a terminator, or may at least comprise a coding sequence.

The additional polynucleotide element has sequence identity with the target sequence in the target genome. The sequence identity of the additional polynucleotide element in the CTEC according to the invention is preferably such that the additional polynucleotide element and the target sequence in the target genome can recombine in vivo such as within a cell such that the CTEC according to the invention integrates into the target genome. Typically, however, only the additional polynucleotide element integrates into the genome; the guide-RNA expression cassette is typically and preferably not integrated into the genome. The person skilled in the art will comprehend that the additional polynucleotide element may not physically integrate into the genome but at least the sequence of the additional polynucleotide element is introduced into the genome at the target site. If the additional polynucleotide element has a sequence that has sequence identity with the protospacer adjacent motif (PAM) in the target sequence in the target genome, the part in the additional polynucleotide element that has sequence identity with the PAM may comprise a mutation in view of the PAM, such that when the sequence of the additional polynucleotide element integrates into the genome, it will not be recognized and cut by the genome editing enzyme complex. If the additional polynucleotide element has a sequence that has sequence identity with the guide-RNA target sequence in the target genome, the part in the additional polynucleotide element that has sequence identity with the guide-RNA target sequence may comprise a mutation in view of the guide-RNA target, such that when the sequence of the additional polynucleotide element integrates into the genome, it will not be recognized and cut by the genome editing enzyme complex.

The additional polynucleotide element does not need to have sequence identity over its entire length, it suffices that a part (or multiple parts) of the additional polynucleotide element has/have (sufficient) sequence identity to allow recombination with the target sequence in the target genome. The person skilled in the art knows that some mismatches are permissible while still allowing recombination. Preferably, the sequence identity of the additional polynucleotide element of the CTEC as disclosed herein and the target sequence in the target genome is at least 80, 81, 82, 83, 84, 85, 86, 87, 88, 89, 90, 91, 92, 93, 94, 95, 97, 98 or 99% and most preferably 100%. The additional polynucleotide element according to the invention may have any length as long as allowing recombination in vivo such as within a cell such that the additional polynucleotide element of the CTEC or the CTEC as disclosed herein integrates into the target genome. In the embodiments of the invention, the additional polynucleotide element may have a length of at least 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 350, 400, 450, 500, 600, 700, 800, 900 or 1000 nucleotides. Preferably, the additional polynucleotide element has a length of at most 1000, 900, 800, 700, 600, 500, 450, 400, 350, 300, 290, 280, 270, 260, 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 25, 20, 15 or 10 nucleotides. The additional polynucleotide element may have a length such as larger than 40 nucleotides or 50 nucleotides and in the range of about 40 nucleotides, or about 50 nucleotides to about 1 kilonucleotides, about 40 nucleotides or about 50 nucleotides to about 500 nucleotides, about 40 nucleotides or about 50 nucleotides to about 300 nucleotides, about 40 nucleotides or about 50 nucleotides to about 250 nucleotides, or about 40 nucleotides or about 50 nucleotides to about 200 nucleotides. The additional polynucleotide element may have a length of 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 203, 204, 205, 206, 207, 208, 209, 210, 220, 221, 222, 223, 224, 225, 226, 227, 228, 229, 230, 231, 232, 233, 234, 235, 236, 237, 238, 239, 240, 241, 242, 243, 244, 245, 246, 247, 248 or 250 nucleotides.

Included in the invention is the use where two or more CTEC's are provided comprising the same guide-RNA expression cassette and an additional polynucleotide element, and wherein said additional polynucleotide elements have sequence identity with target sequences in the target genome which are different for each of the two or more CTECs. A non-limiting example of such CTEC is inter alia depicted in FIG. 9E

Included in the invention is the use where two or more CTEC's are provided each comprising a different guide-RNA expression cassette and an additional polynucleotide element, that has sequence identity with the target sequence in the target genome which are the same for each of the two or more CTECs. In this embodiment, the frequency of NHEJ repair is reduced since if a break mediated by the first CTEC and a polynucleotide guided editing enzyme is repaired by NHEJ, the target site will still be present and will be the target for a further CTEC. In such iteration, the chance of NHEJ will be the square of the chance on NHEJ for a single CTEC mediated editing event. A non-limiting example of such CTEC is inter alia depicted in FIGS. 9F and 9G.

The additional polynucleotide element in the CTEC has sequence identity with the target sequence in the target genome. The sequence identity of the additional polynucleotide element may be with the target sequence itself, i.e. the sequence in the genome where the complex of a functional polynucleotide-guided genome editing enzyme and a guide-RNA binds. The sequence identity of the additional polynucleotide element in the CTEC may also be with sequences flanking the target sequence or with the target sequence and with sequences flanking the target sequence, as long as recombination between the additional polynucleotide element and the target sequence and, if the case, sequences flanking the target sequence, is enabled. As an example, it is possible that an additional polynucleotide element of 200 bp has a part at its 5′-end of 50 bp that has sequence identity with a 50 bp part adjacent to the 3′-end of the target sequence in the target genome and that the additional polynucleotide element has a part at its 3′-end of 50 bp that has sequence identity with a 50 bp part adjacent to the 5′-end of the target sequence in the target genome. In this case recombination between the additional polynucleotide element and the region around the target sequence in the target genome can effectively occur when a double strand break is initiated by the complex of a functional polynucleotide-guided genome editing enzyme and a guide-RNA encoded by the CTEC. As another example, it is possible that an additional polynucleotide element of 100 bp has a part at its 5′-end of 50 bp that has sequence identity with a 50 bp part adjacent to the 3′-end of the target sequence in the target genome and that the additional polynucleotide element has a part at its 3′-end of 50 bp that has sequence identity with a 50 bp part adjacent to the 5′-end of the target sequence in the target genome. In this case recombination between the additional polynucleotide element and the region around the target sequence in the target genome can effectively occur when a double strand break is initiated by the complex of a functional polynucleotide-guided genome editing enzyme and a guide-RNA encoded by the CTEC. The person skilled in the art will comprehend that many variations are possible, some of these are depicted in the examples and the figures herein, but are not limited thereto. Herein, said 5′ and 3′ parts of the additional polynucleotide element may be depicted as flanks

The parts adjacent to the target sequence in the target genome may be located immediately adjacent to the target sequence in the target genome. The parts adjacent to the target sequence in the target genome may also be located away from the target sequence. The parts adjacent to the target sequence in the genome may be at about 1, 2, 3, 4, 5, 6, 7, 8, 9, 10, 15, 20, 30, 40, 50 100, 200, 300, 400, 500, 1000, 5000, 10000 nucleotides away from the target sequence.

In the embodiments of the invention, a marker may be used to facilitate selection of a host cell comprising the CTEC according to the invention or to facilitate selection of a host cell that has been edited by a CTEC according to the invention. Such marker may be present on the CTEC, but is preferably present on a separate polynucleotide such as a plasmid, such as an autonomously replicating plasmid.

In the use according to the invention, the functional guide-RNA, or part thereof, according to the invention may be exclusively expressed from the self-guiding integration construct, meaning that there is no other guide-RNA expression construct present in the host cell (not in the genome and not on a vector). The guide-RNA, or part thereof that is specific for a target sequence in a target genome, is initially expressed from the self-guiding integration construct. The expressed guide-RNA facilitates induction of a break into the target genome at the target sequence and subsequently the self-guiding integration construct integrates into the target genome.

In the use according to the invention, the CTEC may be comprised of two or more polynucleotides capable of recombining with each other to yield a CTEC according to the invention comprising:
a guide-RNA expression cassette, and
an additional polynucleotide element,
   wherein the guide-RNA expression cassette is capable of expressing a functional guide-RNA, or a part thereof, that is specific for a target sequence in a target genome, wherein the additional polynucleotide element has sequence identity with the target sequence in the target genome. A non-limiting example of such CTEC is inter alia depicted in FIGS. 9B and 9C,

In the embodiments of the invention, the additional polynucleotide element in the CTEC may be located directly at the 5′-terminal side or at the 3′-terminal side of the guide-RNA expression cassette or a linker may be present between the additional polynucleotide element and the guide-RNA expression construct. In the embodiments of the invention, the linker is also referred to as a connector. The linker may have any length and may be a non-coding region. The length of the linker may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides. The linker may be at least about 5, 10, 15, 20, 25 or 30 nucleotides in length. The linker may be at most about 30, 25, 20, 15, 10 or 5 nucleotides in length. A non-limiting example of such CTEC is inter alia depicted in FIG. 3 (CTEC-2 and CTEC 3).

In the embodiments of the invention, the linker may be a special linker; the CTEC, the guide-RNA expression cassette and the additional polynucleotide element may be linked by a polynucleotide that comprises a target sequence that corresponds to the guide sequence of the guide-RNA, allowing in vivo cleavage of the guide-RNA expression cassette from the additional polynucleotide element. Without being bound by theory, the separation of the guide-RNA expression cassette from the additional polynucleotide element may increase the chances that the additional polynucleotide element integrates into the genome at the target site whereas the guide-RNA expression cassette from the additional polynucleotide element remains episomal. A non-limiting example of such CTEC is inter alia depicted in FIG. 3 (CTEC-5, CTEC-6A and CTEC-6B).

In the embodiments of the invention, the CTEC preferably comprises a guide-RNA expression cassette that capable of expressing a functional guide-RNA.

The guide-RNA expression cassette of the embodiments of the invention is a polynucleotide expression construct that comprises all components, except for the RNA polymerase, needed to express a functional guide-RNA or a part thereof in vivo such as within a cell. The components include, but are not limited to, a promoter, a coding sequence encoding a guide-RNA or a part thereof and a terminator. There are several ways to express a guide-RNA in vivo, such as within a cell. The guide-RNA may be expressed from any suitable promoter, such as a eukaryotic promoter. The guide-RNA may be expressed from an RNA polymerase II promoter. Such promoter is known to the person skilled in the art. Preferred RNA polymerase II promoters are listed in WO2016/50136, WO2016/50135 and WO2016/110453. The guide-RNA may be expressed from RNA polymerase III promoter. Such a promoter is known to the person skilled in the art. Preferred RNA polymerase III promoters are listed in WO2016/50136, WO2016/50135 and WO2016/110453. When using an RNA polymerase III promoter, a self-processing ribozyme is preferably used to convert the raw transcription product into a mature guide-RNA. The guide-RNA may be expressed from a single-subunit DNA-dependent RNA polymerase promoter. Such promoter is known to the person skilled in the art. Preferred single-subunit DNA-dependent RNA polymerase promoters are viral single-subunit DNA-dependent RNA polymerase promoters, such as a T3, SP6, K11 or T7 RNA polymerase promoter. Such preferred single-subunit DNA-dependent RNA polymerase promoters are listed in U.S. 62/399,127.

The CTEC in the embodiments of the invention may comprise two or more polynucleotide sequences capable of recombining with a vector, preferably a plasmid, to in vivo yield the CTEC integrated into the vector. A non-limiting example of such CTEC is inter alia depicted in FIGS. 14A and 14B.

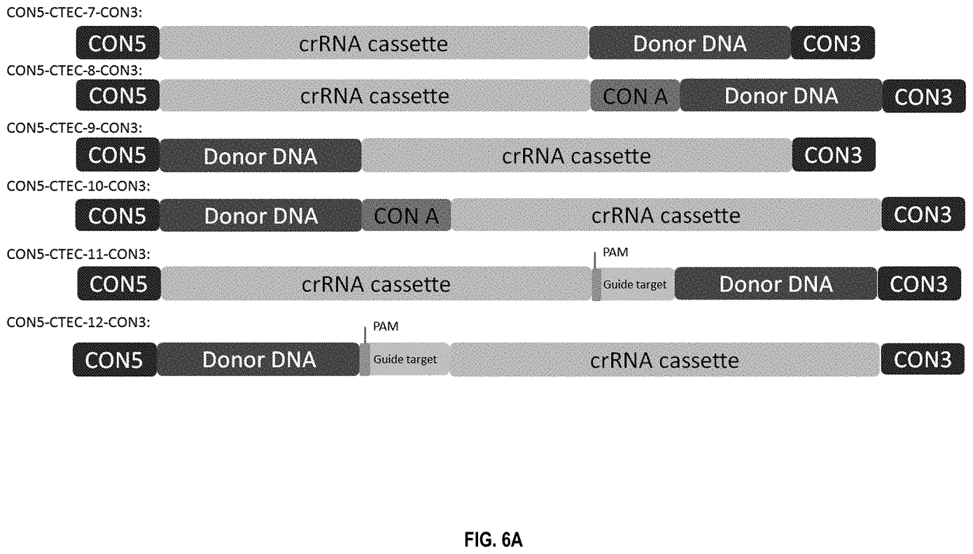

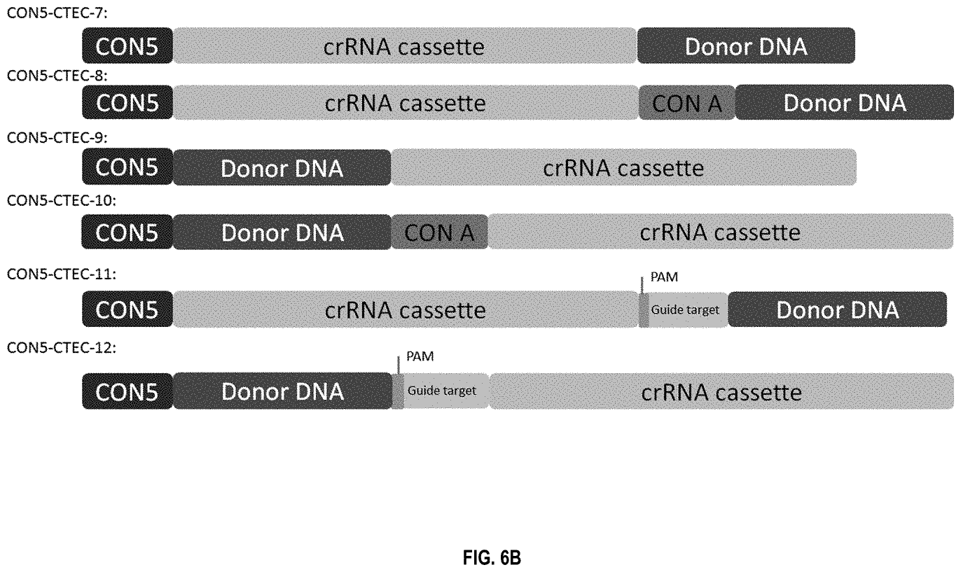

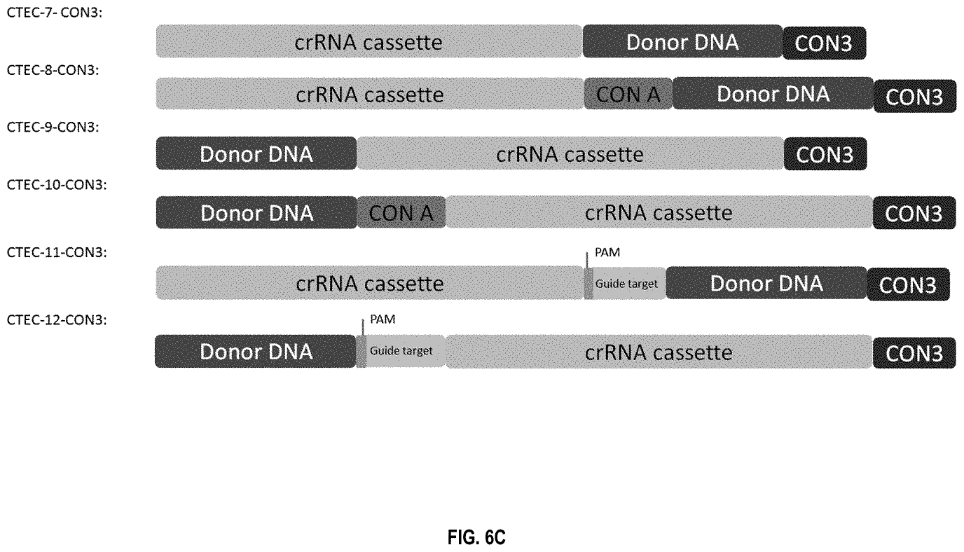

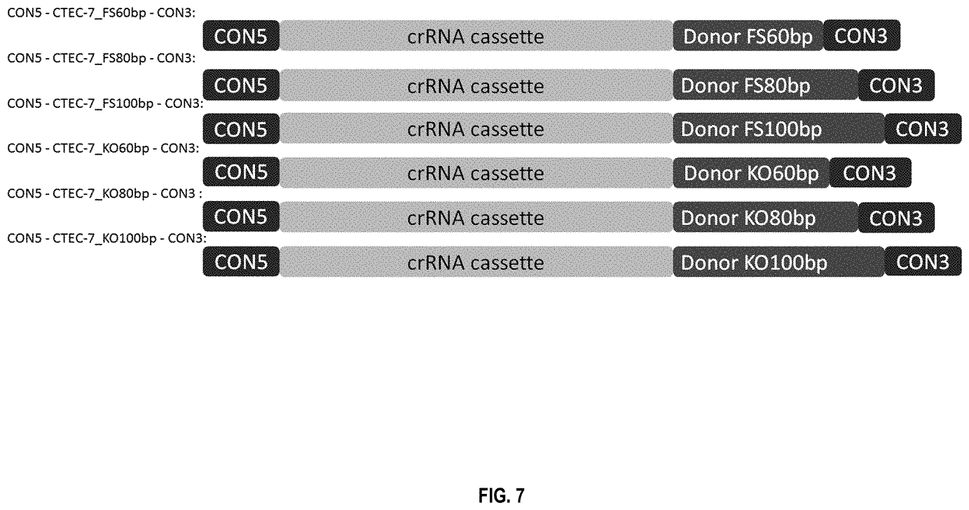

In order to facilitate synthesis of a CTEC according to the invention using e.g. polymerase chain reaction (PCR), the CTEC may be flanked by sequences where PCR primers can anneal to. These sequences may be located in the guide-RNA expression construct or in the additional polynucleotide element, or may be added as separate sequences. The added sequences may be depicted as 5′-flanks and 3′-flanks. A non-limiting example of such CTEC is inter alia depicted in FIGS. 6A-C. It is preferred that these flanks have little or no homology with either of the guide-RNA expression construct, the additional polynucleotide element or the genome. The 5′-flanks and 3′-flanks may have any length while still being able to anneal to PCR primers. A 5′-flank or 3′-flank may have a length of e.g. 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length.

The invention further provides for the ex vivo use of a composition comprising a CTEC according to the invention, or comprising a library of CTECs according to the invention, for expression in a host cell of a functional guide-RNA or part thereof that is specific for one or more target sequence(s) in a target genome. Such use encompasses but is not limited to introduction of the CTEC or library of CTECs into a host cell. The CTEC library in the embodiments of the invention may contain CTECs that are all specific for the same target sequence and e.g. each comprise a different additional polynucleotide element. The CTEC library may contain CTECs that are all specific for a different target sequence and e.g. each comprise identical additional polynucleotide elements.

The ex vivo use according to the invention of the CTEC as defined herein or of the composition comprising a CTEC or a library of CTECs may further comprise the use of a functional polynucleotide-guided genome editing enzyme or an expression construct capable of expressing a functional polynucleotide-guided genome editing enzyme and wherein the functional polynucleotide-guided genome editing enzyme preferably is a Cas9 or a Cpf1, all as defined herein above.

In the ex vivo use according to the embodiments of the invention of a CRISPR transient expression construct (CTEC) for expression in a host cell of a functional guide-RNA or part thereof, the host cell may be deficient in Non-Homologous End Joining (NHEJ).

In a second aspect, the invention provides for a host cell comprising a CTEC as defined in the first aspect and other embodiments of the invention. In this aspect of the invention, all features are preferably those as defined in the first aspect of the invention. The host cell may be any host cell. Preferred host cells are a fungus, an algae, a microalgae or a marine eukaryote, more preferably a yeast cell, a filamentous fungal cell and a Labyrinthulomycetes cell; all as defined herein in the section “General Definitions”. A host cell is to be construed as at least one host cell and a CTEC according to the invention is to be construed as at least one CTEC according to the invention. Within the scope of the invention is thus a population of host cells comprising a library of CTECs according to the invention and preferably comprising 2, 3, 4, 5, 6, 7, 8, 9, 10 or more CTEC. The host cell and the population of host cells are herein referred to as a host cell according to the invention.

The host cell according to this aspect of the invention may further comprise an expression construct capable of expressing a functional polynucleotide-guided genome editing enzyme, such as a functional polynucleotide-guided heterologous genome editing enzyme, wherein the functional polynucleotide-guided genome editing enzyme preferably is a Cas9 or a Cpf1, all as defined herein above.

In the host cell according to the invention, the sequence of the additional polynucleotide element may be introduced into the genome at the site where the additional polynucleotide element has sequence identity with the sequences flanking the target sequence in the target genome.

The host cell according to this aspect of the invention may be deficient in Non-Homologous End Joining (NHEJ).

In a third aspect, the invention provides for an ex vivo method for the production of a host cell, comprising introducing into the host cell a CTEC according to the invention and defined herein above or a composition as defined hereinabove. In the method, the guide-RNA expression cassette from the CTEC may not integrate into the genome of the host cell. In this aspect of the invention, all features are preferably those as defined in the first and second aspects of the invention.

A host cell is to be construed as at least one host cell and a CTEC according to the invention is to be construed as at least one CTEC according to the invention. Accordingly, in an embodiment, of the ex vivo method according to the invention, a library of a CRISPR transient expression constructs (CTECs) is introduced into a population of host cells. Such method can conveniently be used for screening purposes.

In the ex vivo method according to the invention, in the host cell a functional polynucleotide-guided genome editing enzyme may be present or may be introduced separately or simultaneously with the CRISPR transient expression construct (CTEC) or library of CRISPR transient expression constructs (CTECs); the functional polynucleotide-guided genome editing enzyme preferably may be a Cas9 or a Cpf1, all as defined herein above.

In an embodiment of this aspect of the invention, in the host cell a vector such as a plasmid is present, to which the CTEC comprising two or more polynucleotide sequences capable of recombining with the vector to yield the CTEC integrated into the vector, can integrate.

In the ex vivo method according to the invention, the sequence of the additional polynucleotide element may be introduced into the genome at the site where the additional polynucleotide element has sequence identity with the sequences flanking the target sequence in the target genome.

In the ex vivo method according to the invention, the functional guide-RNA, or part thereof that is specific for a target sequence in a target genome, may be exclusively expressed from the introduced CRISPR transient expression construct (CTEC).

In the ex vivo method according to the invention, the method may further comprise determining whether and/or where the sequence of the additional polynucleotide element of the CRISPR transient expression construct (CTEC) has been introduced into the genome of the host cell. Such determination may be performed using any technique known to the person skilled in the art, such as but not limited to PCR analysis and sequencing such as next generation sequencing allowing easy screening when using libraries of a self-guiding integration constructs. Said determination may be made by analysis of a gene product produced by the generated host cell, preferably by using selective growth conditions. Such selective growth conditions may e.g. allow for the positive selection of a host with the property of interest, allowing screening of a population of host cells wherein a library of self-guiding integration constructs has been introduced. The gene product may e.g. be a metabolite, enzyme (such as glucoamylase or an enzyme that resolves an auxotrophy) or a marker). In this aspect of the invention, the host cell that is generated and has properties of interest may be isolated.

The host cell according to the invention may be a host cell that is deficient in Non-Homologous End Joining (NHEJ).

In a fourth aspect, the invention provides for a host cell according to the second aspect of the invention or a host cell obtainable by or obtained by a method according to the third aspect of the invention, wherein the host cell comprises a polynucleotide encoding a compound of interest. In an embodiment of this aspect, the host cell expresses the compound of interest. In this aspect of the invention, all features are preferably those as defined in the first and second and third aspect of the invention. Said compound of interest is preferably one as defined in the section “General Definitions”.

Further provided is a method for the production of a compound of interest, comprising culturing the host cell of this aspect under conditions conducive to the production of the compound of interest, and, optionally, purifying or isolating the compound of interest.

The invention further provides for a linear CRISPR transient expression construct (CTEC) as defined herein above and as defined in the figures, sequence listing and examples herein. Non-limiting exemplary examples of CTECs according to the invention are listed here below.

A linear CRISPR transient expression construct (CTEC) comprising:
a guide-RNA expression cassette, and
an additional polynucleotide element,
   wherein the guide-RNA expression cassette is capable of expressing a functional guide-RNA, or a part thereof, that is specific for a target sequence in a target genome, wherein the additional polynucleotide element has sequence identity with the target sequence in the target genome.

A CRISPR transient expression construct (CTEC) comprising:

two or more linear polynucleotides capable of recombining with each other to yield:
a guide-RNA expression cassette, and
an additional polynucleotide element,
   wherein the guide-RNA expression cassette is capable of expressing a functional guide-RNA, or a part thereof, that is specific for a target sequence in a target genome, wherein the additional polynucleotide element has sequence identity with the target sequence in the target genome.

A CRISPR transient expression construct (CTEC) as listed here above, wherein the guide-RNA expression cassette and the additional polynucleotide element are linked by a polynucleotide that comprises a target sequence that corresponds to the guide sequence of the guide-RNA, allowing in vivo cleavage of the guide-RNA expression cassette from the additional polynucleotide element.

A CRISPR transient expression construct (CTEC) as listed here above, wherein the guide-RNA expression cassette is capable of expressing a functional guide-RNA.

A composition comprising two or more polynucleotide members, wherein these members have sequence identity with each other which allows them to recombine in vivo, such as in a host cell, to yield a CRISPR transient expression construct (CTEC) as listed here above.

A CRISPR transient expression construct (CTEC) as listed here above or a composition as listed here above, wherein the guide-RNA expression cassette comprises a eukaryotic promoter.

A CRISPR transient expression construct (CTEC) as listed here above or a composition as listed here above, wherein the functional guide-RNA, or the part thereof, is encoded by a polynucleotide on the guide-RNA expression cassette and the polynucleotide is operably linked to an RNA polymerase II promoter, to an RNA polymerase III promoter as well as a self-processing ribozyme or to a single-subunit DNA-dependent RNA polymerase promoter, preferably a viral single-subunit DNA-dependent RNA polymerase promoter, more preferably a T3, SP6, K11 or T7 RNA polymerase promoter.

A CRISPR transient expression construct (CTEC) as listed here above or a composition as listed here above, wherein the guide-RNA expression cassette is located 3′-of the additional polynucleotide element.

A CRISPR transient expression construct (CTEC) as listed here above or a composition as listed here above, wherein the guide-RNA expression cassette is located 5′-of the additional polynucleotide element.

A CRISPR transient expression construct (CTEC) as listed here above or a composition as listed here above, wherein the CTEC comprises two or more polynucleotide sequences capable of recombining with a vector, preferably a plasmid, to in vivo yield the CTEC integrated into the vector.

### Embodiments

The following embodiments of the invention are provided; the features in these embodiments are preferably those as defined previously herein.

1. Ex vivo use of a CRISPR transient expression construct (CTEC) for expression in a host cell of a functional guide-RNA or part thereof that is specific for a target sequence in a target genome, wherein the CRISPR transient expression construct is linear and comprises:
a guide-RNA expression cassette, and
an additional polynucleotide element, and,
   wherein the guide-RNA expression cassette is capable of expressing a functional guide-RNA, or a part thereof, that is specific for a target sequence in a target genome, and wherein the additional polynucleotide element has sequence identity with the target sequence in the target genome.

2. Ex vivo use of a CRISPR transient expression construct (CTEC) according to embodiment 1, wherein the functional guide-RNA, or part thereof that is specific for a target sequence in a target genome, is exclusively expressed from the CTEC.

3. Ex vivo use of a CRISPR transient expression construct (CTEC) according to embodiment 1 or 2, wherein the CTEC is comprised of two or more polynucleotides capable of recombining with each other to yield:
a guide-RNA expression cassette, and
an additional polynucleotide element,
   wherein the guide-RNA expression cassette is capable of expressing a functional guide-RNA, or a part thereof, that is specific for a target sequence in a target genome, wherein the additional polynucleotide element has sequence identity with the target sequence in the target genome.

4. Ex vivo use of a CRISPR transient expression construct (CTEC) according to any one of embodiments 1 to 3, wherein in the CTEC, the guide-RNA expression cassette and the additional polynucleotide element are linked by a polynucleotide that comprises a target sequence that corresponds to the guide sequence of the guide-RNA, allowing in vivo cleavage of the guide-RNA expression cassette from the additional polynucleotide element.

5. Ex vivo use of a CRISPR transient expression construct (CTEC) according to any one of embodiments 1 to 4, wherein the guide-RNA expression cassette is capable of expressing a functional guide-RNA.

6. Ex vivo use of a CRISPR transient expression construct (CTEC) according to any one of embodiments 1 to 5, wherein the guide-RNA expression cassette comprises a eukaryotic promoter.

7. Ex vivo use of a CRISPR transient expression construct (CTEC) according to any one of embodiments 1 to 5, wherein the functional guide-RNA, or the part thereof, is encoded by a polynucleotide on the guide-RNA expression cassette and the polynucleotide is operably linked to an RNA polymerase II promoter, to an RNA polymerase III promoter as well as a self-processing ribozyme or to a single-subunit DNA-dependent RNA polymerase promoter, preferably a viral single-subunit DNA-dependent RNA polymerase promoter, more preferably a T3, SP6, K11 or T7 RNA polymerase promoter.

8. Ex vivo use of a CRISPR transient expression construct (CTEC) according to any one of embodiments 1 to 7, wherein the guide-RNA expression cassette is located 3′-of the additional polynucleotide element.

9. Ex vivo use of CRISPR transient expression construct (CTEC) according to any one of embodiments 1 to 7, wherein the guide-RNA expression cassette is located 5′-of the additional polynucleotide element.

10. Ex vivo use of a CRISPR transient expression construct (CTEC) according to any one of embodiments 1 to 9, wherein the CTEC comprises two or more polynucleotide sequences capable of recombining with a vector, preferably a plasmid, to in vivo yield the CTEC integrated into the vector.

11. Ex vivo use of a composition comprising a CRISPR transient expression construct (CTEC) as defined in any one of embodiments 1 to 10, or comprising a library of CRISPR transient expression constructs (CTECs) as defined in any one of embodiments 1 to 10, for expression in a host cell of a functional guide-RNA or part thereof that is specific for one or more target sequence(s) in a target genome.

12. Ex vivo use of a CRISPR transient expression construct (CTEC) according to any one of embodiments 1 to 10 or ex vivo use of the composition according to embodiment 11, further comprising the use of a functional polynucleotide-guided genome editing enzyme or an expression construct capable of expressing a functional polynucleotide-guided genome editing enzyme and wherein the functional polynucleotide-guided genome editing enzyme preferably is a Cas9 or a Cpf1.

13. Ex vivo use according to any one of embodiments 1 to 12, wherein the host cell is deficient in Non-Homologous End Joining (NHEJ).

14. A host cell comprising a CRISPR transient expression construct (CTEC) as defined in any one of embodiments 1-10 or comprising a composition as defined in embodiment 11.

15. A host cell according to embodiment 14, further comprising:

a functional polynucleotide-guided genome editing enzyme, preferably a functional polynucleotide-guided heterologous genome editing enzyme, or
further comprising an expression construct capable of expressing a functional polynucleotide-guided genome editing enzyme, preferably a functional polynucleotide-guided heterologous genome editing enzyme,
wherein the functional polynucleotide-guided genome editing enzyme preferably is a Cas9 or a Cpf1.

16. A host cell according to embodiment 15, wherein the sequence of the additional polynucleotide element is introduced into the genome at the site where the additional polynucleotide element has sequence identity with the sequences flanking the target sequence in the target genome.

17. A host cell according to any one of embodiments 14 to 16, wherein the host cell is deficient in Non-Homologous End Joining (NHEJ).

18. An ex vivo method for the production of a host cell, comprising introducing into the host cell a CRISPR transient expression construct (CTEC) as defined in any one of embodiments 1 to 10 or a composition as defined as in embodiment 11, wherein the guide-RNA expression cassette from the CTEC preferably does not integrate into the genome of the host cell.

19. An ex vivo method according to embodiment 18, wherein a library of a CRISPR transient expression constructs (CTECs) is introduced into a population of host cells.

20. An ex vivo method according to embodiment 18 or 19, wherein in the host cell a functional polynucleotide-guided genome editing enzyme is present or is introduced separately or simultaneously with the CRISPR transient expression construct (CTEC) or library of CRISPR transient expression constructs (CTECs) and wherein the functional polynucleotide-guided genome editing enzyme preferably is a Cas9 or a Cpf1.

21. An ex vivo method according to any one of embodiments 18 to 20, wherein in the host cell a vector, preferably a plasmid, is present, to which the CTEC comprising two or more polynucleotide sequences capable of recombining with the vector to yield the CTEC integrated into the vector, can integrate.

22. An ex vivo method according to any one of embodiments 18 to 21, wherein the sequence of the additional polynucleotide element is introduced into the genome at the site where the additional polynucleotide element has sequence identity with the sequences flanking the target sequence in the target genome.

23. An ex vivo method according to any one of embodiments 18 to 22, wherein the functional guide-RNA, or part thereof that is specific for a target sequence in a target genome, is exclusively expressed from the introduced CRISPR transient expression construct (CTEC).

24. An ex vivo method according to any one of embodiments 18 to 23, further comprising determining whether and/or where the sequence of the additional polynucleotide element of the CRISPR transient expression construct (CTEC) has been introduced into the genome of the host cell.

25. An ex vivo method according to embodiment 24, wherein the determination is made by analysis of a gene product produced by the generated host cell, preferably by using selective growth conditions.

26. An ex vivo method according to any one of embodiments 18 to 25, wherein the host cell is deficient in Non-Homologous End Joining (NHEJ).

27. A host cell according to any one of embodiments 14 to 17 or a host cell obtainable or obtained by a method according to any one of embodiments 18 to 26, the host cell comprising a polynucleotide encoding a compound of interest.

28. A host cell according to embodiment 27, expressing the compound of interest.

29. A method for the production of a compound of interest, comprising culturing the host cell according to embodiment 27 or 28 under conditions conducive to the production of the compound of interest, and, optionally, purifying or isolating the compound of interest.

30. A linear CRISPR transient expression construct (CTEC) comprising:
a guide-RNA expression cassette, and
an additional polynucleotide element,
   wherein the guide-RNA expression cassette is capable of expressing a functional guide-RNA, or a part thereof, that is specific for a target sequence in a target genome, wherein the additional polynucleotide element has sequence identity with the target sequence in the target genome.

31. A CRISPR transient expression construct (CTEC) comprising:

two or more linear polynucleotides capable of recombining with each other to yield:
a guide-RNA expression cassette, and
an additional polynucleotide element, wherein the guide-RNA expression cassette is capable of expressing a functional guide-RNA, or a part thereof, that is specific for a target sequence in a target genome, wherein the additional polynucleotide element has sequence identity with the target sequence in the target genome.

32. A CRISPR transient expression construct (CTEC) according to embodiment 30, or 31, wherein the guide-RNA expression cassette and the additional polynucleotide element are linked by a polynucleotide that comprises a target sequence that corresponds to the guide sequence of the guide-RNA, allowing in vivo cleavage of the guide-RNA expression cassette from the additional polynucleotide element.

33. A CRISPR transient expression construct (CTEC) according to any one of embodiments 30 to 32, wherein the guide-RNA expression cassette is capable of expressing a functional guide-RNA.

34. A composition comprising two or more polynucleotide members, wherein these members have sequence identity with each other which allows them to recombine in vivo, such as in a host cell, to yield a CRISPR transient expression construct (CTEC) according to any one of embodiments 30 to 33.

35. A CRISPR transient expression construct (CTEC) according to any one of embodiments 30 to 33 or a composition according to embodiment 34, wherein the guide-RNA expression cassette comprises a eukaryotic promoter.

36. A CRISPR transient expression construct (CTEC) according to any one of embodiments 30 to 33 and 35 or a composition according to embodiment 34, wherein the functional guide-RNA, or the part thereof, is encoded by a polynucleotide on the guide-RNA expression cassette and the polynucleotide is operably linked to an RNA polymerase II promoter, to an RNA polymerase III promoter as well as a self-processing ribozyme or to a single-subunit DNA-dependent RNA polymerase promoter, preferably a viral single-subunit DNA-dependent RNA polymerase promoter, more preferably a T3, SP6, K11 or T7 RNA polymerase promoter.

37. A CRISPR transient expression construct (CTEC) according to any one of embodiments 30 to 33 and 35 to 36 or a composition according to embodiment 34, wherein the guide-RNA expression cassette is located 3′-of the additional polynucleotide element.

38. A CRISPR transient expression construct (CTEC) according to any one of embodiments 30 to 33 and 35 to 36 or a composition according to embodiment 34, wherein the guide-RNA expression cassette is located 5′-of the additional polynucleotide element.

39. A CRISPR transient expression construct (CTEC) according to any one of embodiments 30 to 33 and 35 to 38 or a composition according to embodiment 34, wherein the CTEC comprises two or more polynucleotide sequences capable of recombining with a vector, preferably a plasmid, to in vivo yield the CTEC integrated into the vector.

### General Definitions

Throughout the present specification and the accompanying claims, the words “comprise”, “include” and “having” and variations such as “comprises”, “comprising”, “includes” and “including” are to be interpreted inclusively. That is, these words are intended to convey the possible inclusion of other elements or integers not specifically recited, where the context allows.

The terms “a” and “an” are used herein to refer to one or to more than one (i.e. to one or at least one) of the grammatical object of the article. By way of example, “an element” may mean one element or more than one element.

The word “about” or “approximately” when used in association with a numerical value (e.g. about 10) preferably means that the value may be the given value (of 10) more or less 1% of the value. CRISPR interference (CRISPRi) is a genetic perturbation technique that allows for sequence-specific repression or activation of gene expression in prokaryotic and eukaryotic cells.

When herein is mentioned the term “0 kbp” deletion, this is not exactly a “0 kbp”; depending on the specifics of the SGIC several base pairs, such as e.g. about 80, 90, 100, 110, 120, 130, 140 or 150 will be deleted from the genome upon integration of the SGIC.

A polynucleotide refers herein to a polymeric form of nucleotides of any length or a defined specific length-range or length, of either deoxyribonucleotides or ribonucleotides, or mixes or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, oligonucleotides and primers. A polynucleotide may comprise natural and non-natural nucleotides and may comprise one or more modified nucleotides, such as a methylated nucleotide and a nucleotide analogue or nucleotide equivalent wherein a nucleotide analogue or equivalent is defined as a residue having a modified base, and/or a modified backbone, and/or a non-natural internucleoside linkage, or a combination of these modifications. As desired, modifications to the nucleotide structure may be introduced before or after assembly of the polynucleotide. A polynucleotide may be further modified after polymerization, such as by conjugation with a labeling compound.

In general, codon optimization refers to a process of modifying a nucleic acid sequence for enhanced expression in a host cell of interest by replacing at least one codon (e.g. more than 1, 2, 3, 4, 5, 10, 15, 20, 25, 50, or more codons) of a native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See e.g. Nakamura, Y., et al., 2000. Computer algorithms for codon optimizing a particular sequence for expression in a particular host cell are also available, such as Gene Forge (Aptagen; Jacobus, PA), are also available. Preferably, one or more codons (e.g. 1, 2, 3, 4, 5, 10, 15, 20, 25, 50, or more, or all codons) in a sequence encoding a Cas protein correspond to the most frequently used codon for a particular amino acid. Preferred methods for codon optimization are described in WO2006/077258 and WO2008/000632). WO2008/000632 addresses codon-pair optimization. Codon-pair optimization is a method wherein the nucleotide sequences encoding a polypeptide have been modified with respect to their codon-usage, in particular the codon-pairs that are used, to obtain improved expression of the nucleotide sequence encoding the polypeptide and/or improved production of the encoded polypeptide. Codon pairs are defined as a set of two subsequent triplets (codons) in a coding sequence. The amount of Cas protein in a source in a composition according to the invention may vary and may be optimized for optimal performance. In an RNA molecule with a 5′-cap, a 7-methylguanylate residue is located on the 5′ terminus of the RNA (such as typically in mRNA in eukaryotes). RNA polymerase II (Pol II) transcribes mRNA in eukaryotes. Messenger RNA capping occurs generally as follows: The most terminal 5′ phosphate group of the mRNA transcript is removed by RNA terminal phosphatase, leaving two terminal phosphates. A guanosine monophosphate (GMP) is added to the terminal phosphate of the transcript by a guanylyl transferase, leaving a 5′-5′ triphosphate-linked guanine at the transcript terminus. Finally, the 7-nitrogen of this terminal guanine is methylated by a methyl transferase. The terminology “not having a 5′-cap” herein is used to refer to RNA having, for example, a 5′-hydroxyl group instead of a 5′-cap. Such RNA can be referred to as “uncapped RNA”, for example. Uncapped RNA can better accumulate in the nucleus following transcription, since 5′-capped RNA is subject to nuclear export.

A ribozyme refers to one or more RNA sequences that form secondary, tertiary, and/or quaternary structure(s) that can cleave RNA at a specific site. A ribozyme includes a “self-cleaving ribozyme, or self-processing ribozyme” that is capable of cleaving RNA at a c/s-site relative to the ribozyme sequence (i.e., auto-catalytic, or self-cleaving). The general nature of ribozyme nucleolytic activity is known to the person skilled in the art. The use of self-processing ribozymes in the production of guide-RNA's for RNA-guided nuclease systems such as CRISPR/Cas is inter alia described by Gao et al, 2014.

A nucleotide analogue or equivalent typically comprises a modified backbone. Examples of such backbones are provided by morpholino backbones, carbamate backbones, siloxane backbones, sulfide, sulfoxide and sulfone backbones, formacetyl and thioformacetyl backbones, methyleneformacetyl backbones, riboacetyl backbones, alkene containing backbones, sulfamate, sulfonate and sulfonamide backbones, methyleneimino and methylenehydrazino backbones, and amide backbones. It is further preferred that the linkage between a residue in a backbone does not include a phosphorus atom, such as a linkage that is formed by short chain alkyl or cycloalkyl internucleoside linkages, mixed heteroatom and alkyl or cycloalkyl internucleoside linkages, or one or more short chain heteroatomic or heterocyclic internucleoside linkages.

A preferred nucleotide analogue or equivalent comprises a Peptide Nucleic Acid (PNA), having a modified polyamide backbone (Nielsen et al., 1991. Science 254, 1497-1500). PNA-based molecules are true mimics of DNA molecules in terms of base-pair recognition. The backbone of the PNA is composed of N-(2-aminoethyl)-glycine units linked by peptide bonds, wherein the nucleobases are linked to the backbone by methylene carbonyl bonds. An alternative backbone comprises a one-carbon extended pyrrolidine PNA monomer (Govindaraju and Kumar, 2005. Chem. Commun, 495-497). Since the backbone of a PNA molecule contains no charged phosphate groups, PNA-RNA hybrids are usually more stable than RNA-RNA or RNA-DNA hybrids, respectively (Egholm et al., 1993. Nature 365, 566-568).

A further preferred backbone comprises a morpholino nucleotide analog or equivalent, in which the ribose or deoxyribose sugar is replaced by a 6-membered morpholino ring. A most preferred nucleotide analog or equivalent comprises a phosphorodiamidate morpholino oligomer (PMO), in which the ribose or deoxyribose sugar is replaced by a 6-membered morpholino ring, and the anionic phosphodiester linkage between adjacent morpholino rings is replaced by a non-ionic phosphorodiamidate linkage.

A further preferred nucleotide analogue or equivalent comprises a substitution of at least one of the non-bridging oxygens in the phosphodiester linkage. This modification slightly destabilizes base-pairing but adds significant resistance to nuclease degradation. A preferred nucleotide analogue or equivalent comprises phosphorothioate, chiral phosphorothioate, phosphorodithioate, phosphotriester, aminoalkylphosphotriester, H-phosphonate, methyl and other alkyl phosphonate including 3′-alkylene phosphonate, 5′-alkylene phosphonate and chiral phosphonate, phosphinate, phosphoramidate including 3′-amino phosphoramidate and aminoalkylphosphoramidate, thionophosphoramidate, thionoalkylphosphonate, thionoalkylphosphotriester, selenophosphate or boranophosphate.

A further preferred nucleotide analogue or equivalent comprises one or more sugar moieties that are mono- or disubstituted at the 2′, 3′ and/or 5′ position such as a —OH; —F; substituted or unsubstituted, linear or branched lower (C1-C10) alkyl, alkenyl, alkynyl, alkaryl, allyl, aryl, or aralkyl, that may be interrupted by one or more heteroatoms; O-, S-, or N-alkyl; O-, S-, or N-alkenyl; O-, S- or N-alkynyl; O-, S-, or N-allyl; O-alkyl-O-alkyl, -methoxy, -aminopropoxy; aminoxy, methoxyethoxy; -dimethylaminooxyethoxy; and -dimethylaminoethoxyethoxy. The sugar moiety can be a pyranose or derivative thereof, or a deoxypyranose or derivative thereof, preferably a ribose or a derivative thereof, or deoxyribose or derivative thereof. Such preferred derivatized sugar moieties comprise Locked Nucleic Acid (LNA), in which the 2′-carbon atom is linked to the 3′ or 4′ carbon atom of the sugar ring thereby forming a bicyclic sugar moiety. A preferred LNA comprises 2′-O,4′-C-ethylene-bridged nucleic acid (Morita et al. 2001. Nucleic Acid Res Supplement No. 1: 241-242). These substitutions render the nucleotide analogue or equivalent RNase H and nuclease resistant and increase the affinity for the target.

“Sequence identity” or “identity” in the context of the invention of an amino acid- or nucleic acid-sequence is herein defined as a relationship between two or more amino acid (peptide, polypeptide, or protein) sequences or two or more nucleic acid (nucleotide, oligonucleotide, polynucleotide) sequences, as determined by comparing the sequences. In the art, “identity” also means the degree of sequence relatedness between amino acid or nucleotide sequences, as the case may be, as determined by the match between strings of such sequences. Within the invention, sequence identity with a particular sequence preferably means sequence identity over the entire length of said particular polypeptide or polynucleotide sequence.

“Similarity” between two amino acid sequences is determined by comparing the amino acid sequence and its conserved amino acid substitutes of one peptide or polypeptide to the sequence of a second peptide or polypeptide. In a preferred embodiment, identity or similarity is calculated over the whole sequence (SEQ ID NO:) as identified herein. “Identity” and “similarity” can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215:403-410 (1990). The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, Wis. The aforementioned parameters are the default parameters for amino acid comparisons (along with no penalty for end gaps). Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons. Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Arg to lys; Asn to gln or his; Asp to glu; Cys to ser or ala; Gln to asn; Glu to asp; Gly to pro; His to asn or gln; Ile to leu or val; Leu to ile or val; Lys to arg; gln or glu; Met to leu or ile; Phe to met, leu or tyr; Ser to thr; Thr to ser; Trp to tyr; Tyr to trp or phe; and, Val to ile or leu.

A polynucleotide according to the invention is represented by a nucleotide sequence. A polypeptide according to the invention is represented by an amino acid sequence. A nucleic acid construct according to the invention is defined as a polynucleotide which is isolated from a naturally occurring gene or which has been modified to contain segments of polynucleotides which are combined or juxtaposed in a manner which would not otherwise exist in nature.

The sequence information as provided herein should not be so narrowly construed as to require inclusion of erroneously identified bases. The skilled person is capable of identifying such erroneously identified bases and knows how to correct for such errors.

A compound of interest in the context of all embodiments of the invention may be any biological compound. The biological compound may be biomass or a biopolymer or a metabolite. The biological compound may be encoded by a single polynucleotide or a series of polynucleotides composing a biosynthetic or metabolic pathway or may be the direct result of the product of a single polynucleotide or products of a series of polynucleotides, the polynucleotide may be a gene, the series of polynucleotide may be a gene cluster. In all embodiments of the invention, the single polynucleotide or series of polynucleotides encoding the biological compound of interest or the biosynthetic or metabolic pathway associated with the biological compound of interest, are preferred targets for the compositions and methods according to the invention. The biological compound may be native to the host cell or heterologous to the host cell.

The term “heterologous biological compound” is defined herein as a biological compound which is not native to the cell; or a native biological compound in which structural modifications have been made to alter the native biological compound.

The term “biopolymer” is defined herein as a chain (or polymer) of identical, similar, or dissimilar subunits (monomers). The biopolymer may be any biopolymer. The biopolymer may for example be, but is not limited to, a nucleic acid, polyamine, polyol, polypeptide (or polyamide), or polysaccharide.

The biopolymer may be a polypeptide. The polypeptide may be any polypeptide having a biological activity of interest. The term “polypeptide” is not meant herein to refer to a specific length of the encoded product and, therefore, encompasses peptides, oligopeptides, and proteins. The term polypeptide refers to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics. Polypeptides further include naturally occurring allelic and engineered variations of the above-mentioned polypeptides and hybrid polypeptides. The polypeptide may be native or may be heterologous to the host cell. The polypeptide may be a collagen or gelatine, or a variant or hybrid thereof. The polypeptide may be an antibody or parts thereof, an antigen, a clotting factor, an enzyme, a hormone or a hormone variant, a receptor or parts thereof, a regulatory protein, a structural protein, a reporter, or a transport protein, protein involved in secretion process, protein involved in folding process, chaperone, peptide amino acid transporter, glycosylation factor, transcription factor, synthetic peptide or oligopeptide, intracellular protein. The intracellular protein may be an enzyme such as, a protease, ceramidases, epoxide hydrolase, aminopeptidase, acylases, aldolase, hydroxylase, aminopeptidase, lipase. The polypeptide may also be an enzyme secreted extracellularly. Such enzymes may belong to the groups of oxidoreductase, transferase, hydrolase, lyase, isomerase, ligase, catalase, cellulase, chitinase, cutinase, deoxyribonuclease, dextranase, esterase. The enzyme may be a carbohydrase, e.g. cellulases such as endoglucanases, β-glucanases, cellobiohydrolases or β-glucosidases, hemicellulases or pectinolytic enzymes such as xylanases, xylosidases, mannanases, galactanases, galactosidases, pectin methyl esterases, pectin lyases, pectate lyases, endo polygalacturonases, exopolygalacturonases rhamnogalacturonases, arabanases, arabinofuranosidases, arabinoxylan hydrolases, galacturonases, lyases, or amylolytic enzymes; hydrolase, isomerase, or ligase, phosphatases such as phytases, esterases such as lipases, proteolytic enzymes, oxidoreductases such as oxidases, transferases, or isomerases. The enzyme may be a phytase. The enzyme may be an aminopeptidase, asparaginase, amylase, a maltogenic amylase, carbohydrase, carboxypeptidase, endo-protease, metallo-protease, serine-protease catalase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, protein deaminase, invertase, laccase, lipase, mannosidase, mutanase, oxidase, pectinolytic enzyme, peroxidase, phospholipase, galactolipase, chlorophyllase, polyphenoloxidase, ribonuclease, transglutaminase, or glucose oxidase, hexose oxidase, monooxygenase.

According to the invention, a compound of interest can be a polypeptide or enzyme with improved secretion features as described in WO2010/102982. According to the invention, a compound of interest can be a fused or hybrid polypeptide to which another polypeptide is fused at the N-terminus or the C-terminus of the polypeptide or fragment thereof. A fused polypeptide is produced by fusing a nucleic acid sequence (or a portion thereof) encoding one polypeptide to a nucleic acid sequence (or a portion thereof) encoding another polypeptide.

Techniques for producing fusion polypeptides are known in the art, and include, ligating the coding sequences encoding the polypeptides so that they are in frame and expression of the fused polypeptide is under control of the same promoter(s) and terminator. The hybrid polypeptides may comprise a combination of partial or complete polypeptide sequences obtained from at least two different polypeptides wherein one or more may be heterologous to the host cell. Example of fusion polypeptides and signal sequence fusions are for example as described in WO2010/121933.

The biopolymer may be a polysaccharide. The polysaccharide may be any polysaccharide, including, but not limited to, a mucopolysaccharide (e.g., heparin and hyaluronic acid) and nitrogen-containing polysaccharide (e.g., chitin). In a preferred option, the polysaccharide is hyaluronic acid. A polynucleotide coding for the compound of interest or coding for a compound involved in the production of the compound of interest according to the invention may encode an enzyme involved in the synthesis of a primary or secondary metabolite, such as organic acids, carotenoids, (beta-lactam) antibiotics, and vitamins. Such metabolite may be considered as a biological compound according to the invention.

The term “metabolite” encompasses both primary and secondary metabolites; the metabolite may be any metabolite. Preferred metabolites are citric acid, gluconic acid, adipic acid, fumaric acid, itaconic acid and succinic acid.

A metabolite may be encoded by one or more genes, such as in a biosynthetic or metabolic pathway. Primary metabolites are products of primary or general metabolism of a cell, which are concerned with energy metabolism, growth, and structure. Secondary metabolites are products of secondary metabolism (see, for example, R. B. Herbert, The Biosynthesis of Secondary Metabolites, Chapman and Hall, New York, 1981).

A primary metabolite may be, but is not limited to, an amino acid, fatty acid, nucleoside, nucleotide, sugar, triglyceride, or vitamin.

A secondary metabolite may be, but is not limited to, an alkaloid, coumarin, flavonoid, polyketide, quinine, steroid, peptide, or terpene. The secondary metabolite may be an antibiotic, antifeedant, attractant, bacteriocide, fungicide, hormone, insecticide, or rodenticide. Preferred antibiotics are cephalosporins and beta-lactams. Other preferred metabolites are exo-metabolites. Examples of exo-metabolites are Aurasperone B, Funalenone, Kotanin, Nigragillin, Orlandin, Other naphtho-γ-pyrones, Pyranonigrin A, Tensidol B, Fumonisin B2 and Ochratoxin A.

The biological compound may also be the product of a selectable marker. A selectable marker is a product of a polynucleotide of interest which product provides for biocide or viral resistance, resistance to heavy metals, prototrophy to auxotrophs, and the like. Selectable markers include, but are not limited to, amdS (acetamidase), argB (ornithinecarbamoyltransferase), bar (phosphinothricinacetyltransferase), hygB (hygromycin phosphotransferase), niaD (nitrate reductase), pyrG (orotidine-5′-phosphate decarboxylase), sC (sulfate adenyltransferase), trpC (anthranilate synthase), ble (phleomycin resistance protein), hyg (hygromycin), NAT or NTC (Nourseothricin) as well as equivalents thereof.

According to the invention, a compound of interest is preferably a polypeptide as described in the list of compounds of interest.

According to another embodiment of the invention, a compound of interest is preferably a metabolite.

A cell according to the invention may already be capable of producing a compound of interest. A cell according to the invention may also be provided with a homologous or heterologous nucleic acid construct that encodes a polypeptide wherein the polypeptide may be the compound of interest or a polypeptide involved in the production of the compound of interest. The person skilled in the art knows how to modify a microbial host cell such that it is capable of producing a compound of interest.

All embodiments of the invention refer to a cell, not to a cell-free in vitro system; in other words, the systems according to the invention are cell systems, not cell-free in vitro systems.

In all embodiments of the invention, e.g., the cell according to the invention may be a haploid, diploid or polyploid cell.

A cell according to the invention is interchangeably herein referred as “a cell”, “a cell according to the invention”, “a host cell”, and as “a host cell according to the invention”; said cell may be any cell, a prokaryotic or a eukaryotic cell. Preferably, the cell is not a mammalian cell. Preferably the cell is a fungus, i.e. a yeast cell or a filamentous fungus cell. Preferably, the cell is deficient in an NHEJ (non-homologous end joining). The cell can be deficient in NHEJ due to the cell being deficient in a component associated with NHEJ. Said component associated with NHEJ is may be a homologue or orthologue of the yeast Ku70, Ku80, MRE11, RAD50, RAD51, RAD52, XRS2, SIR4, and/or LIG4. Alternatively, in the cell according to the invention NHEJ may be rendered deficient by use of a compound that inhibits DNA ligase IV, such as SCR7 (Vartak S V and Raghavan, 2015). The person skilled in the art knows how to modulate NHEJ and its effect on RNA-guided nuclease systems, see e.g. WO2014130955A1; Chu et al., 2015; et al., 2015; Song et al., 2015 and Yu et al., 2015; all are herein incorporated by reference. The term “deficiency” is defined elsewhere herein.

When the cell according to the invention is a yeast cell, a preferred yeast cell is from a genus selected from the group consisting of Candida, Hansenula, Issatchenkia, Kluyveromyces, Pichia, Saccharomyces, Schizosaccharomyces, Yarrowia or Zygosaccharomyces; more preferably a yeast host cell is selected from the group consisting of Kluyveromyces lactis, Kluyveromyces lactis NRRL Y-1140, Kluyveromyces marxianus, Kluyveromyces. thermotolerans, Candida krusei, Candida sonorensis, Candida glabrata, Saccharomyces cerevisiae, Saccharomyces cerevisiae CEN.PK113-7D, Schizosaccharomyces pombe, Hansenula polymorpha, Issatchenkia orientalis, Yarrowia lipolytica, Yarrowia lipolytica CLIB122, Yarrowia lipolytica ATCC18943, Pichia stipidis and Pichia pastoris.

The host cell according to the invention is a filamentous fungal host cell. Filamentous fungi as defined herein include all filamentous forms of the subdivision Eumycota and Oomycota (as defined by Hawksworth et al., In, Ainsworth and Bisby's Dictionary of The Fungi, 8th edition, 1995, CAB International, University Press, Cambridge, UK).

The filamentous fungal host cell may be a cell of any filamentous form of the taxon Trichocomaceae (as defined by Houbraken and Samson in Studies in Mycology 70: 1-51.2011). In another preferred embodiment, the filamentous fungal host cell may be a cell of any filamentous form of any of the three families Aspergillaceae, Thermoascaceae and Trichocomaceae, which are accommodated in the taxon Trichocomaceae.

The filamentous fungi are characterized by a mycelial wall composed of chitin, cellulose, glucan, chitosan, mannan, and other complex polysaccharides. Vegetative growth is by hyphal elongation and carbon catabolism is obligatory aerobic. Filamentous fungal strains include, but are not limited to, strains of Acremonium, Agaricus, Aspergillus, Aureobasidium, Chrysosporium, Coprinus, Cryptococcus, Filibasidium, Fusarium, Humicola, Magnaporthe, Mortierella, Mucor, Myceliophthora, Neocallimastix, Neurospora, Paecilomyces, Penicillium, Piromyces, Panerochaete, Pleurotus, Schizophyllum, Talaromyces, Rasamsonia, Thermoascus, Thielavia, Tolypocladium, and Trichoderma. A preferred filamentous fungal host cell according to the invention is from a genus selected from the group consisting of Acremonium, Aspergillus, Chrysosporium, Myceliophthora, Penicillium, Talaromyces, Rasamsonia, Thielavia, Fusarium and Trichoderma; more preferably from a species selected from the group consisting of Aspergillus niger, Acremonium alabamense, Aspergillus awamori, Aspergillus foetidus, Aspergillus sojae, Aspergillus fumigatus, Talaromyces emersonii, Rasamsonia emersonii, Rasamsonia emersonii CBS393.64, Aspergillus oryzae, Chrysosporium lucknowense, Fusarium oxysporum, Mortierella alpina, Mortierella alpina ATCC 32222, Myceliophthora thermophila, Trichoderma reesei, Thielavia terrestris, Penicillium chrysogenum and P. chrysogenum Wisconsin 54-1255 (ATCC28089); even more preferably the filamentous fungal host cell according to the invention is an Aspergillus niger. When the host cell according to the invention is an Aspergillus niger host cell, the host cell preferably is CBS 513.88, CBS124.903 or a derivative thereof.

Several strains of filamentous fungi are readily accessible to the public in a number of culture collections, such as the American Type Culture Collection (ATCC), Deutsche Sammlung von Mikroorganismen and Zellkulturen GmbH (DSM), Centraalbureau Voor Schimmelcultures (CBS), Agricultural Research Service Patent Culture Collection, Northern Regional Research Center (NRRL), and All-Russian Collection of Microorganisms of Russian Academy of Sciences, (abbreviation in Russian—VKM, abbreviation in English—RCM), Moscow, Russia. Preferred strains as host cells according to the present invention are Aspergillus nigerCBS 513.88, CBS124.903, Aspergillus oryzae ATCC 20423, IFO 4177, ATCC 1011, CBS205.89, ATCC 9576, ATCC14488-14491, ATCC 11601, ATCC12892, P. chrysogenum CBS 455.95, P. chrysogenum Wisconsin54-1255 (ATCC28089), Penicillium citrinum ATCC 38065, Penicillium chrysogenum P2, Thielavia terrestris NRRL8126, Rasamsonia emersonfiCBS393.64, Talaromyces emersonii CBS 124.902, Acremonium chrysogenum ATCC 36225 or ATCC 48272, Trichoderma reesei ATCC 26921 or ATCC 56765 or ATCC 26921, Aspergillus sojae ATCC11906, Myceliophthora thermophila Cl, Garg 27K, VKM-F 3500 D, Chrysosporium lucknowense Cl, Garg 27K, VKM-F 3500 D, ATCC44006 and derivatives thereof.

Preferably, a host cell according to the invention has a modification, preferably in its genome which results in a reduced or no production of an undesired compound as defined herein if compared to the parent host cell that has not been modified, when analysed under the same conditions.

A modification can be introduced by any means known to the person skilled in the art, such as but not limited to classical strain improvement, random mutagenesis followed by selection. Modification can also be introduced by site-directed mutagenesis.

Modification may be accomplished by the introduction (insertion), substitution (replacement) or removal (deletion) of one or more nucleotides in a polynucleotide sequence. A full or partial deletion of a polynucleotide coding for an undesired compound such as a polypeptide may be achieved. An undesired compound may be any undesired compound listed elsewhere herein; it may also be a protein and/or enzyme in a biological pathway of the synthesis of an undesired compound such as a metabolite. Alternatively, a polynucleotide coding for said undesired compound may be partially or fully replaced with a polynucleotide sequence which does not code for said undesired compound or that codes for a partially or fully inactive form of said undesired compound. In another alternative, one or more nucleotides can be inserted into the polynucleotide encoding said undesired compound resulting in the disruption of said polynucleotide and consequent partial or full inactivation of said undesired compound encoded by the disrupted polynucleotide.

In an embodiment the host cell according to the invention comprises a modification in its genome selected from
a) a full or partial deletion of a polynucleotide encoding an undesired compound,
b) a full or partial replacement of a polynucleotide encoding an undesired compound with a polynucleotide sequence which does not code for said undesired compound or that codes for a partially or fully inactive form of said undesired compound.
c) a disruption of a polynucleotide encoding an undesired compound by the insertion of one or more nucleotides in the polynucleotide sequence and consequent partial or full inactivation of said undesired compound by the disrupted polynucleotide.

This modification may for example be in a coding sequence or a regulatory element required for the transcription or translation of said undesired compound. For example, nucleotides may be inserted or removed so as to result in the introduction of a stop codon, the removal of a start codon or a change or a frame-shift of the open reading frame of a coding sequence. The modification of a coding sequence or a regulatory element thereof may be accomplished by site-directed or random mutagenesis, DNA shuffling methods, DNA reassembly methods, gene synthesis (see for example Young and Dong, (2004), Nucleic Acids Research 32(7) or Gupta et al. (1968), Proc. Natl. Acad. Sci USA, 60: 1338-1344; Scarpulla et al. (1982), Anal. Biochem. 121: 356-365; Stemmer et al. (1995), Gene 164: 49-53), or PCR generated mutagenesis in accordance with methods known in the art. Examples of random mutagenesis procedures are well known in the art, such as for example chemical (NTG for example) mutagenesis or physical (UV for example) mutagenesis. Examples of site-directed mutagenesis procedures are the QuickChange™ site-directed mutagenesis kit (Stratagene Cloning Systems, La Jolla, Calif.), the ‘The Altered Sites® II in vitro Mutagenesis Systems’ (Promega Corporation) or by overlap extension using PCR as described in Gene. 1989 Apr. 15; 77(1):51-9. (Ho S N, Hunt H D, Horton R M, Pullen J K, Pease L R “Site-directed mutagenesis by overlap extension using the polymerase chain reaction”) or using PCR as described in Molecular Biology: Current Innovations and Future Trends. (Eds. A. M. Griffin and H. G. Griffin. ISBN 1-898486-01-8; 1995 Horizon Scientific Press, PO Box 1, Wymondham, Norfolk, U.K.).

Preferred methods of modification are based on recombinant genetic manipulation techniques such as partial or complete gene replacement or partial or complete gene deletion.

For example, in case of replacement of a polynucleotide, nucleic acid construct or expression cassette, an appropriate DNA sequence may be introduced at the target locus to be replaced. The appropriate DNA sequence is preferably present on a cloning vector. Preferred integrative cloning vectors comprise a DNA fragment, which is homologous to the polynucleotide and/or has homology to the polynucleotides flanking the locus to be replaced for targeting the integration of the cloning vector to this pre-determined locus. In order to promote targeted integration, the cloning vector is preferably linearized prior to transformation of the cell. Preferably, linearization is performed such that at least one but preferably either end of the cloning vector is flanked by sequences homologous to the DNA sequence (or flanking sequences) to be replaced. This process is called homologous recombination and this technique may also be used in order to achieve (partial) gene deletion.

For example, a polynucleotide corresponding to the endogenous polynucleotide may be replaced by a defective polynucleotide; that is a polynucleotide that fails to produce a (fully functional) polypeptide. By homologous recombination, the defective polynucleotide replaces the endogenous polynucleotide. It may be desirable that the defective polynucleotide also encodes a marker, which may be used for selection of transformants in which the nucleic acid sequence has been modified.

Alternatively, or in combination with other mentioned techniques, a technique based on recombination of cosmids in an E. coli cell can be used, as described in: A rapid method for efficient gene replacement in the filamentous fungus Aspergillus nidulans (2000) Chaveroche, M-K., Ghico, J-M. and d′Enfert C; Nucleic acids Research, vol 28, no 22.

Alternatively, modification, wherein said host cell produces less of or no protein such as the polypeptide having amylase activity, preferably α-amylase activity as described herein and encoded by a polynucleotide as described herein, may be performed by established anti-sense techniques using a nucleotide sequence complementary to the nucleic acid sequence of the polynucleotide. More specifically, expression of the polynucleotide by a host cell may be reduced or eliminated by introducing a nucleotide sequence complementary to the nucleic acid sequence of the polynucleotide, which may be transcribed in the cell and is capable of hybridizing to the mRNA produced in the cell. Under conditions allowing the complementary anti-sense nucleotide sequence to hybridize to the mRNA, the amount of protein translated is thus reduced or eliminated. An example of expressing an antisense-RNA is shown in Appl. Environ. Microbiol. 2000 February; 66(2):775-82. (Characterization of a foldase, protein disulfide isomerase A, in the protein secretory pathway of Aspergillus niger. Ngiam C, Jeenes D J, Punt P J, Van Den Hondel C A, Archer D B) or (Zrenner R, Willmitzer L, Sonnewald U. Analysis of the expression of potato uridinediphosphate-glucose pyrophosphorylase and its inhibition by antisense RNA. Planta. (1993); 190(2):247-52).

A modification resulting in reduced or no production of undesired compound is preferably due to a reduced production of the mRNA encoding said undesired compound if compared with a parent microbial host cell which has not been modified and when measured under the same conditions.

A modification which results in a reduced amount of the mRNA transcribed from the polynucleotide encoding the undesired compound may be obtained via the RNA interference (RNAi) technique (Mouyna et al., 2004). In this method identical sense and antisense parts of the nucleotide sequence, which expression is to be affected, are cloned behind each other with a nucleotide spacer in between, and inserted into an expression vector. After such a molecule is transcribed, formation of small nucleotide fragments will lead to a targeted degradation of the mRNA, which is to be affected. The elimination of the specific mRNA can be to various extents. The RNA interference techniques described in e.g. WO2008/053019, WO2005/05672A1 and WO2005/026356A1.

A modification which results in decreased or no production of an undesired compound can be obtained by different methods, for example by an antibody directed against such undesired compound or a chemical inhibitor or a protein inhibitor or a physical inhibitor (Tour O. et al, (2003) Nat. Biotech: Genetically targeted chromophore-assisted light inactivation. Vol. 21. no. 12:1505-1508) or peptide inhibitor or an anti-sense molecule or RNAi molecule (R. S. Kamath et al, (2003) Nature: Systematic functional analysis of the Caenorhabditis elegans genome using RNAi. Vol. 421, 231-237).

In addition of the above-mentioned techniques or as an alternative, it is also possible to inhibiting the activity of an undesired compound, or to re-localize the undesired compound such as a protein by means of alternative signal sequences (Ramon de Lucas, J., Martinez O, Perez P., Isabel Lopez, M., Valenciano, S. and Laborda, F. The Aspergillus nidulans carnitine carrier encoded by the acuH gene is exclusively located in the mitochondria. FEMS Microbiol Lett. 2001 Jul. 24; 201(2):193-8.) or retention signals (Derkx, P. M. and Madrid, S. M. The foldase CYPB is a component of the secretory pathway of Aspergillus niger and contains the endoplasmic reticulum retention signal HEEL. Mol. Genet. Genomics. 2001 December; 266 (4):537-545), or by targeting an undesired compound such as a polypeptide to a peroxisome which is capable of fusing with a membrane-structure of the cell involved in the secretory pathway of the cell, leading to secretion outside the cell of the polypeptide (e.g. as described in WO2006/040340).

Alternatively, or in combination with above-mentioned techniques, decreased or no production of an undesired compound can also be obtained, e.g. by UV or chemical mutagenesis (Mattern, I. E., van Noort J. M., van den Berg, P., Archer, D. B., Roberts, I. N. and van den Hondel, C. A., Isolation and characterization of mutants of Aspergillus niger deficient in extracellular proteases. Mol Gen Genet. 1992 August; 234(2):332-6.) or by the use of inhibitors inhibiting enzymatic activity of an undesired polypeptide as described herein (e.g. nojirimycin, which function as inhibitor for β-glucosidases (Carrel F. L. Y. and Canevascini G. Canadian Journal of Microbiology (1991) 37(6): 459-464; Reese E. T., Parrish F. W. and Ettlinger M. Carbohydrate Research (1971) 381-388)).

In an embodiment of the invention, the modification in the genome of the host cell according to the invention is a modification in at least one position of a polynucleotide encoding an undesired compound.

A deficiency of a cell in the production of a compound, for example of an undesired compound such as an undesired polypeptide and/or enzyme is herein defined as a mutant microbial host cell which has been modified, preferably in its genome, to result in a phenotypic feature wherein the cell: a) produces less of the undesired compound or produces substantially none of the undesired compound and/or b) produces the undesired compound having a decreased activity or decreased specific activity or the undesired compound having no activity or no specific activity and combinations of one or more of these possibilities as compared to the parent host cell that has not been modified, when analysed under the same conditions.

Preferably, a modified host cell according to the invention produces 1% less of the un-desired compound if compared with the parent host cell which has not been modified and measured under the same conditions, at least 5% less of the un-desired compound, at least 10% less of the un-desired compound, at least 20% less of the un-desired compound, at least 30% less of the un-desired compound, at least 40% less of the un-desired compound, at least 50% less of the un-desired compound, at least 60% less of the un-desired compound, at least 70% less of the un-desired compound, at least 80% less of the un-desired compound, at least 90% less of the un-desired compound, at least 91% less of the un-desired compound, at least 92% less of the un-desired compound, at least 93% less of the un-desired compound, at least 94% less of the un-desired compound, at least 95% less of the un-desired compound, at least 96% less of the un-desired compound, at least 97% less of the un-desired compound, at least 98% less of the un-desired compound, at least 99% less of the un-desired compound, at least 99.9% less of the un-desired compound, or most preferably 100% less of the un-desired compound.

A reference herein to a patent document or other matter which is given as prior art is not to be taken as an admission that that document or matter was known or that the information it contains was part of the common general knowledge as at the priority date of any of the claims.

The disclosure of each reference set forth herein is incorporated herein by reference in its entirety.

The invention is further illustrated by the following examples:

### Examples

In the following Examples, various embodiments of the invention are illustrated. From the above description and these Examples, one skilled in the art can make various changes and modifications of the invention to adapt it to various usages and conditions.

### Example 1: Cas9 Genome Editing by CRISPR Transient Editing Construct (CTEC) in S. cerevisiae

This example describes genome editing of Saccharomyces cerevisiae by the integration of a donor DNA fragment encoding desired mutations making use a CRISPR/Cas9 system and transient expression of guide RNA. The CTEC DNA fragment(s) that are used comprise a guide-RNA expression cassette with control elements as previously described by DiCarlo et al., 2013 for the expression of guide-RNA's in S. cerevisiae and a donor DNA sequence for editing the targeted genomic sequence. The Cas9 guide-RNA expression cassettes used in this example comprise the SNR52 promoter, a guide-RNA sequence consisting of the guide-sequence (also referred to as genomic target sequence) and the guide-RNA structural component followed by the SUP4 terminator. The donor DNA is 100 bp when targeting the INT1 locus in the genome and encodes a DNA base substitution changing the PAM sequence from AGG to ATG. The donor DNA is 111 bp when the YFP gene is targeted and encodes a frameshift; deletion of one DNA base in the genomic target sequence, causing loss of fluorescence. This set-up is visually shown in FIG. 15.

Construction of a Cas9-Expressing Saccharomyces cerevisiae Strain

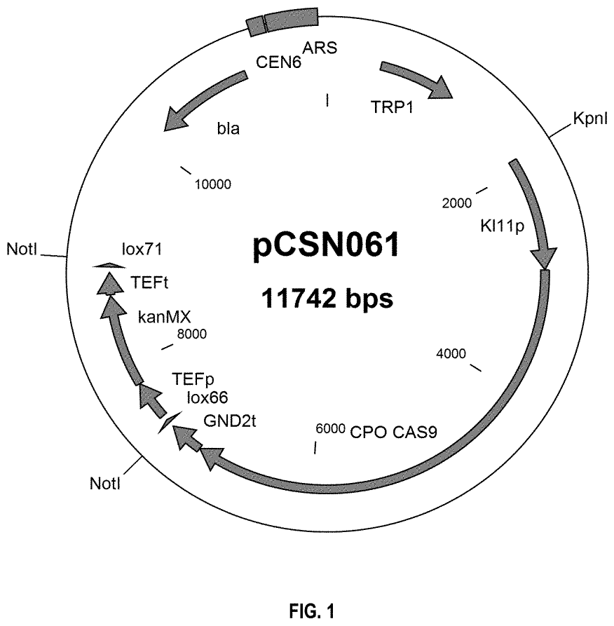

Yeast vector pCSN061 is a single copy vector (CEN/ARS) that contains a Cas9 expression cassette consisting of a Cas9 codon optimized variant (WO2016/110512) expressed from the KI11 promoter (Kluyveromyces lactis promoter of KLLA0F20031g), the S. cerevisiae GND2 terminator, and a functional KanMX marker cassette conferring resistance against G418. The Cas9 expression cassette was KpnI/NotI ligated into pRS414 (Sikorski and Hieter, 1989), resulting in intermediate vector pCSN004. Subsequently, a functional expression cassette conferring G418 resistance (see: www.euroscarf.de) was NotI restricted from vector pUG7-KanMX and NotI ligated into pCSN004, resulting in vector pCSN061 that is depicted in FIG. 1; the sequence is set out in SEQ ID NO: 2. Vector pCSN061 containing the Cas9 expression cassette was first transformed to S. cerevisiae strain CEN.PK113-7D (MATa URA3 HIS3 LEU2 TRP1 MAL2-8 SUC2) using the LiAc/salmon sperm (SS) carrier DNA/PEG method (Gietz and Woods, 2002). Strain CEN.PK113-7D is available from the EUROSCARF collection (http://www.euroscarf.de, Frankfurt, Germany). The origin of the CEN.PK family of strains is described by van Dijken et al., 2000. In the transformation mixture one microgram of vector pCNS061 was used. The transformation mixture was plated on YPD-agar (10 grams per liter of yeast extract, 20 grams per liter of peptone, 20 grams per liter of dextrose, 20 grams per liter of agar) containing 200 microgram (μg) G418 (Sigma Aldrich, Zwijndrecht, the Netherlands) per ml. After two to four days of growth at 30° C. transformants appeared on the transformation plate. A transformant conferring resistance to G418 on the plate, further referred to as strain CSN001, was inoculated on YPD-G418 medium (10 grams per liter of yeast extract, 20 grams per liter of peptone, 20 grams per liter of dextrose, 200 μg G418 (Sigma Aldrich, Zwijndrecht, the Netherlands) per ml, was used in subsequent transformation experiments.

### Double-Stranded DNA (Ds-DNA) YFP Donor DNA Cassette

A double-stranded donor DNA cassette coding for the Yellow Fluorescent Protein (YFP) variant Venus (Nagai et al., 2002), was prepared via a Golden-Gate assembly reaction of individual promoter (P), orf (O) and terminator (T) sequences in an appropriate E. coli vector. The assembled POT cassette was amplified via a PCR reaction with primers indicated in SEQ ID NO: 4 and SEQ ID NO: 5. In a second PCR, 50 bp connector sequences are added using primer sets indicated in SEQ ID NO: 6 and SEQ ID NO: 7. This resulted in an YFP expression cassette that included 50 bp connector sequences at the 5′ and 3′ ends of the expression cassette (SEQ ID NO: 8). The YFP expression cassette in between connector sequences is used as template in the subsequent PCR reaction using primer set (SEQ ID NO: 9 and SEQ ID NO: 10). In this PCR reaction 50 bp genomic flanks are added for integration into the genomic locus, INT1, of S. cerevisiae strain CSN001. The sequence of the resulting YFP cassette flanked by 50 bp genomic sequences is presented in SEQ ID NO: 11.

The Q5 DNA polymerase (part of the Q5® High-Fidelity 2X Master Mix, New England Biolabs, supplied by Bioké, Leiden, the Netherlands. Cat no. M0492S) was used in the PCR reactions described above. PCR reactions were performed according to manufacturer's instructions.

### PCR Purification

Purification of PCR reactions was performed using NucleoSpin Gel and PCR Clean-up kit (Machery-Nagel, distributed by Bioké, Leiden, the Netherlands) according to manufacturer's instructions.

Guide-RNA (sgRNA) Expression Cassette INT1

Guide-RNA expression cassettes were ordered as synthetic DNA (gBlocks) at Integrated DNA Technologies (IDT, Leuven, Belgium). The guide-RNA expression cassettes consisted of the SNR52p RNA polymerase III promoter, a guide-sequence (also referred to as genomic target sequence; SEQ ID NO:12), the gRNA structural component and the SUP4 3′ flanking region as described in DiCarlo et al. For in vivo homologous recombination into the linearized pRN1120 (XhoI, EcoRI) vector backbone, 50 bp homology to pRN1120 was added on either side of the guide-RNA expression cassette, resulting in a fragment of 488 bp in total (SEQ ID NO: 13).

pRN1120 Vector Construction (Multi-Copy Expression Vector, NatMX Marker)

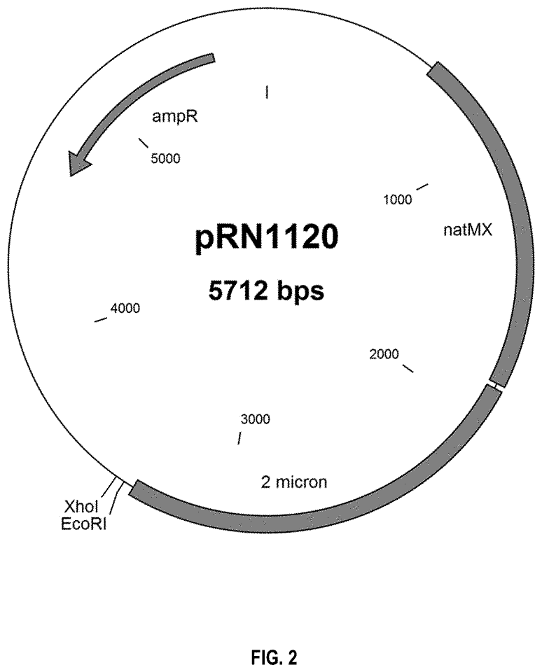

Yeast vector pRN1120 is a multi-copy vector (2 micron) that contains a functional NatMX marker cassette conferring resistance against nourseothricin. The backbone of this vector is based on pRS305 (Sikorski and Hieter, 1989), and includes a functional 2 micron ORI sequence and a functional NatMX marker cassette (see www.euroscarf.de). Vector pRN1120 is depicted in FIG. 2 and the sequence is set out in SEQ ID NO: 3.

Construction of a Cas9-Expressing Saccharomyces cerevisiae Strain with YFP Expression Cassette Integrated at INT1 Locus in the Genome

S. cerevisiae strain CSN001 was transformed using the LiAc/salmon sperm (SS) carrier DNA/PEG method (Gietz and Woods, 2002). Prior to transformation strain CSN001 was cultivated in YPD liquid medium (10 grams per liter of yeast extract, 20 grams per liter of peptone, 20 grams per liter of dextrose) supplemented with 200 microgram (μg) G418 (Sigma Aldrich, Zwijndrecht, the Netherlands) per ml. Strain CSN001 was transformed with XhoI/EcoRI restricted pRN1120 and a sgRNA expression cassette, targeting INT1 SEQ ID NO: 13. The linearized pRN1120 is a recipient for the sgRNA expression cassette which contains homology with pRN1120 at both ends to allow in vivo recombination into a circular plasmid. Cas9, that is pre-expressed in the cells, is directed to the genomic target, INT1, to create a double stranded break. In the transformation mixture, YFP donor DNA cassette for integration at INT1 locus (100 ng) is also included.

The transformation mixture was plated on YPD-agar (10 grams per liter of yeast extract, 20 grams per liter of peptone, 20 grams per liter of dextrose, 20 grams per liter of agar) containing 200 microgram (μg) G418 (Sigma Aldrich, Zwijndrecht, the Netherlands) and 200 microgram (μg) nourseothricin (NTC, Jena Bioscience, Germany) per ml. After two to four days of growth at 30° C. transformants appeared on the transformation plate. A transformant conferring resistance to G418 and nourseothricin on the plate, and expressing YFP is selected. YFP expression is assessed using the Qpix450 (Molecular Devices; Filter: Ex/Em: 457/536 nm-FITC/GFP). This strain is to be used in additional Cas9 experiments therefor it is cured from its guide RNA plasmid (nourseothricin marker) while maintaining its Cas9 expression plasmid (KanMX marker). The strain is grown for 24 hours in YPD liquid medium (10 grams per liter of yeast extract, 20 grams per liter of peptone, 20 grams per liter of dextrose) supplemented with 200 microgram (μg) G418 (Sigma Aldrich, Zwijndrecht, the Netherlands) per ml at 30° C., shaking speed: 250 rpm. Dilutions of the culture were made in milliQ and subsequently plated onto YPD-agar medium (10 grams per liter of yeast extract, 20 grams per liter of peptone, 20 grams per liter of dextrose, 20 grams per liter of agar) containing 200 microgram (μg) G418 (Sigma Aldrich, Zwijndrecht, the Netherlands). After two to four days of growth at 30° C., colonies appeared on the agar plate. Single colonies were subsequently checked for nourseothricin sensitivity by streaking them on YPD-agar (10 grams per liter of yeast extract, 20 grams per liter of peptone, 20 grams per liter of dextrose, 20 grams per liter of agar) containing 200 microgram (μg) nourseothricin (NTC, Jena Bioscience, Germany) per ml. A nourseothricin sensitive strain was selected and designated CSN009. This strain was used in further transformation experiments.

### CRISPR Transient Editing Construct (CTEC) DNA Fragments

CTEC DNA fragments containing guide-RNA expression cassettes as well as donor DNA were ordered as synthetic DNA (gBlocks) at Integrated DNA Technologies (IDT, Leuven, Belgium). Six designs were made per targeted genomic region, INT1, or YFP ORF, an overview of the designs is provided in FIG. 3. The designs of the CTEC DNA's, of which the sequences are set out in SEQ ID NO's: 14, 15, 16, 17, 18, and 19 (targeting INT1) and SEQ ID NO's: 20, 21, 22, 23, 24 and 25 (targeting YFP) consist of the SNR52p RNA polymerase III promoter, a guide-sequence (also referred to as genomic target sequence; SEQ ID NO's: 26 (INT1) and 27 (YFP), the gRNA structural component and the SUP4 3′ flanking region as described in DiCarlo et al., 2013, and the donor DNA that encodes a DNA base substitution (INT1) or DNA base deletion causing a frameshift (YFP). The effect of a 50 bp connector, connector A, sequence (SEQ ID NO: 28) as well as the presence of guide target and PAM sequence for separation of donor DNA and guide RNA expression cassette (sgRNA) are also evaluated. Connector A is a random DNA sequence of 50 bp without any homology to the genome. When a guide target and PAM sequence were included in the CTEC fragment the guide sequence for creating the ds break is encoded by the sgRNA cassette of that same CTEC fragment.

An overview of the sequences is provided in Table 1.

TABLE 1
Overview of the sequences of the CTEC DNA's used in transformation. The CTEC
fragments were used as a template in PCR reactions using the primers indicated
in this table. PCR reactions were set-up to obtain CTEC DNA fragments in higher
quantities that are later to be used in the transformation experiments.

| | | | | Primers | |
|---|---|---|---|---|---|
| | | | | used to | Sequence |
| | guide-RNA | | | obtain | of the |
| | expression | Guide | Donor | CTEC DNA | CTEC DNA |
| CTEC design | cassette | sequence | DNA | fragment | fragment |
| YFP target + 3′ donor | SEQ ID | SEQ ID | SEQ ID | SEQ ID | SEQ ID |
| | NO: 29 | NO: 27 | NO: 31 | NO: 33 | NO: 20 |
| | | | | SEQ ID | |
| | | | | NO: 35 | |
| YFP target + connector | SEQ ID | SEQ ID | SEQ ID | SEQ ID | SEQ ID |
| A + 3′ donor | NO: 29 | NO: 27 | NO: 31 | NO: 33 | NO: 21 |
| | | | | SEQ ID | |
| | | | | NO: 35 | |
| 5′ donor + YFP target | SEQ ID | SEQ ID | SEQ ID | SEQ ID | SEQ ID |
| | NO: 29 | NO: 27 | NO: 31 | NO: 34 | NO: 22 |
| | | | | SEQ ID | |
| | | | | NO: 36 | |
| 5′ donor + connector | SEQ ID | SEQ ID | SEQ ID | SEQ ID | SEQ ID |
| A + YFP target | NO: 29 | NO: 27 | NO: 31 | NO: 34 | NO: 23 |
| | | | | SEQ ID | |
| | | | | NO: 36 | |
| 5′ donor + PAM_guide | SEQ ID | SEQ ID | SEQ ID | SEQ ID | SEQ ID |
| target + YFP target | NO: 29 | NO: 27 | NO: 31 | NO: 34 | NO: 24 |
| | | | | SEQ ID | |
| | | | | NO: 36 | |
| YFP target + guide | SEQ ID | SEQ ID | SEQ ID | SEQ ID | SEQ ID |
| target_PAM + 3′ donor | NO: 29 | NO: 27 | NO: 31 | NO: 33 | NO: 25 |
| | | | | SEQ ID | |
| | | | | NO: 35 | |
| INT1 target + 3′ donor | SEQ ID | SEQ ID | SEQ ID | SEQ ID | SEQ ID |
| | NO: 30 | NO: 26 | NO: 32 | NO: 33 | NO: 14 |
| | | | | SEQ ID | |
| | | | | NO: 38 | |
| INT1 target + connector | SEQ ID | SEQ ID | SEQ ID | SEQ ID | SEQ ID |
| A + 3′ donor | NO: 30 | NO: 26 | NO: 32 | NO: 33 | NO: 15 |
| | | | | SEQ ID | |
| | | | | NO: 38 | |
| 5′ donor + INT1 target | SEQ ID | SEQ ID | SEQ ID | SEQ ID | SEQ ID |
| | NO: 30 | NO: 26 | NO: 32 | NO: 36 | NO: 16 |
| | | | | SEQ ID | |
| | | | | NO: 37 | |
| 5′ donor + connector | SEQ ID | SEQ ID | SEQ ID | SEQ ID | SEQ ID |
| A + INT1 target | NO: 30 | NO: 26 | NO: 32 | NO: 36 | NO: 17 |
| | | | | SEQ ID | |
| | | | | NO: 37 | |
| 5′ donor + PAM_guide | SEQ ID | SEQ ID | SEQ ID | SEQ ID | SEQ ID |
| target + INT1 target | NO: 30 | NO: 26 | NO: 32 | NO: 36 | NO: 18 |
| | | | | SEQ ID | |
| | | | | NO: 37 | |
| INT1 target + PAM_guide | SEQ ID | SEQ ID | SEQ ID | SEQ ID | SEQ ID |
| target + 3′ donor | NO: 30 | NO: 26 | NO: 32 | NO: 33 | NO: 19 |
| | | | | SEQ ID | |
| | | | | NO: 38 | |

The CTEC fragments (gBlock) were used as a template in PCR reactions using the primers indicated in this table. PCR reactions were set-up to obtain CTEC DNA fragments in higher quantities that are later to be used in the transformation experiments. PrimeSTAR GXL DNA Polymerase (Takara/Cat no. R050A) was used in the PCR reactions according to the manufacturer's instructions. The PCR generated CTEC DNA's were purified using a NucleoSpin Gel and PCR Clean-up kit (Machery-Nagel, distributed by Bioké, Leiden, the Netherlands) according to manufacturer's instructions. Subsequently, DNA concentrations were measured using a NanoDrop (ND-1000 Spectrophotometer, Thermo Scientific, Bleiswijk, the Netherlands).

### DNA Concentrations

All DNA concentrations, including the CTEC DNA fragments (PCR product) and pRN1120, were determined using a NanoDrop device (ThermoFisher, Life Technologies, Bleiswijk, the Netherlands), providing the concentrations in nanogram per microliter. Based on these measurements, an amount of 1 μg CTEC DNA and 100 ng of circular plasmid pRN1120 were used in the transformation experiments.

### Target Sites

The INT1 integration site is located in the non-coding region between NTR1 (YOR071c) and GYP1 (YOR070c), located on chromosome XV.

The YFP expression cassette, of strain S. cerevisiae CSN009, is located on the INT1 integration locus which means that is in the non-coding region between NTR1 (YOR071c) and GYP1 (YOR070c), located on chromosome XV.

### Yeast Transformation

Strain CSN001 which is pre-expressing Cas9 and strain CSN009 which is pre-expressing Cas9 and YFP, were inoculated in YPD-G418 medium (10 grams per liter of yeast extract, 20 grams per liter of peptone, 20 grams per liter of dextrose, 200 μg G418 (Sigma Aldrich, Zwijndrecht, the Netherlands) per ml. Subsequently, strain CSN001 and CSN009 were transformed with 1 μg of CTEC DNA, as indicated in Table 2, and 100 ng vector pRN1120, using the LiAc/SS carrier DNA/PEG method (Gietz and Woods, 2002).

The transformation mixtures were plated on YPD-agar (10 grams per liter of yeast extract, 20 grams per liter of peptone, 20 grams per liter of dextrose, 20 grams per liter of agar) containing 200 μg nourseothricin (NTC, Jena Bioscience, Germany) and 200 μg G418 (Sigma Aldrich, Zwijndrecht, the Netherlands) per ml. The plates were incubated at 30 degrees Celsius until colonies appeared on the plates.

TABLE 2
Overview of CTEC DNA's used in the different transformation experiments.

| | | | CTEC DNA | |
|---|---|---|---|---|
| Transformation | Description | Strain | sequence | FIG. |
| #1 | YFP target + 3′ donor | CSN009 | SEQ ID | FIG. 3 |
| | | | NO: 20 | CTEC-1 |
| #2 | YFP target + connector | CSN009 | SEQ ID | FIG. 3 |
| | A + 3′ donor | | NO: 21 | CTEC-2 |
| #3 | 5′ donor + YFP target | CSN009 | SEQ ID | FIG. 3 |
| | | | NO: 22 | CTEC-3 |
| #4 | 5′ donor + connector | CSN009 | SEQ ID | FIG. 3 |
| | A + YFP target | | NO: 23 | CTEC-4 |
| #5 | 5′ donor + PAM_guide | CSN009 | SEQ ID | FIG. 3 |
| | target + YFP target | | NO: 24 | CTEC-5 |
| #6 | YFP target + guide | CSN009 | SEQ ID | FIG. 3 |
| | target_PAM + 3′ donor | | NO: 25 | CTEC-6A |
| #7 | INT1 target + 3′ donor | CSN001 | SEQ ID | FIG. 3 |
| | | | NO: 14 | CTEC-1 |
| #8 | INT1 target + connector | CSN001 | SEQ ID | FIG. 3 |
| | A + 3′ donor | | NO: 15 | CTEC-2 |
| #9 | 5′ donor + INT1 target | CSN001 | SEQ ID | FIG. 3 |
| | | | NO: 16 | CTEC-3 |
| #10 | 5′ donor + connector | CSN001 | SEQ ID | FIG. 3 |
| | A + INT1 target | | NO: 17 | CTEC-4 |
| #11 | 5′ donor + PAM_guide | CSN001 | SEQ ID | FIG. 3 |
| | target + INT1 target | | NO: 18 | CTEC-5 |
| #12 | INT1 target + PAM_guide | CSN001 | SEQ ID | FIG. 3 |
| | target + 3′ donor | | NO: 19 | CTEC-6B |
| #13 | pRN1120 | CSN001 | — | |
| #14 | pRN1120 | CSN009 | — | |

### Results

The colonies resulting from the transformation experiment outlined above in Table 2 were checked for incorporation of the donor DNA after transient expression of the guide RNA that is encoded on the CTEC DNA fragment. Incorporation of the donor DNA that is targeted towards the YFP cassette, results in a frameshift in the YFP ORF, resulting in loss of fluorescence. The YFP fluorescence of the colonies after transformation was visualized by the QPix450 (Molecular Devices, Filter: Ex/Em: 457/536 nm-FITC/GFP). The success rate of YFP editing by the CTEC DNA fragment based on phenotype is summarized below in Table 3.

TABLE 3
Overview of YFP editing frequencies based on phenotype (loss of fluorescence) by different
CTEC fragment designs. The counted transformants are from a transformation mix that
is diluted 10 times before plating on the YPD-agar (10 grams per liter of yeast extract,
20 grams per liter of peptone, 20 grams per liter of dextrose, 20 grams per liter
of agar) containing 200 μg nourseothricin (NTC, Jena Bioscience, Germany) and
200 μg G418 (Sigma Aldrich, Zwijndrecht, the Netherlands) per ml.

| | | | | | Percentage of |
|---|---|---|---|---|---|
| | | | | Number of | non- |
| | | | Total | non- | fluorescent, |
| | | | number of | fluorescent | edited |
| Transformation | Description | Strain | transformants | transformants | colonies |
| #1 | YFP target + 3′ | CSN009 | 67 | 53 | 79% |
| | donor | | | | |
| #2 | YFP target + connector | CSN009 | 70 | 61 | 87% |
| | A + 3′ donor | | | | |
| #3 | 5′ donor + YFP | CSN009 | 100 | 98 | 98% |
| | target | | | | |
| #4 | 5′ donor + | CSN009 | 110 | 99 | 90% |
| | connector A + | | | | |
| | YFP target | | | | |
| #5 | 5′ donor + | CSN009 | 89 | 85 | 96% |
| | PAM_guide target + | | | | |
| | YFP target | | | | |
| #6 | YFP target + | CSN009 | 109 | 82 | 75% |
| | guide target_PAM + | | | | |
| | 3′ donor | | | | |
| #14 | pRN1120 | CSN009 | 121 | 0 | 0% |

Of each transformation, 12 non-fluorescent colonies were analyzed by Sanger sequencing for correct integration of the donor DNA without incorporation of additional bases from the CTEC DNA fragment. Genomic DNA of the transformants was isolated as described by Lōoke et al., 2011 and was used as template in a PCR reaction. The primer set (SEQ ID NO: 39 and SEQ ID NO:40) used to confirm the integration of the donor DNA was designed to hybridize outside the donor DNA, 138 bp up- and 465 bp down-stream. PCR reactions were performed using Phusion® High Fidelity Polymerase (Catno. M0530L, New England Biolabs-USA) according to manufacturer's instructions and a standard PCR program known to the person skilled in the art. The resulting PCR product was purified using a NucleoSpin Gel and PCR Clean-up kit (Machery-Nagel, distributed by Bioké, Leiden, the Netherlands), subsequently the PCR fragment was used as template in a sequencing reaction. Sequencing reactions were set-up making use of a BigDye® Terminator v3.1 Cycle Sequencing Kit (Catno. 4337456, ThermoFisher Scientific, Bleiswijk, the Netherlands) according to supplier's instructions. The sequencing reactions were purified by NucleoSEQ columns (Catno. 740523.250, Machery-Nagel, distributed by Bioké, Leiden, the Netherlands) according supplier's instructions and subsequently analyzed by the 3500XL Genetic Analyzer (ThermoFisher Scientific-Bleiswijk, the Netherlands). Sequencing reads were analyzed in Clone Manager software v9.4 (Sci-Ed software-USA). An overview of the sequencing results is presented in Table 4. The sequencing results demonstrated that no other bases than that of the donor DNA were incorporated (flawless) and the loss of fluorescence was indeed caused by the frameshift which is encoded by the donor DNA.

TABLE 4
Overview of the sequencing results confirming loss of fluorescence due to intended frameshift
in the YFP gene as is encoded in the donor DNA part of the CTEC DNA fragment.

| | | | | Flawless |
|---|---|---|---|---|
| | PCR | Sequencing | Confirmed | (no additional |
| CTEC DNA fragment | primerset | primer | frameshift | bases incorporated) |
| YFP target + 3′ donor | SEQ ID NO: 39 | SEQ ID NO: 41 | 100% | 100% |
| | SEQ ID NO: 40 | | | |
| YFP target + connector | SEQ ID NO: 39 | SEQ ID NO: 41 | 100% | 100% |
| A + 3′ donor | SEQ ID NO: 40 | | | |
| 5′ donor + YFP target | SEQ ID NO: 39 | SEQ ID NO: 41 | 100% | 100% |
| | SEQ ID NO: 40 | | | |
| 5′ donor + connector | SEQ ID NO: 39 | SEQ ID NO: 41 | 100% | 100% |
| A + YFP target | SEQ ID NO: 40 | | | |
| 5′ donor + PAM_guide | SEQ ID NO: 39 | SEQ ID NO: 41 | 100% | 100% |
| target + YFP target | SEQ ID NO: 40 | | | |
| YFP target + guide | SEQ ID NO: 39 | SEQ ID NO: 41 | 100% | 100% |
| target_PAM + 3′ donor | SEQ ID NO: 40 | | | |

To confirm correct integration of the donor DNA that is part of the CTEC DNA fragment targeting INT1, 8 colonies of each transformation were checked by Sanger sequencing. The primers (SEQ ID NO: 41 and SEQ ID NO: 42) used to confirm the integration were designed to hybridize in the genome outside (372 bp upstream and 400 bp downsteam) the donor DNA that is present in the CTEC DNA fragment. PCR reactions were performed using Phusion® High Fidelity Polymerase (Catno. M0530L, New England Biolabs-USA) according to manufacturer's instructions and a standard PCR program known to the person skilled in the art. The resulting PCR product was purified using a NucleoSpin Gel and PCR Clean-up kit (Machery-Nagel, distributed by Bioké, Leiden, the Netherlands), subsequently the PCR fragment was used as template in a sequencing reaction. Sequencing reactions were set-up making use of a BigDye® Terminator v3.1 Cycle Sequencing Kit (Catno. 4337456, ThermoFisher Scientific, Bleiswijk, the Netherlands) according to supplier's instructions. The sequencing reactions were purified by NucleoSEQ columns (Catno. 740523.250, Machery-Nagel, distributed by Bioké, Leiden, the Netherlands) according supplier's instructions and subsequently analyzed by the 3500XL Genetic Analyzer (ThermoFisher Scientific-Bleiswijk, the Netherlands). Sequencing reads were analyzed in Clone Manager software v9.4 (Sci-Ed software-USA). An overview of the sequencing results is presented in Table 5.

TABLE 5
Overview of the sequencing results confirming the change of the PAM sequence (AGG to
ATG) in the INT1 locus as is encoded in the donor DNA part of the CTEC DNA fragment.

| | | | | Flawless |
|---|---|---|---|---|
| | PCR | Sequencing | Confirmed | (no additional |
| CTEC DNA fragment | primerset | primer | frameshift | bases incorporated) |
| INT1 target + 3′ donor | SEQ ID NO: 42 | SEQ ID NO: 44 | 13% | 100% |
| | SEQ ID NO: 43 | | | |
| INT1 target + | SEQ ID NO: 42 | SEQ ID NO: 44 | 43% | 100% |
| connector A + 3′ donor | SEQ ID NO: 43 | | | |
| 5′ donor + INT1 target | SEQ ID NO: 42 | SEQ ID NO: 44 | 63% | 100% |
| | SEQ ID NO: 43 | | | |
| 5′ donor + connector | SEQ ID NO: 42 | SEQ ID NO: 44 | 38% | 100% |
| A + INT1 target | SEQ ID NO: 43 | | | |
| 5′ donor + PAM_guide | SEQ ID NO: 42 | SEQ ID NO: 44 | 88% | 100% |
| target + INT1 target | SEQ ID NO: 43 | | | |
| INT1 target + PAM_guide | SEQ ID NO: 42 | SEQ ID NO: 44 | 50% | 100% |
| target + 3′ donor | SEQ ID NO: 43 | | | |

The PAM change as encoded by the donor DNA that is part of the CTEC fragment is confirmed, at a success rate of 13-88%. By sequencing it was also confirmed that there are no additional base changes than the ones encoded by the donor DNA, independent of the type of CTEC DNA fragment that is used. The editing efficiency of INT1 compared to YFP that is based on the sequencing results is lower, this is the consequence of not having a pre-selection on phenotype (loss of fluorescence) as is the case for the YFP target.

### Example 2. LbCpf1 Genome Editing by CRISPR Transient Editing Construct (CTEC) in S. cerevisiae

This example describes genome editing of Saccharomyces cerevisiae by the integration of a donor DNA fragment encoding desired mutations making use a CRISPR/LbCpf1 (Cpf1 orthologue from Lachnospiraceae bacterium ND2006) system and transient expression of guide RNA. The CTEC DNA fragment(s) that are used comprise a guide-RNA expression cassette with control elements as previously described by Zetsche et al., 2015 (LbCpf1) for the expression of guide-RNA's in S. cerevisiae and a donor DNA sequence for editing the targeted genomic sequence. The LbCpf1 guide-RNA expression cassettes comprise the SNR52 promoter, a guide-RNA sequence consisting of the direct repeat and the genomic target sequence followed by the SUP4 terminator. The donor DNA which is also part of the CTEC fragment is 109 bp long when the YFP gene is targeted and encodes a 2 bp deletion whereby the original PAM sequence is modified (TTTG=>TG). Upon incorporation of the donor DNA, a frameshift is introduced in the YFP gene resulting in the loss of fluorescence of the strain. The donor DNA for the INT1 locus is 100 bp in size and encodes a 3 bp change of the PAM converting the TTTG sequence to CCGG. The experimental set-up is depicted in FIG. 15.

### Construction of LbCpf1 Expression Vector

Single copy yeast vectors to express LbCpf1 was constructed as follows: Yeast vector pCSN061 is a single copy vector (CEN/ARS) that contains a CAS9 expression cassette consisting of a CAS9 codon optimized variant expressed from the KI11 promoter (Kluyveromyces lactis promoter of KLLA0F20031g) and the S. cerevisiae GND2 terminator, and a functional KanMX marker cassette conferring resistance against G418. The CAS9 expression cassette was KpnI/NotI ligated into pRS414 (Sikorski and Hieter, 1989), resulting in intermediate vector pCSN004. Subsequently, a functional expression cassette conferring G418 resistance (http://www.euroscarf.de) was NotI restricted from vector pUG7-KanMX and NotI ligated into pCSN004, resulting in vector pCSN061 that is depicted in FIG. 1 and the sequence is set out in SEQ ID NO: 2.

A linear PCR fragment of the pCSN061 vector omitting the CAS9 expression cassette, thus including the KL11p, the pCSN061 single copy vector backbone and a KanMX marker cassette, was obtained by PCR using vector pCSN061 as template by including a forward (SEQ ID NO: 45) and reverse primer (SEQ ID NO: 46) and Phusion as DNA polymerase (New England Biolabs, USA) in the reaction. The PCR reaction was performed according to manufacturer's instructions.

The LbCpf1 from Lachnospiraceae bacterium ND2006 used in this example (Zetsche et al, 2015) was obtained as follows: A linker protein sequence (SRAD) and a SV40 nuclear localization signal (PKKKRKV) were added to the carboxy terminus of the LbCpf1 gene, resulting in the LbCpf1 protein sequence (SEQ ID NO: 47). This protein sequence were codon pair optimized for expression in S. cerevisiae as described in WO2008/000632, resulting in the nucleotide sequences as set out in SEQ ID NO: 48 for LbCpf1. The nucleotide sequence was ordered as synthetic DNA at Thermo Fisher Scientific (GeneArt Gene Synthesis and Services).

The synthetic LbCpf1 (SEQ ID NO: 48) sequences were used as template in a PCR reaction with primerset (SEQ ID NO: 49 and SEQ ID NO: 50) using Phusion as DNA polymerase (New England Biolabs, USA) in the reaction. The PCR reaction was performed according to manufacturer's instructions. The obtained LbCpf1 PCR fragment has homology at its 5′ end (part of KI11p sequence) and 3′ end (part of GND2t sequence) with the linear PCR fragment of the pCSN061 vector.

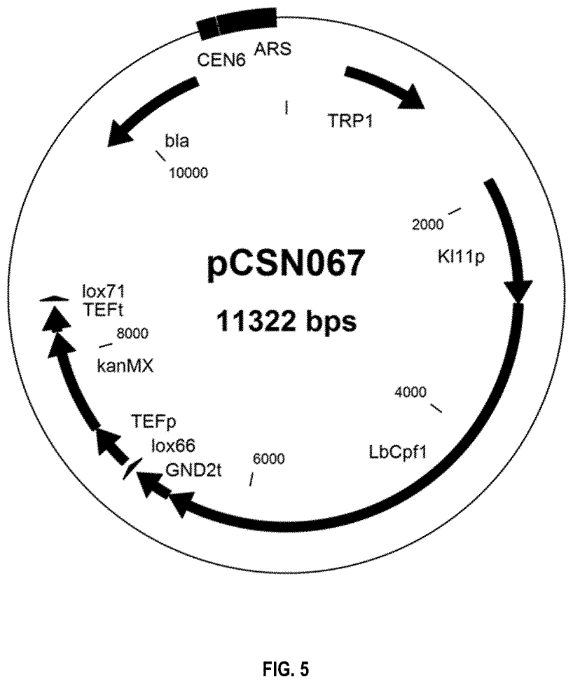

All PCR fragments were purified using the NucleoSpin Gel and PCR Clean-up kit (Machery-Nagel, distributed by Bioké, Leiden, the Netherlands) according to manufacturer's instructions. Subsequently the purified LbCpf1 PCR fragment was assembled into the purified linear PCR fragment of the pCSN061 vector using Gibson assembly (Gibson et al., 2009). The resulting single copy yeast expression vector was pCSN067 (LbCpf1, FIG. 5, SEQ ID NO: 51).

Construction of a Cpf1-Expressing Saccharomyces cerevisiae Strain

Yeast vector pCSN067 is a single copy vector (CEN/ARS) that contains a LbCpf1 expression cassette consisting of a LbCpf1 codon optimized variant (WO2008/000632) expressed from the KI11 promoter (Kluyveromyces lactis promoter of KLLA0F20031g), the S. cerevisiae GND2 terminator, and a functional KanMX marker cassette conferring resistance against G418.

Vector pCSN067 containing the LbCpf1 expression cassette was first transformed to S. cerevisiae strain CEN.PK113-7D (MATa URA3 HIS3 LEU2 TRP1 MAL2-8 SUC2) using the LiAc/salmon sperm (SS) carrier DNA/PEG method (Gietz and Woods, 2002). Strain CEN.PK113-7D is available from the EUROSCARF collection (http://www.euroscarf.de, Frankfurt, Germany). The origin of the CEN.PK family of strains is described by van Dijken et al., 2000. In the transformation mixture one microgram of vector pCNS067 was used. The transformation mixture was plated on YPD-agar (10 grams per liter of yeast extract, 20 grams per liter of peptone, 20 grams per liter of dextrose, 20 grams per liter of agar) containing 200 microgram (μg) G418 (Sigma Aldrich, Zwijndrecht, the Netherlands) per ml. After two to four days of growth at 30° C. transformants appeared on the transformation plate. A transformant conferring resistance to G418 on the plate, was selected. This transformant has by obtaining pCSN067, expression of LbCpf1, and is designated as strain CSN004 which was used in subsequent transformation experiments.

### Double-Stranded DNA (Ds-DNA) YFP Donor DNA Cassette

A double-stranded donor DNA cassette coding for the Yellow Fluorescent Protein (YFP) variant Venus (Nagai et al., 2002), was prepared via a Golden-Gate assembly reaction of individual promoter (P), orf (O) and terminator (T) sequences in an appropriate E. coli vector. The assembled POT cassette was amplified via a PCR reaction with primers indicated in SEQ ID NO: 4 and SEQ ID NO: 5. In a second PCR, 50 bp connector sequences are added using primer sets indicated in SEQ ID NO: 6 and SEQ ID NO: 7. This resulted in an YFP expression cassette that included 50 bp connector sequences at the 5′ and 3′ ends of the expression cassette (SEQ ID NO: 8). The YFP expression cassette in between connector sequences is used as template in the subsequent PCR reaction using primerset (SEQ ID NO: 9 and SEQ ID NO: 10). In this PCR reaction 50 bp genomic flanks are added for integration into the genomic locus, INT1, of S. cerevisiae strain CSN004. The sequence of the resulting YFP cassette flanked by 50 bp genomic sequences is presented in SEQ ID NO: 11.

The Q5 DNA polymerase (part of the Q5® High-Fidelity 2X Master Mix, New England Biolabs, supplied by Bioké, Leiden, the Netherlands. Cat no. M0492S) was used in the PCR reactions described above. PCR reactions were performed according to manufacturer's instructions.

### PCR Purification

Purification of PCR reactions was performed using NucleoSpin Gel and PCR Clean-up kit (Machery-Nagel, distributed by Bioké, Leiden, the Netherlands) according to manufacturer's instructions.

Guide-RNA (crRNA) Expression Cassette INT1

Guide-RNA expression cassettes were ordered as synthetic DNA (gBlocks) at Integrated DNA Technologies (IDT, Leuven, Belgium). The guide-RNA expression cassettes consisted of the SNR52p RNA polymerase III promoter, a guide-RNA sequence consisting of the direct repeat (SEQ ID NO: 52) and the genomic target sequence (SEQ ID NO: 53) followed by the SUP4 terminator as described in Zetsche et al., 2015. For in vivo homologous recombination into the linearized pRN1120 (XhoI, EcoRI) vector backbone, 50 bp homology to pRN1120 was added on either side of the guide-RNA expression cassette, resulting in a fragment of 430 bp in total (SEQ ID NO: 54).

pRN1120 Vector Construction (Multi-Copy Expression Vector, NatMX Marker)

Yeast vector pRN1120 is a multi-copy vector (2 micron) that contains a functional NatMX marker cassette conferring resistance against nourseothricin. The backbone of this vector is based on pRS305 (Sikorski and Hieter, 1989), and includes a functional 2 micron ORI sequence and a functional NatMX marker cassette (see www.euroscarf.de). Vector pRN1120 is depicted in FIG. 2 and the sequence is set out in SEQ ID NO: 3.

Construction of a LbCpf1-Expressing Saccharomyces cerevisiae Strain with YFP Expression Cassette Integrated at INT1 Locus in the Genome

S. cerevisiae strain CSN004 was transformed using the LiAc/salmon sperm (SS) carrier DNA/PEG method (Gietz and Woods, 2002). Prior to transformation strain CSN004 was cultivated in YPD liquid medium (10 grams per liter of yeast extract, 20 grams per liter of peptone, 20 grams per liter of dextrose) supplemented with 200 microgram (μg) G418 (Sigma Aldrich, Zwijndrecht, the Netherlands) per ml. Strain CSN004 was transformed with XhoI/EcoRI restricted pRN1120 and a crRNA expression cassette, targeting INT1 (SEQ ID NO: 54). The linearized pRN1120 is a recipient for the crRNA expression cassette which contains homology with pRN1120 at both ends to allow in vivo recombination into a circular plasmid. LbCpf1, that is pre-expressed in the cells, is directed to the genomic target, INT1, to create a double stranded break. In the transformation mixture, YFP donor DNA cassette for integration at INT1 locus is included.

The transformation mixture was plated on YPD-agar (10 grams per liter of yeast extract, 20 grams per liter of peptone, 20 grams per liter of dextrose, 20 grams per liter of agar) containing 200 microgram (μg) G418 (Sigma Aldrich, Zwijndrecht, the Netherlands) and 200 microgram (μg) nourseothricin (NTC, Jena Bioscience, Germany) per ml. After two to four days of growth at 30° C. transformants appeared on the transformation plate. A transformant conferring resistance to G418 and nourseothricin on the plate, and expressing YFP is selected. YFP expression is assessed using the Qpix450 (Molecular Devices; Filter: Ex/Em: 457/536 nm-FITC/GFP). This strain is to be used in additional LbCpf1 experiments therefor it is cured from its guide RNA plasmid (nourseothricin marker) while maintaining its LbCpf1 expression plasmid (KanMX marker). The strain is grown for 24 hours in YPD liquid medium (10 grams per liter of yeast extract, 20 grams per liter of peptone, 20 grams per liter of dextrose) supplemented with 200 microgram (μg) G418 (Sigma Aldrich, Zwijndrecht, the Netherlands) per ml at 30° C., shaking speed: 250 rpm. Dilutions of the culture were made in milliQ and subsequently plated onto YPD-agar medium (10 grams per liter of yeast extract, 20 grams per liter of peptone, 20 grams per liter of dextrose, 20 grams per liter of agar) containing 200 microgram (μg) G418 (Sigma Aldrich, Zwijndrecht, the Netherlands). After two to four days of growth at 30° C., colonies appeared on the agar plate. Single colonies were subsequently checked for nourseothricin sensitivity by streaking them on YPD-agar (10 grams per liter of yeast extract, 20 grams per liter of peptone, 20 grams per liter of dextrose, 20 grams per liter of agar) containing 200 microgram (μg) nourseothricin (NTC, Jena Bioscience, Germany) per ml. A nourseothricin sensitive strain was selected and designated CSN010. This strain was used in further transformation experiments.

### CRISPR Transient Editing Construct (CTEC) DNA Fragments

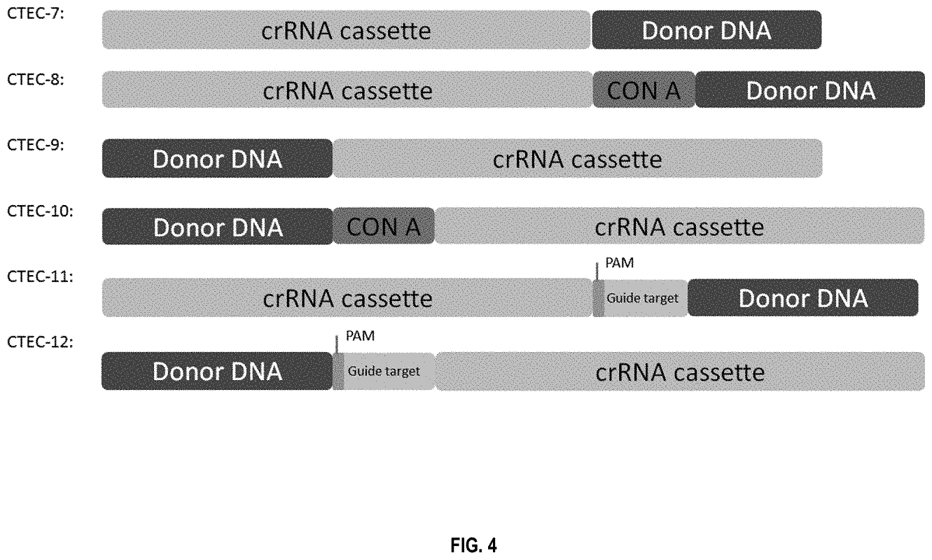

Synthetic DNA's containing guide-RNA expression cassettes were ordered as synthetic DNA (gBlocks) at Integrated DNA Technologies (IDT, Leuven, Belgium). Four to eight designs were made per targeted genomic region (INT1) or YFP ORF, an overview of the designs is provided in FIG. 4. The designs of the CTEC DNA's, of which the sequences are set out in SEQ ID NO's: 55, 56, 57, 58, 59, 60, 61 and 62 (YFP) and SEQ ID NO: 63, 64, 67 and 68 (INT1), consist of the SNR52p RNA polymerase III promoter, a guide-RNA sequence consisting of the direct repeat and the genomic target sequence followed by the SUP4 terminator as described in Zetsche et al., 2015., and the donor DNA that encodes 3 bp substitution (INT1) or DNA 2 basepair deletion causing a frameshift (YFP). The effect of a 50 bp connector, connector A, sequence (SEQ ID NO: 28) as well as the presence of PAM sequence and guide target for separation of donor DNA and guide RNA expression cassette (crRNA) are also evaluated. Connector A is a random DNA sequence of 50 bp without any homology to the genome. When a PAM sequence and guide target were included in the CTEC fragment the guide sequence for creating the ds break is encoded by the crRNA cassette of that same CTEC fragment. When including the PAM sequence and the guide target in the CTEC fragment it was decided to test guide target sequences of different length, 18 bp (SEQ ID NO: 75 (INT1) SEQ ID NO: 76 (YFP)) as well as 20 bp (SEQ ID NO: 77 (INT1) SEQ ID NO: 78 (YFP)). These guide sequences of 18 bp and 20 bp including PAM sequence are presented in SEQ ID NO: 79 (18 bp, INT1), 80 (20 bp, INT1), 81 (18 bp, YFP) and 82 (20 bp, YFP).

An overview of the sequences is provided in Table 6.

TABLE 6
Overview of the sequences of the CTEC DNA's used in transformation. The CTEC
fragments were used as a template in PCR reactions using the primers indicated
in this table. PCR reactions were set-up to obtain CTEC DNA fragments in higher
quantities that are later to be used in the transformation experiments.

| | | Guide | | | |
|---|---|---|---|---|---|
| | | (genomic | | Primers | |
| | | target) | | used to | Sequence |
| | guide-RNA | sequence | | obtain | of |
| | expression | crRNA | Donor | CTEC DNA | CTEC DNA |
| CTEC design | cassette | cassette | DNA | fragment | fragment |
| YFP target + 3′ | SEQ ID | SEQ ID | SEQ ID | SEQ ID | SEQ ID |
| donor | NO: 73 | NO: 69 | NO: 71 | NO: 33 | NO: 55 |
| | | | | SEQ ID | |
| | | | | NO: 35 | |
| YFP target + | SEQ ID | SEQ ID | SEQ ID | SEQ ID | SEQ ID |
| connector A + 3′ | NO: 73 | NO: 69 | NO: 71 | NO: 33 | NO: 56 |
| donor | | | | SEQ ID | |
| | | | | NO: 35 | |
| 5′ donor + YFP | SEQ ID | SEQ ID | SEQ ID | SEQ ID | SEQ ID |
| target | NO: 73 | NO: 69 | NO: 71 | NO: 34 | NO: 57 |
| | | | | SEQ ID | |
| | | | | NO: 83 | |
| 5′ donor + | SEQ ID | SEQ ID | SEQ ID | SEQ ID | SEQ ID |
| connector A + | NO: 73 | NO: 69 | NO: 71 | NO: 34 | NO: 58 |
| YFP target | | | | SEQ ID | |
| | | | | NO: 83 | |
| YFP target + | SEQ ID | SEQ ID | SEQ ID | SEQ ID | SEQ ID |
| PAM_guide | NO: 73 | NO: 69 | NO: 71 | NO: 33 | NO: 59 |
| target + 3′ donor | | | | SEQ ID | |
| (2 × 18 bp | | | | NO: 35 | |
| guide) | | | | | |
| YFP target + | SEQ ID | SEQ ID | SEQ ID | SEQ ID | SEQ ID |
| PAM_guide | NO: 73 | NO: 69 | NO: 71 | NO: 33 | NO: 60 |
| target + 3′ donor | | | | SEQ ID | |
| (2 × 20 bp guide) | | | | NO: 35 | |
| 5′ donor + | SEQ ID | SEQ ID | SEQ ID | SEQ ID | SEQ ID |
| PAM_guide | NO: 73 | NO: 69 | NO: 71 | NO: 34 | NO: 61 |
| target + YFP | | | | SEQ ID | |
| target (2 × 18 | | | | NO: 84 | |
| bp guide) | | | | | |
| 5′ donor + | SEQ ID | SEQ ID | SEQ ID | SEQ ID | SEQ ID |
| PAM_guide | NO: 73 | NO: 69 | NO: 71 | NO: 34 | NO: 62 |
| target + YFP | | | | SEQ ID | |
| target (2 × 20 | | | | NO: 83 | |
| bp guide) | | | | | |
| INT1 target + 3′ | SEQ ID | SEQ ID | SEQ ID | SEQ ID | SEQ ID |
| donor | NO: 74 | NO: 70 | NO: 72 | NO: 33 | NO: 63 |
| | | | | SEQ ID | |
| | | | | NO: 86 | |
| INT1 target + | SEQ ID | SEQ ID | SEQ ID | SEQ ID | SEQ ID |
| connector A + 3′ | NO: 74 | NO: 70 | NO: 72 | NO: 33 | NO: 64 |
| donor | | | | SEQ ID | |
| | | | | NO: 86 | |
| INT1 target + | SEQ ID | SEQ ID | SEQ ID | SEQ ID | SEQ ID |
| PAM_guide | NO: 74 | NO: 70 | NO: 72 | NO: 33 | NO: 67 |
| target + 3′ donor | | | | SEQ ID | |
| (1 × 20 bp, 1 × 18 | | | | NO: 86 | |
| bp guide) | | | | | |
| INT1 target + | SEQ ID | SEQ ID | SEQ ID | SEQ ID | SEQ ID |
| PAM_guide | NO: 74 | NO: 70 | NO: 72 | NO: 33 | NO: 68 |
| target + 3′ donor | | | | SEQ ID | |
| (2 × 20 bp | | | | NO: 86 | |
| guide) | | | | | |

### Yeast Transformation

Strain CSN004 which is pre-expressing Cpf1 and strain CSN010, which is fluorescent due to the presence of an YFP expression cassette and is pre-expression of Cpf1, were inoculated in YPD-G418 medium (10 grams per liter of yeast extract, 20 grams per liter of peptone, 20 grams per liter of dextrose, 200 μg G418 (Sigma Aldrich, Zwijndrecht, the Netherlands) per ml. Subsequently, strain CSN004 and CSN010 were transformed with 1 μg of CTEC DNA, as indicated in Table 7, and 100 ng vector pRN1 120, using the LiAc/SS carrier DNA/PEG method (Gietz and Woods, 2002). The transformation mixtures were plated on YPD-agar (10 grams per liter of yeast extract, 20 grams per liter of peptone, 20 grams per liter of dextrose, 20 grams per liter of agar) containing 200 μg nourseothricin (NTC, Jena Bioscience, Germany) and 200 μg G418 (Sigma Aldrich, Zwijndrecht, the Netherlands) per ml. The plates were incubated at 30 degrees Celsius until colonies appeared on the plates.

TABLE 7
Overview of CTEC DNA's used in S. cerevisiae transformation experiments.

| | | | CTEC DNA | |
|---|---|---|---|---|
| Transformation | Description | Strain | sequence | FIG. |
| #1 | YFP target + 3′ donor | CSN010 | SEQ ID | FIG. 4 |
| | | | NO: 55 | CTEC-7 |
| #2 | YFP target + connector | CSN010 | SEQ ID | FIG. 4 |
| | A + 3′ donor | | NO.: 56 | CTEC-8 |
| #3 | 5′ donor + YFP target | CSN010 | SEQ ID | FIG. 4 |
| | | | NO: 57 | CTEC-9 |
| #4 | 5′ donor + connector A + YFP | CSN010 | SEQ ID | FIG. 4 |
| | target | | NO: 58 | CTEC-10 |
| #5 | YFP target + PAM_guide target + | CSN010 | SEQ ID | FIG. 4 |
| | 3′ donor (2 × 18 bp guide) | | NO: 59 | CTEC-11 |
| #6 | YFP target + PAM_guide target + | CSN010 | SEQ ID | FIG. 4 |
| | 3′ donor (2 × 20 bp guide) | | NO: 60 | CTEC-11 |
| #7 | 5′ donor + PAM_guide target + | CSN010 | SEQ ID | FIG. 4 |
| | YFP target (2 × 18 bp guide) | | NO: 61 | CTEC-12 |
| #8 | 5′ donor + PAM_guide target + | CSN010 | SEQ ID | FIG. 4 |
| | YFP target (2 × 20 bp guide) | | NO: 62 | CTEC-12 |
| #9 | INT1 target + 3′ donor | CSN004 | SEQ ID | FIG. 4 |
| | | | NO: 63 | CTEC-7 |
| #10 | INT1 target + connector | CSN004 | SEQ ID | FIG. 4 |
| | A + 3′ donor | | NO.: 64 | CTEC-8 |
| #11 | INT1 target + PAM_guide target + | CSN004 | SEQ ID | FIG. 4 |
| | 3′ donor (1 × 20 bp, 1 × 18 bp | | NO.: 67 | CTEC-11 |
| | guide) | | | |
| #12 | INT1 target + PAM guide target + | CSN004 | SEQ ID | FIG. 4 |
| | 3′ donor (2 × 20 bp guide) | | NO.: 68 | CTEC-11 |
| #13 | pRN1120 | CSN004 | — | FIG. 2 |
| #14 | pRN1120 | CSN010 | — | FIG. 2 |

### Results

The colonies resulting from the transformation experiment outlined above in Table 7 were checked for incorporation of the donor DNA after transient expression of the guide RNA that is encoded on the CTEC DNA fragment. Incorporation of the donor DNA that is targeted towards the YFP cassette, results in a frameshift in the YFP ORF, resulting in loss of fluorescence. The YFP fluorescence of the colonies after transformation was visualized by the QPIX450 (Filter: Ex/Em: 457/536 nm-FITC/GFP). The success rate of YFP editing by the CTEC DNA fragment based on phenotype is summarized below in Table 8.

The efficiency of introducing the encoded frameshift in the YFP ORF by incorporation of the donor DNA which is part of the CTEC construct is scored based on loss of fluorescent phenotype. The efficiencies at which YFP fluorescence is lost after transformation is depicted below in Table 8.

TABLE 8
Overview of YFP gene editing after transformation of CTEC
DNA fragment encoding a crRNA for LbCpf1 and donor DNA.

| | | | | | Percentage |
|---|---|---|---|---|---|
| | | | | Number of | non- |
| | | | | non- | fluorescent/ |
| | | | Total | fluorescent | edited |
| Transformation | Description | Strain | colonies | colonies | colonies |
| #1 | YFP target + 3′ donor | CSN010 | 57 | 40 | 70% |
| #2 | YFP target + connector | CSN010 | 50 | 31 | 62% |
| | A + 3′ donor | | | | |
| #3 | 5′ donor + YFP target | CSN010 | 57 | 9 | 16% |
| #4 | 5′ donor + connector | CSN010 | 55 | 9 | 16% |
| | A + YFP target | | | | |
| #5 | YFP target + PAM_guide | CSN010 | 54 | 22 | 41% |
| | target + 3′ donor | | | | |
| | (2 × 18 bp guide) | | | | |
| #6 | YFP target + PAM_guide | CSN010 | 53 | 36 | 68% |
| | target + 3′ donor | | | | |
| | (2 × 20 bp guide) | | | | |
| #7 | 5′ donor + PAM_guide | CSN010 | 68 | 9 | 13% |
| | target + YFP target | | | | |
| | (2 × 18 bp guide) | | | | |
| #8 | 5′ donor + PAM_guide | CSN010 | 29 | 14 | 48% |
| | target + YFP target | | | | |
| | (2 × 20 bp guide) | | | | |
| #16 | pRN1120 | CSN010 | 71 | 0 | 0% |

To confirm correct integration of the donor DNA that is part of the CTEC DNA fragment targeting INT1, 8 colonies of each transformation were checked by Sanger sequencing. The primers used to confirm the integration (SEQ ID NO: 42 and SEQ ID NO: 43) were designed to hybridize in the genome outside (400 bp up- and 372 bp down-stream) the donor DNA that is present in the CTEC DNA. PCR reactions were performed using Phusion® High Fidelity Polymerase (Catno. M0530L, New England Biolabs-USA) according to manufacturer's instructions and a standard PCR program known to the person skilled in the art. The resulting PCR product was purified using a NucleoSpin Gel and PCR Clean-up kit (Machery-Nagel, distributed by Bioké, Leiden, the Netherlands), subsequently the PCR fragment was used as template in a sequencing reaction. Sequencing reactions were set-up making use of a BigDye® Terminator v3.1 Cycle Sequencing Kit (Catno. 4337456, ThermoFisher Scientific, Bleiswijk, the Netherlands) according to supplier's instructions. The sequencing reactions were purified by NucleoSEQ columns (Catno. 740523.250, Machery-Nagel, distributed by Bioké, Leiden, the Netherlands) according supplier's instructions and subsequently analyzed by the 3500XL Genetic Analyzer (ThermoFisher Scientific-Bleiswijk, the Netherlands). Sequencing reads were analyzed in Clone Manager software v9.4 (Sci-Ed software-USA). An overview of the sequencing results is presented in Table 9 below.

TABLE 9
Overview of INT1 editing as a consequence of LbCpf1 mediated incorporation
of donor DNA after transient expression of the crRNA. Both donor DNA
and crRNA expression cassette are encoded on the CTEC DNA fragment.

| | | | | Flawless |
|---|---|---|---|---|
| | PCR | Sequencing | Confirmed | (no additional |
| CTEC DNA fragment | primer set | primer | frameshift | bases incorporated) |
| INT1 target + 3′ donor | SEQ ID NO: 42 | SEQ ID NO: 44 | 25% | 100% |
| | SEQ ID NO: 43 | | | |
| INT1 target + | SEQ ID NO: 42 | SEQ ID NO: 44 | 63% | 100% |
| connector A + 3′ donor | SEQ ID NO: 43 | | | |
| INT1 target + | SEQ ID NO: 42 | SEQ ID NO: 44 | 57% | 100% |
| PAM guide target + 3′ | SEQ ID NO: 43 | | | |
| donor (1 × 20 bp, 1 × 18 | | | | |
| bp guide) | | | | |
| INT1 target + | SEQ ID NO: 42 | SEQ ID NO: 44 | 43% | 100% |
| PAM_guide target + 3′ | SEQ ID NO: 43 | | | |
| donor (2 × 20 bp guide) | | | | |

The PAM change by LbCpf1 as encoded by the donor DNA that is part of the CTEC fragment is confirmed, at a success rate of 13-68%. The editing frequencies of the YFP gene are based on phenotype; scoring of the non-fluorescent vs fluorescent transformants as a result of donor DNA incorporation. The editing efficiency of INT1 by LbCpf1 is confirmed by sequencing. By sequencing it is demonstrated that the donor DNA is incorporated in the genome, resulting in a 3 bp modification of the PAM sequence, as well as no additional base changes than encoded by the donor DNA are present.

### Example 3. Effect of Connector Sequences, on Both Sides or One Side of the CTEC DNA Fragment, on the Frequency of YFP Gene Editing in Saccharomyces cerevisiae

This example evaluates the effect of connector sequences, on either side or one side of the CTEC DNA fragment, on the frequency of YFP gene editing in Saccharomyces cerevisiae mediated by CRISPR/LbCpf1. The CTEC DNA fragments comprise a guide-RNA expression cassette with control elements as previously described by Zetsche et al., 2015 (LbCpf1) for the expression of guide-RNA's in S. cerevisiae and a donor DNA sequence for editing the targeted sequence. The LbCpf1 guide-RNA expression cassettes comprise the SNR52 promoter, a guide-RNA sequence consisting of the direct repeat and the genomic target sequence followed by the SUP4 terminator. The donor DNA which is also part of the CTEC fragment is 109 bp in size and targets the YFP gene that is integrated on the INT1 locus of S. cerevisiae strain CSN010. The donor DNA encodes a 2 bp deletion whereby the original PAM sequence is modified (TTTG=>TG). Upon incorporation of the donor DNA, a frameshift is introduced in the YFP gene resulting in the loss of fluorescence of the strain. To be able to PCR amplify different CTEC cassettes with the same primer set the CTEC DNA fragment is flanked by so called connector sequences; random DNA sequences without homology to the genome, at the 5′ and 3′ end.

### Experimental Details:

The components used in this example were as follows:
Yeast strain CSN010 which is pre-expressing LbCpf1 and has a fluorescent phenotype due to YFP expression cassette that is present on the INT1 locus. Construction of S. cerevisiae strain CSN010 is described in Example 2.
pRN1120, multi-copy expression vector containing NatMX marker. Construction and details of the plasmid are described in Example 1.

### CRISPR Transient Editing Construct (CTEC) DNA Fragments Flanked by Connector Sequences.

Synthetic DNA's containing guide-RNA expression cassettes were ordered as synthetic DNA (gBlocks) at Integrated DNA Technologies (IDT, Leuven, Belgium). Eight designs were made for editing the YFP ORF, an overview of the designs is provided in FIG. 6. The designs of the CTEC DNA's, of which the sequences are set out in SEQ ID NO's: 87, 88, 89, 90, 91, 92, 93 and 94, consist of the SNR52p RNA polymerase III promoter, a guide-RNA sequence consisting of the direct repeat and the genomic target sequence followed by the SUP4 terminator as described in Zetsche et al., 2015., and the donor DNA that encodes a 2 basepair deletion causing a frameshift (YFP). To be able to PCR amplify different CTEC DNA fragments with the same primer set (SEQ ID NO: 95 and SEQ ID NO: 96) the CTEC DNA fragments are flanked by so called connector sequences; random DNA sequences without homology to the genome, at the 5′ and 3′ end. The CTEC DNA fragments are flanked by connector 5 (CON5, SEQ ID NO: 97) on the 5′ side and connector 3 (CON3, SEQ ID NO: 98) on the 3′ side.

An overview of the sequences is provided in Table 10.

TABLE 10
Overview of the sequences of the CTEC DNA's used in transformation.
The template guide-RNA expression cassettes were used as a template
for PCR using the primers indicated in this table to obtain CTEC
DNA's (CTEC DNA fragments) used in the transformation experiments.

| | | Guide | | | |
|---|---|---|---|---|---|
| | | sequence | | Primers used | Sequence |
| | guide-RNA | (genomic | | to obtain | of the |
| | expression | target | Donor | CTEC DNA | CTEC DNA |
| CTEC design | cassette | sequence) | DNA | fragment | fragment |
| CON5 − YFP target + | SEQ ID | SEQ ID | SEQ ID | SEQ ID | SEQ ID |
| 3′ donor − CON3 | NO: 74 | NO: 69 | NO: 71 | NO: 95 | NO: 87 |
| | | | | SEQ ID | |
| | | | | NO: 96 | |
| CON5 − YFP target + | SEQ ID | SEQ ID | SEQ ID | SEQ ID | SEQ ID |
| connector A + 3′ | NO: 74 | NO: 69 | NO: 71 | NO: 95 | NO: 88 |
| donor − CON3 | | | | SEQ ID | |
| | | | | NO: 96 | |
| CON5 − 5′ donor + | SEQ ID | SEQ ID | SEQ ID | SEQ ID | SEQ ID |
| YFP target − CON3 | NO: 74 | NO: 69 | NO: 71 | NO: 95 | NO: 89 |
| | | | | SEQ ID | |
| | | | | NO: 96 | |
| CON5 − 5′ donor + | SEQ ID | SEQ ID | SEQ ID | SEQ ID | SEQ ID |
| connector A + YFP | NO: 74 | NO: 69 | NO: 71 | NO: 95 | NO: 90 |
| target − CON3 | | | | SEQ ID | |
| | | | | NO: 96 | |
| CON5 − YFP target + | SEQ ID | SEQ ID | SEQ ID | SEQ ID | SEQ ID |
| PAM_guide target + | NO: 74 | NO: 69 | NO: 71 | NO: 95 | NO: 91 |
| 3′ donor − CON3 (2 × | | | | SEQ ID | |
| 18 bp guide) | | | | NO: 96 | |
| CON5 − YFP target + | SEQ ID | SEQ ID | SEQ ID | SEQ ID | SEQ ID |
| PAM_guide target + | NO: 74 | NO: 69 | NO: 71 | NO: 95 | NO: 92 |
| 3′ donor − CON3 (2 × | | | | SEQ ID | |
| 20 bp guide) | | | | NO: 96 | |
| CON5 − 5′ donor + | SEQ ID | SEQ ID | SEQ ID | SEQ ID | SEQ ID |
| PAM_guide target + | NO: 74 | NO: 69 | NO: 71 | NO: 95 | NO: 93 |
| YFP target − CON3 | | | | SEQ ID | |
| (2 × 18 bp guide) | | | | NO: 96 | |
| CON5 − 5′ donor + | SEQ ID | SEQ ID | SEQ ID | SEQ ID | SEQ ID |
| PAM_guide target + | NO: 74 | NO: 69 | NO: 71 | NO: 95 | NO: 94 |
| YFP target − CON3 | | | | SEQ ID | |
| (2 × 20 bp guide) | | | | NO: 96 | |
| YFP target + 3′ donor | SEQ ID | SEQ ID | SEQ ID | SEQ ID | SEQ ID |
| | NO: 74 | NO: 69 | NO: 71 | NO: 33 | NO: 55 |
| | | | | SEQ ID | |
| | | | | NO: 35 | |
| YFP target + | SEQ ID | SEQ ID | SEQ ID | SEQ ID | SEQ ID |
| connector A + 3′ | NO: 74 | NO: 69 | NO: 71 | NO: 33 | NO: 56 |
| donor | | | | SEQ ID | |
| | | | | NO: 35 | |
| 5′ donor + YFP target | SEQ ID | SEQ ID | SEQ ID | SEQ ID | SEQ ID |
| | NO: 74 | NO: 69 | NO: 71 | NO: 34 | NO: 57 |
| | | | | SEQ ID | |
| | | | | NO: 83 | |
| 5′ donor + connector | SEQ ID | SEQ ID | SEQ ID | SEQ ID | SEQ ID |
| A + YFP target | NO: 74 | NO: 69 | NO: 71 | NO: 34 | NO: 58 |
| | | | | SEQ ID | |
| | | | | NO: 83 | |
| YFP target + | SEQ ID | SEQ ID | SEQ ID | SEQ ID | SEQ ID |
| PAM_guide target + | NO: 74 | NO: 69 | NO: 71 | NO: 33 | NO: 59 |
| 3′ donor (2 × 18 bp | | | | SEQ ID | |
| guide) | | | | NO: 35 | |
| YFP target + | SEQ ID | SEQ ID | SEQ ID | SEQ ID | SEQ ID |
| PAM_guide target + | NO: 74 | NO: 69 | NO: 71 | NO: 33 | NO: 60 |
| 3′ donor (2 × 20 bp | | | | SEQ ID | |
| guide) | | | | NO: 35 | |
| 5′ donor + | SEQ ID | SEQ ID | SEQ ID | SEQ ID | SEQ ID |
| PAM_guide target + | NO: 74 | NO: 69 | NO: 71 | NO: 34 | NO: 61 |
| YFP target (2 × 18 bp | | | | SEQ ID | |
| guide) | | | | NO: 84 | |
| 5′ donor + | SEQ ID | SEQ ID | SEQ ID | SEQ ID | SEQ ID |
| PAM_guide target + | NO: 74 | NO: 69 | NO: 71 | NO: 34 | NO: 62 |
| YFP target (2 × 20 bp | | | | SEQ ID | |
| guide) | | | | NO: 83 | |
| CON5 − YFP target + | SEQ ID | SEQ ID | SEQ ID | SEQ ID | SEQ ID |
| 3′ donor | NO: 74 | NO: 69 | NO: 71 | NO: 95 | NO: 99 |
| | | | | SEQ ID | |
| | | | | NO: 35 | |
| CON5 − YFP target + | SEQ ID | SEQ ID | SEQ ID | SEQ ID | SEQ ID |
| connector A + 3′ | NO: 74 | NO: 69 | NO: 71 | NO: 95 | NO: 100 |
| donor | | | | SEQ ID | |
| | | | | NO: 35 | |
| CON5 − 5′ donor + | SEQ ID | SEQ ID | SEQ ID | SEQ ID | SEQ ID |
| YFP target | NO: 74 | NO: 69 | NO: 71 | NO: 95 | NO: 101 |
| | | | | SEQ ID | |
| | | | | NO: 83 | |
| CON5 − 5′ donor + | SEQ ID | SEQ ID | SEQ ID | SEQ ID | SEQ ID |
| connector A + YFP | NO: 74 | NO: 69 | NO: 71 | NO: 95 | NO: 102 |
| target | | | | SEQ ID | |
| | | | | NO: 83 | |
| CON5 − YFP target + | SEQ ID | SEQ ID | SEQ ID | SEQ ID | SEQ ID |
| PAM_guide target + | NO: 74 | NO: 69 | NO: 71 | NO: 95 | NO: 103 |
| 3′ donor (2 × 18 bp | | | | SEQ ID | |
| guide) | | | | NO: 35 | |
| CON5 − YFP target + | SEQ ID | SEQ ID | SEQ ID | SEQ ID | SEQ ID |
| PAM_guide target + | NO: 74 | NO: 69 | NO: 71 | NO: 95 | NO: 104 |
| 3′ donor (2 × 20 bp | | | | SEQ ID | |
| guide) | | | | NO: 35 | |
| CON5 − YFP target + | SEQ ID | SEQ ID | SEQ ID | SEQ ID | SEQ ID |
| PAM_guide target + | NO: 74 | NO: 69 | NO: 71 | NO: 95 | NO: 105 |
| 5′ donor (2 × 18 bp | | | | SEQ ID | |
| guide) | | | | NO: 84 | |
| CON5 − YFP target + | SEQ ID | SEQ ID | SEQ ID | SEQ ID | SEQ ID |
| PAM guide target + | NO: 74 | NO: 69 | NO: 71 | NO: 95 | NO: 106 |
| 5′ donor (2 × 20 bp | | | | SEQ ID | |
| guide) | | | | NO: 83 | |
| YFP target + 3′ | SEQ ID | SEQ ID | SEQ ID | SEQ ID | SEQ ID |
| donor − CON3 | NO: 74 | NO:69 | NO: 71 | NO: 33 | NO: 107 |
| | | | | SEQ ID | |
| | | | | NO: 96 | |
| YFP target + | SEQ ID | SEQ ID | SEQ ID | SEQ ID | SEQ ID |
| connector A + 3′ | NO: 74 | NO: 69 | NO: 71 | NO: 33 | NO: 108 |
| donor − CON3 | | | | SEQ ID | |
| | | | | NO: 96 | |
| 5′ donor + YFP | SEQ ID | SEQ ID | SEQ ID | SEQ ID | SEQ ID |
| target − CON3 | NO: 74 | NO: 69 | NO: 71 | NO: 34 | NO: 109 |
| | | | | SEQ ID | |
| | | | | NO: 96 | |
| 5′ donor + connector | SEQ ID | SEQ ID | SEQ ID | SEQ ID | SEQ ID |
| A + YFP target − CON3 | NO: 74 | NO: 69 | NO: 71 | NO: 34 | NO: 110 |
| | | | | SEQ ID | |
| | | | | NO: 96 | |
| YFP target + | SEQ ID | SEQ ID | SEQ ID | SEQ ID | SEQ ID |
| PAM_guide target + | NO: 74 | NO: 69 | NO: 71 | NO: 33 | NO: 111 |
| 3′ donor − CON3 (2 × | | | | SEQ ID | |
| 18 bp guide) | | | | NO: 96 | |
| YFP target + | SEQ ID | SEQ ID | SEQ ID | SEQ ID | SEQ ID |
| PAM_guide target + | NO: 74 | NO: 69 | NO: 71 | NO: 33 | NO: 112 |
| 3′ donor − CON3 (2 × | | | | SEQ ID | |
| 20 bp guide) | | | | NO: 96 | |
| 5′ donor + | SEQ ID | SEQ ID | SEQ ID | SEQ ID | SEQ ID |
| PAM_guide target + | NO: 74 | NO: 69 | NO: 71 | NO: 34 | NO: 113 |
| YFP target − CON3 | | | | SEQ ID | |
| (2 × 18 bp guide) | | | | NO: 96 | |
| 5′ donor − | SEQ ID | SEQ ID | SEQ ID | SEQ ID | SEQ ID |
| PAM_guide target + | NO: 74 | NO: 69 | NO: 71 | NO: 34 | NO: 114 |
| YFP target − CON3 | | | | SEQ ID | |
| (2 × 20 bp guide) | | | | NO: 96 | |

The CTEC fragments (gBlock) were used as a template in PCR reactions using the primers indicated in this table. PCR reactions were set-up to obtain CTEC DNA fragments in higher quantities that are later to be used in the transformation experiments. PrimeSTAR GXL DNA Polymerase (Takara/Cat no. R050A) was used in the PCR reactions according to the manufacturer's instructions. The PCR generated CTEC DNA's were purified using a NucleoSpin Gel and PCR Clean-up kit (Machery-Nagel, distributed by Bioké, Leiden, the Netherlands) according to manufacturer's instructions. Subsequently, DNA concentrations were measured using a Nano Drop (ND-1000 Spectrophotometer, Thermo Scientific, Bleiswijk, the Netherlands).

### Yeast Transformation

Strain CSN010 which is pre-expressing LbCpf1 and fluorescent due to the presence of an YFP expression cassette, was inoculated in YPD-G418 medium (10 grams per liter of yeast extract, 20 grams per liter of peptone, 20 grams per liter of dextrose, 200 μg G418 (Sigma Aldrich, Zwijndrecht, the Netherlands) per ml. Subsequently, strain CSN010 was transformed with 1 μg of CTEC DNA, as indicated in Table 11, and 100 ng vector pRN1120, using the LiAc/SS carrier DNA/PEG method (Gietz and Woods, 2002).

The transformation mixtures were plated on YPD-agar (10 grams per liter of yeast extract, 20 grams per liter of peptone, 20 grams per liter of dextrose, 20 grams per liter of agar) containing 200 μg nourseothricin (NTC, Jena Bioscience, Germany) and 200 μg G418 (Sigma Aldrich, Zwijndrecht, the Netherlands) per ml. The plates were incubated at 30 degrees Celsius until colonies appeared on the plates.

TABLE 11
Overview of CTEC DNA's used in the different transformation experiments.

| | | CTEC DNA | |
|---|---|---|---|
| Transformation | Description | sequence | FIG. |
| #1 | CON5 − YFP target + 3′ donor − | SEQ ID NO: 87 | FIG. 6 |
| | CON3 | | CON5 − CTEC-7 − CON3 |
| #2 | CON5 − YFP target + connector | SEQ ID NO: 88 | FIG. 6 |
| | A + 3′ donor − CON3 | | CON5 − CTEC-8 − CON3 |
| #3 | CON5 − 5′ donor + YFP target − | SEQ ID NO: 89 | FIG. 6 |
| | CON3 | | CON5 − CTEC-9 − CON3 |
| #4 | CON5 − 5′ donor + connector | SEQ ID NO: 90 | FIG. 6 |
| | A − YFP target − CON3 | | CON5 − CTEC-10 − CON3 |
| #5 | CON5 − YFP target + PAM_guide | SEQ ID NO: 91 | FIG. 6 |
| | target + 3′ donor − CON3 | | CON5 − CTEC--11 − CON3 |
| | (2 × 18 bp guide) | | |
| #6 | CON5 − YFP target + PAM_guide | SEQ ID NO: 92 | FIG. 6 |
| | target + 3′ donor − CON3 (2 × 20 | | CON5 − CTEC-11 − CON3 |
| | bp guide) | | |
| #7 | CON5 − 5′ donor + PAM_guide | SEQ ID NO: 93 | FIG. 6 |
| | target + YFP target − CON3 (2 × | | CON5 − CTEC-12 − CON3 |
| | 18 bp guide) | | |
| #8 | CON5 − 5′ donor + PAM_guide | SEQ ID NO: 94 | FIG. 6 |
| | target + YFP target − CON3 (2 × | | CON5 − CTEC-12 − CON3 |
| | 20 bp guide) | | |
| #9 | YFP target + 3′ donor | SEQ ID NO: 55 | FIG. 4 |
| | | | CTEC-7 |
| #10 | YFP target + connector A + 3′ | SEQ ID NO: 56 | FIG. 4 |
| | donor | | CTEC-8 |
| #11 | 5′ donor + YFP target | SEQ ID NO: 57 | FIG. 4 |
| | | | CTEC-9 |
| #12 | 5′ donor + connector A + YFP | SEQ ID NO: 58 | FIG. 4 |
| | target | | CTEC-10 |
| #13 | YFP target + PAM_guide target + | SEQ ID NO: 59 | FIG. 4 |
| | 3′ donor | | CTEC-11 |
| | (2 × 18 bp guide) | | |
| #14 | YFP target + PAM_guide target + | SEQ ID NO: 60 | FIG. 4 |
| | 3′ donor | | CTEC-11 |
| | (2 × 20 bp guide) | | |
| #15 | 5' donor + PAM_guide target + | SEQ ID NO: 61 | FIG. 4 |
| | YFP target | | CTEC-12 |
| | (2 × 18 bp guide) | | |
| #16 | 5′ donor + PAM_guide target + | SEQ ID NO: 62 | FIG. 4 |
| | YFP target | | CTEC-12 |
| | (2 × 20 bp guide) | | |
| #17 | CON5 − YFP target + 3′ donor | SEQ ID NO: 99 | FIG. 6 |
| | | | CON5 − CTEC-7 |
| #18 | CON5 − YFP target + connector | SEQ ID NO: 100 | FIG. 6 |
| | A + 3′ donor | | CON5 − CTEC-8 |
| #19 | CON5 − 5′ donor + YFP target | SEQ ID NO: 101 | FIG. 6 |
| | | | CON5 − CTEC-9 |
| #20 | CON5 − 5′ donor + connector | SEQ ID NO: 102 | FIG. 6 |
| | A + YFP target | | CON5 − CTEC-10 |
| #21 | CON5 − YFP target + PAM_guide | SEQ ID NO: 103 | FIG. 6 |
| | target + 3′ donor | | CON5 − CTEC--11 |
| | (2 × 18 bp guide) | | |
| #22 | CON5 − YFP target + PAM_guide | SEQ ID NO: 104 | FIG. 6 |
| | target + 3′ donor | | CON5 − CTEC-11 |
| | (2 × 20 bp guide) | | |
| #23 | CON5 − YFP target + PAM_guide | SEQ ID NO: 105 | FIG. 6 |
| | target + 5′ donor | | CON5 − CTEC-12 |
| | (2 × 18 bp guide) | | |
| #24 | CON5 − YFP target + PAM_guide | SEQ ID NO: 106 | FIG. 6 |
| | target + 5′ donor | | CON5 − CTEC-12 |
| | (2 × 20 bp guide) | | |
| #25 | YFP target + 3′ donor − CON3 | SEQ ID NO: 107 | FIG. 6 |
| | | | CTEC-7 − CON3 |
| #26 | YFP target + connector A + 3′ | SEQ ID NO: 108 | FIG. 6 |
| | donor − CON3 | | CTEC-8 − CCON3 |
| #27 | 5′ donor + YFP target − CON3 | SEQ ID NO: 109 | FIG. 6 |
| | | | CTEC-9 − CCON3 |
| #28 | 5′ donor + connector A + YFP | SEQ ID NO: 110 | FIG. 6 |
| | target − CON3 | | CTEC-10 − CCON3 |
| #29 | YFP target + PAM_guide target + | SEQ ID NO: 111 | FIG. 6 |
| | 3′ donor − CON3 (2 × 18 | | CTEC--11 − CON3 |
| | bp guide) | | |
| #30 | YFP target + PAM_guide target + | SEQ ID NO: 112 | FIG. 6 |
| | 3′ donor − CON3 | | CTEC-11 − CON3 |
| | (2 × 20 bp guide) | | |
| #31 | 5′ donor + PAM_guide target + | SEQ ID NO: 113 | FIG. 6 |
| | YFP target − CON3 | | CTEC-12 − CON3 |
| | (2 × 18 bp guide) | | |
| #32 | 5′ donor + PAM_guide target + | SEQ ID NO: 114 | FIG. 6 |
| | YFP target − CON3 | | CTEC-12 − CON3 |
| | (2 × 20 bp guide) | | |

### Results

The colonies resulting from the transformation experiment outlined above in Table 11 were checked for incorporation of the donor DNA after transient expression of the guide RNA that is encoded on the CTEC DNA fragment. Incorporation of the donor DNA that is targeted towards the YFP cassette, results in a frameshift in the YFP ORF, resulting in loss of fluorescence. The YFP fluorescence of the colonies after transformation was visualized by the QPIX450 (Filter: Ex/Em: 457/536 nm-FITC/GFP). The success rate of YFP editing by the CTEC DNA fragment with connectors based on phenotype is summarized below in Table 12.

TABLE 12
Overview of YFP gene editing frequencies in Saccharomyces cerevisiae
CSN010 by CTEC DNA fragments flanked by one or two connector sequences.
Editing frequencies established based on phenotype, in case the
YFP gene is not edited, YFP fluorescence is visible. In case of
editing of the YFP gene by donor DNA, fluorescence is lost.

| | | Percentage non- |
|---|---|---|
| | | fluorescent, |
| Transformation | Description | edited colonies |
| #1 | CON5 − YFP target + 3′ donor − CON3 | 65% |
| #2 | CON5 − YFP target + connector A + 3′ donor − | 78% |
| | CON3 | |
| #3 | CON5 − 5′ donor + YFP target − CON3 | 65% |
| #4 | CON5 − 5′ donor + connector A − YFP target − | 68% |
| | CON3 | |
| #5 | CON5 − YFP target + PAM guide target + 3′ | 39% |
| | donor − CON3 (2 × 18 bp guide) | |
| #6 | CON5 − YFP target + PAM guide target + 3′ | 82% |
| | donor − CON3 (2 × 20 bp guide) | |
| #7 | CON5 − 5′ donor + PAM guide target + YFP | 51% |
| | target − CON3 (2 × 18 bp guide) | |
| #8 | CON5 − 5′ donor + PAM guide target + YFP | 51% |
| | target − CON3 (2 × 20 bp guide) | |
| #9 | YFP target + 3′ donor | 70% |
| #10 | YFP target + connector A + 3′ donor | 62% |
| #11 | 5′ donor + YFP target | 16% |
| #12 | 5′ donor + connector A + YFP target | 16% |
| #13 | YFP target + PAM_guide target + 3′ donor | 41% |
| | (2 × 18 bp guide) | |
| #14 | YFP target + PAM_guide target + 3′ donor | 68% |
| | (2 × 20 bp guide) | |
| #15 | 5′ donor + PAM_guide target + YFP target | 13% |
| | (2 × 18 bp guide) | |
| #16 | 5′ donor + PAM_guide target + YFP target | 48% |
| | (2 × 20 bp guide) | |
| #17 | CON5 − YFP target + 3′ donor | 81% |
| #18 | CON5 − YFP target + connector A + 3′ donor | 82% |
| #19 | CON5 − 5′ donor + YFP target | 59% |
| #20 | CON5 − 5′ donor + connector A + YFP target | 68% |
| #21 | CON5 − YFP target + PAM_guide target + 3′ | 53% |
| | donor (2 × 18 bp guide) | |
| #22 | CON5 − YFP target + PAM_guide target + 3′ | 57% |
| | donor (2 × 20 bp guide) | |
| #23 | CON5 − 5′ donor + PAM_guide target + YFP | 41% |
| | target (2 × 18 bp guide) | |
| #24 | CON5 − 5′ donor + PAM_guide target + YFP | 65% |
| | target (2 × 20 bp guide) | |
| #25 | YFP target + 3′ donor − CON3 | 80% |
| #26 | YFP target + connector A + 3′ donor − CON3 | 71% |
| #27 | 5′ donor + YFP target − CON3 | 57% |
| #28 | 5′ donor + connector A + YFP target − CON3 | 63% |
| #29 | YFP target + PAM_guide target + 3′ donor − | 47% |
| | CON3 (2 × 18 bp guide) | |
| #30 | YFP target + PAM_guide target + 3′ donor − CON3 | 62% |
| | (2 × 20 bp guide) | |
| #31 | 5′ donor + PAM_guide target + YFP target − CON3 | 45% |
| | (2 × 18 bp guide) | |
| #32 | 5′ donor − PAM_guide target + YFP target − | 58% |
| | CON3 (2 × 20 bp guide) | |
| #33 | No CTEC fragment | 0% |

Editing efficiencies are not negatively influenced by the presence of connector sequences on either side or both sides of the CTEC DNA fragments.

### Example 4. Crispr/Cas9 Mediated Knock-Out by CTEC Constructs

This example describes Cas9 mediated knockout of the YFP gene with 100% efficiency in S. cerevisiae strain CSN009. Strain CSN009 pre-expresses Cas9 and contains an YFP expression cassette integrated as fluorescent marker. By transformation of a CTEC DNA fragment which consists of a guide RNA expression cassette as well as donor DNA, the YFP ORF is edited in the strain after transient expression of the guide RNA sequence. In case the donor DNA consists out of 2 flanking regions just outside the YFP expression cassette, the YFP expression cassette is completely deleted. In case the donor DNA encodes a DNA base deletion whereby the genomic target is modified from TTAGTCACTACTTTAGGTTA (SEQ ID NO: 132) to TTAGTCACTACTTTAGTTA (SEQ ID NO: 133), a frameshift is introduced upon incorporation of the donor DNA. In both cases upon incorporation of the donor DNA the YFP fluorescence of the strain is lost. By addition of sequences homologous to plasmid backbone pRN1120 to either side of the CTEC fragment and combining these CTEC fragments with EcoRI and XhoI digested pRN1120 as linear vector backbone in transformation the non-edited background transformants are eliminated. In-vivo circularization results in a plasmid with a continuously expressed guide RNA targeting the YFP gene that is located in the genome. Transformants in which the YFP gene is edited resulting in a changed genomic target site (frameshift) or complete loss of the YFP expression cassette (deletion) are viable.

### CRISPR Transient Editing Construct (CTEC) DNA Fragments

Synthetic DNA's containing guide-RNA expression cassettes were ordered as synthetic DNA (gBlocks) at Integrated DNA Technologies (IDT, Leuven, Belgium). Six designs were made for editing the YFP ORF, an overview of the designs is provided in FIG. 17. The designs of the CTEC DNA's, of which the sequences are set out in SEQ ID NO's: 115, 116, 117, 118, 119 and 120, consist of the SNR52p RNA polymerase III promoter, a guide-sequence (also referred to as genomic target sequence (SEQ ID NO: 122), the gRNA structural component and the SUP4 3′ flanking region as described in DiCarlo et al., 2013, and the donor DNA. In this example two different types of donor fragments are used, both varying in length from 60 to 100 bp. One donor DNA encodes a frameshift in the YFP gene by modification of the genomic target sequence from SEQ ID NO: 132: TTAGTCACTACTTTAGGTTA to SEQ ID NO: 133: TTAGTCACTACTTTAGTTA (SEQ ID NO: 115, 116 and 117), the other donor DNA encodes 2 flanking regions just outside the YFP expression cassette that are adjacent to one another resulting in the full knockout of the YFP expression cassette (SEQ ID NO: 118, 119 and 120). The length of the donor DNA varies from 60 to 100 bp in size, for complete knock out of the YFP gene as well as introduction of a frameshift, in both cases when the donor DNA is incorporated the YFP fluorescence is lost. The CTEC fragments used in this example have a 50 bp sequence homologous to linearized pRN1120 vector backbone (digested by EcoRI and XhoI) on either side for in-vivo circularization of the pRN1120 plasmid containing the CTEC fragment. On the 3′ side connector F (CONF, SEQ ID NO: 131) is included in between the donor DNA and the 50 bp sequence homologous to the linearized pRN1120 fragment. An overview of the CTEC DNA designs is provided in FIG. 17.

An overview of the sequences is provided in Table 13.

TABLE 13
Overview of the sequences of the CTEC DNA's used in transformation.
The template guide-RNA expression cassettes were used as a template
for PCR using the primers indicated in this table to obtain CTEC
DNA's (CTEC DNA fragments) used in the transformation experiments.

| | | Guide | | | |
|---|---|---|---|---|---|
| | | sequence | | Primers used | Sequence |
| | guide-RNA | (genomic | | to amplify | of the |
| | expression | target | Donor | CTEC DNA | CTEC DNA |
| CTEC design | cassette | sequence) | DNA | fragment | fragment |
| pRN1120 − YFP | SEQ ID | SEQ ID | SEQ ID | SEQ ID | SEQ ID |
| target + 3′ | NO: 121 | NO: 122 | NO: 123 | NO: 129 | NO: 115 |
| donor_FS60bp − | | | | SEQ ID | |
| CONF − pRN1120 | | | | NO: 130 | |
| pRN1120 − YFP | SEQ ID | SEQ ID | SEQ ID | SEQ ID | SEQ ID |
| target + 3′ | NO: 121 | NO: 122 | NO: 124 | NO: 129 | NO: 116 |
| donor_FS80bp − | | | | SEQ ID | |
| CONF − pRN1120 | | | | NO: 130 | |
| pRN1120 − YFP | SEQ ID | SEQ ID | SEQ ID | SEQ ID | SEQ ID |
| target + 3′ | NO: 121 | NO: 122 | NO: 125 | NO: 129 | NO: 117 |
| donor_FS100bp − | | | | SEQ ID | |
| CONF − pRN1120 | | | | NO: 130 | |
| pRN1120 − YFP | SEQ ID | SEQ ID | SEQ ID | SEQ ID | SEQ ID |
| target + 3′ | NO: 121 | NO: 122 | NO: 126 | NO: 129 | NO: 118 |
| donor_KO60bp − | | | | SEQ ID | |
| CONF − pRN1120 | | | | NO: 130 | |
| pRN1120 − CON5 − | SEQ ID | SEQ ID | SEQ ID | SEQ ID | SEQ ID |
| YFP target + 3′ | NO: 121 | NO: 122 | NO: 127 | NO: 129 | NO: 119 |
| donor_KO80bp − | | | | SEQ ID | |
| CONF − pRN1120 | | | | NO: 130 | |
| pRN1120 − CON5 − | SEQ ID | SEQ ID | SEQ ID | SEQ ID | SEQ ID |
| YFP target + 3′ | NO: 121 | NO: 122 | NO: 128 | NO: 129 | NO: 120 |
| donor_KO100bp − | | | | SEQ ID | |
| CONF − pRN1120 | | | | NO: 130 | |

The CTEC fragments (gBlock) were used as a template in PCR reactions using the primers indicated in this table. PCR reactions were set-up to obtain CTEC DNA fragments in higher quantities that are later to be used in the transformation experiments. PrimeSTAR GXL DNA Polymerase (Takara/Cat no. R050A) was used in the PCR reactions according to the manufacturer's instructions. The PCR generated CTEC DNA's were purified using a NucleoSpin Gel and PCR Clean-up kit (Machery-Nagel, distributed by Bioké, Leiden, the Netherlands) according to manufacturer's instructions. Subsequently, DNA concentrations were measured using a Nano Drop (ND-1000 Spectrophotometer, Thermo Scientific, Bleiswijk, the Netherlands).

### Experimental Details:

The components applied in this example were as follows:
Yeast strain CSN009 which is pre-expressing Cas9 and has a fluorescent phenotype due to YFP expression cassette that is present on the INT1 locus. Construction of S. cerevisiae strain CSN009 is described in Example 1.
pRN1120, multi-copy expression vector containing NatMX marker. Construction and details of the plasmid are described in Example 1.

### Yeast Transformation

Strain CSN009 which is pre-expressing Cas9 and fluorescent due to the presence of an YFP expression cassette, was inoculated in YPD-G418 medium (10 grams per liter of yeast extract, 20 grams per liter of peptone, 20 grams per liter of dextrose, 200 μg G418 (Sigma Aldrich, Zwijndrecht, the Netherlands) per ml. Subsequently, strain CSN009 was transformed with 1 μg of CTEC DNA, as indicated in Table 14, and 100 ng vector pRN1 120 circular or 100 ng linearized pRN1120 vector backbone (obtained by EcoRI and XhoI digestion) using the LiAc/SS carrier DNA/PEG method (Gietz and Woods, 2002).

The transformation mixtures were plated on YPD-agar (10 grams per liter of yeast extract, 20 grams per liter of peptone, 20 grams per liter of dextrose, 20 grams per liter of agar) containing 200 μg nourseothricin (NTC, Jena Bioscience, Germany) and 200 μg G418 (Sigma Aldrich, Zwijndrecht, the Netherlands) per ml. The plates were incubated at 30 degrees Celsius until colonies appeared on the plates.

TABLE 14
Overview of the sequences of the CTEC DNA's used in transformation.

| | | Sequence of | | |
|---|---|---|---|---|
| | | CTEC DNA | | |
| Transformation | CTEC fragment | fragment | Plasmid | FIG. |
| #1 | pRN1120 − YFP target + | SEQ ID | pRN1120 | FIG. 17 |
| | 3′ donor_FS60bp − | NO: 115 | circular | pRN1120 − CTEC- |
| | CONF − pRN1120 | | | 1_FS60bp − CONF − |
| | | | | pRN1120 |
| #2 | pRN1120 − YFP target + | SEQ ID | pRN1120 | FIG. 17 |
| | 3′ donor_FS80bp − | NO: 116 | circular | pRN1120 − CTEC- |
| | CONF − pRN1120 | | | 1_FS80bp − CONF − |
| | | | | pRN1120 |
| #3 | pRN1120 − YFP target + | SEQ ID | pRN1120 | FIG. 17 |
| | 3′ donor_FS100bp − | NO: 117 | circular | pRN1120 − CTEC- |
| | CONF − pRN1120 | | | 1_FS100bp − |
| | | | | CONF − pRN1120 |
| #4 | pRN1120 − YFP target + | SEQ ID | pRN1120 | FIG. 17 |
| | 3′ donor_KO60bp − | NO: 118 | circular | pRN1120 − CTEC- |
| | CONF − pRN1120 | | | 1_KO60bp − CONF − |
| | | | | pRN1120 |
| #5 | pRN1120 − YFP target + | SEQ ID | pRN1120 | FIG. 17 |
| | 3′ donor_KO80bp − | NO: 119 | circular | pRN1120 − CTEC- |
| | CONF − pRN1120 | | | 1_KO80bp − CONF − |
| | | | | pRN1120 |
| #6 | pRN1120 − YFP target + | SEQ ID | pRN1120 | FIG. 17 |
| | 3′ donor_KO100bp − | NO: 120 | circular | pRN1120 − CTEC- |
| | CONF − pRN1120 | | | 1_KO100bp − |
| | | | | CONF − pRN1120 |
| #7 | pRN1120 − YFP target + | SEQ ID | pRN1120 | FIG. 17 |
| | 3′ donor_FS60bp − | NO: 115 | linear | pRN1120 − CTEC- |
| | CONF − pRN1120 | | | 1_FS60bp − CONF − |
| | | | | pRN1120 |
| #8 | pRN1120 − YFP target + | SEQ ID | pRN1120 | FIG. 17 |
| | 3′ donor_FS80bp − | NO: 116 | linear | pRN1120 − CTEC- |
| | CONF − pRN1120 | | | 1_FS80bp − CONF − |
| | | | | pRN1120 |
| #9 | pRN1120 − YFP target + | SEQ ID | pRN1120 | FIG. 17 |
| | 3′ donor_FS100bp − | NO: 117 | linear | pRN1120 − CTEC- |
| | CONF − pRN1120 | | | 1_FS100bp − |
| | | | | CONF − pRN1120 |
| #10 | pRN1120 − YFP target + | SEQ ID | pRN1120 | FIG. 17 |
| | 3′ donor_KO60bp − | NO: 118 | linear | pRN1120 − CTEC- |
| | CONF − pRN1120 | | | 1_KO60bp − CONF − |
| | | | | pRN1120 |
| #11 | pRN1120 − YFP target + | SEQ ID | pRN1120 | FIG. 17 |
| | 3′ donor_KO80bp − | NO: 119 | linear | pRN1120 − CTEC- |
| | CONF − pRN1120 | | | 1_KO80bp − CONF − |
| | | | | pRN1120 |
| #12 | pRN1120 − YFP target + | SEQ ID | pRN1120 | FIG. 17 |
| | 3′ donor_KO100bp − | NO: 120 | linear | pRN1120 − CTEC- |
| | CONF − pRN1120 | | | 1_K0100bp − |
| | | | | CONF − pRN1120 |
| #13 | — | — | pRN1120 | — |
| | | | circular | |
| #14 | — | — | pRN1120 | — |
| | | | linear | |

### Results

The colonies resulting from the transformation experiment outlined above in Table 14 were checked for incorporation of the donor DNA after transient expression of the guide RNA that is encoded on the CTEC DNA fragment. Incorporation of the donor DNA that is targeted towards the YFP cassette, results in a frameshift in the YFP ORF or full deletion of the YFP expression cassette, in both cases resulting in loss of fluorescence. The YFP fluorescence of the colonies after transformation was visualized by the QPIX450 (Filter: Ex/Em: 457/536 nm-FITC/GFP). The success rate of YFP editing by the CTEC DNA fragment on phenotype is summarized below in Table 15.

TABLE 15
YFP editing frequency based on phenotype by CTEC DNA fragments in strain S. cerevisiae CSN009. The
counted transformants are from a transformation mix that is undiluted, diluted 10 times or diluted 25
times before plating on the YPD-agar (10 grams per liter of yeast extract, 20 grams per liter of peptone,
20 grams per liter of dextrose, 20 grams per liter of agar) containing 200 μg nourseothricin (NTC,
Jena Bioscience, Germany) and 200 μg G418 (Sigma Aldrich, Zwijndrecht, the Netherlands) per ml.

| | | | | | | Percentage |
|---|---|---|---|---|---|---|
| | | | | | Number of | non- |
| | | | Dilution | Total | non- | fluorescent/ |
| | | | transformation | number of | fluorescent | edited |
| Transformation | Description | Plasmid | mix | transformants | transformants | colonies |
| #1 | pRN1120 − YFP | pRN1120 | undiluted | 42 | 37 | 88% |
| | target + 3′ | circular | 10x | 6 | 6 | 100% |
| | donor_FS60bp − | | diluted | | | |
| | CONF − pRN1120 | | | | | |
| #2 | pRN1120 − YFP | pRN1120 | undiluted | 321 | 271 | 84% |
| | target + 3′ | circular | 10x | 41 | 32 | 78% |
| | donor_FS80bp − | | diluted | | | |
| | CONF − pRN1120 | | | | | |
| #3 | pRN1120 − YFP | pRN1120 | undiluted | 615 | 552 | 90% |
| | target + 3′ | circular | 10x | 54 | 47 | 87% |
| | donor_FS100bp − | | diluted | | | |
| | CONF − pRN1120 | | | | | |
| #4 | pRN1120 − YFP | pRN1120 | undiluted | 54 | 1 | 2% |
| | target + 3′ | circular | 10x | 7 | 0 | 0% |
| | donor_KO60bp − | | diluted | | | |
| | CONF − pRN1120 | | | | | |
| #5 | pRN1120 − YFP | pRN1120 | undiluted | 59 | 1 | 2% |
| | target + 3′ | circular | 10x | 13 | 0 | 0% |
| | donor_KO80bp − | | diluted | | | |
| | CONF − pRN1120 | | | | | |
| #6 | pRN1120 − YFP | pRN1120 | undiluted | 58 | 4 | 7% |
| | target + 3′ | circular | 10x | 9 | 0 | 0% |
| | donor_KO100bp − | | diluted | | | |
| | CONF − pRN1120 | | | | | |
| #7 | pRN1120 − YFP | pRN1120 | 25x | 201 | 201 | 100% |
| | target + 3′ | linear | diluted | >1000 | >1000 | 100% |
| | donor_FS60bp − | | 10x | | | |
| | CONF − pRN1120 | | diluted | | | |
| #8 | pRN1120 − YFP | pRN1120 | 25x | 248 | 248 | 100% |
| | target + 3′ | linear | diluted | >1000 | >1000 | 100% |
| | donor_FS80bp − | | 10x | | | |
| | CONF − pRN1120 | | diluted | | | |
| #9 | pRN1120 − YFP | pRN1120 | 25x | 330 | 330 | 100% |
| | target + 3′ | linear | diluted | >1000 | >1000 | 100% |
| | donor_FS100bp − | | 10x | | | |
| | CONF − pRN1120 | | diluted | | | |
| #10 | pRN1120 − YFP | pRN1120 | undiluted | 32 | 28 | 88% |
| | target + 3′ | linear | 10x | 3 | 3 | 100% |
| | donor_KO60bp − | | diluted | | | |
| | CONF − pRN1120 | | | | | |
| #11 | pRN1120 − YFP | pRN1120 | undiluted | 96 | 92 | 95% |
| | target + 3′ | linear | 10x | 11 | 11 | 100% |
| | donor_KO80bp − | | diluted | | | |
| | CONF − pRN1120 | | | | | |
| #12 | pRN1120 − YFP | pRN1120 | undiluted | 131 | 121 | 92% |
| | target + 3′ | linear | 10x | 23 | 23 | 100% |
| | donor_KO100bp − | | diluted | | | |
| | CONF − pRN1120 | | | | | |
| #13 | — | pRN1120 | undiluted | 843 | 0 | 0% |
| | | circular | 10x | 81 | 0 | 0% |
| | | | diluted | | | |
| #14 | — | pRN1120 | undiluted | 45 | 0 | 0% |
| | | linear | 10x | 6 | 0 | 0% |
| | | | diluted | | | |

Loss of fluorescence of the CSN009 strain due to YFP editing, as a consequence of the CTEC DNA fragment, is demonstrated. The CTEC fragments contain donor DNA of 60, 80 or 100 bp which encode either a frameshift in the YFP gene or flanks for full knockout of the YFP expression cassette are functional for both types of donor DNA. In addition, the lengths tested, ranging from 60 to 100 bp, are all functional. The efficiency at which full knock outs are created is highly increased when the CTEC fragment is assembled within the cell into the pRN1120 vector backbone, resulting in constitutively expressed guide RNA thereby eliminating background strains in which no editing of the targeted YFP gene has taken place. Striking is that the number of transformants is highly increased when the CTEC DNA fragment, of which the donor DNA encodes a frameshift, is assembled in the pRN1120 vector backbone. These large number of transformants obtained all have the edited YFP gene, as is demonstrated by the loss of fluorescence.

### Example 5. Crispr/Cas9 Mediated Genome Editing by CTEC Constructs in Yarrowia lipolytica

This example describes Cas9 mediated editing of the GFP gene in Yarrowia strain ML3244. Strain ML3244 pre-expresses Cas9 and contains an integrated GFP expression cassette as fluorescent marker. By transformation of a CTEC DNA fragment which consists of a guide RNA expression cassette as well as donor DNA, the GFP ORF is edited in the strain after transient expression of the guide RNA sequence. In this example, four different donor DNA's were tested, each encoding a different modification in the GFP gene. To completely delete the GFP gene, the first donor DNA consists out of two flanking regions just outside the GFP ORF. A second donor DNA encodes a DNA base deletion whereby the PAM sequence is modified from CGG to CG, which means a frameshift is introduced upon incorporation of the donor DNA. The third donor DNA encodes a 2 base pair change in the PAM, changing it from CGG to TAG whereby a STOP codon is introduced. The fourth type of donor DNA that is used for editing of the GFP gene encodes a silent mutation in the GFP gene by changing the PAM sequence from CGG to CGA and encodes a stop codon just outside the PAM and genomic target sequence by a base change from T to A. The described four donor DNA fragments result in a modification of the GFP gene that results in loss of fluorescence of the strain. The CTEC DNA fragment is a linear DNA fragment that does not contain a marker for selection of transformants. To select for transformants, plasmid pSTV077, containing the hygromycin B marker was added in the transformation. Colonies that appeared on the selective plates with hygromycin B were analyzed for GFP fluorescence and loss thereof, confirming the editing of the GFP gene as a consequence of the CTEC DNA fragment.

### CRISPR Transient Editing Construct (CTEC) DNA Fragments

Synthetic DNA's containing guide-RNA expression cassettes were ordered as synthetic DNA (gBlocks) at Integrated DNA Technologies (IDT, Leuven, Belgium). Four designs were made for editing the GFP ORF, an overview of the designs is provided in Table 16. The designs of the CTEC DNA's, of which the sequences are set out in SEQ ID NO's: 170, 171, 134 and 135, consist of the guide RNA expression cassette and donor DNA of 100-bp in size. The guide-RNA expression cassette targets the GFP gene in the Yarrowia genome of strain ML3244 and was comprised of the YI_HYPO promoter (SEQ ID NO: 136) followed by a 6 bp inverted repeat of the GFP genomic target (SEQ ID NO: 137), a hammerhead (HH) ribozyme (SEQ ID NO: 138) and Hepatitis delta virus (HDV) ribozyme (SEQ ID NO: 139) on the 5′ and 3′ side of the 20 bp genomic target sequence of GFP (SEQ ID NO: 140) and the YI_PGM terminator (SEQ ID NO: 141), as described by Gao and Zhao. In this example four different types of donor DNA fragments were used, each being 100-bp in size and when incorporated GFP fluorescence of strain ML3244 is lost. The donor DNA of CTEC DNA fragment 1 (SEQ ID NO: 170) consisted of two flanking regions, 50-bp on the 5′ side and 50-bp on the 3′ side, just outside the GFP ORF to completely delete the GFP gene. The donor DNA of CTEC DNA fragment 2 (SEQ ID NO: 171) encoded a DNA base deletion whereby the PAM sequence was modified from CGG to CG, which means a frameshift was introduced upon incorporation of the donor DNA. The donor DNA of CTEC DNA fragment 3 (SEQ ID NO: 134) encodes a two base modification in the PAM, changing it from CGG to TAG whereby a STOP codon was introduced. The donor DNA of CTEC DNA fragment 4 (SEQ ID NO: 135) encodes a silent mutation in the GFP gene by changing the PAM sequence from CGG to CGA and encoded a stop codon by a base change from T to A, just outside the PAM and genomic target sequence. An overview of the sequences is provided in Table 16.

TABLE 16
Overview of the sequences of the CTEC DNA fragments used in transformation
of Yarrowia strain ML3244 targeting the GFP gene.

| | | Guide | | |
|---|---|---|---|---|
| | | sequence | | Sequence |
| | guide-RNA | (genomic | | of the |
| | expression | target | Donor | CTEC DNA |
| CTEC design | cassette | sequence) | DNA | fragment |
| CTEC DNA fragment 1 | SEQ ID | SEQ ID | SEQ ID | SEQ ID |
| GFP target_full KO | NO: 142 | NO: 140 | NO: 143 | NO: 170 |
| CTEC DNA fragment 2 | SEQ ID | SEQ ID | SEQ ID | SEQ ID |
| GFP target_base deletion PAM | NO: 142 | NO: 140 | NO: 144 | NO: 171 |
| CTEC DNA fragment 3 | SEQ ID | SEQ ID | SEQ ID | SEQ ID |
| GFP target_2 base modification PAM | NO: 142 | NO: 140 | NO: 145 | NO: 134 |
| CTEC DNA fragment 4 | SEQ ID | SEQ ID | SEQ ID | SEQ ID |
| GFP target_silent mutation PAM and | NO: 142 | NO: 140 | NO: 146 | NO: 135 |
| base modification | | | | |

### Construction Yarrowia Strain ML3244

The Yarrowia plasmid for expression of Cas9, MB7452 (FIG. 18, SEQ ID NO: 147), was transferred to Yarrowia strain ML324 (MATa; deposited under number ATCC18943). Yarrowia vector MB7452 contains a Cas9 expression cassette (SEQ ID NO: 148) consisting of a codon optimized Cas9 gene expressed from the YI_007 promoter (Yarrowia lipolytica promoter of YALI0B14377g, SEQ ID NO: 149), the YI_GPD terminator (Yarrowia lipolytica terminator of YALI0C06369g, SEQ ID NO: 150), and a functional NatMX marker cassette conferring resistance against nourseothricin.

Vector MB7452 containing the Cas9 expression cassette was transformed to Yarrowia lipolytica strain ML324 (MATa) using the LiAc/salmon sperm (SS) carrier DNA/PEG method (Gietz and Woods, 2002) with a heat shock temperature of 39 degrees Celsius. In the transformation mixture 1 microgram of vector MB7452 was used. The transformation mixture was plated on YPD-agar (10 grams per liter of yeast extract, 20 grams per liter of peptone, 20 grams per liter of dextrose, 20 grams per liter of agar) containing 150 microgram (rig) nourseothricin (NTC, Jena Bioscience, Germany) per ml. After two to four days of cultivation at 30 degrees Celsius, transformants appeared on the transformation plate. A transformant conferring resistance to nourseothricin on the plate, designated strain ML3242 (MATa, Cas9), was inoculated in YPD-nourseothricin medium (10 grams per liter of yeast extract, 20 grams per liter of peptone, 20 grams per liter of dextrose, 150 nourseothricin (NTC, Jena Bioscience, Germany) per ml), and used in a subsequent transformation to knock out the KU70 gene.

The CRISPR/Cas mediated knockout of the KU70 gene in Yarrowia strain ML3242 was performed by transformation of plasmid pSTV089 and a 100-bp KU70 knock out donor DNA fragment to the strain. Yarrowia plasmid pSTV089 (SEQ ID NO: 151, FIG. 19) is equipped with a guide-RNA expression cassette and a functional HygB marker cassette conferring resistance to hygromycin B. The guide-RNA expression cassette targets the KU70 gene in the Yarrowia genome and is comprised of the YI_HYPO promoter (SEQ ID NO: 136) followed by a 6 bp inverted repeat of the KU70 genomic target (SEQ ID NO: 167), a hammerhead (HH) (SEQ ID NO: 138) and Hepatitis delta virus (HDV) ribozyme (SEQ ID NO: 139) on the 5′ and 3′ side of the 20 bp genomic target sequence of the KU70 gene (SEQ ID NO: 152) and the YI_PGM terminator (SEQ ID NO: 141), as described by Gao and Zhao. In addition to the guide-RNA expression cassette and HygB marker cassette, plasmid pSTV089 contains a Cas9 expression cassette. Cas9 was codon optimized for expression in Y. lipolytica and was expressed from the Yarrowia lipolytica 007 promoter (SEQ ID NO: 149) and the Yarrowia lipolytica GPD terminator (SEQ ID NO: 150). The 100-bp KU70 knock out donor DNA fragment (SEQ ID NO: 153) is a double stranded DNA fragment and comprises 50-bp upstream and 50-bp downstream of the KU70 gene. Upon incorporation of the KU70 knock out donor DNA fragment the KU70 gene that is in between the 50-bp sequences was deleted from the genome.

Plasmid pSTV089 and the donor DNA fragment were transformed to Yarrowia lipolytica strain ML3242 (MATa Cas9) using the LiAc/salmon sperm (SS) carrier DNA/PEG method (Gietz and Woods, 2002) with a heat shock temperature of 39 degrees Celsius. In the transformation mixture 500 nanogram of plasmid pSTV089 was used and 500 ng of the 100-bp KU70 knock out donor DNA fragment. The transformation mixture was plated on YPD-agar (10 grams per liter of yeast extract, 20 grams per liter of peptone, 20 grams per liter of dextrose, 20 grams per liter of agar) containing 150 microgram (μg) hygromycin B (Thermo Fisher Scientific, The Netherlands, Cat no: 10687010) per ml and 150 microgram (μg) nourseothricin (NTC, Jena Bioscience, Germany) per ml. After two to four days of cultivation at 30 degrees Celsius, transformants appeared on the transformation plate. Transformants were selected for presence of the Cas9 expression plasmid (MB7452) by nourseothricin resistance and presence of plasmid pSTV089 by hygromycin B resistance.

The knock out of the KU70 gene was confirmed by PCR. As template, genomic DNA isolated using the YeaStar genomic DNA kit (D2002, ZymoResearch, BaseClear, The Netherlands) according to supplier's manual, was used. Primer set (SEQ ID NO: 154 and SEQ ID NO: 155), located on the genome just outside the 50-bp sequences upstream and downstream of the KU70 gene used for the knock out, was used with PrimeStar polymerase according to supplier's manual. The knock out was confirmed by amplification of a 964-bp fragment that confirms deletion of the KU70 gene and integration of the KU70 knock out donor DNA.

Since an ML3242 transformant in which the KU70 knock out was confirmed by PCR was to be used in additional Cas9 experiments, it was cured from plasmid pSTV089 (hygromycin B marker) while maintaining its Cas9 expression plasmid, MB7452 (nourseothricin marker). The strain was cultured for 24 hours in YPD liquid medium (10 grams per liter of yeast extract, 20 grams per liter of peptone, 20 grams per liter of dextrose) supplemented with 150 microgram (μg) nourseothricin (NTC, Jena Bioscience, Germany) per ml at 30 degrees C., shaking speed: 250 rpm. Dilutions of the culture were made in milliQ and subsequently plated onto YPD-agar medium (10 grams per liter of yeast extract, 20 grams per liter of peptone, 20 grams per liter of dextrose, 20 grams per liter of agar) containing 150 microgram (μg) nourseothricin (NTC, Jena Bioscience, Germany) per ml. After two to four days of cultivation at 30 degrees Celsius, colonies appeared on the agar plate. Single colonies were subsequently checked for hygromycin B sensitivity by streaking them on YPD-agar (10 grams per liter of yeast extract, 20 grams per liter of peptone, 20 grams per liter of dextrose, 20 grams per liter of agar) containing 150 microgram (μg) hygromycin B (Thermo Fisher Scientific, The Netherlands, Cat no: 10687010) per ml. A hygromycin B sensitive strain was selected and designated ML3243 (MATa ΔKU70 Cas9). Strain ML3243 was used in a subsequent transformation to add a GFP expression cassette (SEQ ID NO: 156) on the INT05 locus of this strain.

The CRISPR/Cas mediated integration of a GFP expression cassette in the INT05 locus of Yarrowia strain ML3242 was performed by transformation of plasmid pSTV086 and a GFP expression cassette that is flanked by 50-bp genomic DNA sequences of the INT05 locus. Yarrowia plasmid pSTV086 (SEQ ID NO: 157, FIG. 20) is equipped with a guide-RNA expression cassette and a functional HygB marker cassette conferring resistance to hygromycin B. The guide-RNA expression cassette targets the INT05 locus in the Yarrowia genome and is comprised of the YI_HYPO promoter (SEQ ID NO: 136) followed by a 6 bp inverted repeat of the INT05 genomic target (SEQ ID NO: 168), a hammerhead (HH) (SEQ ID NO: 138) and Hepatitis delta virus (HDV) ribozyme (SEQ ID NO: 139) on the 5′ and 3′ side of the 20-bp genomic target sequence of the INT05 locus (SEQ ID NO: 169) and the YI_PGM terminator (SEQ ID NO: 141), as described by Gao and Zhao. In addition to the guide-RNA expression cassette and HygB marker cassette, plasmid pSTV086 contains a Cas9 expression cassette. Cas9 was codon optimized for expression in Y. lipolytica and is expressed from the Yarrowia lipolytica 007 promoter (SEQ ID NO: 149) and the Yarrowia lipolytica GPD terminator (SEQ ID NO: 150). The GFP expression cassette that was integrated on the INT05 locus of Yarrowia strain ML3243 comprises the Yarrowia YI_HSP promoter (SEQ ID NO: 162), the Aequorea victoria eGFP (A. vic_eGFP) ORF (SEQ ID NO: 163) and Yarrowia YI_GPD terminator (SEQ ID NO: 164). The GFP expression cassette is flanked by 50-bp genomic DNA flanks for targeted integration at the INT05 locus of Yarrowia strain ML3243.

Plasmid pSTV086 (SEQ ID NO: 157, FIG. 20) and a GFP expression cassette that is flanked by 50-bp genomic DNA sequences of the INT05 locus (SEQ ID NO: 158) were transformed to Yarrowia lipolytica strain ML3243 (MATa ΔKU70 Cas9) using the LiAc/salmon sperm (SS) carrier DNA/PEG method (Gietz and Woods, 2002) with a heat shock temperature of 39 degrees Celsius. In the transformation mixture 500 nanogram of plasmid pSTV086 was used and 500 ng of the GFP expression cassette flanked by 50-bp genomic DNA sequences of the INT05 locus for targeted integration. The transformation mixture was plated on YPD-agar (10 grams per liter of yeast extract, 20 grams per liter of peptone, 20 grams per liter of dextrose, 20 grams per liter of agar) containing 150 microgram (μg) hygromycin B (Thermo Fisher Scientific, The Netherlands, Cat no: 10687010) per ml and 150 microgram (μg) nourseothricin (NTC, Jena Bioscience, Germany) per ml. After two to four days of cultivation at 30 degrees Celsius, transformants appeared on the transformation plate. Transformants were selected for presence of the Cas9 expression plasmid (MB7452) by nourseothricin resistance and presence of plasmid pSTV086 by hygromycin B resistance.

The integration of the GFP expression cassette was confirmed by fluorescence that was visualized by the QPIX450 (Filter: Ex/Em: 457/536 nm-FITC/GFP). To confirm the integration of the GFP expression cassette in the INT05 locus, a PCR was set up using genomic DNA of a fluorescent transformant as template and PrimeStar polymerase according to supplier's manual. Primer set (SEQ ID NO: 159 and SEQ ID NO: 160), that is located on the INT05 locus in the genome just outside the 50-bp genomic sequences that were used for integration of the GFP expression cassette, was used in the PCR reaction. Genomic DNA was isolated using the YeaStar genomic DNA kit (D2002, ZymoResearch, BaseClear, The Netherlands) according to supplier's manual. Targeted integration of the GFP cassette in the INT05 locus was confirmed by amplification of a 3412-bp fragment.

Since a ML3243 transformant in which the integration of the GFP expression cassette at the INT05 locus was confirmed by PCR and fluorescence of the strain, was to be used in additional Cas9 experiments, it was cured from plasmid pSTV086 (hygromycin B marker) while maintaining its Cas9 expression plasmid, MB7452 (nourseothricin marker). The strain was cultured for 24 hours in YPD liquid medium (10 grams per liter of yeast extract, 20 grams per liter of peptone, 20 grams per liter of dextrose) supplemented with 150 microgram (μg) nourseothricin (NTC, Jena Bioscience, Germany) per ml at 30 degrees C., shaking speed: 250 rpm. Dilutions of the culture were made in milliQ and subsequently plated onto YPD-agar medium (10 grams per liter of yeast extract, 20 grams per liter of peptone, 20 grams per liter of dextrose, 20 grams per liter of agar) containing 150 microgram (μg) nourseothricin (NTC, Jena Bioscience, Germany) per ml. After two to four days of cultivation at 30 degrees C., colonies appeared on the agar plate. Single colonies were subsequently checked for hygromycin B sensitivity by streaking them on YPD-agar (10 grams per liter of yeast extract, 20 grams per liter of peptone, 20 grams per liter of dextrose, 20 grams per liter of agar) containing 150 microgram (μg) hygromycin B (Thermo Fisher Scientific, The Netherlands, Cat no: 10687010) per ml. A hygromycin B sensitive strain was selected and designated ML3244 (MATa ΔKU70 Cas9, GFP). This strain was used in further transformation experiments.

### Integration Site INT05

The INT05 integration site is a non-coding region between gene YALI0F11275g and YALI0F11297g, located on chromosome NC_006072.

pSTV077 Vector (Yarrowia Expression Vector, HygB Marker)

Yarrowia vector pSTV077 (FIG. 21, SEQ ID NO: 161) is equipped with a functional HygB marker cassette conferring resistance to hygromycin B to allow selection of Yarrowia lipolytica transformants on agar plate or in liquid cultures. The beta lactamase marker allows for selection of the plasmid in E. coli.

### GFP Expression Cassette

The GFP expression cassette that is integrated on the INT05 locus of Yarrowia strain ML3244 comprises the Yarrowia YI_HSP promoter, the Aequorea victoria eGFP (A. vic_eGFP) ORF and Yarrowia YI_GPD terminator. The GFP expression cassette is flanked by 50-bp genomic DNA flanks for targeted integration at the INT05 locus of Yarrowia strain ML3243. The sequence of the eGFP expression cassette including the 50-bp genomic DNA flanks is set out in SEQ ID NO: 158, the sequence of the YI_HSP promoter is set out in SEQ ID NO: 162, the sequence of the A. viceGFP ORF is set out in SEQ ID NO: 163 and that of the YI_GPD terminator is set out in SEQ ID NO: 164.

### DNA Concentrations

All DNA concentrations, including the donor DNA fragments and plasmid pSTV086, were determined using a NanoDrop device (ThermoFisher, Life Technologies, Bleiswijk, the Netherlands), providing the concentrations in nanogram per microliter. Based on these measurements, an amount of 250 ng pSTV077 plasmid and 1000 ng CTEC DNA fragment were used in the transformation experiments.

### PCR Reactions

The PrimeSTAR GXL DNA polymerase (TaKaRa, supplied by VWR, Amsterdam Leiden, the Netherlands. Cat no. R050A) was used in the PCR reactions described above. PCR reactions were performed according to manufacturer's instructions.

### PCR Purification

Purification of PCR reactions was performed using NucleoSpin Gel and PCR Clean-up kit (Machery-Nagel, distributed by Bioké, Leiden, the Netherlands) according to manufacturer's instructions.

### Yarrowia Transformation

Strain ML3244 expressing Cas9 and is fluorescent due to the presence of a GFP expression cassette, was inoculated in YPD-G418 medium (10 grams per liter of yeast extract, 20 grams per liter of peptone, 20 grams per liter of dextrose, 150 μg nourseothricin (Sigma Aldrich, Zwijndrecht, the Netherlands) per ml. Subsequently, strain ML3244 was transformed with 1 μg of CTEC DNA fragment, as indicated in Table 17, and 250 ng vector pSTV077 using the LiAc/SS carrier DNA/PEG method (Gietz and Woods, 2002).

The transformation mixtures were plated on YPD-agar (10 grams per liter of yeast extract, 20 grams per liter of peptone, 20 grams per liter of dextrose, 20 grams per liter of agar) containing 150 μg nourseothricin (NTC, Jena Bioscience, Germany) and 150 μg hygromycin B (Thermo Fisher Scientific, the Netherlands) per ml. The plates were incubated at 30 degrees Celsius until colonies appeared on the plates.

TABLE 17
Overview of the sequences of the CTEC DNA fragments
and plasmid used in transformation.

| | | Sequence of | |
|---|---|---|---|
| | | CTEC DNA | |
| Transformation | CTEC fragment | fragment | Plasmid |
| #1 | CTEC DNA fragment 1 | SEQ ID | pSTV077 |
| | GFP target_full KO | NO: 170 | |
| #2 | CTEC DNA fragment 2 | SEQ ID | pSTV077 |
| | GFP target_base deletion PAM | NO: 171 | |
| #3 | CTEC DNA fragment 3 | SEQ ID | pSTV077 |
| | GFP target_2 base modification PAM | NO: 134 | |
| #4 | CTEC DNA fragment 4 | SEQ ID | pSTV077 |
| | GFP target_silent mutation PAM | NO: 135 | |
| | and base modification | | |
| #5 | — | — | pSTV077 |
| #6 | — | — | — |
| no DNA control | | | |

### Results

The colonies resulting from the transformation experiment outlined above in Table 17 were checked for incorporation of the donor DNA after transient expression of the guide RNA that is encoded on the CTEC DNA fragment. Incorporation of the donor DNA that is targeted towards the GFP cassette, results in loss of fluorescence of the strain. The GFP fluorescence of the colonies after transformation was visualized by the QPIX450 (Filter: Ex/Em: 457/536 nm-FITC/GFP). The success rate of GFP editing by the CTEC DNA fragment on phenotype is summarized below in Table 18.

TABLE 18
GFP editing frequency based on phenotype by CTEC DNA fragments in strain Yarrowia strain
ML3244. The counted transformants are from a transformation mix that is undiluted before
plating on the YPD-agar (10 grams per liter of yeast extract, 20 grams per liter of
peptone, 20 grams per liter of dextrose, 20 grams per liter of agar) supplemented with
150 μg hygromycin B (Hygromycin B, ThermoFisher, The Netherlands) per ml.

| | | | | | Percentage |
|---|---|---|---|---|---|
| | | | | | non- |
| | | | | | fluorescent |
| | | | | Number of | colonies on |
| | | | Total | non- | the total |
| | | | number of | fluorescent | number of |
| Transformation | Description | Plasmid | transformants | transformants | colonies |
| #1 | CTEC DNA | pSTV077 | 68 | 30 | 44% |
| | fragment 1 | | | | |
| | GFP target_full | | | | |
| | KO | | | | |
| #2 | CTEC DNA | pSTV077 | 111 | 74 | 67% |
| | fragment 2 | | | | |
| | GFP target_base | | | | |
| | deletion PAM | | | | |
| #3 | CTEC DNA | pSTV077 | 78 | 43 | 55% |
| | fragment 3 | | | | |
| | GFP target_2 base | | | | |
| | modification | | | | |
| | PAM | | | | |
| #4 | CTEC DNA | pSTV077 | 123 | 34 | 28% |
| | fragment 4 | | | | |
| | GFP target_silent | | | | |
| | mutation PAM | | | | |
| | and base | | | | |
| | modification | | | | |
| #5 | — | pSTV077 | 456 | 0 | 0% |
| #6 | — | — | 0 | 0 | 0% |
| No DNA control | | | | | |

Loss of fluorescence of Yarrowia strain ML3244 due to GFP editing, as a consequence of the CTEC DNA fragments, was demonstrated. The full knock out of the GFP ORF as a consequence of CTEC DNA fragment 1 was confirmed by PCR. Genomic DNA of non-fluorescent strains was isolated using the YeaStar genomic DNA kit (D2002, ZymoResearch, BaseClear, The Netherlands) according to supplier's manual. The isolated genomic DNA was used as template in a PCR reaction using PrimeStar GXL polymerase according to supplier's manual and primer set (SEQ ID NO: 159 and SEQ ID NO: 160). From the genomic DNA of the non-fluorescent strains a 2670-bp fragment was amplified by PCR instead of the 3412-bp fragment that was present in the fluorescent ML3244 strain.

Editing of the GFP gene by CTEC DNA fragment 2, CTEC DNA fragment 3 and CTEC DNA fragment 4 was confirmed by sequencing. Genomic DNA of non-fluorescent strains was isolated using the YeaStar genomic DNA kit (D2002, ZymoResearch, BaseClear, The Netherlands) according to supplier's manual. The genomic DNA was subsequently used as template in a PCR reaction using PrimeStar GXL polymerase according to supplier's manual and primer set (SEQ ID NO: 165 and SEQ ID NO: 166). The resulting PCR fragment represents the edited GFP ORF and was purified using a NucleoSpin Gel and PCR Clean-up kit (Machery-Nagel, distributed by Bioké, Leiden, The Netherlands) according to supplier's instructions. Subsequently the PCR fragment was used as template in a sequencing reaction. Sequencing reactions were set-up making use of a BigDye® Terminator v3.1 Cycle Sequencing Kit (Catno. 4337456, ThermoFisher Scientific, Bleiswijk, the Netherlands) according to supplier's instructions and primer SEQ ID NO: 165. The sequencing reactions were purified by NucleoSEQ columns (Catno. 740523.250, Machery-Nagel, distributed by Bioké, Leiden, the Netherlands) according to supplier's instructions and subsequently analyzed by the 3500XL Genetic Analyzer (ThermoFisher Scientific-Bleiswijk, the Netherlands). Sequencing reads were analyzed in Clone Manager software v9.4 (Sci-Ed software-USA) and confirmed that the loss of fluorescence was caused by the editing of the GFP ORF as was encoded by the donor DNA part of the CTEC DNA fragment that was used in transformation.

By change of the phenotype of Yarrowia ML3244 transformants; being the loss of GFP fluorescence, and by sequencing of the edited GFP ORF or by PCR confirming the full deletion of the GFP ORF, the functionality of the CTEC DNA fragments for genome editing was demonstrated.

### REFERENCES

Altschul S F et al., J. Mol. Biol. 215:403-410 (1990)
Carillo H and Lipman D. SIAM J. Applied Math., 48:1073 (1988)
Carrel F. L. Y. and Canevascini G. Canadian Journal of Microbiology (1991) 37(6): 459-464; Reese E. T., Parrish F. W. and Ettlinger M. Carbohydrate Research (1971) 381-388.
Chaveroche, M K., Ghico, J-M. and d′Enfert C. A rapid method for efficient gene replacement in the filamentous fungus Aspergillus nidulans (2000); Nucleic acids Research, vol 28, no 22.
Cong L, Ran F A, Cox D, Lin S, Barretto R, Habib N, Hsu P D, Wu X, Jiang W, Marraffini L A, Zhang F. Science. Multiplex genome engineering using CRISPR/Cas systems. 2013 Feb. 15; 339(6121):819-23. doi: 10.1126/science.1231143. Epub 2013 Jan. 3.
Crook N C, Schmitz A C, Alper H S. Optimization of a yeast RNA interference system for controlling gene expression and enabling rapid metabolic engineering. ACS Synth Biol. 2014 May 16; 3(5):307-13.
Devereux, J., et al., Nucleic Acids Research 12 (1): 387 (1984).
Derkx, P M and Madrid S M. The foldase CYPB is a component of the secretory pathway of Aspergillus niger and contains the endoplasmic reticulum retention signal HEEL. Mol. Genet. Genomics. 2001 December; 266 (μg):537-545
DiCarlo J E, Norville J E, Mali P, Rios X, Aach J, Church G M. Nucleic Acids Res. 2013 April; 41(7):4336-43. Genome engineering in Saccharomyces cerevisiae using CRISPR-Cas systems.
DiCarlo J E, Chavez A, Dietz S L, Esvelt K M, Church G M. Safeguarding CRISPR-Cas9 gene drives in yeast. Nat Biotechnol. 2015 December; 33(12):1250-1255. doi: 10.1038/nbt.3412.
Egholm M, Buchardt O, Christensen L, Behrens C, Freier S M, Driver D A, Berg R H, Kim S K, Norden B, Nielsen P E., 1993. Nature 365, 566-568.
Flagfeldt D B, Siewers V, Huang L, Nielsen J. Characterization of chromosomal integration sites for heterologous gene expression in Saccharomyces cerevisiae. Yeast. 2009 October; 26(10):545-51. doi: 10.1002/yea.1705.
Gao F, Shen X Z, Jiang F, Wu Y, Han C. DNA-guided genome editing using the Natronobacterium gregoryi Argonaute. Nat Biotechnol. 2016 July; 34(7):768-73. doi: 10.1038/nbt.3547.
Gietz R D, Woods R A. Transformation of yeast by lithium acetate/single-stranded carrier DNA/polyethylene glycol method. Methods Enzymol. 2002; 350:87-96.
Govindaraju and Kumar, 2005. Chem. Commun, 495-497.
Gribskov M and Devereux J, eds., Sequence Analysis Primer, M Stockton Press, New York, 1991.
Griffin H M and Griffin H G, eds., Computer Analysis of Sequence Data, Part I, Humana Press, New Jersey, 1994.
Griffin H M and Griffin H G, eds., Molecular Biology: Current Innovations and Future Trends. ISBN 1-898486-01-8; 1995 Horizon Scientific Press, PO Box 1, Wymondham, Norfolk, U.K
Gupta et al. (1968), Proc. Natl. Acad. Sci USA, 60: 1338-1344.
Hawksworth D L et al., In, Ainsworth and Bisby's Dictionary of The Fungi, 8th edition, 1995, CAB International, University Press, Cambridge, UK
Herbert R B. The Biosynthesis of Secondary Metabolites, Chapman and Hall, New York, 1981.
Ho S N, Hunt H D, Horton R M, Pullen J K, Pease L R “Site-directed mutagenesis by overlap extension using the polymerase chain reaction. Gene. 1989 Apr. 15; 77(1):51-9.
Jørgensen T R, Park J, Arentshorst M, van Welzen A M, Lamers G, Vankuyk P A, Damveld R A, van den Hondel C A, Nielsen K F, Frisvad J C, Ram A F. Fungal Genet Biol. 2011 May; 48(5):544-53. The molecular and genetic basis of conidial pigmentation in Aspergillus niger.
Kamath R S et al, (2003) Systematic functional analysis of the Caenorhabditis elegans genome using RNAi. Nature. Vol. 421, 231-237.
Lesk A. M. ed. Computational Molecular Biology, Oxford University Press, New York, 1988.
Lōoke M, Kristjuhan K, Kristjuhan A. Biotechniques. 2011 May; 50(5):325-8. Extraction of genomic DNA from yeasts for PCR-based applications.
Mali P, Yang L, Esvelt K M, Aach J, Guell M, DiCarlo J E, Norville J E, Church G M. RNA-guided human genome engineering via Cas9. Science. 2013 Feb. 15; 339(6121):823-6. doi: 10.1126/science.1232033. Epub 2013 Jan. 3.
Maruyana et al. Nat Biotechnol. 2015 May; 33(5): 538-542.
Song et al. Nature communications doi: 10.1038/ncomms10548
Yu et al. Cell Stem Cell. 2015 Feb. 5; 16(2): 142-147.
Mattern, I. E., van Noort J. M., van den Berg, P., Archer, D. B., Roberts, I. N. and van den Hondel, C. A., Isolation and characterization of mutants of Aspergillus niger deficient in extracellular proteases. Mol Gen Genet. 1992 August; 234(2):332-6.
Morita et al. 2001. Nucleic Acid Res Supplement No. 1: 241-242.
Mouyna I, Henry C, Doering T L, Latgé J P. Gene silencing with RNA interference in the human pathogenic fungus Aspergillus fumigatus. FEMS Microbiol Lett. 2004 Aug. 15; 237(2):317-24.
Nakamura Y, Gojobori T, Ikemura T. Codon usage tabulated from international DNA sequence databases: status for the year 2000. Nucleic Acids Res. 2000 Jan. 1; 28(1):292.
Needleman and Wunsch, J. Mol. Biol. 48:443-453 (1970).
Ngiam C, Jeenes D J, Punt P J, Van Den Hondel C A, Archer D B. Appl. Environ. Microbiol. 2000 February; 66(2):775-82. Characterization of a foldase, protein disulfide isomerase A, in the protein secretory pathway of Aspergillus niger.
Nielsen et al., 1991. Science 254, 1497-1500.
Pel et al. Genome sequencing and analysis of the versatile cell factory Aspergillus niger CBS 513.88. Nat Biotechnol. 2007 February; 25 (2):221-231.
Ramon de Lucas, J., Martinez O, Perez P., Isabel Lopez, M., Valenciano, S. and Laborda, F. The Aspergillus nidulans carnitine carrier encoded by the acuH gene is exclusively located in the mitochondria. FEMS Microbiol Lett. 2001 Jul. 24; 201(2):193-8.
Scarpulla et al. (1982), Anal. Biochem. 121: 356-365.
Sikorski R S, Hieter P. Genetics. A system of shuttle vectors and yeast host strains designed for efficient manipulation of DNA in Saccharomyces cerevisiae. 1989 May; 122(1):19-27.
Smith D W, ed., Biocomputing: Informatics and Genome Projects, Smith, Academic Press, New York, 1993.
Stemmer et al. (1995), Gene 164: 49-53.
Tour O. et al, (2003) Nat. Biotech: Genetically targeted chromophore-assisted light inactivation. Vol. 21. no. 12:1505-1508.
van Dijck et al, 2003, Regulatory Toxicology and Pharmacology 28; 27-35: On the safety of a new generation of DSM Aspergillus niger enzyme production strains.
van Dijken J P, Bauer J, Brambilla L, Duboc P, Francois J M, Gancedo C, Giuseppin M L, Heijnen J J, Hoare M, Lange H C, Madden E A, Niederberger P, Nielsen J, Parrou J L, Petit T, Porro D, Reuss M, van Riel N, Rizzi M, Steensma H Y, Verrips C T, Vindelov J, Pronk J T. An interlaboratory comparison of physiological and genetic properties of four Saccharomyces cerevisiae strains. Enzyme Microb Technol. 2000 Jun. 1; 26(9-10):706-714.
Vartak S V and Raghavan S C. Inhibition of nonhomologous end joining to increase the specificity of CRISPR/Cas9 genome editing. FEBS J. 2015 November; 282(22):4289-94. doi: 10.1111/febs.13416. Epub 2015 Sep. 9.
von Heine G. Sequence Analysis in Molecular Biology, Academic Press, 1987.
Young and Dong, (2004), Nucleic Acids Research 32(7).
Zrenner R, Willmitzer L, Sonnewald U. Analysis of the expression of potato uridinediphosphate-glucose pyrophosphorylase and its inhibition by antisense RNA. Planta. (1993); 190(2):247-52.
Zetsche et al., Cpf1 is a single RNA-guided endonuclease of a class 2 CRISPR-Cas system. Cell. 2015 Oct. 22; 163(3):759-71.

1. A CRISPR transient expression construct (CTEC) adapted for ex vivo use and for expression in a host cell of a functional guide-RNA or part thereof that is specific for a target sequence in a target genome, wherein the CRISPR transient expression construct is linear and comprises:
a guide-RNA expression cassette, and
an additional polynucleotide element, and,
wherein the guide-RNA expression cassette is capable of expressing a functional guide-RNA, or a part thereof, that is specific for a target sequence in a target genome, and wherein the additional polynucleotide element has sequence identity with the target sequence in the target genome.

2. The CRISPR transient expression construct (CTEC) according to claim 1, wherein the functional guide-RNA, or part thereof that is specific for a target sequence in a target genome, is exclusively expressed from the CTEC.

3. The CRISPR transient expression construct (CTEC) according to claim 1, wherein the CTEC is comprised of two or more polynucleotides capable of recombining with each other to yield:
a guide-RNA expression cassette, and
an additional polynucleotide element,
wherein the guide-RNA expression cassette is capable of expressing a functional guide-RNA, or a part thereof, that is specific for a target sequence in a target genome, wherein the additional polynucleotide element has sequence identity with the target sequence in the target genome.

4. The CRISPR transient expression construct (CTEC) according to claim 1, wherein in the CTEC, the guide-RNA expression cassette and the additional polynucleotide element are linked by a polynucleotide that comprises a target sequence that corresponds to the guide sequence of the guide-RNA, allowing in vivo cleavage of the guide-RNA expression cassette from the additional polynucleotide element.

5. The CRISPR transient expression construct (CTEC) according to claim 1, wherein the guide-RNA expression cassette is capable of expressing a functional guide-RNA.

6. The CRISPR transient expression construct (CTEC) according to claim 1, wherein the guide-RNA expression cassette comprises a eukaryotic promoter.

7. The CRISPR transient expression construct (CTEC) according to claim 1, wherein the functional guide-RNA, or the part thereof, is encoded by a polynucleotide on the guide-RNA expression cassette and the polynucleotide is operably linked to an RNA polymerase II promoter, to an RNA polymerase III promoter as well as a self-processing ribozyme or to a single-subunit DNA-dependent RNA polymerase promoter, optionally a viral single-subunit DNA-dependent RNA polymerase promoter, optionally a T3, SP6, K11 or T7 RNA polymerase promoter.

8. The CRISPR transient expression construct (CTEC) according to claim 1, wherein the guide-RNA expression cassette is located 3′-of the additional polynucleotide element.

9. The CRISPR transient expression construct (CTEC) according to claim 1, wherein the guide-RNA expression cassette is located 5′-of the additional polynucleotide element.

10. The CRISPR transient expression construct (CTEC) according to claim 1, wherein the CTEC comprises two or more polynucleotide sequences capable of recombining with a vector, optionally a plasmid, to in vivo yield the CTEC integrated into the vector.

11. A composition comprising the CRISPR transient expression construct (CTEC) as defined in claim 1, or a library thereof CRISPR, for expression in a host cell of a functional guide-RNA or part thereof that is specific for one or more target sequence(s) in a target genome.

12. The CRISPR transient expression construct (CTEC) according to claim 1 or a composition thereof composition, further comprising a functional polynucleotide-guided genome editing enzyme or an expression construct capable of expressing a functional polynucleotide-guided genome editing enzyme and wherein the functional polynucleotide-guided genome editing enzyme optionally is a Cas9 or a Cpf1.

13. The construct according to claim 1, wherein the host cell is deficient in Non-Homologous End Joining (NHEJ).

14. A host cell comprising the CRISPR transient expression construct (CTEC) as defined in claim 1 or comprising a composition comprising said construct.

15. The host cell according to claim 14, further comprising:
a functional polynucleotide-guided genome editing enzyme, optionally a functional polynucleotide-guided heterologous genome editing enzyme, or
further comprising an expression construct capable of expressing a functional polynucleotide-guided genome editing enzyme, optionally a functional polynucleotide-guided heterologous genome editing enzyme,
wherein the functional polynucleotide-guided genome editing enzyme optionally is a Cas9 or a Cpf1.

16. The host cell according to claim 15, wherein the sequence of the polynucleotide element has been introduced into the genome at the site where the additional polynucleotide element has sequence identity with the sequences flanking the target sequence in the target genome.

17. The host cell according to claim 14, wherein the host cell is deficient in Non-Homologous End Joining (NHEJ).

18. The host cell according to claim 14 comprising a polynucleotide encoding a compound of interest.

19. A method for production of a compound of interest, comprising culturing the host cell according to claim 18 under conditions conducive to the production of the compound of interest, and, optionally, purifying or isolating the compound of interest.
